SEQUENCE LISTING (208 sequences)

SEQ ID NO: 1 - protein, 612 aa, unidentified
  source 1..612: note = MG64-1 active effector
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 2 - protein, 563 aa, unidentified
  source 1..563: note = MG64-1-B active transposition protein
MKNANSPPSSSVDDHQKEQHLVIPSELSDEAQLKLEVIQTLLEPCDRRTYGQRLREAAEKLGKSKRTVQRLVKKWEEEGLEAIAPTNRSDKGDFRIEEQLQEFIIKTYQNGNKGSLRVTRKQVYLKTKAKAEELSINPPSHMTVYRILQPLIEKQEKKKSIRSPGWRGSQLSVKTRAGQDLSVEYSNHVWQCDHTRADLLLVDQYGELLGRPWLTTVIDTYSRCIIGINLGFDAPSSQVVALALRHAILPKYYTPDYLLGEEWGTYGKPEHFYTDGGKDFRSNHLQQISVQLGFVCHLRDRPSEGGIVERPFKTLNLEFFSTLPGYTGSNVQERPEDAEKEACLTLRQLEQKLVRYIVDNYNQRMDARMGDQTRFQRWESGLIATPDVISERELDICLMKQTRRKVQRGGYLQFENLMYRGENLAGYAGESVILRFDPRDITTVLVYQKESNHEVFLTRAYAMDLETEQMSLDEAKASSKRVRDAGKTVGNRSILSEVRDRQIFPKAKKSKKERYQEEQKAITSKPLEITEWESEETDFSPPSSETPQVEVFDYETLQEDYGF

SEQ ID NO: 3 - protein, 278 aa, unidentified
  source 1..278: note = MG64-1-C active transposition protein
MTIQEAQAVAKQLGDIQLTSEKLQAEIQRLNRKTVVTLSHVEALHNWLEGKRQAKQSCRVVGESRTGKTIACNAYRLRHKPIQTPGKPPIVPVVYIQVTQECGAKDLFGAIIEHLKYQMTKGTVAEIRQRTFKVLQRCGVEMLIIDEADRLKPKTFAEVRDIFDKLNIAVVLVGTDRLDAVIKRDEQVYNRFRACHRFGKLAGDEFSQTVNIWERQVLKLPVASNLSSKRMLKILGQATGGYLGLLDMILRESAIRALKKGLQKIDLDTLKEVTEEYR

SEQ ID NO: 4 - protein, 171 aa, unidentified
  source 1..171: note = MG64-1-Q active transposition protein
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 5 - RNA, 319 nt, synthetic construct
  source 1..319: mol_type = other RNA; note = MG64-1 active effector tested sgRNA sequence
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaagnnnnnnnnnnnnnnnnnnnnnnnn

SEQ ID NO: 6 - RNA, 277 nt, synthetic construct
  source 1..277: mol_type = other RNA; note = MG64-1 active effector tracrRNA sequence
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccact

SEQ ID NO: 7 - DNA, 38 bp, synthetic construct
  source 1..38: mol_type = other DNA; note = MG64-1 active effector's target CRISPR repeat
cgtcacaatctattttggttaatgagatggattgaaag

SEQ ID NO: 8 - DNA, 449 bp, synthetic construct
  source 1..449: mol_type = other DNA; note = MG64-1 active transposon end RE
aatgaacaaaaatgtctgattattacataattgtttatttaatataattgtatcgtaatacttgaagtttggagacaagtaatttgttaatactgctccagtccctaaaaaagtgccattcgggtaaatgacacttaatctgttaatttactggaaaatgacagttaatttgttaatatagtaagcaataacttttgtcaaagattaatgctataattcagctaaagcagtgattatataaagctttcactctcaaatagttcggcgacacgattttgttaagacgacaaataattagttactgtacatttacccataactttgccgttttggtaaggttatcgttcaaaacacaagtggcaagattatggttcaaaacctaagtcccgtttagtttgcttgaacacttcacgaacttgagggtaacgaagaaagctagggtgagtcaa

SEQ ID NO: 9 - DNA, 319 bp, synthetic construct
  source 1..319: mol_type = other DNA; note = MG64-1 active transposon end LE
ctcttaaggaaatctatgtaagtttgttgggttagttgcgttttcagtaaatactgtgttatagtaagaacttgtgcggacgtatagctcagttggttagagtacatcgttgacatcgatggggtcactggttcgagtccagttacgtccatatttttttgaagtgtgtataatattaactatgtgactttatgtacattaacagattatttgtcatcggtaacaaattgttgtcatcttaacaaaatatttgtcatcaataacatattatgtgtcgtgtgcttattactgaaactaatcctagacgatggtaaaaaat

SEQ ID NO: 10 - DNA, 308 bp, synthetic construct
  source 1..308: mol_type = other DNA; note = MG64-1 active transposon outer bounds of RE
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 11 - DNA, 127 bp, synthetic construct
  source 1..127: mol_type = other DNA; note = MG64-1 active transposon minimal LE
tgtacattaacagattatttgtcatcggtaacaaattgttgtcatcttaacaaaatatttgtcatcaataacatattatgtgtcgtgtgcttattactgaaactaatcctagacgatggtaaaaaat

SEQ ID NO: 12 - protein, 643 aa, unidentified
  source 1..643: note = MG64-3 effector
MSKITIQCRLVASEATRQYLWHLMADIYTPFVNEMLRQIREDDNFEQWRQAGKIPAGVFEDYRKALKTESRFQGMPGRWYYAGREEVKRIYKSWLALRRRLRNQLSGQNRWLEVLQSDETLMAVSGLDLPALRAASTKLLDLLSSQIAKPAKGSKKTNRGKGKKQAKQTQGKSLYQSLWDLYKETEDILQKCAIAYLLKNKSQVPDKPEDPEKFRHRRRKAEIRTERLNEQLTKTRLPKGRDLTNEQWLEALAIATEQIPKDETEAAIWQSRLLTDAASLPFPVAYETNEDLKWFLNGKGRLCVSFNGLSEHTFEIYCDKRQLHWFKRFLEDQQIKKEHQGKRSSGLFTLRSGRISWTSPSDIDKSPCWTANRLTLHCSVDTRLWTQEGTEEVRQEKATNIAKIIAGTKAKGNLNQKQQDFITKRETTLKLLHNPFPRPSKPLYQGNPSIIAAVSFGLEKPATLAIVDITTGKAITYRSIRQLLDQNYKLFTKHRLQQQQRAHQRHQNQKESAENRISEGGLGEHVDSLIAKAILETAAEYGASSIVLPELGNIREIIQAEVIAKAERKIPGLKEKQDEYAAKFRASVHRWSYGRLAQKITTKAALQGLETESTRQPLQGSPQEKARNLAIAAYESRKVDQRA

SEQ ID NO: 13 - protein, 542 aa, unidentified
  source 1..542: note = MG64-3-B transposition protein
MELVNPDDLNSVEARLKLEIVEKLSEPCDRRTYGERLRSAAQKLECSVRTVQRLMKKWEQEGIHALIDSGRKDKGNPRISEDWQSFIKDAYDNGKCTPAQVFTKVRQRARQEGLDSHPSHMTVYRILNPLIEAKEQKNNIRNVGWKGARLALKTRDGEVLEIDYSNQVWQCDHTRADILLVDKYGYQMGRPWLTTVVDTYSRAIVGINLGYDAPSSRVVALALRHAILPKQYGAEYKLYAEWPTCGVPDHMFTDGGKDFRSNHLQQIGVQLGFICHLRDRPSEGGIVERPFGTINTQFFSTLPGYTGSNVQDRPPEAEAEACLTLHELEKLLVAYIVNTYNQRLDARMGDQTRIQRWEAGLLKQPPTISERELDICLMKQTRRTIYRGGYLQFENLTYWGETLAEQAGENIVLRYDPRDITRLLVYRYESDREVYLGVAQAQDLEGEVLALDDAKAHSRRIREDGKAVSNDAMLDEMRDREAFVDEKKKSRKERQKEEQEDLRQTPLPVIEADSFDEDESGEPQDNLEIPEFEIWEFDNNDI

SEQ ID NO: 14 - protein, 272 aa, unidentified
  source 1..272: note = MG64-3-C transposition protein
MVTTTLSEQEIQAHIERLRKDKTVALESVQQAHTWLDRKRNARQCGRIIGESRTGKTKACESYLKKNGLPDLSGKIPNIPISYFVPKQDCTSRELFRAILEHYGDELPRGTVGDARSKTFKVLRECQTEMLIIDEADRLKPKTFADVRDIFDNLEISVVLVGTKKRLDKVVKADEQVFNRFRSSYKIGTIPSSQLATIVGVWERDILKLPLPSNLTSESMLKEIRRATGKSRKGYYIGLIDMVLRAAAVMALEKGQMKVDKATLKVAVEDYL

SEQ ID NO: 15 - protein, 165 aa, unidentified
  source 1..165: note = MG64-3-Q transposition protein
MTMPIVPTWVFPVDPYSGESLSHFLGRFCRENHATLNQLGEKTGLGAVLGRWEKFRFIPPPNDAQLAALAKLVRLEVDQIKQMLPQEAMQNRVIRLCAACYAEEPYHRIEWQYKLANRCDRHHLLLLLECPNCKAKLPMPSKWANGTCKRCLTPFDQMVALQKGV

SEQ ID NO: 16 - protein, 692 aa, unidentified
  source 1..692: note = MG64-5 effector
MKTIRCCLCANPETRRYFWKIMVTYTLLVNELLAAMPQRPEFAQWKQRGTIAREAVRIVLTPLKTDPTYADLPKRFFSSAELLVCYVYKSWLALQKRRYWKLVGKQRWLQVIEDDLQSLLTDNFSLESVQSKAHQILEQAHKELEKQPQRFKKKGKKSRPLFGYLLDLYGTTADKLERRAIGHLLHHDLKVSDTEDFPETIQFSIDQQQVEIARLKEQLQSRLPDGRDPTQARFLEKLRIATALPELELEGFDEEHFSEWRTQKQIPLLNPLPYPVLFGSSSDLHWKLEPQKATTEANISPEVPTARSERVKERIQVRFKGDELQDSWFKLQCDRRQLPIFRQFVTDYLCQKQAPDHEKFGEGLFTLRSACLVWKEDPQGARKRKKRRKQGACQDEPWETHRLYLHCTIDTRFLTQEGTEQVRATKLDLAQKALEGIENKTALETVTQEPSAEQQKHLKRKQTTVHRLETQKPPVRPTIQPYEGKSNIVVGVSLSRHEPVTLIVFDTAQNKVLECMGTQALLKIHGIQSPRKNRSIGKLQQEQSQLLRRWRRKRKQNPHRRADGQRQDNYRSGNSESKLGDYLDRLIAARLVALATKRQASVIVLPELGDIRESVECSLQAKAQRKYPQHKKLQAKYAKHFRHEFHRWSYGRLQQYIAERATQQNLALLKGRQPKQGTEQEKVLEIISSACL

SEQ ID NO: 17 - protein, 636 aa, unidentified
  source 1..636: note = MG64-5-B transposition protein
MTNSRLGGTMKNLSDHPDSEKLTYEQEHCLVDELSPELQRKVELIQAIVDAPDRKTERQRIAIAAQELGRCTKTIRSYRDALREDGIVALTRTERSDKGQRRNISQPWIDLVLALYKRGQRSFCRSRNQVWLLIQGMTSKLLSDDWKTPEKRAELMEWYAQKLGAAAENAKSKLNKILGSIRKELEVGICMPPRSHMSVYGIIDDYLEQQHRKARHPGQGPEQVIQTTGELLVIEVTNGIFQADHSGIDILLKDKDGNEIGYPFLTVIIECASGCVTGFYLGFRQPGSHEVALALRHAILPKQYGPEYKLEKQWQCVGIPRYLVTDRAKEFKSKHLQQIAAELGFELRYRAYPSQGGLVESVFDKINKEVLSNLPGYKGSNVQKRPKNAEKYACLTIEDLEQELVRYFCDHYNQHFYPRMKDRTRAMQWEERLVEPPVIPDERELDLCLLKRKQTAKVQKYGTIQFQNEIYQGNCLLGRETEKISFRYNPSNIIHVLAYTVEETDKPSKFLGVLKARDRKEEKLSLHSLKLEQKLIRARGKKLDQSSIYNDALKRNERAERELHGLRKQQRRKEHERTGRSEGLGNVIDFKRQENEAIGTKNSQIGPIQKLVKRLKPKRKAKVAAKNWQQKLSENW

SEQ ID NO: 18 - protein, 291 aa, unidentified
  source 1..291: note = MG64-5-C transposition protein
MAQPQLISQQLQTQPSPFPLPDKEAEIDRLRAGAPFLTTDRDTALEQWLDTQRKSGNPGFICSAKGSGLSDSCQDYRMNRVRSKGMIQQLPVPVVYVRVPPICSVSHFHTTLLTALNHPITTGRLKDKRPRVRGRLKSIQTRQLIIDDADFLSFEALSEIAQIYDDLKIPSILCGTYYLEKRLQQRYWDRIGNSFLDFYEYPPMSQDEVVEVIDTWETEFLQWPEESDLLIEDVLKAVYVKTGGLRDALNEVLRKVAIQALKQDSYKITTEIIVSVLNGRVQPRIKPAQEE

SEQ ID NO: 19 - protein, 169 aa, unidentified
  source 1..169: note = MG64-5-Q transposition protein
MTDQQTVWMEHVEPYEGESISHYFGRFRRVEGNSFSAPTTLSAAVGIGPALSRWEKFRFNPFPSPQELEAMGKLIGLTVEQLRTMLPAKGERLVMRSTRLCGACYREAPYHRIHWQYESTEGCDKHRLRLISRCPVCDEKFALPVEWIEGACKQCGMKFTSMHKKQKPY

SEQ ID NO: 20 - protein, 640 aa, unidentified
  source 1..640: note = MG64-33 effector
MKESLYFVIRCLLSADAETRRTMWLLMQRYTLLVNKLLEVIPGSSEFPVWREQGYIPDNALAEFIDTIKPDLDYSGLPGRFYTSAKILVKNIYKSWFALQRKYSRKITGKIRWIKIINSEIDLIKNTEFELDQITNAANAALKLAKKKKEESEKSNSESSTSLLGILIEMQFKTKSPLKKRGINHLLLNNLNIEYKDFTLDSLEARVEVAFLEIEALEKRLRSRLPKGRDPDGYRYVLALSKAASLPEEALTPEKFDEIHADIPIYNELPYPLIYEGASNIVWTLIKPEGNRSNFGRLQIHFNGISELKFLIQCGRRQLPVFKGFYHDAIENKGRISRGEIPYNEGLNRFRSAQILWKPDPSLDFRKKKKNIPSTPWEVNRLYLHCSVDKATLSAEGTECLRQMKIKKIEEKKEKPLSPRKQTELERLQSAAPPPRPSIQPYVGDPDMVVCICFSPDEPVIVVPVDLAKEAALYALNTKALLNRATKAIWRMGKLETLSDNGKALCHDNGGKLNIRNPRTYSVQKPYGLVTRLNTLSEQQVKRRTREQSKGKYRGSQSLSNLSLSVCRLIAARLVDLSLQLNAGRVIIPDFEGIRDWVQAFIAAKAVKAFPDSKQQQKKFRQEFRAKYHRWSYRKLAQEI

SEQ ID NO: 21 - protein, 612 aa, unidentified; identical to SEQ ID NO: 1
  source 1..612: note = MG64-34 effector

SEQ ID NO: 22 - protein, 525 aa, unidentified
  source 1..525: note = MG64-35 effector
REVLSQLSTQSTIEGPPDTQTKRPKAKSRKSKKKQSTAQNKDLIGKLYKAYEATNDLTQRCILAYLIKDAGTISEEEETPEAFTHRIHRKQKDIARLEDRLQARLPKGRDLTGDIFTDTLFIAQHQEPEDVNQMRDWQAKLLMRPADLPDPIRYDSSTDMMWKPDDQGRITVNFNGLEKFLKNSDLEVKSWLKEHQAYPFRIQCDQRQLPYFQRFLADWQAYTADAENYPAGLLTLSSAMLAWRKGKKNRKGEPWNIHQLVLYCSFDTRLLTAEGTAAVQQQKIEKAQKQAESAQNKKLNDNQRQARNRSATTLRKLDNLPTRPSQKAYQAKPELLLGLSIGLSEPITVAVVDASTQQVLTYRTSHTLLGEQHRLLRRQRQKQQQNRLKRQQNQKKGIRHQPSESELGQYVDRLLAKAITQLAQSHQVSSIVLPNLLNRRDLLDSEIQARAEQQCPGSISAQEKYAKAFRQSLHSWDYRRLIEAIRGSAGKHDIPLEEAFLTASSDPKEQAKEIAIAAYQARTED

SEQ ID NO: 23 - protein, 724 aa, unidentified
  source 1..724: note = MG64-36 effector
METREMSQPNLPACMKTIMCCLCASPETRRYFWETMVSYTLLVNELLEAVPNRPEFPQWQRRGTIDREAVRIVLKPLKAKPNYAQLPKRFFTSAELIVCYVYKSWLALQKRRQWQLEGKHRWLAAIASDLKSILSSDLSFETVQAKARQILEQAEQDLEPPPPEVTKKGKKSKRRKKSKSLLKYLLDRHDETTQELERRAICHLLRHDLKVIEEEDTPETIQHVIDRKRIEIERLTEQLQSRLPKGRDPNHERFMERLEMAIALPDGSPKHWDPEEFDEWRIQKQIPELNTLPYPILFGSASDLYWDILNDTTSAATVSAKKKSRKSKRPNERLQVRFKGLDEHKCKIQCDRRQLKTFRQFATDYISNQQLPKDEKFGEGLFALRSACLIWKVDPDASASRRNRQKAVLRKDSHLKASLEKGEVCLIDYPWETHRLYLHCTFDIRLLTQQGTEQVRLKKLDAAQKSVEKTQERQAADPSITMTANQASRFKAKQTSISRLEKNRPAERPECQIYQPNPNIVVGISLSRHEPVTVVVFNKEKNQASEYWSTESLLKMRGITSPRNNQSIVQLQHEQQQLLRRWRRQRHYNIYQRPEGQKQGDYHQHDAESRLGDYLDRLIAARVTELAVRRQAAAIALPELQNIRESVESDIQARAEKKHPHHANLQAQYAKQYRREFHRWSFGRFEQYITEAAKQRGIAVYKGRQPKHGNEQEKALAVVTNVIA

SEQ ID NO: 24 - protein, 618 aa, unidentified
  source 1..618: note = MG64-37 effector
MSQKTVRARLIVPEETRKAFWELTAGDNTPLVNEALRLLPTHSDFSKWRQKGNLPDKIAEDLVKTLKEDLRFVGQPFWSYISAHKQVTYTFRSWLALQHRKQWKLAGKRLWLEILQPDEILAESVGYTPDALIKAAKKNLADIEAQDNPFDALFSAYRKTKSLKRKSAIAYLLKRSAKLLPEEEDIAKLAQRYRKTEIFIQRLEAQLKASLPKGRDMSGDRQLEALQQIIQAPPMDDVSYNAWKNALTTEPAAFPFPISIETAAWLIWSQDDKGRLLLQLSGWGQHTFKVYFDKAHQHWFWRFLQDQETNQNGGDQHSAALFTLRAAKIMWFPSKKHKDAPEPWHRYHLNLLCTIDTRAWTQEGTEIIAQEKAVKTAKQLASMRKKESLTQNQQGYIRRLESTLNRLQVPYPRPSRPIYQGKPEILVGVSMGLEKVATVAVVNALTGRVLTYRSEKQLLGENYPLLRQARAEIAKKSHQGHRQRLRGVKSISKESDKGKQVDRLFAKAIVELVVEHQAGSIVLPDLAYKREIIEAEFQQRAIEKVPDFVDGQKEYAKAYLSQVHRWPYARLQGCTTSKAEQSGISCEITKQQYSGTPQDKAKGLGFLAYSQRSTALAE

SEQ ID NO: 25 - protein, 661 aa, unidentified
  source 1..661: note = MG64-38 effector
MSIITIHCHLHTTEAIRRLLWQVMAASNTPLISTLLRHVAEHPDFDTWQTNGSVPVKTVRNIAEPLKAHYPSQPGRFYASAYQMVSYTYESWLATQKMIKLRLDGTRRWLSIFKSDAELLELTGLSLESLRQSAREVLSQLSTQSTAGRPSDTQTKPPKAKSRKSKKKQATAQDKDLIGKLFKAYEATDDLTQRCILAYLIKNGGTITDEAETPEAFAHRLHRKQKDIAQLENRLQARLPKGRDLTGDTFIDTLLIAQQQEPEDVAQMRDWQAKLLMRPADLPYPIRYDSSTDMMWKPDDQERITVNFNGLEKFLKNSDPAVKAWLKEHKEYPFRIQCDQRQLPYFQRFLTDWQAYTADKANYPAGLLTLSSAMLAWRKSKKKRKGEPWNTYQLALYCSFDTRLLTAEGTVEVQQEKLRKAQKQANSTKDKKLDENQLQAQTRSATSLRKLENLPARPSRKPYEGKSELLLGISIGFSEPVTVAIVDASTQQAITYRTSRTLLGDQHRLLRRQRQQKQQNRLKRQQNQKKGIRHQPSESELGQYVDRLLAKAIIQLAQTHQVSSIVLPNLTNDRDILNSEIQARAEQKCPGAIAAQAKYAKEVRISIHSWDYRRLSDAIRSSASKQGIPLEEAFLTVRTNPKEQARELAIAAYQARTENRN

SEQ ID NO: 26 - protein, 625 aa, unidentified
  source 1..625: note = MG64-39 effector
ASSTHKPMSQKTIRCRLVASEETRRAIWQLMAERNTPLVNEVLRQLPEHPDFPKWQQRGKLPDLPVKRLIDSLKPDPRFCDQPVWYYISAQKQVAYTFRSWLSLQKRKQWRLEGKRRWLDILQPDAELAEQAKCSVEALRLAASNMLKKVDDPDPFKLLFKEYGTSKSTKRQCALAYLLKRDAKLEPEAEDLEKLDQRRSKAEIQIKQLETQLKASLPKGRDLTGQIQAQALTQSVQSPPLDDEAYSTWHASLAREPAIFPFPIIYETIESLVWSKNSKGRYSVCFQGQGTGIHTFKIYCDKPHQHWFERFWIDQETKRSGNDRHSAGLFTLRSARLSWIPSKKHQDEPEPWNRYYLSLSCTVDTALWTQEGTQTVIQEKAVATASKLQSMQEKESLNKNQQGYVRRLESTLTRLQTPYPRPSRALYQGRSDILVGVSMGLDKPATVAVVNALTGEVLTYRSTKQLLGEQYPLLQRARSERAKVAHQGHRQRRKGGKRVNQESNLGKHVDRLLAKAIVEVAQQYQAGSIVLPDLAHIREIVESEVKQKAAKKVPDFLDGQKQYAKAYRTQVHQWSYHRLQDAITSKAGQSSIATEVAKQDYSGSPQEKAKSLCLAGYEQRLALSS

SEQ ID NO: 27 - protein, 595 aa, unidentified
  source 1..595: note = MG64-40 effector
MAERNTPLVNEVLRQLPEHPDFAKWQQKGNLPDVAVKRIIDALKSDPHFSDQPFWYYTSAQKQVTYTFKSWLSIQRRKQWRLQGKRFWLEILLPDAKLAELAECSVEKLRTEAAKILTKVGDVDPFKHLLEQYRHEKKLLRKYAIAFLLKRNTGIDREEDLEQLKQRSRRVELQIRRLEIQLQASLPKGRDLTGERQAAALAQSVLASPDDDESYELWRNTVTREPAQFPFPVICETSEWLKWQRDQNGRISVGFSALSEHVFKIYCDKPHQHWFNRFFEDQETKRSGGKQHSAGLFTLRSAKLTWVPSNKHANASEPWNCYYLNLSCTVDTRLWTQEGTQIVIQEKAAEKAGKLESMRRKENLSKTQQGYIKRLEATLDKLQTPYPRPSRQLYSGKANILAGVSMGLDKPATVAVVDALTGEVLTYRSVKQLLGENHQLLRRAQIEKTKIAHRGHKNRRQGGRKVSEESNVAQQVDRLLAKSIVEIARKYQASSIVVPDLADIREIVETEVKARAQDKVPDFVEGQQQYAKAYRTQVHQWSYRRLQEAVRTKAEQSGITIEVVRQGLSGTQHEKAKALALQGYEKRIREHVEMA

SEQ ID NO: 28 - protein, 537 aa, unidentified
  source 1..537: note = MG64-41 effector
QLAGQNRWLEVLQNDETLMAVSGLEIQALRAESTKLLDLLSSQITKPAKGSKKTNRGKGKKQAKQTQGKTLYQSLWDLYRETEDILQKCAIAYLLKNKCQVPGKPEDPEKFQHRRRKAEIRAERLNEQLIETRLPKGRDLTNEQWLEALKIATEQVPKDEEEAAIWQSRLLTNAAKFPFPVAYETNEDLKWFLNGKGRLCVRFNGLSEHTFKIYCDQRQLHWFKRFLENKQNKKDNKGKHTSGLFTLRSGRILWKPHSGVVKNAPWTVNHLTLQCSVDTRLWTAEGTEQVRQEKATSIAKVIAGTKAKGNLNRNQLDSISNREKTLELMHNPFPRPSQPIYQGNPSIIAAVSFGLEKPATLAIVDVITGKAITYRSIRQLLGNSYKLFNKQRLKQKQRDYWRHKNQQKSADNRISEGGLGDYVDSLIAKSIVDTAARYEAVSIVLPDQSNIREIIHAEIQAKAERKIPGLKEKQDKYAAQYRRSVHRWSYGRLSQKITTKAAIHGVAIEITRQPLQGTPQEKAVGLAVSAYQSRQVG

SEQ ID NO: 29 - protein, 643 aa, unidentified
  source 1..643: note = MG64-42 effector
MSQITVQCRLVASEETRQYLWYLMADIYTPFVNEMLRQIREDDNFEQWRQAGKIPAGVFEDYRKALKTESRFQGMPGRWYYAGREEVKRIYKSWLALRRRLRNQLSGQNRWLEVLQSDETLMAVSGLDLPALRAASTKLLDLLSSQIAKPAKGSKKTNRGKGKKQAKQTQGKSLYQSLWDLYKETEDILQKCAIAYLLKNKSQVPDKPEDPEKFRHRRRKAEIRTERLNEQLTKTRLPKGRDLTNEQWLEALAIATEQIPKDETEAAIWQSRLLTDAASLPFPVAYETNEDLKWFLNGKGRLCVSFNGLSEHTFEIYCDKRQLHWFKRFLEDQQIKKEHQGKRSSGLFTLRSGRISWTSPSDIDKSPCWTANRLTLHCSVDTRLWTQEGTEEVRQEKATNIAKIIAGTKAKGNLNQKQQDFITKRETTLKLLHNPFPRPSKPLYQGNPSIIAAVSFGLEKPATLAIVDITTGKAITYRSIRQLLDQNYKLFTKHRLQQQQRAHQRHQNQKESAENRISEGGLGEHVDSLIAKAILETAAEYGASSIVLPELGNIREIIQAEVIAKAERKIPGLKEKQDEYAAKFRASVHRWSYGRLAQKITTKAALQGLETESTRQPLQGSPQEKARNLAIAAYESRKVDQRA

SEQ ID NO: 30 - protein, 526 aa, unidentified
  source 1..526: note = MG64-43 effector
MPTTLAPKRPLSKSSDYMKTIRCCLCANPETRRYFWKIMVTYTLLVNELLAAMPQRPEFAQWKQRGTIAREAVRIVLTPLKSDPTYADLPKRFFSSAELLVCYVYKSWLALQKRRYWKLVGKQRWLQVIEDDLQSLLTDNFSLESVQSKAHQILEQAHKELEKQPQRFKKKGKKSRPLFGYLLDLYGTTADKLERRAIGHLLHHDLKVSDTEDFPETIQFSIDQQQVEIARLKEQLQSRLPDGRDPTQARFLEKLRIATALPELELEGFDEEHFSEWRTQKQIPLLNPLPYPVLFGSSSDLHWKLEPQKATTEANISPEVPTARSERVKERIQVRFKGDELQDSWFKLQCDRRQLPIFRQFVTDYLCQKQAPDHEKFGEGLFTLRSACLVWKEDPQGARKRKKRRKQGACQDEPWETHRLYLHCTIDTRFLTQEGTEQVRATKLDLAQKALEGIENKTALETVTQEPSAEQQKHLKRKQTTVHRLETQKPPVRPTIQPYEGKSNIVVGVSLSRHEPVTLIVFDTAQ

SEQ ID NO: 31 - skipped

SEQ ID NO: 32 - DNA, 417 bp, synthetic construct
  source 1..417: mol_type = other DNA; note = MG64-3 effector intergenic region encoding tracrRNA
aggttcgtatgtaccactaaaataaatgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccaaaacttaaatttggcaaacctaagcgaggtcaaaaaccctgggaggtttgccaaaagactgaagctcctggtctacaaaggtttgagtcatctagtttgtcccaatttctggtctgtcataagaatttagtagaactagattgggctttgccaaattcaactctgcaaagcttgcagggtatgcctttccgatggcaa

SEQ ID NO: 33 - DNA, 468 bp, synthetic construct
  source 1..468: mol_type = other DNA; note = MG64-5 effector intergenic region encoding tracrRNA
ctcagcacagatatctgaaccttgaaaaatgaatatctgatatttcttgtgcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccaaattttggcaaacctcagcgcagtcaatatgcccaggcggtttgccaatctctcaaatccttgtgcaataggtctttcatgcaatctcgtcattgagaagcttcctagaggcgttcggcatccccaaaataattgaggtttgccaaataccccctcgaaaagactgctgtataagcttttcaagctgcg

SEQ ID NO: 34 - DNA, 37 bp, synthetic construct
  source 1..37: mol_type = other DNA; note = MG64-3 effector target CRISPR repeat
gtcgcccaaggcatttcagggcagggcggattgaaag

SEQ ID NO: 35 - DNA, 37 bp, synthetic construct
  source 1..37: mol_type = other DNA; note = MG64-5 effector target CRISPR repeat
gtttcatccctgcatttcaatgcagatgggatgaaag

SEQ ID NO: 36 - DNA, 105 bp, synthetic construct
  source 1..105: mol_type = other DNA; note = MG64-1 LE 105bp
tgtacattaacagattatttgtcatcggtaacaaattgttgtcatcttaacaaaatatttgtcatcaataacatattatgtgtcgtgtgcttattactgaaacta

SEQ ID NO: 37 - DNA, 86 bp, synthetic construct
  source 1..86: mol_type = other DNA; note = MG64-1 LE 86bp
tgtacattaacagattatttgtcatcggtaacaaattgttgtcatcttaacaaaatatttgtcatcaataacatattatgtgtcgt

SEQ ID NO: 38 - DNA, 68 bp, synthetic construct
  source 1..68: mol_type = other DNA; note = MG64-1 LE 68bp
tgtacattaacagattatttgtcatcggtaacaaattgttgtcatcttaacaaaatatttgtcatcaa

SEQ ID NO: 39 - DNA, 242 bp, synthetic construct
  source 1..242: mol_type = other DNA; note = MG64-1 RE 242bp
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcacttttttagggactggagcagtattaacaaattacttgtctccaaac

SEQ ID NO: 40 - DNA, 196 bp, synthetic construct
  source 1..196: mol_type = other DNA; note = MG64-1 RE 196bp
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgtcatttacccgaatggcac

SEQ ID NO: 41 - DNA, 178 bp, synthetic construct
  source 1..178: mol_type = other DNA; note = MG64-1 RE 178bp
tgtacagtaactaattatttgtcgtcttaacaaaatcgtgtcgccgaactatttgagagtgaaagctttatataatcactgctttagctgaattatagcattaatctttgacaaaagttattgcttactatattaacaaattaactgtcattttccagtaaattaacagattaagtgt

SEQ ID NO: 42 - DNA, 260 bp, synthetic construct
  source 1..260: mol_type = other DNA; note = MG64-1 RE internal deletion 50
tgtacagtaactaattatttgtcgtcttaacaaaatcgtgtcgccgaactatttgagagtgaaagaaagttattgcttactatattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 43 - DNA, 231 bp, synthetic construct
  source 1..231: mol_type = other DNA; note = MG64-1 RE internal deletion 81
tgtacagtaactaattatttgtcgtcttaacaaaatcgtgtcgccgaactatttattaacaaattaactgtcattttccagtaaattaacagattaagtgtcatttacccgaatggcacttttttagggactggagcagtattaacaaattacttgtctccaaacttcaagtattacgatacaattatattaaataaacaattatgtaataatcagacatttttgttcatt

SEQ ID NO: 44 - DNA, 96 bp, synthetic construct
  source 1..96: mol_type = other DNA; note = MG64-1 RE internal deletion 81 & 178bp
tgtacagtaactaattatttgtcgtcttaacaaaatcgtgtcgccgaactattaacaaattaactgtcattttccagtaaattaacagattaagtg

SEQ ID NO: 45 - RNA, 237 nt, synthetic construct
  source 1..237: mol_type = other RNA; note = MG64-1 engineered single guide 1
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacctccgaccaaggaggaatccactgaaaagatggattgaaag

SEQ ID NO: 46 - RNA, 214 nt, synthetic construct
  source 1..214: mol_type = other RNA; note = MG64-1 engineered single guide 2
aaaataatcgcgccgtagatcatgttcttgattgaacctctgaactacgaaaaatgagggttagtttgactctcggcagatagtcttgctttctgaccctagtggctgtccaccctgatgctggaaacagcaataagtggcgtgggtttaccacagtgacggctactgaatcacctccgaccaaggaggaatccactaaaagatggattgaaag

SEQ ID NO: 47 - RNA, 170 nt, synthetic construct
  source 1..170: mol_type = other RNA; note = MG64-1 engineered single guide 3
aaaataatcgcgccgtagatcatgttcttgattgaacctctgaactacgaaaaatgagggttagtttgactctcggcagatagtcttgctttctgaccctagtggctgtgaaaacggctactgaatcacctccgaccaaggaggaatccactgaaaagatggattgaaag

SEQ ID NO: 48 - RNA, 276 nt, synthetic construct
  source 1..276: mol_type = other RNA; note = MG64-1 engineered single guide 4
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggatgaaag

SEQ ID NO: 49 - RNA, 281 nt, synthetic construct
  source 1..281: mol_type = other RNA; note = MG64-1 engineered single guide 5
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgaatccactgaaaagatggattgaaag

SEQ ID NO: 50 - RNA, 284 nt, synthetic construct
  source 1..284: mol_type = other RNA; note = MG64-1 engineered single guide 6
gccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 51 - RNA, 283 nt, synthetic construct
  source 1..283: mol_type = other RNA; note = MG64-1 engineered single guide 7
aaaataatcgcgccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 52 - RNA, 273 nt, synthetic construct
  source 1..273: mol_type = other RNA; note = MG64-1 engineered single guide 8
aaaataatcgcgccgtagataactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 53 - RNA, 263 nt, synthetic construct
  source 1..263: mol_type = other RNA; note = MG64-1 engineered single guide 9
aaaataatcgcgccgcgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 54 - RNA, 278 nt, synthetic construct
  source 1..278: mol_type = other RNA; note = MG64-1 engineered single guide 10
aaaataatcgcgccgtagatcatgttcttgattgaacctctgaactacgaaaaatgcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 55 - RNA, 215 nt, synthetic construct
  source 1..215: mol_type = other RNA; note = MG64-1 engineered single guide 11
aaaataatcgcgccgtagatcatgttcttgattgaacctctgaactacgaaaaatgagggttagtttgactctcgcctagtggctgtccaccctgatgctgatttctacatagggtaggtgcgctcccagcaataagtggcgtgggtttaccacagtgacggctactgaatcacctccgaccaaggaggaatccactgaaaagatggattgaaag

SEQ ID NO: 56 - RNA, 221 nt, synthetic construct
  source 1..221: mol_type = other RNA; note = MG64-1 engineered single guide 12
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctgtgggtttaccacagtgacggctactgaatcacctccgaccaaggaggaatccactgaaaagatggattgaaag

SEQ ID NO: 57 - RNA, 226 nt, synthetic construct
  source 1..226: mol_type = other RNA; note = MG64-1 engineered single guide 13
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgcagtgacggctactgaatcacctccgaccaaggaggaatccactgaaaagatggattgaaag

SEQ ID NO: 58 - RNA, 224 nt, synthetic construct
  source 1..224: mol_type = other RNA; note = MG64-1 engineered single guide 14
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcagtgacggctactgaatcacctccgaccaaggaggaatccactgaaaagatggattgaaag

SEQ ID NO: 59 - RNA, 204 nt, synthetic construct
  source 1..204: mol_type = other RNA; note = MG64-1 engineered single guide 15
aaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacgatgaaag

SEQ ID NO: 60 - RNA, 181 nt, synthetic construct
  source 1..181: mol_type = other RNA; note = MG64-1 engineered single guide 16
gccgtagatcatgcctctgaactacgaaaaatgagggttagtttgactctcggcagatagtcttgctttctgaccctagtggctgtccaccctgatgctgatttctacatagggtaggtgcgctcccagcaataagtggcgtgggtttaccacagtgacggctactgaatcacgatgaaag

SEQ ID NO: 61 - RNA, 262 nt, synthetic construct
  source 1..262: mol_type = other RNA; note = MG64-1 engineered single guide 17
aaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaag

SEQ ID NO: 62 - RNA, 272 nt, synthetic construct
  source 1..272: mol_type = other RNA; note = MG64-1 engineered single guide 18
gccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacctccgaccaaggaggaatccactgaaaagatggattgaaag

SEQ ID NO: 63 - RNA, 251 nt, synthetic construct
  source 1..251: mol_type = other RNA; note = MG64-1 engineered single guide 19
gccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaag

SEQ ID NO: 64 - protein, 122 aa, unidentified
  source 1..122: note = MG64-55 effector
MSQITVRARLIAPEETRRAYWDLMAASNTPLINEALRILPTLPDFPKWRQKGNLPDKAAENLIIKLKEDPRFVGQLFWSYISAHKQVTYTFRSWLALQHRKQWKLAGKRLWLEILQPDETLA

SEQ ID NO: 65 - protein, 134 aa, unidentified
  source 1..134: note = MG64-55-B transposition protein
SSSVSKGTMMAQWRNAYTSILKLIDRQSLKAKSVHLGHLHKGISRARSLREWEAAKKALKKQNKNLTSQNVSTYFEDEERTPQKSMRQRRKAAQKANKKPLPMIEDDLQEESNLEDEKNPLLDLEVTYDDDLFE

SEQ ID NO: 66 - protein, 272 aa, unidentified
  source 1..272: note = MG64-55-C transposition protein
MEKQVDQIASALGALPELSPEIHKELERLSKRPYILLPKVESCHIFLEECRLGRAHGRIVGDSGVGKTISAKAYSKRLAEASTEKNVIYTILNPNCTPKEFYEKILEALGFTYTKGSIKFLRNRACQVLSRRQISVLFIDEASFLKMDAIGELIYLEESEVVPSIFLIGTDRLDTLLSGNEQVARRYPRYQYGRLHDKELKDVVDLWEQKVLQLPVKSNLKYKAKLNVITKATSGCLGEIDQLLRRAARKALILGESKISLNILREVAGQFE

SEQ ID NO: 67 - protein, 169 aa, unidentified
  source 1..169: note = MG64-55-Q transposition protein
MNDAQAAQWHFKPEPFEGESFSHFLGRYCAVNCIAPNILAKHIEAGSVAIGRWRKLRYNPSPSERHLQRLADVTGVSQERLLAMLPQEPMQIGTIRLCAACYGEEPCHRIRWQYKSTQFCDRHQLTLLARCPCCKAPFPIPAEWDAGICLRCGKAFVELAEFQKSVLGQ

SEQ ID NO: 68 - RNA, 239 nt, synthetic construct
  source 1..239: mol_type = other RNA; note = MG64-1 active effector engineered sgRNA 11
gaaaataatcgcgccgtagatcatgttcttgattgaacctctgaactacgaaaaatgagggttagtttgactctcgcctagtggctgtccaccctgatgctgatttctacatagggtaggtgcgctcccagcaataagtggcgtgggtttaccacagtgacggctactgaatcacctccgaccaaggaggaatccactgaaaagatggattgaaagnnnnnnnnnnnnnnnnnnnnnnn

SEQ ID NO: 69 - RNA, 245 nt, synthetic construct
  source 1..245: mol_type = other RNA; note = MG64-1 active effector engineered sgRNA 12
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctgtgggtttaccacagtgacggctactgaatcacctccgaccaaggaggaatccactgaaaagatggattgaaagnnnnnnnnnnnnnnnnnnnnnnn

SEQ ID NO: 70 - RNA, 250 nt, synthetic construct
  source 1..250: mol_type = other RNA; note = MG64-1 active effector engineered sgRNA 13
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcgcagtgacggctactgaatcacctccgaccaaggaggaatccactgaaaagatggattgaaagnnnnnnnnnnnnnnnnnnnnnnn

SEQ ID NO: 71 - RNA, 248 nt, synthetic construct
  source 1..248: mol_type = other RNA; note = MG64-1 active effector engineered sgRNA 14
gaaaataatcgcgccgtagatcatgttcttgattgaacctctgaactacgaaaaatgagggttagtttgactctcggcagatagtcttgctttctgaccctagtggctgtccaccctgatgctgatttctacatagggtaggtgcgctcccagcaataagtggcagtgacggctactgaatcacctccgaccaaggaggaatccactgaaaagatggattgaaagnnnnnnnnnnnnnnnnnnnnnnn

SEQ ID NO: 72 - RNA, 263 nt, synthetic construct
  source 1..263: mol_type = other RNA; note = MG64-1 active effector engineered sgRNA v2-1
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaagnnnnnnnnnnnnnnnnnnnnnnn

SEQ ID NO: 73 - RNA, 262 nt, synthetic construct
  source 1..262: mol_type = other RNA; note = MG64-1 active effector engineered sgRNA 20
gccgtagatcatgcctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcattttatagggtaggtgcgctcccagcaataagtggcgtgggtttaccacagtgacggctactgaatcacgatgaaagnnnnnnnnnnnnnnnnnnnnnnn

SEQ ID NO: 74 - RNA, 241 nt, synthetic construct
  source 1..241: mol_type = other RNA; note = MG64-3 effector sgRNA
gaaataaatgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccgaaagggcggattgaaagnnnnnnnnnnnnnnnnnnnnnnn

SEQ ID NO: 75 - RNA, 264 nt, synthetic construct
  source 1..264: mol_type = other RNA; note = MG64-5 effector sgRNA
gcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccgaaaggatgaaagnnnnnnnnnnnnnnnnnnnnnnn

SEQ ID NO: 76 - DNA, 447 bp, synthetic construct
  source 1..447: mol_type = other DNA; note = MG64-3 putative transposon end LE
aaggcgagtttgggaacggatactcctgggcatttctaactcaacgtggtatcgtcataaatccgagggcgattagcacagtggtagcgcgcttccttcacacggaagaggtcactggttcgaacccagtatcgcccatacatttgtcgaataacgctttatttgtcgtcggtaacacatttgtgtcgtccataacgcttagatgtcatcgataacagttttttgtcaccttctcggtaagtttgccgttaagcgatcgttcacacaatgctgtcgtccaaaattaggcttttgatgtgtgaaggaagcgcgcctttcaatccacctatgcgtatattaacgccaaaacttgatccaagtacaaatgttttaagactgtattctggtctgcatgaaaagctttaggcgtctagatatgagaagccacctaatctacatgcggtcgac

SEQ ID NO: 77 - DNA, 430 bp, synthetic construct
  source 1..430: mol_type = other DNA; note = MG64-3 putative transposon end RE
ggagtcatgcagtacaagtgaattaaatcaaatgtactatgactaccaagatatgtcatctaatttgttagatccgataaagccgccaagaggcgacaaagagtgtgttaatgccaaggtgatctcagattccaagagacgacactgattgtgttcaacttccaagagacgtcagttattctgttagtcaaagagactcattgagatatgagtggaaacgctgaagagtctgatctgaattggttctggctatctcaagctcgatgacaagattgcgttatgacgacattttacgtgttactcgacaacagcggctgacggtggcaccctcagcaattttctcttccacaaagtcgggatcatctgaccccgtgacgaagggccgccagggacgtataccatgtcactgaggcagcaagatagggatcaa

SEQ ID NO: 78 - DNA, 450 bp, synthetic construct
  source 1..450: mol_type = other DNA; note = MG64-5 putative transposon end LE
ttgagacgaaggttgatatctatcgagccgtaactgcttgagctgagttttcaacctctaagcgcaagaaaaaacgactcgtaattcgcgaaaaacgcacatatgaactttcgacttccgatttgcgaaaaatgactttgcgactcgcaatctgcgaatctgtacttgtttactgatttcgggttggagtgccaaaactctctctgggcagggactttacggattatttacaagcgatattaatctgcgaatcgcgacatttaatgtgcgaacgtacaccagatttaaaggattaatttcccaaaaaacacggaagaataggcattttagccgtcaaaccgctattacagtcggtcaattgatgttaaatccgccatttttgccagatttaaggaattagtttccaaaatcctaggccctagttggatgttgggtgcgatcgcagcgctc

SEQ ID NO: 79 - DNA, 583 bp, synthetic construct
  source 1..583: mol_type = other DNA; note = MG64-5 putative transposon end RE
tttagttgtcgcaaagacaactgtaaaagtggtatcacaggtgccctggaatcgtcaaaccctctctattagatcctttttgcttgttttgcggtctgatcaggatcagatttatcgattccaagcttgtcataatgacaattaacgaatagcgatatacgtatacgtacatcgacatgttgatagttgctcacccacacagtggccgcgagcgacttaccgaaaggctcctaggtgtttgtgagtggtattggaaagcagcaaaagcgatcgcaccccctaatggagtctgccgaaacaggcgcaaaacaaagatggcgacgattgcagaaacgcctgacagcagcagccgaggatgggaatactaggctgctaatgtcgatcgcccacaagtacccaaatgcctgaaacccacatcaccgttggagcccatcaagattttgagacttacctgacggaacctttcggtgacgggcctgccagtgatgaacgtgcggccgactttagaaataggctgaaatcagcccagacgctccaagatgctggtttgccaatagtcgaaaatgttttggttggtcgctga

SEQ ID NO: 80 - protein, 803 aa, synthetic construct
  source 1..803: note = MG64-1 effector - MG64-1-Q fusion
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSAGGGSGGGSGGGSGGGSGGGSMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 81 - protein, 803 aa, synthetic construct
  source 1..803: note = MG64-1-Q - MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 82 - protein, 831 aa, synthetic construct
  source 1..831: note = MG64-1-Q- 48aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 83 - protein, 851 aa, synthetic construct
  source 1..851: note = MG64-1-Q- 68aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 84 - protein, 855 aa, synthetic construct
  source 1..855: note = MG64-1-Q- 72aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGGGSGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 85 - protein, 860 aa, synthetic construct
  source 1..860: note = MG64-1-Q- 77aa Linker- MG64-1 effector fusion
MESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYLGTKYLGGGSGGGSGGGSGGGSGGGSGGGSGGGSGGGSGGGSGGGSGGGSGGGSGGGSGGGSGGGSGGGSGGGSGGGSMSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSA

SEQ ID NO: 86 - protein, 16 aa, synthetic construct
  source 1..16: note = Nucleoplasmin NLS
KRPAATKKAGQAKKKK

SEQ ID NO: 87 - protein, 19 aa, synthetic construct
  source 1..19: note = SV40 2x NLS
PKKKRKVDGSPKKKRKVDS

SEQ ID NO: 88 - protein, 19 aa, synthetic construct
  source 1..19: note = P2A
ATNFSLLKQAGDVEENPGP

SEQ ID NO: 89 - protein, 18 aa, synthetic construct
  source 1..18: note = T2A
EGRGSLLTCGDVEENPGP

SEQ ID NO: 90 - protein, 9 aa, synthetic construct
  source 1..9: note = HA
YPYDVPDYA

SEQ ID NO: 91 - protein, 10 aa, synthetic construct
  source 1..10: note = Myc
EQKLISEEDL

SEQ ID NO: 92 - protein, 8 aa, synthetic construct
  source 1..8: note = FLAG
DYKDDDDK

SEQ ID NO: 93 - DNA, 486 bp, synthetic construct
  source 1..486: mol_type = other DNA; note = MG64-1 active transposon end LE
ctggctaatgcacccagtaaggcagcggtatcatcaacggggtctgacgctcagtggaacgaaaactcacgttaggctctcttaaggaaatctatgtaagtttgttgggttagttgcgttttcagtaaatactgtgttatagtaagaacttgtgcggacgtatagctcagttggttagagtacatcgttgacatcgatggggtcactggttcgagtccagttacgtccatatttttttgaagtgtgtataatattaactatgtgactttatgtacattaacagattatttgtcatcggtaacaaattgttgtcatcttaacaaaatatttgtcatcaataacatattatgtgtcgtgtgcttattactgaaactaatcctagacgatggtaaaaaatagaacacatttccccgaaaagtgccacctgacgtctaagaaaccattattatcatgacattaacctataaaaataggcgtatcacgagg

SEQ ID NO: 94 - RNA, 199 nt, synthetic construct
  source 1..199: mol_type = other RNA; note = MG64-3 effector putative tracrRNA
tgcagcgcctaagttcatgtcgtcagcggcctctgtgcttagaaaaagggctagtttgactgtctgaacgcagtcttgctttctgacctagataactgtccatccccaaagctgtgagcgcacgcagcaagagggcacgggttccggagtgatggttatcaaattcacctccgagcaaggaggaatccacccaaaactt

SEQ ID NO: 95 - RNA, 237 nt, synthetic construct
  source 1..237: mol_type = other RNA; note = MG64-5 effector putative tracrRNA
tcttgtgcgcgccggttctttagggactgagcgataagttagggcgagtttaattgctttccagcccgtgtagttgtccgctctcttgtgcagcttgctgcatgctaggtgtcgggtcgcgccgacatccaagaggccatgtttctgtagttagaggctatctcttcaattatagggatacaggtgtacgtgtcgtggcagctaccaaacagccccgagcaagggggcccatccaaa

SEQ ID NO: 96 - RNA, 239 nt, synthetic construct; identical to SEQ ID NO: 68
  source 1..239: mol_type = other RNA; note = MG64-1 active effector engineered sgRNA 11

SEQ ID NO: 97 - RNA, 245 nt, synthetic construct; identical to SEQ ID NO: 69
  source 1..245: mol_type = other RNA; note = MG64-1 active effector engineered sgRNA 12

SEQ ID NO: 98 - RNA, 250 nt, synthetic construct; identical to SEQ ID NO: 70
  source 1..250: mol_type = other RNA; note = MG64-1 active effector engineered sgRNA 13

SEQ ID NO: 99 - RNA, 248 nt, synthetic construct; identical to SEQ ID NO: 71
  source 1..248: mol_type = other RNA; note = MG64-1 active effector engineered sgRNA 14

SEQ ID NO: 100 - RNA, 263 nt, synthetic construct; identical to SEQ ID NO: 72
  source 1..263: mol_type = other RNA; note = MG64-1 active effector engineered sgRNA v2-1

SEQ ID NO: 101 - RNA, 262 nt, synthetic construct; identical to SEQ ID NO: 73
  source 1..262: mol_type = other RNA; note = MG64-1 active effector engineered sgRNA 20

SEQ ID NO: 102 - RNA, 241 nt, synthetic construct; identical to SEQ ID NO: 74
  source 1..241: mol_type = other RNA; note = MG64-3 effector sgRNA

SEQ ID NO: 103 - RNA, 264 nt, synthetic construct; identical to SEQ ID NO: 75
  source 1..264: mol_type = other RNA; note = MG64-5 effector sgRNA

SEQ ID NO: 104 - RNA, 199 nt, synthetic construct; identical to SEQ ID NO: 94
  source 1..199: mol_type = other RNA; note = MG64-3 effector putative tracrRNA sequence

SEQ ID NO: 105 - RNA, 237 nt, synthetic construct; identical to SEQ ID NO: 95
  source 1..237: mol_type = other RNA; note = MG64-5 effector putative tracrRNA sequence

SEQ ID NO: 106 - RNA, 38 nt, synthetic construct
  source 1..38: mol_type = other RNA; note = MG64-1 active effector's crRNA sequence
cgtcacaatctattttggttaatgagatggattgaaag

SEQ ID NO: 107 - RNA, 37 nt, synthetic construct
  source 1..37: mol_type = other RNA; note = MG64-3 effector crRNA sequence
gtcgcccaaggcatttcagggcagggcggattgaaag

SEQ ID NO: 108 - RNA, 37 nt, synthetic construct
  source 1..37: mol_type = other RNA; note = MG64-5 effector crRNA sequence
gtttcatccctgcatttcaatgcagatgggatgaaag

SEQ ID NO: 109 - protein, 63 aa, Saccharolobus solfataricus
ATVKFKYKGEEKEVDISKIKKVWRVGKMISFTYDEGGGKTGRGAVSEKDAPKELLQMLEKQKK

SEQ ID NO: 110 - protein, 51 aa, Saccharomyces cerevisiae
EEYVVEKVLDRRVVKGQVEYLLKWKGFSEEHNTWEPEKNLDCPELISEFMK

SEQ ID NO: 111 - protein, 99 aa, Homo sapiens
PKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSD

SEQ ID NO: 112 - protein, 80 aa, Homo sapiens
STDHPKYSDMIVAAIQAEKNRAGSSRQSIQKYIKSHYKVGENADSQIKLSIKRLVTTGVLKQTKGVGASGSFRLAKSDEP

SEQ ID NO: 113 - protein, 736 aa, synthetic construct
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSGGGGSGGGGSATVKFKYKGEEKEVDISKIKKVWRVGKMISFTYDEGGGKTGRGAVSEKDAPKELLQMLEKQKKSGGKRPAATKKAGQAKKKKYPYDVPDYAGSGSPKKKRKVDGSPKKKRKVDSG

SEQ ID NO: 114 - protein, 719 aa, synthetic construct
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSGGGGSEEYVVEKVLDRRVVKGQVEYLLKWKGFSEEHNTWEPEKNLDCPELISEFMKSGGKRPAATKKAGQAKKKKYPYDVPDYAGSGSPKKKRKVDGSPKKKRKVDSG

SEQ ID NO: 115 - protein, 748 aa, synthetic construct
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSGGGGSSTDHPKYSDMIVAAIQAEKNRAGSSRQSIQKYIKSHYKVGENADSQIKLSIKRLVTTGVLKQTKGVGASGSFRLAKSDEPSGGKRPAATKKAGQAKKKKYPYDVPDYAGSGSPKKKRKVDGSPKKKRKVDSG

SEQ ID NO: 116 - protein, 767 aa, synthetic construct
MSQITIQCRLVAKEPIRHTLWQLMADLNTPFINELLQKVAQHPDFEKWKQRGRLKVKVIEQLGNELKKDPRFLGQPARFYTSGINLVKYIFKSWLKLQQRLQQKLDRKRRWLEVLKSDDQLIKDGQTDLETIRQKATEILQSYEGTEQLFNTLFQAYNSEEDILTRTALNYLLKNRCKLPQKPEDAKKFAKRRRQVEIAIKRLQEQIKARLPQGRDVTNENWLETLNLACYTDPENIEEARSWQDKLLTKSSSIPFPINYETNEDLIWSKNEKGHLCVQFNGISDLKFKIYCDKRQLKWFQRFYEDQQIKKSNNNQHSSALFTLRSGRILWQEDKGKGQLWDIHRLTLQCTLDTRTWTQEGTEQVKEEKADEIAGILTRMNEKGDLTKNQQAFIQRKQSTLDKLENPFPRPSRPVYRGQSNILLGVSMELKKPATIAVIDGMTRKVLTYRNIKQLLGKNYPLLNRQRRQKQLQSHQRNVAQRKEAFNQFGDSELGEYIDRLLAKAIIAIAKQYQARSIVVPHLKDIREAIQSEIQALAEAKIPNCIEAQAEYAKKYRIQVHQWSYGRLIDNIQAQASKLGIVIEESQQPLQGTPLQKAAELAFKAYQSRLSGGGGSPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSDSGGKRPAATKKAGQAKKKKYPYDVPDYAGSGSPKKKRKVDGSPKKKRKVDSG

SEQ ID NO: 117 - protein, 281 aa, synthetic construct
MKRPAATKKAGQAKKKKGGGGSGGGGSGGGGSATVKFKYKGEEKEVDISKIKKVWRVGKMISFTYDEGGGKTGRGAVSEKDAPKELLQMLEKQKKSGGYPYDVPDYANATMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 118 - protein, 271 aa, synthetic construct
MKRPAATKKAGQAKKKKGGGGSEEYVVEKVLDRRVVKGQVEYLLKWKGFSEEHNTWEPEKNLDCPELISEFMKGGGGSGGGGSGGGGSYPYDVPDYANATMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 119 - protein, 300 aa, synthetic construct
MKRPAATKKAGQAKKKKGGGGSSTDHPKYSDMIVAAIQAEKNRAGSSRQSIQKYIKSHYKVGENADSQIKLSIKRLVTTGVLKQTKGVGASGSFRLAKSDEPGGGGSGGGGSGGGGSYPYDVPDYANATMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 120 - protein, 319 aa, synthetic construct
MKRPAATKKAGQAKKKKGGGGSPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSDGGGGSGGGGSGGGGSYPYDVPDYANATMESREIQPWWFLVEPLAGESISHFLGRFRRENELTVTMMGKITGLGGTITRWEKFRFIPIPTEEELTALSEVVQVEVERLWQMFPPKGVGMKHQPIRLCGACYEEERCHKIEWQLKTTQFCSQHGLTLLSECPNCGARFQFPALWVNGWCHRCFLTFGEMVEGQSNKKKYL

SEQ ID NO: 121 - DNA, 335 bp, synthetic construct
  source 1..335: mol_type = other DNA
aatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacacgtatgtttattgcggcactattcacaatagca

SEQ ID NO: 122 - DNA, 485 bp, synthetic construct
  source 1..485: mol_type = other DNA
ctctccctctccctctccctctccctctccctctccctctccctctcccctccctccacggtctccctctgatgccgagccaaagctggacggtactgctgccatctcggctcactgcaacctccctgcctgattctcctgcctcagcctgccgagtgcctgcgattgcaggcgcgcgccgccacgcctgactggttttcgtttttttttggtggagacggggtctcgctgtgttgnccgggctggtctccagctcctggccgcgagtgatccgccngcctcggcctcccgaggtgccgggattgcagacggagtctcgttcactcagtgctcaatggtgcccaggctggagtgcagtggcgtgatctcggctcgctacaaccacctcccagccgcctgccttggcctcccaaagagccggattgcagcctctgcccggccgccgccccgtccgggaggtgaggagcgcctctgcccggccgccc

SEQ ID NO: 123 - DNA, 323 bp, synthetic construct
  source 1..323: mol_type = other DNA
ttcaaccggcagctgaccaggccaagtcctcccccttctcgtggttaactttaatctcagaaggtgcacaattgctccaatccacaggggtacaaaacctctcccactgcttcctctgtgcagccctcggaagacctcccttagtagcagttcctctccctaccccctttaattatacaagaaattcatccacccctataccaccggtcccgaaaggacaggtcccactattctcagaccctacaagacacaagttcccgttctgttactctaccccaaatgcctcttggtgtaaccagaccaggatgcttaccagcgccccg

SEQ ID NO: 124 - DNA, 24 bp, synthetic construct
  source 1..24: mol_type = other DNA
atgggatggctgggtcaaatggta

SEQ ID NO: 125 - DNA, 24 bp, synthetic construct
  source 1..24: mol_type = other DNA
aaatggtatttctagttctagatc

SEQ ID NO: 126 - DNA, 24 bp, synthetic construct
  source 1..24: mol_type = other DNA
gaactagtttacagtcccaccaac

SEQ ID NO: 127 - DNA, 24 bp, synthetic construct
  source 1..24: mol_type = other DNA
tatacccagtaatgggatggctgg

SEQ ID NO: 128 - DNA, 24 bp, synthetic construct
  source 1..24: mol_type = other DNA
aggaaacaacaggtgctggagagg

SEQ ID NO: 129 - DNA, 24 bp, synthetic construct
  source 1..24: mol_type = other DNA
tacagtcccaccaacagtgtaaaa

SEQ ID NO: 130 - DNA, 23 bp, synthetic construct
  source 1..23: mol_type = other DNA
caccaacagtgtaaaagtgttcc

SEQ ID NO: 131 - DNA, 24 bp, synthetic construct
  source 1..24: mol_type = other DNA
ggactgtaaactagttcaaccatt

SEQ ID NO: 132 - DNA, 24 bp, synthetic construct
  source 1..24: mol_type = other DNA
tttctagttctagatccctgagga

SEQ ID NO: 133 - DNA, 24 bp, synthetic construct
  source 1..24: mol_type = other DNA
caaccattgtggaagtcagtgtgg

SEQ ID NO: 134 - DNA, 24 bp, synthetic construct
  source 1..24: mol_type = other DNA
tggcgattcctcagggatctagaa

SEQ ID NO: 135 - DNA, 24 bp, synthetic construct
  source 1..24: mol_type = other DNA
ctagatccctgaggaatcgccaca

SEQ ID NO: 136 - DNA, 24 bp, synthetic construct
  source 1..24: mol_type = other DNA
agaatggcaatcattaaaaagtca

SEQ ID NO: 137 - DNA, 24 bp, synthetic construct
  source 1..24: mol_type = other DNA
agtgtggcgattcctcagggatct

SEQ ID NO: 138 - DNA, 24 bp, synthetic construct
  source 1..24: mol_type = other DNA
tatacccaaaggactataaatcat

SEQ ID NO: 139 - DNA, 24 bp, synthetic construct
  source 1..24: mol_type = other DNA
gtagcgagccgagatcacgccact

SEQ ID NO: 140 - DNA, 24 bp, synthetic construct
  source 1..24: mol_type = other DNA
ctcaatggtgcccaggctggagtg

SEQ ID NO: 141 - DNA, 24 bp, synthetic construct
  source 1..24: mol_type = other DNA
cccaggctggagtgcagtggcgtg

SEQ ID NO: 142 - DNA, 24 bp, synthetic construct
  source 1..24: mol_type = other DNA
gcgtgatctcggctcgctacaacc

SEQ ID NO: 143 - DNA, 24 bp, synthetic construct
  source 1..24: mol_type = other DNA
cagtggcgtgatctcggctcgcta

SEQ ID NO: 144 - DNA, 24 bp, synthetic construct
  source 1..24: mol_type = other DNA
tcgttcactcagtgctcaatggtg

SEQ ID NO: 145 - DNA, 24 bp, synthetic construct
  source 1..24: mol_type = other DNA
aacgagactccgtctgcaatcccg

SEQ ID NO: 146 - DNA, 24 bp, synthetic construct
  source 1..24: mol_type = other DNA
cctctccctaccccctttaattat

SEQ ID NO: 147 - DNA, 24 bp, synthetic construct
  source 1..24: mol_type = other DNA
agcatcctggtctggttacaccaa

SEQ ID NO: 148 - DNA, 24 bp, synthetic construct
  source 1..24: mol_type = other DNA
acagaacgggaacttgtgtcttgt

SEQ ID NO: 149 - DNA, 24 bp, synthetic construct
  source 1..24: mol_type = other DNA
gtataggggtggatgaatttcttg

SEQ ID NO: 150 - DNA, 24 bp, synthetic construct
  source 1..24: mol_type = other DNA
taggggtggatgaatttcttgtat

SEQ ID NO: 151 - DNA, 24 bp, synthetic construct
  source 1..24: mol_type = other DNA
gatgaatttcttgtataattaaag

SEQ ID NO: 152 - DNA, 24 bp, synthetic construct
  source 1..24: mol_type = other DNA
acaccaagaggcatttggggtaga

SEQ ID NO: 153 - DNA, 24 bp, synthetic construct
  source 1..24: mol_type = other DNA
taaccagaccaggatgcttaccag

SEQ ID NO: 154 - DNA, 24 bp, synthetic construct
  source 1..24: mol_type = other DNA
tggttacaccaagaggcatttggg

SEQ ID NO: 155 - DNA, 24 bp, synthetic construct
  source 1..24: mol_type = other DNA
gagtaacagaacgggaacttgtgt

SEQ ID NO: 156 - DNA, 24 bp, synthetic construct
  source 1..24: mol_type = other DNA
gggagaggaactgctactaaggga

SEQ ID NO: 157 - DNA, 24 bp, synthetic construct
  source 1..24: mol_type = other DNA
aactttaatctcagaaggtgcaca

SEQ ID NO: 158 - DNA, 24 bp, synthetic construct
  source 1..24: mol_type = other DNA
tgagaatagtgggacctgtccttt

SEQ ID NO: 159 - DNA, 24 bp, synthetic construct
  source 1..24: mol_type = other DNA
ccgaaaggacaggtcccactattc

SEQ ID NO: 160 - DNA, 24 bp, synthetic construct
  source 1..24: mol_type = other DNA
cacaattgctccaatccacagggg

SEQ ID NO: 161 - DNA, 387 bp, synthetic construct
  source 1..387: mol_type = other DNA
atgaagaggcccgcagcgactaagaaagcaggacaggcgaagaagaagaagtacccatatgacgtgccagattatgctggtggcggcggctctggtggtggcggcagcggtggcggcggcagttctctgtcaacggaagcaaccgcaaagattgtatcagagtttggcagagacgcgaacgatacggggagtacggaagtgcaggtcgccctgctcaccgcacaaattaaccaccttcagggacatttcgctgaacataaaaaagatcatcattcgcgcagaggtttgcttcgtatggtgtcgcagcgtagaaaacttttagattatctgaaacggaaagatgtggcacgctatacgcagttaatagaacgactgggtctgcgtcgc

SEQ ID NO: 162 - DNA, 414 bp, synthetic construct
  source 1..414: mol_type = other DNA
atgtctctgtcaacggaagcaaccgcaaagattgtatcagagtttggcagagacgcgaacgatacggggagtacggaagtgcaggtcgccctgctcaccgcacaaattaaccaccttcagggacatttcgctgaacataaaaaagatcatcattcgcgcagaggtttgcttcgtatggtgtcgcagcgtagaaaacttttagattatctgaaacggaaagatgtggcacgctatacgcagttaatagaacgactgggtctgcgtcgcggtggcggcggctctggtggtggcggcagcggtggcggcggcagttatccatatgatgttccagactatgcaggcagcggcagccctaagaagaagcgaaaagtcgacggaagccctaagaagaagcgcaaggtcgacagtggatga

SEQ ID NO: 163 - DNA, 471 bp, synthetic construct
  source 1..471: mol_type = other DNA
ggcgccaccaacttctccctgctgaagcaggccggcgacgtggaggagaaccccggcccctctctgtcaacggaagcaaccgcaaagattgtatcagagtttggcagagacgcgaacgatacggggagtacggaagtgcaggtcgccctgctcaccgcacaaattaaccaccttcagggacatttcgctgaacataaaaaagatcatcattcgcgcagaggtttgcttcgtatggtgtcgcagcgtagaaaacttttagattatctgaaacggaaagatgtggcacgctatacgcagttaatagaacgactgggtctgcgtcgcggtggcggcggctctggtggtggcggcagcggtggcggcggcagttatccatatgatgttccagactatgcaggcagcggcagccctaagaagaagcgaaaagtcgacggaagccctaagaagaagcgcaaggtcgacagtggatga

SEQ ID NO: 164 - DNA, 2514 bp, synthetic construct
  source 1..2514: mol_type = other DNA
tttacagagaaacctcctcacacaaaaggcgtagtgtacattaacagattatttgtcatcggtaacaaattgttgtcatcttaacaaaatatttgtcatcaataacatattatgtgtcgtgtgcttattactgaaactaatcctagacgatggtaaaaaatcctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagaatgaacaaaaatgtctgattattacataattgtttatttaatataattgtatcgtaatacttgaagtttggagacaagtaatttgttaatactgctccagtccctaaaaaagtgccattcgggtaaatgacacttaatctgttaatttactggaaaatgacagttaatttgttaatatagtaagcaataacttttgtcaaagattaatgctataattcagctaaagcagtgattatataaagctttcactctcaaatagttcggcgacacgattttgttaagacgacaaataattagttactgtaca

SEQ ID NO: 165 - RNA, 295 nt, synthetic construct
  source 1..295: mol_type = other RNA
gaaataatcgcgccgtagatcatgttcttgattgaacctctgaactacgaaaaatgagggttagtttgactctcggcagatagtcttgctttctgaccctagtggctgtccaccctgatgctgatttctacaatttaggttgtagagatgattaacctgtaacttgaggttagctaataatttcatcttatagggtaggtgcgctcccagcaataagtggcgtgggtttaccacagtgacggctactgaatcacctccgaccaaggaggaatccactgaaaagatggattgaaag

SEQ ID NO: 166 - DNA, 633 bp, synthetic construct
  source 1..633: mol_type = other DNA
tgactcgagcttattccagatgcgtgcggatggaattcgagctcggtaccatgccaaaagcaaagcgctatcgcgccttacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataataac

SEQ ID NO: 167 - protein, 129 aa, synthetic construct
MKRPAATKKAGQAKKKKYPYDVPDYAGGGGSGGGGSGGGGSSLSTEATAKIVSEFGRDANDTGSTEVQVALLTAQINHLQGHFAEHKKDHHSRRGLLRMVSQRRKLLDYLKRKDVARYTQLIERLGLRR

SEQ ID NO: 168 - protein, 137 aa, synthetic construct
MSLSTEATAKIVSEFGRDANDTGSTEVQVALLTAQINHLQGHFAEHKKDHHSRRGLLRMVSQRRKLLDYLKRKDVARYTQLIERLGLRRGGGGSGGGGSGGGGSYPYDVPDYAGSGSPKKKRKVDGSPKKKRKVDSG

SEQ ID NO: 169 - protein, 156 aa, synthetic construct
GATNFSLLKQAGDVEENPGPSLSTEATAKIVSEFGRDANDTGSTEVQVALLTAQINHLQGHFAEHKKDHHSRRGLLRMVSQRRKLLDYLKRKDVARYTQLIERLGLRRGGGGSGGGGSGGGGSYPYDVPDYAGSGSPKKKRKVDGSPKKKRKVDSG

SEQ ID NO: 170 - DNA, 1305 bp, synthetic construct
  source 1..1305: mol_type = other DNA
aactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcaaaggaggtaccgcgatatctacctcgag

SEQ ID NO: 171 - DNA, 288 bp, synthetic construct
  source 1..288: mol_type = other DNA
caacctacatcctcaatcccaattaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 172 - protein, 7 aa, synthetic construct
PKKKRKV

SEQ ID NO: 173 - protein, 16 aa, synthetic construct
KRPAATKKAGQAKKKK

SEQ ID NO: 174 - protein, 9 aa, synthetic construct
PAAKRVKLD

SEQ ID NO: 175 - protein, 11 aa, synthetic construct
RQRRNELKRSP

SEQ ID NO: 176 - protein, 38 aa, synthetic construct
NQSSNFGPMKGGNFGGRSSGPYGGGGQYFAKPRNQGGY

SEQ ID NO: 177 - protein, 42 aa, synthetic construct
RMRIZFKNKGKDTAELRRRRVEVSVELRKAKKDEQILKRRNV

SEQ ID NO: 178 - protein, 8 aa, synthetic construct
VSRKRPRP

SEQ ID NO: 179 - protein, 8 aa, synthetic construct
PPKKARED

SEQ ID NO: 180 - protein, 8 aa, synthetic construct
PQPKKKPL

SEQ ID NO: 181 - protein, 12 aa, synthetic construct
SALIKKKKKMAP

SEQ ID NO: 182 - protein, 5 aa, synthetic construct
DRLRR

SEQ ID NO: 183 - protein, 7 aa, synthetic construct
PKQKKRK

SEQ ID NO: 184 - protein, 10 aa, synthetic construct
RKLKKKIKKL

SEQ ID NO: 185 - protein, 10 aa, synthetic construct
REKKKFLKRR

SEQ ID NO: 186 - protein, 20 aa, synthetic construct
KRKGDEVDGVDEVAKKKSKK

SEQ ID NO: 187 - protein, 17 aa, synthetic construct
RKCLQAGMNLEARKTKK

SEQ ID NO: 188 - DNA, 3700 bp, synthetic construct
  source 1..3700: mol_type = other DNA
gaaataattttgtttaactttaagaaggagatatacatatggctagcgcatggagtcatcctcaattcgagaaaggtggaggttctggcggtggatcgggaggttcagcgtggagccacccgcagttcgaaaaaggtggaggttctcttgaagtcctctttcagggacccatgtcccagatcactattcaatgccgacttgtggccaaggaacctattcgtcatacgctttggcaactgatggccgaccttaacactcccttcataaatgagttgttacagaaagtagcacaacacccagatttcgagaagtggaaacaacgtgggcgcctcaaggtaaaggtaattgaacagttgggaaacgagctgaagaaggatcctagattcttaggtcaacctgcccgtttctacacatcgggcatcaacttggtcaagtacatcttcaagtcctggctgaaattacaacaacgtcttcagcagaagctcgaccgaaagcggcgctggttggaagtcctcaagtcagacgaccagctcatcaaggacggacagactgacctggagactatacgccaaaaggccacggagatcctgcaaagttatgagggaactgaacagcttttcaacacactgttccaagcatacaactccgaggaagacatcctgacgcggacagcacttaactatctgttaaagaacagatgcaaattgcctcaaaagcccgaagatgctaagaagttcgccaagcgccgcagacaagtcgagatcgccataaagagattgcaggagcaaataaaggctagattaccgcaggggcgtgacgtgacaaacgagaactggctggagacgctgaacctcgcatgctacactgatcctgaaaacatcgaagaagctcgttcgtggcaggacaagttattaaccaaaagcagttcaatcccattccctataaattacgagacgaatgaggatctgatatggagcaagaatgagaagggtcacttgtgtgtacagtttaatgggatctcagacctgaagtttaagatatattgtgacaaaagacagctcaagtggtttcaacgcttctacgaagaccaacaaattaagaagagcaataataaccagcatagttcggctctgttcacgctgcgctccggacggatactgtggcaggaagataagggcaagggacagctctgggatatacatagactgacattacaatgtacgcttgacacacggacatggacacaagagggaacggagcaggtaaaagaagaaaaggccgacgaaattgccggtatacttacgcggatgaacgagaagggcgacctgacgaagaaccaacaagccttcatacaaagaaagcagtctactctggacaagttggaaaatccattccctcgcccttcgcgaccggtataccgtggtcagagcaacatcttacttggtgtttcgatggaattgaagaaacccgcaactattgcggttatcgatggtatgacccgtaaagtgctgacatatcgtaacataaagcagttattgggtaagaattaccctctcttgaatcgccaacgtcggcagaaacaactgcaaagccaccagcgcaatgtggcccaacgaaaagaggctttcaaccaattcggagacagcgaactgggagagtatatagaccgtctgttagccaaagcgatcatagcaatcgccaaacaatatcaagctcgttctattgtggtgccccacctgaaagacattagagaggccatccaaagcgaaatacaggcactggctgaggccaagatacctaactgtattgaggcacaagccgaatacgccaagaagtaccgtattcaagttcatcaatggtcctacggtcgcctgatcgacaacatccaagcccaagcaagcaagctcggtattgttatcgaagagtcacaacagccgcttcagggtaccccgctccaaaaggctgcagagctggcgttcaaggcttatcaaagtcggctttctgggagcggttcccccaagaagaaacgcaaggtagacggttctcctaagaagaaacgcaaggttgacagcggatgaatgaccattcaagaagcacaagcggttgccaaacagttaggggatattcaattaacctcagagaagttacaggcagaaattcagcgattaaaccgaaaaaccgttgttaccctttcccatgtagaagcattgcataattggttagaaggaaagcgtcaggctaaacaatcttgtcgggtggtgggggagtcacgcaccggaaaaaccattgcttgtaatgcttatcgtttgagacataaacccattcaaacccctggcaaaccgcctatcgttccagtggtgtatattcaagtaactcaagaatgtggggctaaagatttatttggggcaattattgagcatttaaagtatcaaatgaccaaaggaactgttgcagaaatccgtcaacgcacctttaaagtgttgcaacggtgtggcgtagaaatgctgattattgatgaagcggataggttgaaaccgaagacgtttgcagaagttagagatatttttgataagttaaatattgcagtggtattagtgggaacggatcgcttagatgcggtgattaagcgagatgagcaagtttataaccgttttcgggcttgtcatcgctttggtaagttagcaggggatgagtttagtcaaacggtgaatatttgggaacgtcaagtgttgaagttgcctgtagcgtctaatttaagcagtaagcgaatgttaaagattttgggacaagctacggggggttatcttggcttattggatatgattttacgagaatctgctattagagcgttgaaaaaagggttacagaagattgatttagacactttgaaggaagtcacggaggagtaccgataatggaaagtcgagagattcaaccttggtggttcctcgttgagcccttggctggtgagtctatttctcacttcttgggtcgattcagacgcgagaacgaacttaccgttaccatgatgggtaagattaccggactgggtggaacgatcacacgttgggaaaagtttcgattcataccgatcccaacggaagaagaacttacggcactgtcagaagtagttcaagttgaggttgagcgcctgtggcagatgttcccacccaagggtgtaggtatgaaacaccagcctatccgactgtgtggagcgtgctacgaagaggagcgctgccacaaaatagagtggcaactgaaaactacccaattctgttcccaacacggcctcactttactgagcgaatgcccgaactgcggcgctcgcttccaattcccagcactgtgggtaaacggctggtgccacagatgcttcttgacgttcggagaaatggtagagggacaaagtaacaagaaaaaatatttatgaatgtcacttagcacagaggcgactgctaaaatcgttagcgaattcggacgagatgctaatgacactggttccacagaggttcaagttgcacttttgacagcccagatcaatcatctgcaaggtcactttgcggagcacaagaaggaccaccacagtcggcgcgggctgctgcgcatggtttctcaacgccgcaagctgctggactacctcaagcgtaaggacgtcgcgcgttacactcaattgatcgagcggcttggattgcgacgttaa

SEQ ID NO: 189 - protein, 89 aa, unidentified
  source 1..89: note = MG190-178 ribosomal protein S15
MALTQQRKQEIISGFQVHETDTGSADVQIAMLTDRINRLSKHLQANKKDYSSRRGLLKMIGQRKRLLAYVQENSREKYQQLISRLGIRG

SEQ ID NO: 190 - protein, 89 aa, unidentified
  source 1..89: note = MG190-179 ribosomal protein S15
MSLTASEKQELMSSYQVHETDTGSPDLQVALLTKRISQLTGHLQKNPKDHASRRGLLKMIGRRRRLLAYINSKDTNRYKALIKSLGIRR

SEQ ID NO: 191 - protein, 89 aa, unidentified
  source 1..89: note = MG190-180 ribosomal protein S15
MALIQERKQELISEYQVHETDTGSAEVQVAMLTERINKLSQHLRENKKDYSSRRGLLKMIGRRKRLLSYISKKDVERYRELIGRLGIRR

SEQ ID NO: 192 - protein, 89 aa, unidentified
  source 1..89: note = MG190-181 ribosomal protein S15
MSITQERKQELISEYQVHGTDTGSSDVQVAILSDRINSLTQHLKVNKKDHASRLGLLKLIGRRRRLLTYIQQHDYEHYQQLIRRLGIRR

SEQ ID NO: 193 - protein, 89 aa, unidentified
  source 1..89: note = MG190-182 ribosomal protein S15
MSLTANEKQSLISEYQVHETDTGSADLQVAILTKRITQLTDHLKQNPKDHASRRGLLKMIGRRRRLLAYINGKDTNRYQALIKRLGIRR

SEQ ID NO: 194 - protein, 89 aa, unidentified
  source 1..89: note = MG190-183 ribosomal protein S15
MALLQERKQELISEYQVHETDTGSAEVQVAMLTERINKLSQHLQGNKKDYSSRRGLLKMIGRRKRLLSYIAKKDVNQYRALIGRLGIRR

SEQ ID NO: 195 - protein, 89 aa, unidentified
  source 1..89: note = MG190-184 ribosomal protein S15
MTLLQERKQELISEYQIHETDTGSVDLQIAMLTERINRLSAHLQKNKKDYSSRRGLLKMIGQRKRLMAYLLKQDTERYRNLIQKLGIRG

SEQ ID NO: 196 - protein, 89 aa, unidentified
  source 1..89: note = MG190-185 ribosomal protein S15
MALLQERKQELISEYQVHETYTGSAEVHVAMLTERINKLSQHLQSNKKDYSSRRGLLKMIGRRKRLLSYIAKKDVNQYRELIGRLGIRR

SEQ ID NO: 197 - protein, 89 aa, unidentified
  source 1..89: note = MG190-186 ribosomal protein S15
MTLLQERKQEIMSEYQIHETDTGSVDLQIAMLTERINRLSAHLQKNKKDYSSRRGLLKMIGQRKRLMAYLLKQDTDRYRDLIKKLGIRG

SEQ ID NO: 198 - protein, 89 aa, unidentified
  source 1..89: note = MG190-187 ribosomal protein S15
MALLQERKQELISEYQVHETDTGSAEVQVAMLTERINKLSQHLQSNKKDYSSRRGLLKMIGRRKRLLSYIAKKDVNQYRELIGRLGIRR

SEQ ID NO: 199 - protein, 89 aa, unidentified
  source 1..89: note = MG190-188 ribosomal protein S15
MALTQEDKQQIMAEHQAHETDTGSSDVQVAMLTERINRLSAHLKANKKDHSSRRGLLQMIGRRKRLLAYIYKQDQQRYRALITRLGIRG

SEQ ID NO: 200 - protein, 614 aa, unidentified
  source 1..614: note = MG64-128 effector
MSQKTIRCRLIASQKSRKAIWKLMAERNTPLVNEVLRQLPEHPDFAKWQQKGNLPDVAVKRIIDALKSDPHFSDQPFWYYTSAQKQVTYTFKSWLSIQRRKQWRLQGKRWWLEILLPDAELAKLVKCSVEKLRTEAAKILAKVGDVDPFKHLLEQYRHEQKLLRKCAIAFLLKRNTEIDREEDLEQLKQRSRRVELQIRRLEIQLQASLPKGRDLTGERQAAALAQSVLASPDDDESYELWRNTVTREPAQFPFPVICETSEWLKWQRDHNGRISVGFSALSEHVFKIYCDKPHQHWFDRFFEDQETKRTGGKQHSAGLFTLRSAKLTWVPSNKHANASEPWNCYYLNLSCTVDTRLWTQEGTQIVIQEKAAEKAGKLESMRRKENLSKTQQGYIKRLEATLEKLQTPYPRPSRPLYSGKTNILVGVSMGLDKPATVAVVDALAGEVLTYRSVKQLLGNDYRLLKRAQTEKTRIHKNHRRGGRRVSEESNIAQQVDRLLAKSIIDIAREYEASSIVVPDLANIREIVETEVKARAQDKVPDFVEGQKQYAKAYRTQVHQWSYRRLQEAVRTKAEQSGITIEVVRQGLSGTQHEKAKALALQGYEKRIREHVEMA

SEQ ID NO: 201 - RNA, 37 nt, synthetic construct
  source 1..37: mol_type = other RNA
gacgcaatgagcttttaaggctcatgaggattgaaac

SEQ ID NO: 202 - RNA, 265 nt, synthetic construct
  source 1..265: mol_type = other RNA
gtatatttgtacaaaagcgccgcagatcatgcgcaagcctctgttctgtgaaaaatgagggtttgtttgactgtgtgatagcggtcttactttctgaccctggtagctgaccactccgatgctgctgttgaaagtgaacttgttaggacttgggcaccataaaacaggagaggggcgcacccagcaagagagaacggacttactgtagtgttggctgccgaatcaactccgatcaaggagtaatccatgcacccatacccatata

SEQ ID NO: 203 - DNA, 42 bp, synthetic construct
  source 1..42: mol_type = other DNA
gctcttccgatctnnnnngatgagcgcattgttagatttcat

SEQ ID NO: 204 - DNA, 20 bp, synthetic construct
  source 1..20: mol_type = other DNA
aaaccgacatcgcaggcttc

SEQ ID NO: 205 - protein, 33 aa, synthetic construct
KATEILQSYEGTEQLFNTLFQAYNSEEDILTRT

SEQ ID NO: 206 - protein, 22 aa, synthetic construct
RAGQDLSVEYSNHVWQCDHTRA

SEQ ID NO: 207 - protein, 20 aa, synthetic construct
KTVVTLSHVEALHNWLEGKR

SEQ ID NO: 208 - protein, 25 aa, synthetic construct
RFIPIPTEEELTALSEVVQVEVERL

(19) United States
(12) Patent Application Publication
(10) Pub. No.: US 2025/0179484 A1
(43) Pub. Date: Jun. 5, 2025

(54) FUSION PROTEINS

(71) Applicant: Metagenomi, Inc., Emeryville, CA (US)

(72) Inventors: Brian C. Thomas, Berkeley, CA (US); Christopher Brown, Albany, CA (US); Daniela S.A. Goltsman, Oakland, CA (US); Cristina Noel Butterfield, Oakland, CA (US); Lisa Alexander, Albany, CA (US); Jason Liu, Berkeley, CA (US); Gregory J. Cost, Piedmont, CA (US); Khak Khak Khayi, Oakland, CA (US)

(21) Appl. No.: 18/840,719

(22) PCT Filed: Feb. 23, 2023
(86) PCT No.: PCT/US2023/063182

§ 371 Date: Aug. 22, 2024

Related U.S. Application Data
(60) Provisional application No. 63/478,690, filed on Jan. 5, 2023.
(60) Provisional application No. 63/313,151, filed on Feb. 23, 2022.

(51) Int. Cl. C12N 15/11; C12N 9/12; C12N 9/22; C12N 15/90

(52) U.S. Cl. CPC C12N 15/111; C12N 9/1241; C12N 9/22; C12N 15/90; C07K 2319/80; C12N 2310/20

(57) ABSTRACT

The present disclosure provides systems and methods for transposing a cargo nucleotide sequence into a target nucleic acid site. These systems and methods may comprise a double-stranded nucleic acid comprising the cargo nucleotide sequence, wherein the cargo nucleotide sequence is configured to interact with a recombinase complex, an effector complex comprising an effector and at least one engineered guide polynucleotide configured to hybridize to the target nucleic acid site, and the recombinase complex wherein said recombinase complex is configured to recruit the cargo nucleotide to the target nucleic acid site.

### CROSS-REFERENCE

This application is the U.S. National Stage entry of International Application No. PCT/US2023/063182, filed Feb. 23, 2023, which claims the benefit of and priority to U.S. Provisional Patent Application No. 63/313,151, filed Feb. 23, 2022, and U.S. Provisional Patent Application No. 63/478,690, filed Jan. 5, 2023, each of which is incorporated by reference in its entirety herein.

### SEQUENCE LISTING

The contents of the electronic sequence listing (MTG-009WOUS_SL.xml; Size: 254,866 bytes; and Date of Creation: Apr. 4, 2023) is herein incorporated by reference in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for a fusion protein comprising: (a) class 2, type V Cas effector; and (b) a functional domain comprising a DNA Binding domain (DBD) or a chromatin modulating domain (CMD). In some embodiments, said functional domain is derived from a Human histone 1 central globular domain, HMGN1, cbx5, or Saccharolobus solfataricus sso7d. In some embodiments, said Cas effector is derived from a CAST locus. In some embodiments, said Cas effector comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to a Cas domain of any one of SEQ ID NOs: 113-116, or a variant thereof. In some embodiments, said functional domain comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 109-112, or a variant thereof. In some embodiments, said fusion protein comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs:113-116, or a variant thereof.

In some aspects, the present disclosure provides for a fusion protein comprising: (a) a TniQ protein; and (b) a functional domain comprising a DNA Binding domain (DBD) or a chromatin modulating domain (CMD). In some embodiments, said TniQ protein is derived from a CAST locus. In some embodiments, said TniQ protein comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to a TniQ domain of any one of SEQ ID NOs: 117-120, or a variant thereof. In some embodiments, said functional domain comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 109-112, or a variant thereof. In some embodiments, said fusion protein comprises a sequence having at least 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 117-120, or a variant thereof.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises a TnsB subunit. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the target nucleic acid comprises a PAM sequence compatible with said Cas effector complex adjacent to said target nucleic acid site. In some embodiments, said PAM sequence is located 3′ of said target nucleic acid site. In some embodiments, said PAM sequence is located 5′ of said target nucleic acid site. In some embodiments, said engineered guide polynucleotide is configured to bind said class 2, type V Cas effector. In some embodiments, said class 2, type V Cas effector comprises a polypeptide comprising a sequence having at least 80% identity to SEQ ID NO: 1, 12, 16, 20-30, 64, 80-85, and 200, or a variant thereof. In some embodiments, said TnsB subunit comprises a polypeptide having a sequence having at least 80% identity to SEQ ID NO: 2, 13, 17, or 65, or a variant thereof. In some embodiments, said Tn7 type transposase complex comprises at least one or at least two three polypeptide(s) comprising a sequence having at least 80% identity to any one of SEQ ID NOs: 3-4, 14-15, 18-19, or 66-67, or a variant thereof. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% sequence identity to non-degenerate nucleotides of any one of SEQ ID NOs: 106, 107, 108, 5, 45-63, 68-75, or 96-103, or a variant thereof. In some embodiments, said left-hand recombinase sequence comprises a sequence having at least 80% identity to SEQ ID NO: 9, 11, 36-38, 76, or 78, or a variant thereof. In some embodiments, said right-hand recombinase sequence comprises a sequence having at least 80% identity to SEQ ID NO: 8, 10, 39-44, 77, 79, or 93, or a variant thereof. In some embodiments, said class 2, type V Cas effector and said Tn7 type transposase complex are encoded by polynucleotide sequences comprising fewer than about 10 kilobases.

In some aspects, the present disclosure provides for a method for transposing a cargo nucleotide sequence into a target nucleic acid site comprising a target nucleotide sequence comprising expressing the system of any of the aspects or embodiments described herein within a cell or introducing the system of any of the aspects or embodiments described herein to a cell.

In some aspects, the present disclosure provides for a method for transposing a cargo nucleotide sequence into a target nucleic acid site, comprising contacting a first double-stranded nucleic acid comprising said cargo nucleotide sequence with: a Cas effector complex comprising a class 2, type V Cas effector and at least one engineered guide polynucleotide configured to hybridize to said target nucleotide sequence; a Tn7 type transposase complex configured to bind said Cas effector complex, wherein said Tn7 type transposase complex comprises a TnsB subunit; and a second double-stranded nucleic acid comprising said target nucleic acid site. In some embodiments, said cargo nucleotide sequence is flanked by a left-hand transposase recognition sequence and a right-hand transposase recognition sequence. In some embodiments, the target nucleic acid comprises a PAM sequence compatible with said Cas effector complex adjacent to said target nucleic acid site. In some embodiments, said PAM sequence is located 3′ of said target nucleic acid site. In some embodiments, said engineered guide polynucleotide is configured to bind said class 2, type V Cas effector. In some embodiments, said class 2, type V Cas effector comprises a polypeptide comprising a sequence having at least 80% identity to SEQ ID NO: 1, 12, 16, 20-30, 64, 80-85, and 200, or a variant thereof. In some embodiments, said TnsB subunit comprises a polypeptide having a sequence having at least 80% identity to SEQ ID NO: 2, 13, 17, or 65, or a variant thereof. In some embodiments, said Tn7 type transposase complex comprises at least one or at least two polypeptide(s) comprising a sequence having at least 80% identity to any one of SEQ ID NOs: 3-4, 14-15, 18-19, or 66-67, or a variant thereof. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said left-hand recombinase sequence comprises a sequence having at least 80% identity to SEQ ID NO: 9, 11, 36-38, 76, or 78, or a variant thereof. In some embodiments, said right-hand recombinase sequence comprises a sequence having at least 80% identity to SEQ ID NO: 8, 10, 39-44, 77, 79, or 93, or a variant thereof. In some embodiments, said class 2, type V Cas effector and said Tn7 type transposase complex are encoded by polynucleotide sequences comprising fewer than about 10 kilobases.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises TnsB, TnsC, and TniQ components, wherein: (a) said class 2, type V Cas effector comprises a polypeptide having a sequence having at least 80% sequence identity to any one of SEQ ID NO: 1, 12, 16, 20-30, 64, 80-85, and 200, or a variant thereof; or (b) said Tn7 type transposase complex comprises a TnsB, TnsC, or TniQ component having a sequence having at least 80% sequence identity to any one of SEQ ID NOs: 2-4, 13-15, 17-19, and 65-67, or a variant thereof. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class 2, type V Cas effector comprises a polypeptide having a sequence having at least 80% sequence identity to any one of SEQ ID NO: 1, 12, 16, 20-30, 64, 80-85, and 200, or a variant thereof. In some embodiments, said Tn7 type transposase complex comprises a TnsB, TnsC, or TniQ component having a sequence having at least 80% sequence identity to any one of SEQ ID NOs: 2-4, 13-15, 17-19, and 65-67, or a variant thereof. In some embodiments, said class 2, type V Cas effector is a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the target nucleic acid comprises a PAM sequence compatible with said Cas effector complex adjacent to said target nucleic acid site. In some embodiments, said PAM sequence is located 5′ or 3′ of said target nucleic acid site. In some embodiments, said PAM sequence comprises GTN. In some embodiments, said engineered guide polynucleotide is configured to bind said class 2, type V Cas effector. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% sequence identity to non-degenerate nucleotides of any one of SEQ ID NOs: 106, 107, 108, 5, 45-63, 68-75, or 96-103, or a variant thereof. In some embodiments, said left-hand recombinase sequence comprises a sequence having at least 80% identity to any one of SEQ ID NOs: 9, 11, 36-38, 76, or 78, or a variant thereof. In some embodiments, said right-hand recombinase sequence comprises a sequence having at least 80% identity to any one of SEQ ID NO: 8, 10, 39-44, 77, 79, or 93. In some embodiments, said class 2, type V Cas effector and said Tn7 type transposase complex are encoded by polynucleotide sequences comprising fewer than about 10 kilobases. In some embodiments: (a) said class 2, type V Cas effector comprises a sequence having at least 80% sequence identity to any one of SEQ ID NOs:1, 81, 82, 83, or 85, or a variant thereof; (b) said left-hand recombinase sequence comprises a sequence having at least 80% sequence identity to any one of SEQ ID NOs: 9, 11, 36, 37, or 38, or a variant thereof; (c) said right-hand recombinase sequence comprises a sequence having at least 80% identity to any one of SEQ ID NOs: 8, 39, 40, 41, 42, 43, 44, or 93, or a variant thereof; (d) said engineered guide polynucleotide: (i) comprises a sequence having at least 80% sequence identity to at least about 46-80 nucleotides of SEQ ID NO: 6, or a variant thereof; or (ii) comprises a sequence having at least 80% identity to the non-degenerate nucleotides of any one of SEQ ID NO: 5, 45-63, 68-75, or 96-103, or a variant thereof; (e) said TnsB, TnsC, and TniQ components comprise polypeptides having a sequence having at least 80% identity to SEQ ID NO: 2-4, or variants thereof; or (f) said PAM sequence comprises GTN. In some embodiments: (a) said class 2, type V Cas effector comprises a sequence having at least 80% sequence identity to SEQ ID NO:12, or a variant thereof; (b) said left-hand recombinase sequence comprises a sequence having at least 80% sequence identity to SEQ ID NO:76, or a variant thereof; (c) said right-hand recombinase sequence comprises a sequence having at least 80% identity to SEQ ID NO:77, or a variant thereof; (d) said engineered guide polynucleotide: (i) comprises a sequence having at least 80% sequence identity to at least about 46-80 nucleotides of SEQ ID NO: 32 or 104, or a variant thereof; or (ii) comprises a sequence having at least 80% identity to the non-degenerate nucleotides of any one of SEQ ID NO: 107 or 102, or a variant thereof; or (e) said TnsB, TnsC, and TniQ components comprise polypeptides having a sequence having at least 80% identity SEQ ID NO:13-15, or variants thereof. In some embodiments: (a) said class 2, type V Cas effector comprises a sequence having at least 80% sequence identity to SEQ ID NO:16, or a variant thereof; (b) said left-hand recombinase sequence comprises a sequence having at least 80% sequence identity to SEQ ID NO:78, or a variant thereof; (c) said right-hand recombinase sequence comprises a sequence having at least 80% identity to SEQ ID NO:79, or a variant thereof; (d) said engineered guide polynucleotide: (i) comprises a sequence having at least 80% sequence identity to at least about 46-80 nucleotides of SEQ ID NO: 33 or 105, or a variant thereof; or (ii) comprises a sequence having at least 80% identity to the non-degenerate nucleotides of any one of SEQ ID NO: 108 or 103, or a variant thereof; or (e) said TnsB, TnsC, and TniQ components comprise polypeptides having a sequence having at least 80% identity SEQ ID NO: 17-19, or variants thereof.

In some aspects, the present disclosure provides for an engineered nuclease system comprising: an endonuclease comprising a RuvC domain, wherein said endonuclease is derived from an uncultivated microorganism, and wherein said endonuclease is a Class 2, type V-K Cas effector having at least 80% identity to any one SEQ ID NO: 1, 12, 16, 20-30, 64, 80-85, and 200, or a variant thereof; and an engineered guide RNA, wherein said engineered guide RNA is configured to form a complex with said endonuclease and said engineered guide RNA comprises a spacer sequence configured to hybridize into a target nucleic acid sequence. In some embodiments, said engineered guide polynucleotide comprises a sequence comprising at least about 46-80 consecutive nucleotides having at least 80% identity to any one of SEQ ID NOs: 5-6, 32-33, 94-95, or 104-105, or a variant thereof. In some embodiments, said engineered guide polynucleotide comprises a sequence having at least 80% identity to non-degenerate nucleotides of any one of SEQ ID NOs: 106, 107, 108, 5, 45-63, 68-75, or 96-103, or a variant thereof. In some embodiments, the target nucleic acid comprises a PAM sequence compatible with said Cas effector complex adjacent to said target nucleic acid site. In some embodiments, said PAM sequence is located 5′ of said target nucleic acid site. In some embodiments, said PAM sequence comprises GTN. In some embodiments: (a) said class 2, type V-K Cas effector comprises a sequence having at least 80% sequence identity to any one of SEQ ID NOs:1, 81, 82, 83, or 85, or a variant thereof; (b) said left-hand recombinase sequence comprises a sequence having at least 80% sequence identity to any one of SEQ ID NOs: 9, 11, 36, 37, or 38, or a variant thereof; (c) said right-hand recombinase sequence comprises a sequence having at least 80% identity to any one of SEQ ID NOs: 8, 39, 40, 41, 42, 43, 44, or 93, or a variant thereof; (d) said engineered guide polynucleotide: (i) comprises a sequence having at least 80% sequence identity to at least about 46-80 nucleotides of SEQ ID NO: 6, or a variant thereof; or (ii) comprises a sequence having at least 80% identity to the non-degenerate nucleotides of any one of SEQ ID NO: 5, 45-63, 68-75, or 96-103, or a variant thereof; (e) said TnsB, TnsC, and TniQ components comprise polypeptides having a sequence having at least 80% identity to SEQ ID NO: 2-4, or variants thereof; or (f) said PAM sequence comprises GTN.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In some aspects, the present disclosure provides for a system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide configured to hybridize to the target nucleic acid site; a Tn7 type transposase complex configured to bind the Cas effector complex and comprising a TnsB, TnsC, and TniQ component; a double-stranded nucleic acid configured to interact with the Tn7 type transposase complex and comprising the cargo nucleotide sequence; and a functional domain comprising a DNA Binding domain (DBD) or a chromatin modulating domain (CMD).

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex.

In some embodiments, the cargo nucleotide sequence is flanked by a left-hand transposase recognition sequence and a right-hand transposase recognition sequence recognized by the Tn7 type transposase complex. In some embodiments, the left-hand recombinase sequence comprises a sequence having at least 80% identity to any one of SEQ ID NOs: 9, 11, 36-38, 76, and 78. In some embodiments, the right-hand recombinase sequence comprises a sequence having at least 80% identity to any one of SEQ ID NOs: 8, 10, 39-44, 77, 79, and 93.

In some embodiments, the target nucleic acid comprises a PAM sequence compatible with the Cas effector complex. In some embodiments, the PAM sequence comprises GTN. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 80% identity to any one of SEQ ID NOs: 1, 12, 16, 20-30, 64, 80-85, and 200. In some embodiments, the class 2, type V Cas effector comprises a polypeptide comprising a sequence having at least 90% identity to any one of SEQ ID NOs: 1, 12, 16, 20-30, 64, 80-85, and 200. In some embodiments, the class 2, type V Cas effector comprises a polypeptide comprising a sequence of any one of SEQ ID NOs: 1, 12, 16, 20-30, 64, 80-85, and 200.

In some embodiments, the TnsB component comprises a polypeptide having a sequence having at least 80% identity to any one of SEQ ID NOs: 2, 13, 17, and 65. In some embodiments, the TnsB component comprises a polypeptide having a sequence having at least 90% identity to any one of SEQ ID NOs: 2, 13, 17, and 65. In some embodiments, the TnsB component comprises a polypeptide having a sequence of any one of SEQ ID NOs: 2, 13, 17, and 65. In some embodiments, the Tn7 type transposase complex comprises at least a first polypeptide and a second polypeptide each independently comprising a sequence having at least 80% identity to any one of SEQ ID NOs: 3-4, 14-15, 18-19, and 66-67. In some embodiments, the Tn7 type transposase complex comprises at least a first polypeptide and a second polypeptide each independently comprising a sequence having at least 90% identity to any one of SEQ ID NOs: 3-4, 14-15, 18-19, and 66-67. In some embodiments, the Tn7 type transposase complex comprises at least a first polypeptide and a second polypeptide each independently comprising a sequence of any one of SEQ ID NOs: 3-4, 14-15, 18-19, and 66-67.

In some embodiments, the engineered guide polynucleotide comprises a sequence comprising at least about 46-80 consecutive nucleotides having at least 80% identity to any one of SEQ ID NOs: 5-6, 32-33, 94-95, 104-105, and 202. In some embodiments, the engineered guide polynucleotide comprises a sequence having at least 80% sequence identity to any one of SEQ ID NOs: 106, 107, 108, 5, 45-63, 68-75, 96-103, and 165.

In some embodiments, the functional domain is derived from a Human histone 1 central globular domain, HMGN1, cbx5, or Saccharolobus solfataricus sso7d. In some embodiments, the functional domain comprises a sequence having at least 80% sequence identity to any one of SEQ ID NOs: 109-112. In some embodiments, the class 2, type V Cas effector is fused to the functional domain to form a fusion protein. In some embodiments, the fusion protein comprises a sequence having at least 80% identity to any one of SEQ ID NOs: 113-116.

In some embodiments, the Tn7 transposase complex comprises a TniQ protein. In some embodiments, the TniQ protein is fused to the functional domain to form a fusion protein. In some embodiments, the TniQ protein comprises a sequence having at least 80% sequence identity to a TniQ domain of any one of SEQ ID NOs: 117-120.

In some embodiments, the small prokaryotic ribosomal protein subunit S15 comprises a sequence having at least 80% sequence identity to any one of SEQ ID NOs: 167-169. In some embodiments, the small prokaryotic ribosomal protein subunit S15 is encoded by a sequence having at least 80% sequence identity to any one of SEQ ID NOs: 161-163.

In some embodiments, the class 2, type V Cas effector and the Tn7 type transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex comprising a class 2, type V Cas effector and an engineered guide polynucleotide configured to hybridize to the target nucleic acid site, wherein the Cas effector complex comprises a polypeptide comprising a sequence having at least 80% sequence identity to any one of SEQ ID NOs: 1, 12, 16, 20-30, 64, 80-85, and 200; a Tn7 type transposase complex configured to bind the Cas effector complex and comprising a TnsB, TnsC, and TniQ component, the TnsB, TnsC, or TniQ component comprising a sequence having at least 80% sequence identity to any one of SEQ ID NOs: 2-4, 13-15, 17-19, and 65-67; a double-stranded nucleic acid configured to interact with the Tn7 type transposase complex and comprising the cargo nucleotide sequence; and a functional domain comprising a DNA Binding domain (DBD) or a chromatin modulating domain (CMD).

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex.

In some embodiments, the cargo nucleotide sequence is flanked by a left-hand transposase recognition sequence and a right-hand transposase recognition sequence recognized by the Tn7 type transposase complex. In some embodiments, the left-hand recombinase sequence comprises a sequence having at least 80% identity to any one of SEQ ID NOs: 9, 11, 36-38, 76, and 78. In some embodiments, the right-hand recombinase sequence comprises a sequence having at least 80% identity to any one of SEQ ID NOs: 8, 10, 39-44, 77, 79, and 93.

In some embodiments, the target nucleic acid comprises a PAM sequence compatible with the Cas effector complex. In some embodiments, the PAM sequence comprises GTN.

In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 90% identity to any one of SEQ ID NOs: 1, 12, 16, 20-30, 64, 80-85, and 200. In some embodiments, the class 2, type V Cas effector comprises a polypeptide comprising a sequence of any one of SEQ ID Nos: 1, 12, 16, 20-30, 64, 80-85, and 200.

In some embodiments, the TnsB, TnsC, or TniQ component comprises a sequence having at least 90% sequence identity to any one of SEQ ID NOs: 2-4, 13-15, 17-19, and 65-67.

In some embodiments, the TnsB, TnsC, or TniQ component comprises a sequence of any one of SEQ ID NOs: 2-4, 13-15, 17-19, and 65-67. In some embodiments, the engineered guide polynucleotide comprises a sequence comprising at least about 46-80 consecutive nucleotides having at least 80% identity to any one of SEQ ID NOs: 5-6, 32-33, 94-95, 104-105, and 202.

In some embodiments, the engineered guide polynucleotide comprises a sequence having at least 80% sequence identity to any one of SEQ ID NOs: 106, 107, 108, 5, 45-63, 68-75, 96-103, and 165.

In some embodiments, the functional domain is derived from a Human histone 1 central globular domain, HMGN1, cbx5, or Saccharolobus solfataricus sso7d. In some embodiments, the functional domain comprises a sequence having at least 80% sequence identity to any one of SEQ ID NOs: 109-112. In some embodiments, the class 2, type V Cas effector is fused to the functional domain to form a fusion protein. In some embodiments, the fusion protein comprises a sequence having at least 80% identity to any one of SEQ ID NOs: 113-116.

In some embodiments, the Tn7 transposase complex comprises a TniQ protein. In some embodiments, the TniQ protein is fused to the functional domain to form a fusion protein. In some embodiments, the TniQ protein comprises a sequence having at least 80% sequence identity to a TniQ domain of any one of SEQ ID NOs: 117-120.

In some embodiments, the Cas effector complex further comprises a small prokaryotic ribosomal protein subunit S15. In some embodiments, the small prokaryotic ribosomal protein subunit S15 comprises a sequence having at least 80% sequence identity to any one of SEQ ID NOs: 167-169. In some embodiments, the small prokaryotic ribosomal protein subunit S15 is encoded by a sequence having at least 80% sequence identity to any one of SEQ ID NOs: 161-163.

In some embodiments, the class 2, type V Cas effector and the Tn7 type transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a sequence having at least 80% sequence identity to any one of SEQ ID NOs: 1, 81, 82, 83, and 85; and ii) an engineered guide polynucleotide comprising having at least 80% identity to any one of SEQ ID NOs: 5, 6, 45-63, 68-75, and 96-103; a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 2-4; a double-stranded nucleic acid configured to interact with the Tn7 type transposase complex and comprising in 5′ to 3′ order: a left-hand recombinase sequence comprising a sequence having at least 80% sequence identity to any one of SEQ ID NOs: 9, 11, 36, 37, and 38; the cargo nucleotide sequence; and a right-hand recombinase sequence comprising a sequence having at least 80% identity to any one of SEQ ID NOs: 8, 39-44, and 93; and a functional domain comprising a DNA Binding domain (DBD) or a chromatin modulating domain (CMD).

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a sequence having at least 80% sequence identity to SEQ ID NO: 12; and ii) an engineered guide polynucleotide comprising having at least 80% identity to any one of SEQ ID NOs: 32, 102, 104, and 107; a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 13-15; a double-stranded nucleic acid configured to interact with the Tn7 type transposase complex and comprising in 5′ to 3′ order: a left-hand recombinase sequence comprising a sequence having at least 80% sequence identity to SEQ ID NO: 76; the cargo nucleotide sequence; and a right-hand recombinase sequence comprising a sequence having at least 80% identity to SEQ ID NO: 77; and a functional domain comprising a DNA Binding domain (DBD) or a chromatin modulating domain (CMD).

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a sequence having at least 80% sequence identity to SEQ ID NO: 16; and ii) an engineered guide polynucleotide comprising having at least 80% identity to any one of SEQ ID NOs: 33, 103, 105, and 108; a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 17-19; a double-stranded nucleic acid configured to interact with the Tn7 type transposase complex and comprising in 5′ to 3′ order: a left-hand recombinase sequence comprising a sequence having at least 80% sequence identity to SEQ ID NO: 78; the cargo nucleotide sequence; and a right-hand recombinase sequence comprising a sequence having at least 80% identity to SEQ ID NO: 79; and a functional domain comprising a DNA Binding domain (DBD) or a chromatin modulating domain (CMD).

In some embodiments, the target nucleic acid comprises a PAM sequence compatible with the Cas effector complex. In some embodiments, the PAM sequence comprises GTN. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the functional domain is derived from a Human histone 1 central globular domain, HMGN1, cbx5, or Saccharolobus solfataricus sso7d. In some embodiments, the functional domain comprises a sequence having at least 80% sequence identity to any one of SEQ ID NOs: 109-112. In some embodiments, the class 2, type V Cas effector is fused to the functional domain to form a fusion protein. In some embodiments, the fusion protein comprises a sequence having at least 80% identity to any one of SEQ ID NOs: 113-116.

In some embodiments, the Tn7 transposase complex comprises a TniQ protein. In some embodiments, the TniQ protein is fused to the functional domain to form a fusion protein. In some embodiments, the TniQ protein comprises a sequence having at least 80% sequence identity to a TniQ domain of any one of SEQ ID NOs: 117-120.

In some embodiments, the Cas effector complex further comprises a small prokaryotic ribosomal protein subunit S15. In some embodiments, the small prokaryotic ribosomal protein subunit S15 comprises a sequence having at least 80% sequence identity to any one of SEQ ID NOs: 167-169. In some embodiments, the small prokaryotic ribosomal protein subunit S15 is encoded by a sequence having at least 80% sequence identity to any one of SEQ ID NOs: 161-163.

In some aspects, the present disclosure provides for a system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide, the engineered guide polynucleotide capable of hybridizing to the target nucleic acid; a Tn7 type transposase complex operably linked to the Cas effector complex and comprising a TnsB, TnsC, and TniQ component; a double-stranded nucleic acid comprising in 5′ to 3′ order: a left-hand recombinase recognition sequence; the cargo nucleotide sequence; and a right-hand recombinase recognition sequence, the left-hand recombinase recognition sequence and the right-hand recombinase recognition sequence capable of being recognized by the Tn7 type transposase complex; and a functional domain comprising a DNA Binding domain (DBD) or a chromatin modulating domain (CMD).

In some aspects, the present disclosure provides for an engineered nuclease system comprising: an endonuclease comprising a RuvC domain, the endonuclease being derived from an uncultivated microorganism and is a Class 2, type V-K Cas effector comprising at least 80% identity to any one of SEQ ID NOs: 1, 12, 16, 20-30, 64, 80-85, and 200; and an engineered guide RNA configured to form a complex with the endonuclease and comprising a spacer sequence configured to hybridize into a target nucleic acid sequence.

In some embodiments, the engineered guide polynucleotide comprises a sequence comprising at least about 46-80 consecutive nucleotides having at least 80% identity to any one of SEQ ID NOs: 5-6, 32-33, 94-95, 104-105, and 202. In some embodiments, the engineered guide polynucleotide comprises a sequence having at least 80% identity to any one of SEQ ID NOs: 106, 107, 108, 5, 45-63, 68-75, 96-103, and 165.

In some aspects, the present disclosure provides for a method for transposing a cargo nucleotide sequence into a target nucleic acid site comprising introducing a system of the disclosure to a cell.

In some aspects, the present disclosure provides for a cell comprising a system of the disclosure. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell.

In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts typical organizations of CRISPR/Cas loci of different classes and types.

FIG. 2 depicts the architecture of a natural Class 2 Type II crRNA/tracrRNA pair shown e.g., for Cas9, compared to a hybrid sgRNA wherein the crRNA and tracrRNA are joined.

FIG. 3 depicts the two pathways found in Tn7 and Tn7-like elements.

FIGS. 4A-4B depict the genomic context of a Type V Tn7 CAST of the family MG64. FIG. 4A depicts that the MG64-1 CAST system comprises a CRISPR array (CRISPR repeats), a Type V nuclease, and three predicted transposase protein sequences. A tracrRNA was predicted in the intergenic region between the CAST effector and CRISPR array. Bottom: Multiple sequence alignment of the catalytic domain of transposase TnsB. The catalytic residues are indicated by boxes. FIG. 4B depicts that the two transposon ends were predicted for the MG64-1 CAST system.

FIG. 5 depicts predicted structures of corresponding sgRNAs of CAST systems described herein. Panel A of FIG. 5 (left) shows the predicted MG64-1 tracrRNA and crRNA duplex complexes at the repeat-antirepeat stem. Loop was truncated and a tetraloop of GAAA was added to the stem loop structure to produce the designed sgRNA shown in Panel B of FIG. 5 (right).

FIG. 6 depicts the results of a transposition reaction targeted to a plasmid Library comprising NNNNNNNN at the 5′ of the target spacer sequence. Reaction #1 indicates the presence of the target Library, #2 shows presence of Donor fragments in both transposition reactions, #3-5 shows sg specific PCR band that corresponds to proper transposition reactions.

FIGS. 7A-7C depict the results of Sanger sequencing. FIG. 7A shows Sanger sequencing of the donor target junction on the transposon Left End (LE) in LE-closer-to-PAM transposition reactions. Expected sequence is at the top of the panel, with a predicted transposition event 61 bp away from the PAM. Top chromatogram is sequencing result that begins from within the donor fragment. Clear signal is seen on the right end up until the donor/target junction (dotted line). This denotes a mix of transposition products. The bottom chromatogram of the panel is sequencing from the target to the donor/target junction. The signal from the left is clear signal until the point of junction. FIG. 7B shows Sanger sequencing of the donor target junction on the transposon Right End (RE) in LE-closer-to-PAM products. Expected sequence is at the top of the panel, with a predicted transposition event 61 bp away from the PAM. Top chromatogram is sequencing result than begin from within the donor fragment. Clear signal is seen on the left end up until the donor/target junction (dotted line).

FIG. 7C is the SeqLogo analysis on NGS of the LE-closer-to-PAM events which indicates a very strong preference for NGTN in the PAM motif.

FIG. 8 depicts a phylogenetic gene tree of Cas12k effector sequences. The tree was inferred from a multiple sequence alignment of 64 Cas12k sequences recovered here (orange and black branches) and 229 reference Cas12k sequences from public databases (grey branches). Orange branches indicate Cas12k effectors with confirmed association with CAST transposon components.

FIG. 9 shows MG64 family CRISPR repeat alignment. Cas12k CAST CRISPR repeats contain a conserved motif 5′-GNNGGNNTGAAAG-3′. In MG64-1, short repeat-antirepeats (RAR) within the CRISPR repeat motif align with the tracrRNA. MG64 RAR motifs appear to define the start and end of the tracrRNA (5′ end: RAR1 (TTTC); 3′ end: RAR2 (CCNNC)).

FIG. 10A and FIG. 10B depicts secondary structure predicted from folding the CRISPR repeat+tracrRNA for MG64 systems.

FIG. 11A depicts the MG64-3 CRISPR locus. The tracrRNA is encoded upstream from the CRISPR array, while the transposon end is encoded downstream (inner black box). A sequence corresponding to a partial 3′ CRISPR repeat and a partial spacer are encoded within the transposon (outer box). The self-matching spacer is encoded outside of the transposon end.

FIG. 11B depicts tracrRNA sequence alignment for various CASTs provided herein. Alignment of tracrRNA sequences shows regions of conservation. In particular, the sequence “TGCTTTC” at sequence position 92-98 (top box) is suggested to be important for sgRNA tertiary structure and for a non-continuous repeat-anti-repeat pairing with the crRNA. The hairpin “CYCC(n6)GGRG” at positions 265-278 (bottom box) is important for function, possibly positioning the downstream sequence for crRNA pairing.

FIG. 12A depicts the predicted structure of MG64-1 sgRNA.

FIG. 12B depicts the predicted structure of MG64-3 sgRNA.

FIG. 12C depicts the predicted structure of MG64-5 sgRNA.

FIGS. 13A-13C depict PCR data which demonstrate that MG64-1 is active with sgRNA v2-1. Using the protocol described for in vitro targeted integrase activity, the effector protein and its TnsB, TnsC, and TniQ proteins were expressed in an in vitro transcription/translation system. After translation, the target DNA, cargo DNA, and sgRNA were added in reaction buffer. Integration was assayed by PCR across the target/donor junctions. FIG. 13A depicts a diagram illustrating the potential orientation of integrated donor DNA. PCR reactions 3, 4, 5, and 6 represent each integration ligation product depending on the orientation in which the donor was integrated at the target site. FIG. 13B depicts a gel image of PCR 4 (detecting the RE junction to the donor) of transposition showing: lane 1) apo (no sgRNA), lane 2) with sgRNA 1, and lane 3) with sgRNA v2-1. FIG. 13C depicts a gel image of PCR 5 (detecting the LE junction to the donor) of transposition showing: lane 1) apo (no sgRNA), lane 2) with sgRNA 1, and lane 3) with sgRNA v2-1.

FIG. 14 depicts PCR reaction 5 (LE proximal to PAM, top half of plot) and PCR reaction 4 (RE distal to PAM, bottom half of plot) plotted on the sequence and distance from the PAM for MG64-1. Analysis of the integration window indicates that 95% of the integrations that occur at the spacer PAM site are within a 10 bp window between 58 and 68 nucleotides away from the PAM. Differences in the integration distance between the distal and the proximal frequencies reflects the integration site duplication—a 3-5 base pair duplication as a result of staggered nuclease activity of the transposase upon integration.

FIG. 15 depicts the results of a colony PCR screen of Transposition Efficiency. After incubation, 18 colony forming units (CFUs) were visible on the plates; 8 on plate A (no IPTG, lanes labeled as A) and 10 on plate B (with 100 μM IPTG in recovery, lanes labeled as B). All 18 were analyzed by colony PCR, which gave a product band indicative of a successful transposition reaction (arrows).

FIG. 16 depicts sequencing results of select colony PCR products which confirm that they represent transposition events, as they span the junction between the LE and the PAM at the engineered target site, which is in the lacZ gene. The minimal LE sequence is indicated in blue at the top of the screen (min LE), while the target and PAM are indicated in grey. Some sequence variation is observed in the PCR products, but this variation is expected given that insertion can occur at variable distances upstream of the PAM.

FIG. 17 depicts the results of testing of engineered single guides for 64-1 transposition activity. Black boxes are lanes not pertaining to this experiment. Panel A of FIG. 17 depicts a gel image of PCR 4 (detecting the RE junction to the donor) of transposition: lane 1=apo (no sgRNA), lane 2=holo (+sgRNA), lane 3=sgRNA v1-1, lane 4=sgRNA v1-2, lane 5=sgRNA v1-3. Panel B of FIG. 17 depicts a gel image of PCR 5 (detecting the LE junction to the donor) of transposition: lane 1=apo (no sgRNA), lane 2=holo (+sgRNA), lane 3=sgRNA v1-1, lane 4=sgRNA v1-2, lane 5=sgRNA v1-3. Panel C of FIG. 17 depicts a gel image of PCR 4 (detecting the RE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. Panel D of FIG. 17 depicts a gel image of PCR 5 (detecting the LE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. Panel E of FIG. 17 depicts a gel image of PCR 4 (detecting the RE junction to the donor) of transposition: lane 1=apo (no sgRNA), lane 2=holo (+sgRNA), lane 3=sgRNA v1-5, lane 4=skip, lane 5=sgRNA v1-10. Panel F of FIG. 17 depicts a gel image of PCR 5 (detecting the LE junction to the donor) of transposition: lane 1=apo (no sgRNA), lane 2=holo (+sgRNA), lane 3=sgRNA v1-5, lane 4=skip, lane 5=sgRNA v1-10. Panel G of FIG. 17 depicts a gel image of PCR 4 (detecting the RE junction to the donor) of transposition: lane 1=apo (no sgRNA), lane 2=holo (+sgRNA), lane 3=sgRNAvl-17, lane 4=sgRNA v1-18, lane 5=skip, lane 6=sgRNA v1-19, lane 7=skip, lane 8=sgRNA v1-20. Panel H of FIG. 17 depicts a gel image of PCR 5 (detecting the LE junction to the donor) of transposition: lane 1=apo (no sgRNA), lane 2=holo (+sgRNA), lane 3=sgRNAvl-17, lane 4=sgRNA v1-18, lane 5=skip, lane 6=sgRNA v1-19, lane 7=skip, lane 8=sgRNA v1-20

FIG. 18 depicts the results of testing of engineered LE and RE for 64-1 transposition activity. Black boxes are lanes not pertaining to this experiment. Panel A of FIG. 18 depicts a gel image of PCR 4 (detecting the RE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. Panel B of FIG. 18 depicts a gel image of PCR 5 (detecting the LE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. Panel C of FIG. 18 depicts a gel image of PCR 4 (detecting the RE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. Panel D of FIG. 18 depicts a gel image of PCR 5 (detecting the LE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. Panel E of FIG. 18 depicts a gel image of PCR 4 (detecting the RE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. Panel F of FIG. 18 depicts a gel image of PCR 5 (detecting the LE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. Panel G of FIG. 18 depicts a gel image of PCR 6 (detecting the RE junction to the donor) of transposition: lane 1=apo (no sgRNA), lane 2=holo (+sgRNA), lane 3=Obp overhang, lane 4=1 bp overhang, lane 5=2 bp overhang, lane 6=3 bp overhang, lane 7=5 bp overhang, lane 8=10 bp overhang.

FIG. 19 depicts the results of testing of engineered CAST components with an NLS for transposition activity. Black boxes are lanes not pertaining to this experiment. Panel A of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. Panel B of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. Panel C of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. Panel D of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. Panel E of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. Panel F of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. Panel G of FIG. 19 depicts a gel image of PCR 4 (detecting the RE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. Panel H of FIG. 19 depicts a gel image of PCR 5 (detecting the LE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. Panel I of FIG. 19 depicts a gel image of PCR 4 (detecting the RE junction to the donor) of transposition: lane 1=apo (no sgRNA), lane 2=holo (+sgRNA), lane 3=Cas 2x NLS apo (no sgRNA), lane 4=Cas 2x NLS holo (+sgRNA). Panel J of FIG. 19 depicts a gel image of PCR 5 (detecting the LE junction to the donor) of transposition: lane 1=apo (no sgRNA), lane 2=holo (+sgRNA), lane 3=Cas 2x NLS apo (no sgRNA), lane 4=Cas 2x NLS holo (+sgRNA)

FIG. 20 depicts engineered CAST-NLS acting as a single suite. All lanes have Cas12k-NLS and NLS-TniQ, TnsB, TnsC and sgRNA unless otherwise described. Panel A of FIG. 20 depicts a gel image of PCR 4 (detecting the RE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC. Panel B of FIG. 20 depicts gel image of PCR 5 (detecting the LE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC.

FIG. 21 depicts the results of testing of Cas Effector and TniQ protein fusion for transposition activity. Panel A of FIG. 21 depicts a gel image of PCR 4 (detecting the RE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. Panel B of FIG. 21 depicts a gel image of PCR 5 (detecting the LE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. Panel C of FIG. 21 depicts a gel image of PCR 4 (detecting the RE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. Panel D of FIG. 21 depicts a gel image of PCR 5 (detecting the LE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. Panel E of FIG. 21 depicts a gel image of PCR 4 (detecting the RE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. Panel F of FIG. 21 depicts a gel image of PCR 5 (detecting the LE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. Panel G of FIG. 21 depicts a gel image of PCR 4 (detecting the RE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA). Panel H of FIG. 21 depicts a gel image of PCR 5 (detecting the LE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA).

FIG. 22 depicts the results of expression of TnsB and TnsC in human cells, followed by cell fractionation and in vitro transposition reactions. Panel A of FIG. 22 depicts a gel image of PCR 4 (detecting the RE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. Panel B of FIG. 22 depicts a gel image of PCR 5 (detecting the LE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. Panel C of FIG. 22 depicts a gel image of PCR 4 (detecting the RE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. Panel D of FIG. 22 depicts a gel image of PCR 5 (detecting the LE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. Panel E of FIG. 22 depicts a gel image of PCR 4 (detecting the RE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm. Panel F of FIG. 22 depicts a gel image of PCR 5 (detecting the LE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm.

FIG. 23 depicts the results of expression of Cas12k and TniQ linked constructs in human cells, followed by in vitro transposition testing. Panel A of FIG. 23 depicts a gel image of PCR 5 (detecting the LE junction to the donor) of transposition: lane 1=apo (no sgRNA), lane 2=holo (+sgRNA), lane 3=Cas-NLS holo (+sgRNA) cytoplasm, lane 4=Cas-NLS holo (+sgRNA) nucleoplasm, lane 5=Cas-NLS holo (+sgRNA) nucleoplasm+additional sgRNA, lane 6=Cas-NLS-P2A-NLS-TniQ holo (+sgRNA) cytoplasm, lane 7=Cas-NLS-P2A-NLS-TniQ holo (+sgRNA) nucleoplasm, lane 8=Cas-NLS-P2A-NLS-TniQ holo (+sgRNA) nucleoplasm+additional sgRNA. Panel B of FIG. 23 depicts a gel image of PCR 4 (detecting the RE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. Panel C of FIG. 23 depicts a gel image of PCR 5 (detecting the LE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. Panel D of FIG. 23 depicts a gel image of PCR 4 (detecting the RE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. Panel E of FIG. 23 depicts a gel image of PCR 5 (detecting the LE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. Panel F of FIG. 23 depicts a gel image of PCR 4 (detecting the RE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ. Panel G of FIG. 23 depicts a gel image of PCR 5 (detecting the LE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ.

FIG. 24 depicts electrophoretic mobility shift assay (EMSA) results of the 64-1 TnsB and its LE DNA sequence. The EMSA results confirm binding and TnsB recognition. The TnsB protein was expressed in an in vitro transcription/translation system, incubated with FAM-labeled DNA containing the LE sequence, and then separated on a native 5% TBE gel. Binding is observed as a shift upwards in the labeled band. Multiple TnsB binding sites leads to multiple shifts in the EMSA. Lane 1: FAM-labeled DNA only. Lane 2: FAM DNA plus the in vitro transcription/translation system (no TnsB protein). Lane 3: FAM DNA plus TnsB.

FIG. 25 depicts activity of in vitro tested Cas12k and TniQ fusions. Panel A of FIG. 25 depicts a gel image showing transposition activity of left end to donor. Lane 1=apo (no sgRNA), Lane 2=holo (with sgRNA), Lane 3=(Skip) Cas12k-2×NLS apo (no sgRNA), Lane 4=(Skip) Cas12k-2×NLS holo (with sgRNA), Lane 5=Cas12k-Cbx5 fusion protein apo (no sgRNA), Lane 6=Cas12k-Cbx5 fusion protein holo (with sgRNA). Panel B of FIG. 25 depicts a gel image showing transposition activity of right end to donor. Lane 1=apo (no sgRNA), Lane 2=holo (with sgRNA), Lane 3=Cas12k-2×NLS apo (no sgRNA), Lane 4=Cas12k-2×NLS holo (with sgRNA), Lane 5=Cas12k-Cbx5 fusion protein apo (no sgRNA), Lane 6=Cas12k-Cbx5 fusion protein holo (with sgRNA). Panel C of FIG. 25 depicts a gel image showing transposition activity of left end to donor. Lane 1=apo (no sgRNA), Lane 2=holo (with sgRNA), Lane 3=Cbx5-TniQ fusion protein holo (with sgRNA), Lane 4=H1 core-TniQ fusion protein holo (with sgRNA), Lane 5=HMGN1-TniQ fusion protein holo (with sgRNA), Lane 6=Cas12k-H1 core fusion protein holo (with sgRNA), Lane 7=Cas12k-HMGN1 fusion protein holo (with sgRNA). Panel D of FIG. 25 depicts a gel image showing transposition activity of left end to donor under combinatorial sso7d fusion proteins described in lanes. Lane 1=WT Cas12k with WT TniQ apo (no sgRNA), Lane 2=WT Cas12k with WT TniQ holo (with sgRNA), Lane 3=WT Cas12k with sso7d-TniQ fusion protein apo (with sgRNA), Lane 4=WT Cas12k with sso7d-TniQ fusion protein holo (with sgRNA), Lane 5=Cas12k-sso7d fusion protein with WT TniQ apo (without sgRNA), Lane 6=Cas12k-sso7d fusion protein with WT TniQ holo (with sgRNA), Lane 7=Cas12k-sso7d fusion protein with sso7d-TniQ fusion protein apo (without sgRNA), Lane 8=Cas12k-sso7d fusion protein with sso7d-TniQ holo (with sgRNA).

FIGS. 26A-26C depict the functionality of sso7d with Cas12k. FIG. 26A depicts a gel image of LE to target transposition in nuclear extracts with Cas12k-sso7d-NLS. Lane 1=apo (no sgRNA) in vitro transposition reaction with Cas12k-sso7d-NLS with otherwise WT components, Lane 2=holo (with sgRNA) in vitro transposition reaction with Cas12k-sso7d-NLS with otherwise WT components, Lane 3=Cytoplasmic extract of Cas12k-sso7d-NLS cell line with otherwise in vitro expressed CAST components apo condition (without sgRNA), Lane 4=Cytoplasmic extract of Cas12k-sso7d-NLS cell line with otherwise in vitro expressed CAST components holo condition (with sgRNA), Lane 5=Nuclear extract of Cas12k-sso7d-NLS cell line with otherwise in vitro expressed CAST components apo condition (without sgRNA), Lane 6=Nuclear extract of Cas12k-sso7d-NLS cell line with otherwise in vitro expressed CAST components holo condition (with sgRNA), Lane 7=Nuclear extract of Nuclear extract of Cas12k-sso7d-NLS cell line with otherwise in vitro expressed CAST components holo condition (with sgRNA) with additional WT Cas12k, Lane 8=Nuclear extract of Nuclear extract of Cas12k-sso7d-NLS cell line with otherwise in vitro expressed CAST components holo condition (with sgRNA) with additional WT TniQ. Arrow is pointing to active transposition event. FIG. 26B depicts the sequence of gel extracted band from 2A Lane 6 aligned to the predicted transposition product. FIG. 26C depicts a gel image of LE to target transposition in nuclear extracts with the addition of WT or DBD TniQ fusions or WT Cas12k. Nuclear extracts contain WT NLS tagged Cas12k, TnsB, TnsC and TniQ. Lane 1=Nuclear extract with apo WT Cas12k and WT TniQ (no sgRNA), Lane 2=Nuclear extract with holo Cas12k (with sgRNA) and WT TniQ, Lane 3=Nuclear Extract with additional WT TniQ apo condition (no sgRNA), Lane 4=Nuclear Extract with additional WT TniQ holo condition (with sgRNA), Lane 5=Nuclear extract with additional Cbx5-TniQ fusion protein apo condition (no sgRNA), Lane 6=Nuclear extract with additional Cbx5-TniQ fusion protein holo condition (with sgRNA), Lane 7=Nuclear extract with additional H1 core-TniQ fusion protein apo condition (no sgRNA), Lane 8=Nuclear extract with additional H1 core-TniQ fusion protein holo condition (with sgRNA), Lane 9=Nuclear extract with additional HMGN1-TniQ fusion protein apo condition (no sgRNA), Lane 10=Nuclear extract with additional HMGN1-TniQ fusion protein holo condition (with sgRNA), Lane 11=Nuclear extract with additional sso7d-TniQ fusion protein apo condition (no sgRNA), Lane 12=Nuclear extract with additional sso7d-TniQ fusion protein holo condition (with sgRNA).

FIG. 27 depicts gel images of transposition reactions. Panel A of FIG. 27 depicts a gel image of transposition activity of Cas12k-sso7d and HMGN1-TniQ from cellular extracts. All lanes contain in vitro expressed TnsB and TnsC added. Lane 1=in vitro CAST holo (with sgRNA), Lane 2=in vitro CAST apo (without sgRNA), Lane 3=cytoplasmic extract without additional Cas12k, TniQ protein nor sgRNA, Lane 4=cytoplasmic extract without addition Cas12k nor TniQ protein with additional sgRNA, Lane 5=nuclear extract without additional Cas12k nor TniQ protein nor sgRNA, Lane 6=nuclear extract without additional Cas12k nor TniQ with additional sgRNA, Lane 7=nuclear extract with additional Cas12k and sgRNA only, Lane 8=nuclear extract with additional TniQ and sgRNA only. Panel B of FIG. 27 depicts a gel image of transposition activity of Cas12k-sso7d and H1 core-TniQ from cellular extracts. All lanes contain in vitro expressed TnsB and TnsC added. Lane 1=in vitro CAST holo (with sgRNA), Lane 2=in vitro CAST apo (without sgRNA), Lane 3=cytoplasmic extract without additional Cas12k, TniQ protein nor sgRNA, Lane 4=cytoplasmic extract without addition Cas12k nor TniQ protein with additional sgRNA, Lane 5=nuclear extract without additional Cas12k nor TniQ protein nor sgRNA, Lane 6=nuclear extract without additional Cas12k nor TniQ with additional sgRNA, Lane 7=nuclear extract with additional Cas12k and sgRNA only, Lane 8=nuclear extract with additional TniQ and sgRNA only. Panel C of FIG. 27 depicts a gel image of transposition activity of Cas12k-sso7d, HMGN1-TniQ, TnsB, and TnsC cellular extracts. Lane 1=in vitro CAST apo (no sgRNA), Lane 2=in vitro CAST holo (with sgRNA), Lane 3=cytoplasmic extract without additional Cas12k, TniQ protein nor sgRNA, Lane 4=cytoplasmic extract without addition Cas12k nor TniQ protein but with additional sgRNA, Lane 5=nuclear extract without additional Cas12k nor TniQ protein nor sgRNA, Lane 6=nuclear extract without additional Cas12k nor TniQ but with additional sgRNA, Lane 7=nuclear extract with additional Cas12k and sgRNA only, Lane 8=nuclear extract with additional TniQ and sgRNA only. Panel D of FIG. 27 depicts a gel image of transposition activity of Cas12k-sso7d, H1 core-TniQ, TnsB, and TnsC cellular extracts. Lane 1=in vitro CAST apo (no sgRNA), Lane 2=in vitro CAST holo (with sgRNA), Lane 3=cytoplasmic extract without additional Cas12k, TniQ protein nor sgRNA, Lane 4=cytoplasmic extract without addition Cas12k nor TniQ protein but with additional sgRNA, Lane 5=nuclear extract without additional Cas12k nor TniQ protein nor sgRNA, Lane 6=nuclear extract without additional Cas12k nor TniQ but with additional sgRNA, Lane 7=nuclear extract with additional Cas12k and sgRNA only, Lane 8=nuclear extract with additional TniQ and sgRNA only.

FIG. 28A shows a schematic representation of serial dilution of target DNA for in vitro transposition experiments. The CAST components are expressed with PureExpress and added to the reaction with in vitro transcribed sgRNA and donor plasmid. Target plasmid DNA is added at decreasing concentrations and tested for transposition experiments. When the minimum amount of target DNA is determined, transposition reactions are assayed by adding increasing amounts of human genomic DNA.

FIG. 28B shows an illustration of PCR amplification of transposition reactions. An 8N PAM plasmid library (8N-Target, Rxn #1) is targeted with the CAST system to integrate donor DNA (Rxn #2). Upon successful integration, junction PCR reactions are performed with primers to amplify the four putative integration reactions, based on the orientation of cargo integration (Rxn #3, #4, #5, and #6).

FIG. 28C illustrates PCR reaction products from in vitro transposition assays with serial dilutions of target plasmid DNA. Target, donor, and reactions #3, #4, #5, and #6 correspond to PCR integration products as shown in FIG. 28B.

FIG. 28D shows PCR reaction products from in vitro transposition assays with a fixed amount of target plasmid DNA (0.5 ng) while adding increasing amounts of human genomic DNA to increase the search space. Target, donor, and reactions #3, #4, #5, and #6 correspond to PCR integration products as shown in FIG. 28B.

FIG. 29A shows a schematic of transposition reactions across a high copy element. The target PCR product spans the wild-type target element when assayed with CAST proteins and sgRNA targeting one of the multiple arrayed targets. Integration can occur in either the forward orientation, the reverse orientation, or both. The forward transposition product is assayed by junction PCR that amplifies the region encompassing the LE of the donor DNA to the 5′ end of the target site (Fwd PCR). The reverse junction reaction assays the region encompassing the LE of the donor DNA to the 3′ end of the target element (Rev PCR).

FIG. 29B shows PCR reaction products from in vitro transposition assays at 15 target sites (guide) in LINE1 3′ elements in human genomic DNA. Target and reactions Fwd PCR and Rev PCR correspond to PCR integration products as shown in FIG. 29A.

FIG. 29C shows PCR reaction products from in vitro transposition assays at 15 target sites (guide) in SVA elements in human genomic DNA. Target and reactions Fwd PCR and Rev PCR correspond to PCR integration products as shown in FIG. 29A. Bands highlighted with an arrow indicate successful targeted integration.

FIG. 29D shows PCR reaction products from in vitro transposition assays at 15 target sites (guide) in HERV elements in human genomic DNA. Target and reactions Fwd PCR and Rev PCR correspond to PCR integration products as shown in FIG. 29A. Bands highlighted with an arrow indicate successful targeted integration.

FIG. 29E shows Sanger sequencing of the Fwd PCR integration product at multiple target sites of the LINE1 3′ elements. The point at which the sequencing trace stops matching the donor DNA (grey vertical bar) is where integration occurs.

FIG. 29F shows Sanger sequencing of the Rev PCR integration product at multiple target sites of the LINE1 3′ elements. The point at which the sequencing trace stops matching the target DNA (grey vertical bar) is where integration occurs.

FIG. 29G shows Sanger sequencing of the Fwd PCR integration product at SVA target site 3. The point at which the sequencing trace stops matching the donor DNA (grey vertical bar) is where integration occurs.

FIG. 29H shows Sanger sequencing of the Fwd PCR product at HERV target site 5. The point at which the sequencing trace stops matching the donor DNA (grey vertical bar) is where integration occurs.

FIG. 30 shows PCR reaction products from in vitro transposition assays at LINE1 target sites 12 and 15 in human genomic DNA with functional domains. Target and reactions Fwd PCR and Rev PCR correspond to PCR integration products as shown in FIG. 28A. Bands highlighted with an arrow indicate successful targeted integration.

FIGS. 31A-31B illustrate in vitro transposition experiments with CAST, S15, NLS-S15, and S15-NLS expressed from Eukaryotic transcription/translation reactions. FIG. 31A shows in vitro transposition reactions with MG64-1 CAST and S15. Wheat Germ Extract-expressed CAST components promote transposition without addition of S15, albeit at a low rate (faint bands highlighted with arrows). Addition of PURExpress reagent (Spent PUREx) increases transposition efficiency, as shown by the strength of the band at Rxn #5 (PURExpress reagent contains S15). Independent addition of S15 and S15-NLS translated from Wheat Germ Extract reactions increases transposition efficiency by MG64-1 in vitro compared with the other conditions tested (strong bands highlighted with arrows). FIG. 31B shows in vitro reactions of transposition with the NLS-S15 configuration. PURExpress reagent addition increases in vitro transposition (Lane 3) compared with CAST-components only conditions (Lane 2). The NLS-S15 configuration did not improve transposition (Lanes 4-5). Boxed Rxn #5 represents an expected band if transposition activity is detected.

FIGS. 32A-32H show a schematic of fusion plasmids for in cell transposition. FIG. 32A: two targeting complex plasmids and one donor plasmid are assembled for high copy elements Line1, targets 8, 12, and 15, and SVA target 3. FIG. 32B shows in cell transposition to high copy elements with H1core-TniQ or HMGN1-TniQ at LINE1 targets 8, 12, 15, and SVA target 3. Arrows indicate amplified transposition junction reactions in either forward (Fwd PCR) or reverse (Rev PCR) orientation of transposition. Mock control represents a reaction without targeting or donor plasmids. FIG. 32C shows Sanger sequencing of the PCR integration product Fwd PCR at LINE1 3′ target site 8. Integration was mediated by MG64-1 with the NLS-H1core-TniQ fusion. The point at which the sequencing trace stops matching the donor DNA (grey vertical bar) is where integration occurs. FIG. 32D shows Sanger sequencing of the PCR integration product Fwd PCR at LINE1 3′ target site 8. Integration was mediated by MG64-1 with the NLS-HMGN1-TniQ fusion. The point at which the sequencing trace stops matching the donor DNA (grey vertical bar) is where integration occurs. FIG. 32E shows Sanger sequencing of the PCR integration product Rev PCR at LINE1 3′ target site 12. Integration was mediated by MG64-1 with the NLS-H1core-TniQ fusion. The point at which the sequencing trace stops matching the donor DNA (grey vertical bar) is where integration occurs. FIG. 32F shows Sanger sequencing of the PCR integration product Rev PCR at LINE1 3′ target site 12. Integration was mediated by MG64-1 with the NLS-HMGN1-TniQ fusion. The point at which the sequencing trace stops matching the donor DNA (grey vertical bar) is where integration occurs. FIG. 32G shows Sanger sequencing of the PCR integration product Fwd PCR at LINE1 3′ target site 15. Integration was mediated by MG64-1 with the NLS-H1core-TniQ fusion. The point at which the sequencing trace stops matching the donor DNA (grey vertical bar) is where integration occurs. FIG. 32H shows Sanger sequencing of the PCR integration product Fwd PCR at LINE1 3′ target site 15. Integration was mediated by MG64-1 with the NLS-HMGN1-TniQ fusion. The point at which the sequencing trace stops matching the donor DNA (grey vertical bar) is where integration occurs.

FIGS. 33A-33B depict in vitro screening of MG64-1 Cas12k CAST transposition. FIG. 33A: Diagram of the construct used for MG64-1 holocomplex purification. FIG. 33B: Schematic of junction PCR for the detection of transposition products. A target substrate with a 5′ PAM followed by the protospacer (Target, Rxn #1) is targeted with the CAST system to integrate cargo DNA (Rxn #2). Upon successful integration, junction PCR reactions are performed with primers to amplify the four putative integration reactions, based on the orientation of cargo integration. FIGS. 33C-33D depict MG64-1 protein purification. FIG. 33C: Fractions collected during 2 L-scale purification of MG64-1 holocomplex run on stain free denaturing PAGE gel. FIG. 33D: Chromatogram of Size Exclusion Chromatography (SEC) performed on MG64-1 holo complex. The peak centered at 29.3 mL (peak 1) was used for in vitro activity assays.

FIG. 34A depicts in vitro transposition with Peak1-recovered holocomplex supplemented with TnT expressed components. Lane L) Ladder; Lane 1) TnT expressed CAST components apo condition (−sgRNA); Lane 2) TnT expressed CAST components holo condition (+sgRNA); Lane 3) Purified Peak1 complemented with TnT CAST components without additional supplementation of Cas12k (−TnT Cas12k); Lane 4) Purified Peak1 complemented with TnT CAST components without additional supplementation of TnsC (−TnT TnsC); Lane 5) Peak1 complemented with TnT CAST components without additional supplementation of TniQ (−TnT TniQ); Lane 6) Peak1 complemented with TnT CAST components without additional supplementation of S15 (−TnT S15). FIG. 34B depicts Sanger sequencing of Lane 3, Lane 4, Lane 5, and Lane 6 from both pDonor and Target directions of the amplified LE to PAM target-donor junction. Vertical line delineates the transposition junction predicted for MG64-1 in the reference sequence. Degradation of signal from either direction results from a multitude of signals reflected in the PCR amplification.

FIG. 35 depicts the identification of ribosomal protein S15 homologs in Cyanobacterial genomic fragments. Candidate sequences from the same sample from where MG64-1 was recovered are highlighted by dark closed circles. The reference S15 from E. coli is indicated with an arrow.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG64

SEQ ID NOs: 1, 12, 16, 20-30, 64, 80-85, and 200 show the full-length peptide sequences of MG64 Cas effectors.

SEQ ID NOs: 2-4, 13-15, 17-19, and 65-67 show the peptide sequences of MG64 transposition proteins that may comprise a recombinase complex associated with the MG64 Cas effector.

SEQ ID NOs: 5-6, 32-33, 94-95, 104-105, and 202 show nucleotide sequences of MG64 tracrRNAs derived from the same loci as a MG64 Cas effector.

SEQ ID NOs: 7 and 34-35 show nucleotide sequences of MG64 target CRISPR repeats.

SEQ ID NOs: 106-108 and 201 show nucleotide sequences of MG64 crRNAs.

SEQ ID NO: 8, 10, 39-44, 77, 79, and 93 show nucleotide sequences of right-hand transposase recognition sequences associated with a MG64 system.

SEQ ID NO: 9, 11, 36-38, 76, and 78 show nucleotide sequences of left-hand transposase recognition sequences associated with a MG64 system.

SEQ ID NOs: 45-63, 68-75, and 96-103 show nucleotide sequences of single guide RNAs engineered to function with MG64 Cas effectors.

SEQ ID NOs: 113-120 show nucleotide and peptide sequences of MG64 DNA binding domain CAST fusion proteins.

SEQ ID NO: 188 shows the nucleotide sequence of an MG64 expression construct.

### MG190

SEQ ID NOs: 189-199 show the full-length peptide sequences of MG190 ribosomal protein S15 homologs.

### Other Sequences

SEQ ID NOs: 86-87 and 172-187 show peptide sequences of nuclear localizing signals.

SEQ ID NOs: 88-89 show peptide sequences of linkers.

SEQ ID NOs: 90-92 show peptide sequences of epitope tags.

SEQ ID NOs: 109-112 show peptide sequences of DNA binding domains.

SEQ ID NOs: 121-123 show genomic target sequences.

SEQ ID NOs: 124-160 show target guide sequences.

SEQ ID NOs: 161-163 show nucleic acid sequences of S15 fusion proteins.

SEQ ID NO: 164 shows a donor construct.

SEQ ID NO: 165 shows an MG64-1 sgRNA sequence.

SEQ ID NO: 166 shows a linker sequence.

SEQ ID NOs: 167-169 show amino acid sequences of S15 fusion proteins.

SEQ ID NOs: 170-171 show promoter sequences.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. In a polynucleotide when referring to a T, a T means U (Uracil) in RNA and T (Thymine) in DNA. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A “basal promoter”, also referred to as a “core promoter”, may refer to a promoter that contains all the basic necessary elements to promote transcriptional expression of an operably linked polynucleotide. Eukaryotic basal promoters typically, though not necessarily, contain a TATA-box and/or a CAAT box. In some embodiments, different promoters direct the expression of a gene in different tissues or cell types, or at different stages of development, or in response to different environmental or physiological conditions or inducer molecules. Promoters that cause a gene to be expressed in most cell types most of the time are commonly referred to as “constitutive promoters.” Promoters that cause the expression of genes in a particular cell and tissue type are commonly referred to as “cell-specific promoters” or “tissue-specific promoters,” respectively. Promoters that cause the expression of genes at specific stages of development or cell differentiation are commonly referred to as “development-specific promoters” or “cell differentiation-specific promoters.” Promoters that induce and result in the expression of genes after exposing or treating cells with agents, biomolecules, chemicals, ligands, light, etc. that induce the promoters are commonly referred to as “inducible promoters” or “regulatable promoters.” It is further recognized, in some embodiments, that since the exact boundaries of regulatory sequences have not been completely defined in most cases, DNA fragments of different lengths have the same promoter activity.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

The terms “engineered,” “synthetic,” and “artificial” are used interchangeably herein to refer to an object that has been modified by human intervention. For example, the terms may refer to a polynucleotide or polypeptide that is non-naturally occurring. An engineered peptide may have, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

The term “tracrRNA” or “tracr sequence” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid. If the tracrRNA is engineered, it may have about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes, S. aureus, etc.). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes, S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” or “guide polynucleotide” refers to a nucleic acid that may hybridize to a target nucleic acid and thereby directs an associated nuclease to the target nucleic acid. A guide nucleic acid may be RNA (guide RNA or gRNA). A guide nucleic acid may be DNA. A guide nucleic acid may be a mixture of RNA and DNA. A guide nucleic acid may comprise a crRNA or a tracrRNA or a combination of both. A guide nucleic acid may be engineered. The guide nucleic acid may be programmed to specifically bind to the target nucleic acid. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence,” or a “spacer.” A nucleic acid-targeting segment may comprise a sub-segment that may be referred to as a “protein binding segment” or “protein binding sequence” or “Cas protein binding segment.”

As used herein, the terms “gene editing” and “genome editing” can be used interchangeably. Gene editing or genome editing means to change the nucleic acid sequence of a gene or a genome. Genome editing can include, for example, insertions, deletions, and mutations.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

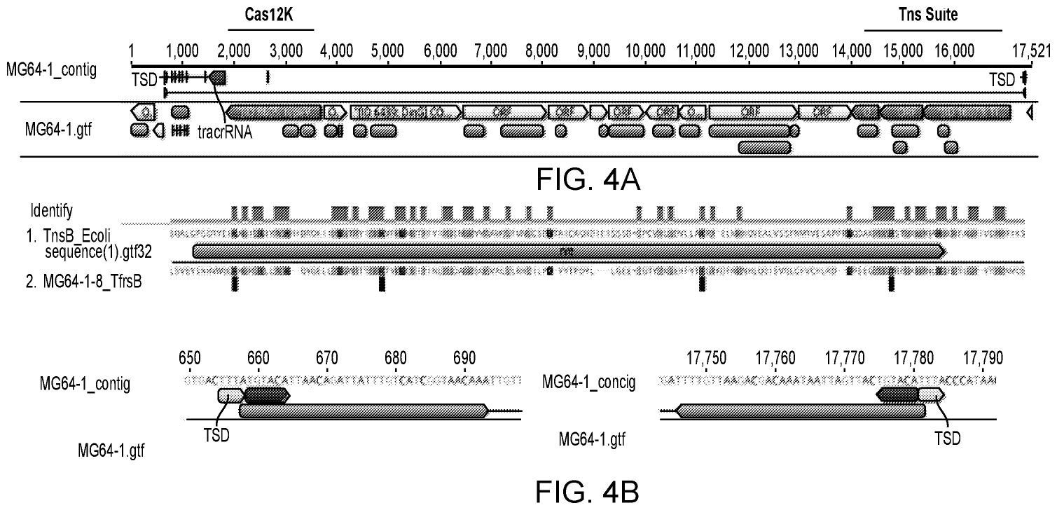

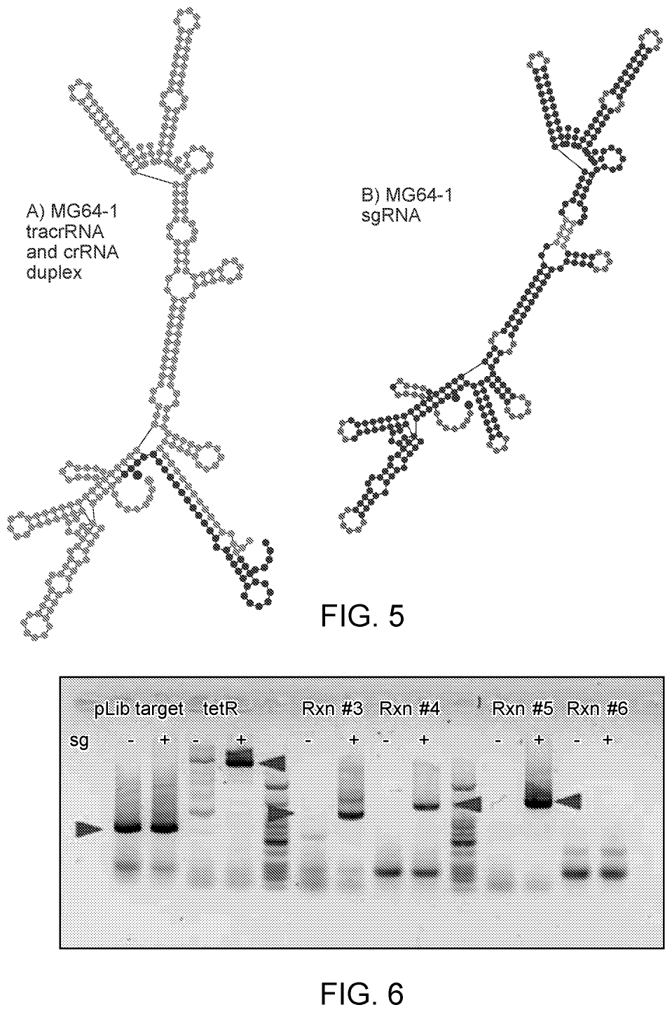

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g., non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein (e.g., MG64 systems described herein). In some embodiments, such conservatively substituted variants are functional variants. Such functional variants can encompass sequences with substitutions such that the activity of critical active site residues of the endonuclease are not disrupted. In some embodiments, a functional variant of any of the systems described herein lack substitution of at least one of the conserved or functional residues called out in FIGS. 4 and 5. In some embodiments, a functional variant of any of the systems described herein lacks substitution of all of the conserved or functional residues called out in FIGS. 4 and 5.

Conservative substitution tables providing functionally similar amino acids are available from a variety of references (see, for example, Creighton, Proteins: Structures and Molecular Properties (W H Freeman & Co.; 2^{nd} Edition (December 1993))). The following eight groups each contain amino acids that are conservative substitutions for one another:
1. Alanine (A), Glycine (G);
2. Aspartic acid (D), Glutamic acid (E);
3. Asparagine (N), Glutamine (Q);
4. Arginine (R), Lysine (K);
5. Isoleucine (I), Leucine (L), Methionine (M), Valine (V);
6. Phenylalanine (F), Tyrosine (Y), Tryptophan (W);
7. Serine (S), Threonine (T); and
8. Cysteine (C), Methionine (M).

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “recombinase” refers to an enzyme that mediates the recombination of DNA fragments located between recombinase recognition sequences, which results in the excision, insertion, inversion, exchange, or translocation of the DNA fragments located between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the excision, insertion, inversion, exchange, or translocation of a nucleic acid sequence, e.g., in or between one or more nucleic acid molecules.

As used herein, the term “transposon,” or “transposable element” refers to a nucleic acid sequence in a genome that is a mobile genetic element that can change its position in a genome. In some cases, the transposon transports additional “cargo DNA” excised from the genome. Transposons comprise, for example retrotransposons, DNA transposons, autonomous and non-autonomous transposons, and class III transposons. Transposon nucleic acid sequences comprise, for example genes coding for a cognate transposase, one or more recognition sequences for the transposase, or combinations thereof. In some cases, these transposons differ on the type of nucleic acid to transpose, the type of repeat at the ends of the transposon, the type of cargo to be carried or by the mode of transposition (i.e. self-repair or host-repair). As used herein, the term “transposase” or “transposases” refers to an enzyme that binds to the recognition sequences of a transposon and catalyzes its movement to another part of the genome. In some cases, the movement is by a cut and paste mechanism or a replicative transposition mechanism.

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

As used herein, the term “complex” refers to a joining of at least two components. The two components may each retain the properties/activities they had prior to forming the complex. The joining may be by covalent bonding, non-covalent bonding (i.e., hydrogen bonding, ionic interactions, Van der Waals interactions, and hydrophobic bond), use of a linker, fusion, or any other suitable method. In some cases, components in a complex are polynucleotides, polypeptides, or combinations thereof. For example, a complex may comprise a Cas protein and a guide nucleic acid.

In some cases, the CAST systems described herein comprise one or more Tn7 or Tn7 like transposases. In certain example embodiments, the Tn7 or Tn7 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

In some cases, the CAST systems described herein comprise one or more Tn5053 or Tn5053-like transposases. In certain example embodiments, the Tn5053 or Tn5053-like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

As used herein, the term “Cas12k” (alternatively “class 2, type V-K”) refers to a subtype of Type V CRISPR systems that have been found to be defective in nuclease activity (e.g., they may comprise at least one defective RuvC domain that lacking at least one catalytic residue important for DNA cleavage). Such subtype of effectors have been generally associated with CAST systems.

As used herein, the term “functional domain (FD)” refers to a small protein that is capable of facilitating the interaction of proteins with DNA. Types of functional domains include, but are not limited to, DNA binding domains (“DBDs”) and chromatin modulating domains (“CMDs”). Non-limiting examples of functional domains include human histone 1 central globular domain (H1 core), high mobility group nucleosome binding domain 1 (HMGN1), chromobox 5 (Cbx5), and Saccharolobus solfataricus sso7d. In some embodiments, a functional domain described herein can be included in a fusion protein with a system described herein, or a component thereof. In some embodiments, said fusion protein displays increased activity in cells compared to the non-fusion protein.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

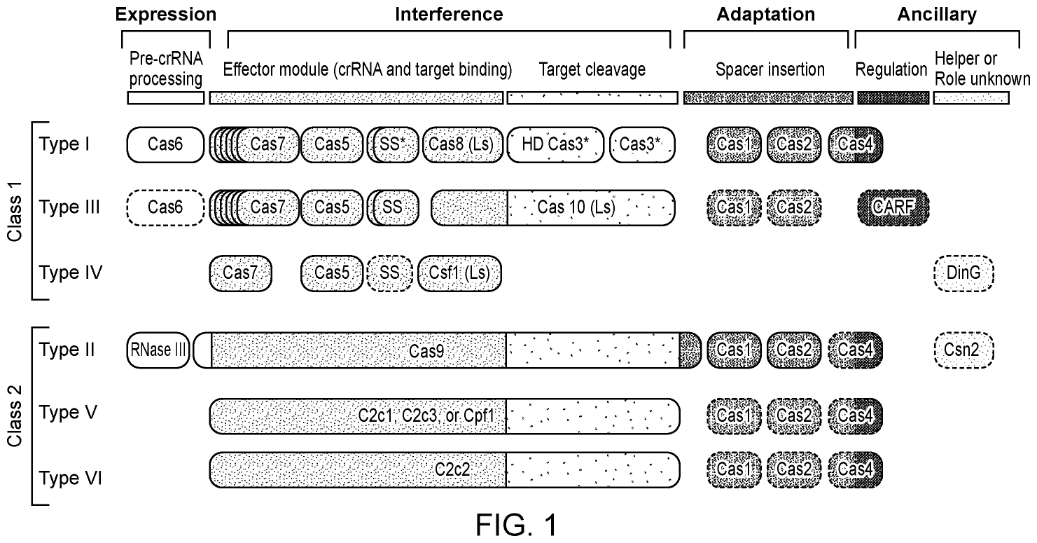

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class 1 CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class 2 CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Type II nucleases are known as DNA nucleases. Type II effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g., Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class 2 CRISPR-Cas have been most widely adopted for engineering and development as designer nuclease/genome editing applications.

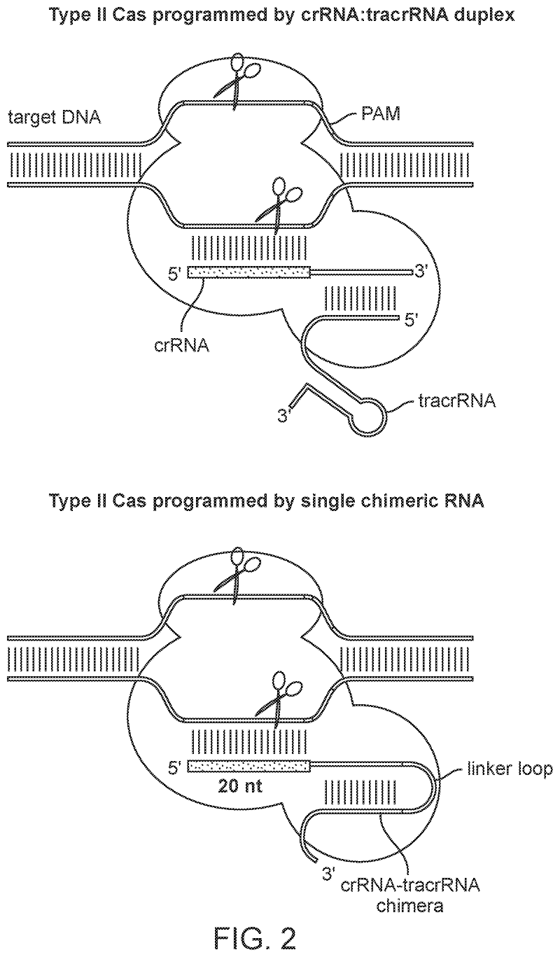

One of the early adaptations of such a system for in vitro use involved (i) recombinantly-expressed, purified full-length Cas9 (e.g., a Class 2, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. A later improved, engineered system involved the crRNA of (ii) joined to the 5′ end of (iii) by a linker (e.g., GAAA) to form a single fused synthetic guide RNA (sgRNA) capable of directing Cas9 to a target by itself (compare top and bottom panel of FIG. 2).

Such engineered systems can be adapted for use in mammalian cells by providing DNA vectors encoding (i) an ORF encoding codon-optimized Cas9 (e.g., a Class 2, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Transposons are mobile elements that can move between positions in a genome. Such transposons have evolved to limit the negative effects they exert on the host. A variety of regulatory mechanisms are used to maintain transposition at a low frequency and sometimes coordinate transposition with various cell processes. Some prokaryotic transposons also can mobilize functions that benefit the host or otherwise help maintain the element. Certain transposons may have also evolved mechanisms of tight control over target site selection, the most notable example being the Tn7 family.

Transposon Tn7 and similar elements may be reservoirs for antibiotic resistance and pathogenesis functions in clinical settings, as well as encoding other adaptive functions in natural environments. The Tn7 system, for example, has evolved mechanisms to almost completely avoid integrating into important host genes, but also maximize dispersal of the element by recognizing mobile plasmids and bacteriophage capable of moving Tn7 between host bacteria.

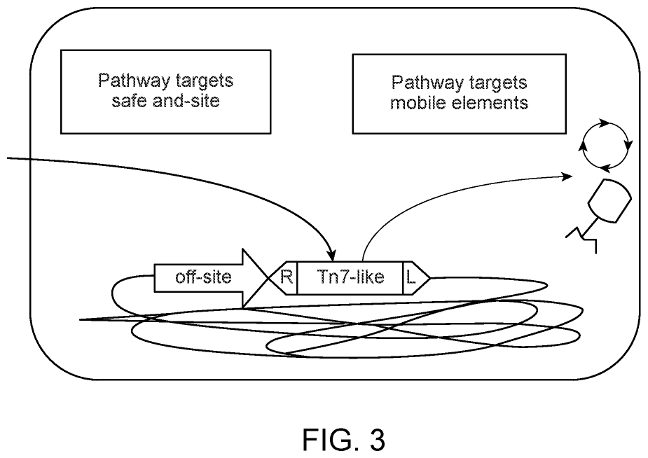

Tn7 and Tn7-like elements may control where and when they insert, possessing one pathway that directs insertion into a single conserved position in bacterial genomes and a second pathway that appears to be adapted to maximizing targeting into mobile plasmids capable of transporting the element between bacteria (see FIG. 3). The association between Tn7-like transposons and CRISPR-Cas systems suggests that the transposons might have hijacked CRISPR effectors to generate R-loops in target sites and facilitate the spread of transposons via plasmids and phages.

### MG64 Systems

Provided herein, in some embodiments, are MG64 systems for transposing a cargo nucleotide sequence into a target nucleic acid site. See FIGS. 4A-4B. In some embodiments, the system comprises a double-stranded nucleic acid comprising a cargo nucleotide sequence. In some embodiments, this cargo nucleotide sequence is configured to interact with a Tn7 type or Tn5053 type transposase complex. In some embodiments, the system comprises a Cas effector complex. In some embodiments, the Cas effector complex comprises a class 2, type V Cas effector and an engineered guide polynucleotide configured to hybridize to the target nucleotide sequence. In some embodiments, the system comprises a Tn7 type or Tn5053 type transposase complex configured to bind the Cas effector complex, wherein the Tn7 type or Tn5053 type transposase complex comprises a TnsB subunit.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class 2, type V Cas effector. In some cases, the class 2, type V Cas effector is a class 2, type V-K effector. In some cases, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, 80-85, and 200. In some cases, the class 2, type V Cas effector comprises a polypeptide comprising a sequence having at least about 70% identity to any one of SEQ ID NOs: 1, 12, 16, 20-30, 64, 80-85, and 200. In some cases, the class 2, type V Cas effector comprises a polypeptide comprising a sequence having at least about 75% identity to any one of SEQ ID NOs: 1, 12, 16, 20-30, 64, 80-85, and 200. In some cases, the class 2, type V Cas effector comprises a polypeptide comprising a sequence having at least about 80% identity to any one of SEQ ID NOs: 1, 12, 16, 20-30, 64, 80-85, and 200. In some cases, the class 2, type V Cas effector comprises a polypeptide comprising a sequence having at least about 85% identity to any one of SEQ ID NOs: 1, 12, 16, 20-30, 64, 80-85, and 200. In some cases, the class 2, type V Cas effector comprises a polypeptide comprising a sequence having at least about 90% identity to any one of SEQ ID NOs: 1, 12, 16, 20-30, 64, 80-85, and 200. In some cases, the class 2, type V Cas effector comprises a polypeptide comprising a sequence having at least about 91% identity to any one of SEQ ID NOs: 1, 12, 16, 20-30, 64, 80-85, and 200. In some cases, the class 2, type V Cas effector comprises a polypeptide comprising a sequence having at least about 92% identity to any one of SEQ ID NOs: 1, 12, 16, 20-30, 64, 80-85, and 200. In some cases, the class 2, type V Cas effector comprises a polypeptide comprising a sequence having at least about 93% identity to any one of SEQ ID NOs: 1, 12, 16, 20-30, 64, 80-85, and 200. In some cases, the class 2, type V Cas effector comprises a polypeptide comprising a sequence having at least about 94% identity to any one of SEQ ID NOs: 1, 12, 16, 20-30, 64, 80-85, and 200. In some cases, the class 2, type V Cas effector comprises a polypeptide comprising a sequence having at least about 95% identity to any one of SEQ ID NOs: 1, 12, 16, 20-30, 64, 80-85, and 200. In some cases, the class 2, type V Cas effector comprises a polypeptide comprising a sequence having at least about 96% identity to any one of SEQ ID NOs: 1, 12, 16, 20-30, 64, 80-85, and 200. In some cases, the class 2, type V Cas effector comprises a polypeptide comprising a sequence having at least about 97% identity to any one of SEQ ID NOs: 1, 12, 16, 20-30, 64, 80-85, and 200. In some cases, the class 2, type V Cas effector comprises a polypeptide comprising a sequence having at least about 98% identity to any one of SEQ ID NOs: 1, 12, 16, 20-30, 64, 80-85, and 200. In some cases, the class 2, type V Cas effector comprises a polypeptide comprising a sequence having at least about 99% identity to any one of SEQ ID NOs: 1, 12, 16, 20-30, 64, 80-85, and 200. In some cases, the class 2, type V Cas effector comprises a polypeptide comprising a sequence having 100% identity to any one of SEQ ID NOs: 1, 12, 16, 20-30, 64, 80-85, and 200.

In some cases, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, and 65. In some cases, the TnsB component comprises a polypeptide comprising a sequence having at least about 70% identity to any one of SEQ ID NOs: 2, 13, 17, and 65. In some cases, the TnsB component comprises a polypeptide comprising a sequence having at least about 75% identity to any one of SEQ ID NOs: 2, 13, 17, and 65. In some cases, the TnsB component comprises a polypeptide comprising a sequence having at least about 80% identity to any one of SEQ ID NOs: 2, 13, 17, and 65. In some cases, the TnsB component comprises a polypeptide comprising a sequence having at least about 85% identity to any one of SEQ ID NOs: 2, 13, 17, and 65. In some cases, the TnsB component comprises a polypeptide comprising a sequence having at least about 90% identity to any one of SEQ ID NOs: 2, 13, 17, and 65. In some cases, the TnsB component comprises a polypeptide comprising a sequence having at least about 91% identity to any one of SEQ ID NOs: 2, 13, 17, and 65. In some cases, the TnsB component comprises a polypeptide comprising a sequence having at least about 92% identity to any one of SEQ ID NOs: 2, 13, 17, and 65. In some cases, the TnsB component comprises a polypeptide comprising a sequence having at least about 93% identity to any one of SEQ ID NOs: 2, 13, 17, and 65. In some cases, the TnsB component comprises a polypeptide comprising a sequence having at least about 94% identity to any one of SEQ ID NOs: 2, 13, 17, and 65. In some cases, the TnsB component comprises a polypeptide comprising a sequence having at least about 95% identity to any one of SEQ ID NOs: 2, 13, 17, and 65. In some cases, the TnsB component comprises a polypeptide comprising a sequence having at least about 96% identity to any one of SEQ ID NOs: 2, 13, 17, and 65. In some cases, the TnsB component comprises a polypeptide comprising a sequence having at least about 97% identity to any one of SEQ ID NOs: 2, 13, 17, and 65. In some cases, the TnsB component comprises a polypeptide comprising a sequence having at least about 98% identity to any one of SEQ ID NOs: 2, 13, 17, and 65. In some cases, the TnsB component comprises a polypeptide comprising a sequence having at least about 99% identity to any one of SEQ ID NOs: 2, 13, 17, and 65. In some cases, the TnsB component comprises a polypeptide comprising a sequence having 100% identity to any one of SEQ ID NOs: 2, 13, 17, and 65.

In some cases, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, and 66-67. In some cases, the Tn7 type transposase complex comprises at least one polypeptide comprising a sequence having at least about 70% identity to any one of SEQ ID NOs: 3-4, 14-15, 18-19, and 66-67. In some cases, the Tn7 type transposase complex comprises at least one polypeptide comprising a sequence having at least about 75% identity to any one of SEQ ID NOs: 3-4, 14-15, 18-19, and 66-67. In some cases, the Tn7 type transposase complex comprises at least one polypeptide comprising a sequence having at least about 80% identity to any one of SEQ ID NOs: 3-4, 14-15, 18-19, and 66-67. In some cases, the Tn7 type transposase complex comprises at least one polypeptide comprising a sequence having at least about 85% identity to any one of SEQ ID NOs: 3-4, 14-15, 18-19, and 66-67. In some cases, the Tn7 type transposase complex comprises at least one polypeptide comprising a sequence having at least about 90% identity to any one of SEQ ID NOs: 3-4, 14-15, 18-19, and 66-67. In some cases, the Tn7 type transposase complex comprises at least one polypeptide comprising a sequence having at least about 91% identity to any one of SEQ ID NOs: 3-4, 14-15, 18-19, and 66-67. In some cases, the Tn7 type transposase complex comprises at least one polypeptide comprising a sequence having at least about 92% identity to any one of SEQ ID NOs: 3-4, 14-15, 18-19, and 66-67. In some cases, the Tn7 type transposase complex comprises at least one polypeptide comprising a sequence having at least about 93% identity to any one of SEQ ID NOs: 3-4, 14-15, 18-19, and 66-67. In some cases, the Tn7 type transposase complex comprises at least one polypeptide comprising a sequence having at least about 94% identity to any one of SEQ ID NOs: 3-4, 14-15, 18-19, and 66-67. In some cases, the Tn7 type transposase complex comprises at least one polypeptide comprising a sequence having at least about 95% identity to any one of SEQ ID NOs: 3-4, 14-15, 18-19, and 66-67. In some cases, the Tn7 type transposase complex comprises at least one polypeptide comprising a sequence having at least about 96% identity to any one of SEQ ID NOs: 3-4, 14-15, 18-19, and 66-67. In some cases, the Tn7 type transposase complex comprises at least one polypeptide comprising a sequence having at least about 97% identity to any one of SEQ ID NOs: 3-4, 14-15, 18-19, and 66-67. In some cases, the Tn7 type transposase complex comprises at least one polypeptide comprising a sequence having at least about 98% identity to any one of SEQ ID NOs: 3-4, 14-15, 18-19, and 66-67. In some cases, the Tn7 type transposase complex comprises at least one polypeptide comprising a sequence having at least about 99% identity to any one of SEQ ID NOs: 3-4, 14-15, 18-19, and 66-67. In some cases, the Tn7 type transposase complex comprises at least one polypeptide comprising a sequence having 100% identity to any one of SEQ ID NOs: 3-4, 14-15, 18-19, and 66-67.

In some cases, the Tn7 type transposase complex comprises at least a first polypeptide and a second polypeptide each independently comprising a sequence having at least about 70% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 75% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 80% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 85% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 90% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 91% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 92% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 93% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 94% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 95% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 96% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 97% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 98% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 99% identity to any one of SEQ ID NOs: 3-4, 14-15, 18-19, and 66-67. In some cases, the Tn7 type transposase complex comprises at least a first polypeptide and a second polypeptide each independently comprising a sequence having 100% identity to any one of SEQ ID NOs: 3-4, 14-15, 18-19, and 66-67.

In some embodiments, a system disclosed herein comprises at least one engineered guide polynucleotide, e.g., a gRNA.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 70% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 75% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 80% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 85% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 90% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 91% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 92% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 93% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 94% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 95% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 96% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 97% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 98% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 99% identity to any one of SEQ ID NOs: 5-6, 32-33, 94-95, 104-105, and 202. In some cases, the engineered guide polynucleotide comprises a sequence comprising at least about 46-80 consecutive nucleotides having 100% identity to any one of SEQ ID NOs: 5-6, 32-33, 94-95, 104-105, and 202.

In some cases, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides identical to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 70% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 75% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 80% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 85% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 90% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 91% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 92% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 93% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 94% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 95% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 96% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 97% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 98% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides having 100% identity to any one of SEQ ID NOs: 45-63, 68-75, 96-103, and 165.

In some embodiments, the guide RNAs comprise various structural elements including but not limited to: a spacer sequence which binds to the protospacer sequence (target sequence), a crRNA, and an optional tracrRNA. In some embodiments, the guide RNA comprises a crRNA comprising a spacer sequence. In some embodiments, the guide RNA additionally comprises a tracrRNA or a modified tracrRNA.

In some embodiments, the systems provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a crRNA is part or considered part of a guide RNA, or is comprised in a guide RNA, e.g., a crRNA:tracrRNA chimera.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur (S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, and 78. In some cases, the left-hand recombinase sequence comprises a sequence having at least about 70% identity to any one of SEQ ID NOs: 9, 11, 36-38, 76, and 78. In some cases, the left-hand recombinase sequence comprises a sequence having at least about 75% identity to any one of SEQ ID NOs: 9, 11, 36-38, 76, and 78. In some cases, the left-hand recombinase sequence comprises a sequence having at least about 80% identity to any one of SEQ ID NOs: 9, 11, 36-38, 76, and 78. In some cases, the left-hand recombinase sequence comprises a sequence having at least about 85% identity to any one of SEQ ID NOs: 9, 11, 36-38, 76, and 78. In some cases, the left-hand recombinase sequence comprises a sequence having at least about 90% identity to any one of SEQ ID NOs: 9, 11, 36-38, 76, and 78. In some cases, the left-hand recombinase sequence comprises a sequence having at least about 91% identity to any one of SEQ ID NOs: 9, 11, 36-38, 76, and 78. In some cases, the left-hand recombinase sequence comprises a sequence having at least about 92% identity to any one of SEQ ID NOs: 9, 11, 36-38, 76, and 78. In some cases, the left-hand recombinase sequence comprises a sequence having at least about 93% identity to any one of SEQ ID NOs: 9, 11, 36-38, 76, and 78. In some cases, the left-hand recombinase sequence comprises a sequence having at least about 94% identity to any one of SEQ ID NOs: 9, 11, 36-38, 76, and 78. In some cases, the left-hand recombinase sequence comprises a sequence having at least about 95% identity to any one of SEQ ID NOs: 9, 11, 36-38, 76, and 78. In some cases, the left-hand recombinase sequence comprises a sequence having at least about 96% identity to any one of SEQ ID NOs: 9, 11, 36-38, 76, and 78. In some cases, the left-hand recombinase sequence comprises a sequence having at least about 97% identity to any one of SEQ ID NOs: 9, 11, 36-38, 76, and 78. In some cases, the left-hand recombinase sequence comprises a sequence having at least about 98% identity to any one of SEQ ID NOs: 9, 11, 36-38, 76, and 78. In some cases, the left-hand recombinase sequence comprises a sequence having at least about 99% identity to any one of SEQ ID NOs: 9, 11, 36-38, 76, and 78. In some cases, the left-hand recombinase sequence comprises a sequence having 100% identity to any one of SEQ ID NOs: 9, 11, 36-38, 76, and 78.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, and 93. In some cases, the right-hand recombinase sequence comprises a sequence having at least about 70% identity to any one of SEQ ID NOs: 8, 10, 39-44, 77, 79, and 93. In some cases, the right-hand recombinase sequence comprises a sequence having at least about 75% identity to any one of SEQ ID NOs: 8, 10, 39-44, 77, 79, and 93. In some cases, the right-hand recombinase sequence comprises a sequence having at least about 80% identity to any one of SEQ ID NOs: 8, 10, 39-44, 77, 79, and 93. In some cases, the right-hand recombinase sequence comprises a sequence having at least about 85% identity to any one of SEQ ID NOs: 8, 10, 39-44, 77, 79, and 93. In some cases, the right-hand recombinase sequence comprises a sequence having at least about 90% identity to any one of SEQ ID NOs: 8, 10, 39-44, 77, 79, and 93. In some cases, the right-hand recombinase sequence comprises a sequence having at least about 91% identity to any one of SEQ ID NOs: 8, 10, 39-44, 77, 79, and 93. In some cases, the right-hand recombinase sequence comprises a sequence having at least about 92% identity to any one of SEQ ID NOs: 8, 10, 39-44, 77, 79, and 93. In some cases, the right-hand recombinase sequence comprises a sequence having at least about 93% identity to any one of SEQ ID NOs: 8, 10, 39-44, 77, 79, and 93. In some cases, the right-hand recombinase sequence comprises a sequence having at least about 94% identity to any one of SEQ ID NOs: 8, 10, 39-44, 77, 79, and 93. In some cases, the right-hand recombinase sequence comprises a sequence having at least about 95% identity to any one of SEQ ID NOs: 8, 10, 39-44, 77, 79, and 93. In some cases, the right-hand recombinase sequence comprises a sequence having at least about 96% identity to any one of SEQ ID NOs: 8, 10, 39-44, 77, 79, and 93. In some cases, the right-hand recombinase sequence comprises a sequence having at least about 97% identity to any one of SEQ ID NOs: 8, 10, 39-44, 77, 79, and 93. In some cases, the right-hand recombinase sequence comprises a sequence having at least about 98% identity to any one of SEQ ID NOs: 8, 10, 39-44, 77, 79, and 93. In some cases, the right-hand recombinase sequence comprises a sequence having at least about 99% identity to any one of SEQ ID NOs: 8, 10, 39-44, 77, 79, and 93. In some cases, the right-hand recombinase sequence comprises a sequence having 100% identity to any one of SEQ ID NOs: 8, 10, 39-44, 77, 79, and 93.

In some cases, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In some embodiments, the class 2, type V effector comprises a nuclear localization sequence (NLS). In some embodiments, the NLS is at an N-terminus of the class 2, type V effector. In some embodiments, the NLS is at a C-terminus of the class 2, type V effector. In some embodiments, the NLS is at an N-terminus and a C-terminus of the class 2, type V effector.

In some embodiments, the NLS comprises a sequence of any one of SEQ ID NOs: 172-187, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 172-187. In some cases, the NLS comprises a sequence having at least about 80% identity to any one of SEQ ID NOs: 172-187. In some cases, the NLS comprises a sequence having at least about 85% identity to any one of SEQ ID NOs: 172-187. In some cases, the NLS comprises a sequence having at least about 90% identity to any one of SEQ ID NOs: 172-187. In some cases, the NLS comprises a sequence having at least about 91% identity to any one of SEQ ID NOs: 172-187. In some cases, the NLS comprises a sequence having at least about 92% identity to any one of SEQ ID NOs: 172-187. In some cases, the NLS comprises a sequence having at least about 93% identity to any one of SEQ ID NOs: 172-187. In some cases, the NLS comprises a sequence having at least about 94% identity to any one of SEQ ID NOs: 172-187. In some cases, the NLS comprises a sequence having at least about 95% identity to any one of SEQ ID NOs: 172-187. In some cases, the NLS comprises a sequence having at least about 96% identity to any one of SEQ ID NOs: 172-187. In some cases, the NLS comprises a sequence having at least about 97% identity to any one of SEQ ID NOs: 172-187. In some cases, the NLS comprises a sequence having at least about 98% identity to any one of SEQ ID NOs: 172-187. In some cases, the NLS comprises a sequence having at least about 99% identity to any one of SEQ ID NOs: 172-187. In some cases, the NLS comprises a sequence having 100% identity to any one of SEQ ID NOs: 172-187.

TABLE 1
Exemplary NLS Sequences

| | | SEQ |
|---|---|---|
| | NLS amino | ID |
| Source | acid sequence | NO: |
| SV40 | PKKKRKV | 172 |
| nucleoplasmin | KRPAATKKAGQAKKKK | 173 |
| bipartite NLS | | |
| c-myc NLS | PAAKRVKLD | 174 |
| c-myc NLS | RQRRNELKRSP | 175 |
| hRNPA1 M9 NLS | NQSSNFGPMKGGNFGGRSS | 176 |
| | GPYGGGGQYFAKPRNQGGY | |
| Importin-alpha | RMRIZFKNKGKDTAELRRR | 177 |
| IBB domain | RVEVSVELRKAKKDEQILK | |
| | RRNV | |
| Myoma T protein | VSRKRPRP | 178 |
| Myoma T protein | PPKKARED | 179 |
| p53 | PQPKKKPL | 180 |
| mouse c-abl IV | SALIKKKKKMAP | 181 |
| influenza virus | DRLRR | 182 |
| NS1 | | |
| influenza virus | PKQKKRK | 183 |
| NS1 | | |
| Hepatitis virus | RKLKKKIKKL | 184 |
| delta antigen | | |
| mouse Mx1 | REKKKFLKRR | 185 |
| protein | | |
| human poly(ADP- | KRKGDEVDGVDEVAKK | 186 |
| ribose) | KSKK | |
| polymerase | | |
| steroid hormone | RKCLQAGMNLEARKTKK | 187 |
| receptors (human) | | |
| glucocorticoid | | |

In some embodiments, the Cas effector complex further comprises a small prokaryotic ribosomal protein subunit S15. In some embodiments, the S15 fusion protein is encoded by a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 161-163. In some cases, the S15 is encoded by a sequence having at least about 70% identity to any one of SEQ ID NOs: 161-163. In some cases, the S15 is encoded by a sequence having at least about 75% identity to any one of SEQ ID NOs: 161-163. In some cases, the S15 is encoded by a sequence having at least about 80% identity to any one of SEQ ID NOs: 161-163. In some cases, the S15 is encoded by a sequence having at least about 85% identity to any one of SEQ ID NOs: 161-163. In some cases, the S15 is encoded by a sequence having at least about 90% identity to any one of SEQ ID NOs: 161-163. In some cases, the S15 is encoded by a sequence having at least about 91% identity to any one of SEQ ID NOs: 161-163. In some cases, the S15 is encoded by a sequence having at least about 92% identity to any one of SEQ ID NOs: 161-163. In some cases, the S15 is encoded by a sequence having at least about 93% identity to any one of SEQ ID NOs: 161-163. In some cases, the S15 is encoded by a sequence having at least about 94% identity to any one of SEQ ID NOs: 161-163. In some cases, the S15 is encoded by a sequence having at least about 95% identity to any one of SEQ ID NOs: 161-163. In some cases, the S15 is encoded by a sequence having at least about 96% identity to any one of SEQ ID NOs: 161-163. In some cases, the S15 is encoded by a sequence having at least about 97% identity to any one of SEQ ID NOs: 161-163. In some cases, the S15 is encoded by a sequence having at least about 98% identity to any one of SEQ ID NOs: 161-163. In some cases, the S15 is encoded by a sequence having at least about 99% identity to any one of SEQ ID NOs: 161-163. In some cases, the S15 is encoded by a sequence having 100% identity to any one of SEQ ID NOs: 161-163.

In some cases, the S15 comprises a sequence having at least about 70% identity to any one of SEQ ID NOs: 167-169. In some cases, the S15 comprises a sequence having at least about 75% identity to any one of SEQ ID NOs: 167-169. In some cases, the S15 comprises a sequence having at least about 80% identity to any one of SEQ ID NOs: 167-169. In some cases, the S15 comprises a sequence having at least about 85% identity to any one of SEQ ID NOs: 167-169. In some cases, the S15 comprises a sequence having at least about 90% identity to any one of SEQ ID NOs: 167-169. In some cases, the S15 comprises a sequence having at least about 91% identity to any one of SEQ ID NOs: 167-169. In some cases, the S15 comprises a sequence having at least about 92% identity to any one of SEQ ID NOs: 167-169. In some cases, the S15 comprises a sequence having at least about 93% identity to any one of SEQ ID NOs: 167-169. In some cases, the S15 comprises a sequence having at least about 94% identity to any one of SEQ ID NOs: 167-169. In some cases, the S15 comprises a sequence having at least about 95% identity to any one of SEQ ID NOs: 167-169. In some cases, the S15 comprises a sequence having at least about 96% identity to any one of SEQ ID NOs: 167-169. In some cases, the S15 comprises a sequence having at least about 97% identity to any one of SEQ ID NOs: 167-169. In some cases, the S15 comprises a sequence having at least about 98% identity to any one of SEQ ID NOs: 167-169. In some cases, the S15 comprises a sequence having at least about 99% identity to any one of SEQ ID NOs: 167-169. In some cases, the S15 comprises a sequence having 100% identity to any one of SEQ ID NOs: 167-169.

In some embodiments, the Cas effector complex comprises one or more linkers linking the class 2, type V effector, the small prokaryotic ribosomal protein subunit S15, the transposase, the gRNA, or combinations thereof. In some embodiments, the linker comprises at least about 10, 20, 30, 40, 50, 60, 70, 80, 90, 100, 200, 300, or 400 amino acids. In some embodiments, the linker comprises at least about 10, 20, 30, 40, 50, 60, 70, 80, 90, 100, 200, 300, 400, 500, 600, 700, 800, 900, or 1000 nucleotides. In some embodiments, the linker is encoded by a sequence of SEQ ID NO: 166, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity of SEQ ID NO: 166. In some embodiments, the linker is encoded by SEQ ID NO: 166.

### Fusion Proteins

Described herein, in some embodiments, are systems for transposing a cargo nucleotide sequence into a target nucleic acid site comprising a fusion protein or a nucleic acid encoding the fusion protein. In some embodiments, the fusion protein or a nucleic acid encoding the fusion protein comprises a class 2, type V effector, a small prokaryotic ribosomal protein subunit S15, a transposase, a gRNA, or combinations thereof. In some embodiments, the fusion protein comprises one or more transposases.

In some embodiments, a nuclear localization sequence (NLS) is fused to the class 2, type V effector. In some embodiments, the NLS is fused at an N-terminus of the class 2, type V effector. In some embodiments, the NLS is fused at a C-terminus of the class 2, type V effector. In some embodiments, the NLS is fused at an N-terminus and a C-terminus of the class 2, type V effector.

In some embodiments, the NLS comprises a sequence of any one of SEQ ID NOs: 172-187, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 172-187. In some cases, the NLS comprises a sequence having at least about 80% identity to any one of SEQ ID NOs: 172-187. In some cases, the NLS comprises a sequence having at least about 85% identity to any one of SEQ ID NOs: 172-187. In some cases, the NLS comprises a sequence having at least about 90% identity to any one of SEQ ID NOs: 172-187. In some cases, the NLS comprises a sequence having at least about 91% identity to any one of SEQ ID NOs: 172-187. In some cases, the NLS comprises a sequence having at least about 92% identity to any one of SEQ ID NOs: 172-187. In some cases, the NLS comprises a sequence having at least about 93% identity to any one of SEQ ID NOs: 172-187. In some cases, the NLS comprises a sequence having at least about 94% identity to any one of SEQ ID NOs: 172-187. In some cases, the NLS comprises a sequence having at least about 95% identity to any one of SEQ ID NOs: 172-187. In some cases, the NLS comprises a sequence having at least about 96% identity to any one of SEQ ID NOs: 172-187. In some cases, the NLS comprises a sequence having at least about 97% identity to any one of SEQ ID NOs: 172-187. In some cases, the NLS comprises a sequence having at least about 98% identity to any one of SEQ ID NOs: 172-187. In some cases, the NLS comprises a sequence having at least about 99% identity to any one of SEQ ID NOs: 172-187. In some cases, the NLS comprises a sequence having 100% identity to any one of SEQ ID NOs: 172-187.

In some embodiments, the fusion protein or a nucleic acid encoding the fusion protein comprises a fusion of S15 and a nuclear localization sequence (NLS). In some embodiments, the NLS is fused at an N-terminus of S15. In some embodiments, the NLS is fused at a C-terminus of S15. In some embodiments, the NLS is fused at an N-terminus and a C-terminus of S15.

In some embodiments, the S15 fusion protein further comprises a cleavable peptide. In some embodiments, the peptide is a 2A peptide.

In some embodiments, the S15 fusion protein is encoded by a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 161-163. In some cases, the S15 is encoded by a sequence having at least about 70% identity to any one of SEQ ID NOs: 161-163. In some cases, the S15 is encoded by a sequence having at least about 75% identity to any one of SEQ ID NOs: 161-163. In some cases, the S15 is encoded by a sequence having at least about 80% identity to any one of SEQ ID NOs: 161-163. In some cases, the S15 is encoded by a sequence having at least about 85% identity to any one of SEQ ID NOs: 161-163. In some cases, the S15 is encoded by a sequence having at least about 90% identity to any one of SEQ ID NOs: 161-163. In some cases, the S15 is encoded by a sequence having at least about 91% identity to any one of SEQ ID NOs: 161-163. In some cases, the S15 is encoded by a sequence having at least about 92% identity to any one of SEQ ID NOs: 161-163. In some cases, the S15 is encoded by a sequence having at least about 93% identity to any one of SEQ ID NOs: 161-163. In some cases, the S15 is encoded by a sequence having at least about 94% identity to any one of SEQ ID NOs: 161-163. In some cases, the S15 is encoded by a sequence having at least about 95% identity to any one of SEQ ID NOs: 161-163. In some cases, the S15 is encoded by a sequence having at least about 96% identity to any one of SEQ ID NOs: 161-163. In some cases, the S15 is encoded by a sequence having at least about 97% identity to any one of SEQ ID NOs: 161-163. In some cases, the S15 is encoded by a sequence having at least about 98% identity to any one of SEQ ID NOs: 161-163. In some cases, the S15 is encoded by a sequence having at least about 99% identity to any one of SEQ ID NOs: 161-163. In some cases, the S15 is encoded by a sequence having 100% identity to any one of SEQ ID NOs: 161-163.

In some cases, the S15 comprises a sequence having at least about 70% identity to any one of SEQ ID NOs: 167-169. In some cases, the S15 comprises a sequence having at least about 75% identity to any one of SEQ ID NOs: 167-169. In some cases, the S15 comprises a sequence having at least about 80% identity to any one of SEQ ID NOs: 167-169. In some cases, the S15 comprises a sequence having at least about 85% identity to any one of SEQ ID NOs: 167-169. In some cases, the S15 comprises a sequence having at least about 90% identity to any one of SEQ ID NOs: 167-169. In some cases, the S15 comprises a sequence having at least about 91% identity to any one of SEQ ID NOs: 167-169. In some cases, the S15 comprises a sequence having at least about 92% identity to any one of SEQ ID NOs: 167-169. In some cases, the S15 comprises a sequence having at least about 93% identity to any one of SEQ ID NOs: 167-169. In some cases, the S15 comprises a sequence having at least about 94% identity to any one of SEQ ID NOs: 167-169. In some cases, the S15 comprises a sequence having at least about 95% identity to any one of SEQ ID NOs: 167-169. In some cases, the S15 comprises a sequence having at least about 96% identity to any one of SEQ ID NOs: 167-169. In some cases, the S15 comprises a sequence having at least about 97% identity to any one of SEQ ID NOs: 167-169. In some cases, the S15 comprises a sequence having at least about 98% identity to any one of SEQ ID NOs: 167-169. In some cases, the S15 comprises a sequence having at least about 99% identity to any one of SEQ ID NOs: 167-169. In some cases, the S15 comprises a sequence having 100% identity to any one of SEQ ID NOs: 167-169.

In some embodiments, an NLS is fused to the transposase. In some embodiments, the transposase is TnsB, TnsC, or TniQ. In some embodiments, the transposase is TnsB. In some embodiments, the transposase is TnsC. In some embodiments, the transposase is TniQ. In some embodiments, the NLS is fused at an N-terminus of the transposase. In some embodiments, the NLS is fused at a C-terminus of the transposase. In some embodiments, the NLS is fused at an N-terminus and a C-terminus of the transposase.

In some embodiments, the NLS is fused at an N-terminus of the transposase. In some embodiments, the NLS is fused at a C-terminus of the transposase. In some embodiments, the NLS is fused at an N-terminus and a C-terminus of the transposase.

In some embodiments, the fusion protein or a nucleic acid encoding the fusion protein comprises a gRNA described herein (for example a dual gRNA or a single gRNA).

In some embodiments, the class 2, type V effector, the small prokaryotic ribosomal protein subunit S15, the transposase, the gRNA, or a fusion protein comprises a tag. In some embodiments, the tag is a polypeptide or a polynucleotide. In some embodiments, the tag is an affinity tag. Exemplary affinity tags include, but are not limited to, a His-tag, a Flag tag, a Myc-tag, an MBP-tag, and a GST-tag.

In some embodiments, the class 2, type V effector, the small prokaryotic ribosomal protein subunit S15, the transposase, the single gRNA, or a fusion protein or gene editing system comprising any combination thereof, comprises a tag. In some embodiments, the tag is an affinity tag. Exemplary affinity tags include, but are not limited to, a His-tag, a Flag tag, a Myc-tag, an MBP-tag, and a GST-tag.

In some embodiments, the class 2, type V effector, the small prokaryotic ribosomal protein subunit S15, the transposase, or a fusion protein, comprises a protease cleavage site. Exemplary protease cleavage sites include, but are not limited to, a TEV site, a C3 site, a Factor Xa site, and an Enterokinase site.

### Cells

Described herein, in certain embodiments, is a cell comprising the systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NS0), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof.

### Delivery and Vectors

Disclosed herein, in some embodiments, are nucleic acid sequences encoding a MG64 system comprising a class 2, type V effector, a small prokaryotic ribosomal protein subunit 515, a transposase, a gRNA, a fusion protein or a gene editing system disclosed herein.

In some embodiments, the nucleic acid encoding the MG64 system is a DNA, for example a linear DNA, a plasmid DNA, or a minicircle DNA. In some embodiments, the nucleic acid encoding the MG64 system is an RNA, for example a mRNA.

In some embodiments, the nucleic acid encoding the MG64 system is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 vector, pEF1a-tdTomato vector, pSF-CMV-FMDV-Hygro, pSF-CMV-PGK-Puro, pMCP-tag(m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter. In some embodiments, the promoter is encoded by a sequence of any one of SEQ ID NOs: 190-191, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity of any one of SEQ ID NOs: 190-191.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the MG64 system delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the fusion protein or genome editing system is introduced into the cell in any suitable way, either stably or transiently. In some embodiments, a fusion protein or genome editing system is transfected into the cell. In some embodiments, the cell is transduced or transfected with a nucleic acid construct that encodes a fusion protein or genome editing system. For example, a cell is transduced (e.g., with a virus encoding a fusion protein or genome editing system), or transfected (e.g., with a plasmid encoding a fusion protein or genome editing system) with a nucleic acid that encodes a fusion protein or genome editing system, or the translated fusion protein or genome editing system. In some embodiments, the transduction is a stable or transient transduction. In some embodiments, cells expressing a fusion protein or genome editing system or containing a fusion protein or genome editing system are transduced or transfected with one or more gRNA molecules, for example when the fusion protein or genome editing system comprises a CRISPR nuclease. In some embodiments, a plasmid expressing a fusion protein or genome editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Methods for Transposition

The present disclosure provides methods for transposing a cargo nucleotide sequence into a target nucleic acid site. In some embodiments, the method comprises expressing a system described herein within a cell or introducing a system described herein to a cell. In some embodiments, the method comprises contacting a cell with a system described herein.

In some embodiments, the method comprises contacting a double-stranded nucleic acid comprising the cargo nucleotide sequence with a Cas effector complex comprising a class 2, type V Cas effector and at least one engineered guide polynucleotide configured to hybridize to the target nucleotide sequence. In some embodiments, the method comprises contacting the double-stranded nucleic acid comprising the cargo nucleotide sequence with a Tn7 type transposase complex configured to bind the Cas effector complex, wherein the Tn7 type transposase complex comprises a TnsB subunit. In some embodiments, the method comprises contacting the double-stranded nucleic acid comprising the cargo nucleotide sequence with a double-stranded target nucleic acid comprising the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the method further comprises a PAM sequence compatible with the Cas effector complex adjacent to the target nucleic acid site. In some cases, the PAM sequence is located 3′ of the target nucleic acid site.

In some cases, the engineered guide polynucleotide is configured to bind the class 2, type V Cas effector. In some cases, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, 80-85, and 200, or a variant thereof. In some cases, the class 2, type V Cas effector comprises a polypeptide comprising a sequence having at least about 70% identity to any one of SEQ ID NOs: 1, 12, 16, 20-30, 64, 80-85, and 200. In some cases, the class 2, type V Cas effector comprises a polypeptide comprising a sequence having at least about 75% identity to any one of SEQ ID NOs: 1, 12, 16, 20-30, 64, 80-85, and 200. In some cases, the class 2, type V Cas effector comprises a polypeptide comprising a sequence having at least about 80% identity to any one of SEQ ID NOs: 1, 12, 16, 20-30, 64, 80-85, and 200. In some cases, the class 2, type V Cas effector comprises a polypeptide comprising a sequence having at least about 85% identity to any one of SEQ ID NOs: 1, 12, 16, 20-30, 64, 80-85, and 200. In some cases, the class 2, type V Cas effector comprises a polypeptide comprising a sequence having at least about 90% identity to any one of SEQ ID NOs: 1, 12, 16, 20-30, 64, 80-85, and 200. In some cases, the class 2, type V Cas effector comprises a polypeptide comprising a sequence having at least about 91% identity to any one of SEQ ID NOs: 1, 12, 16, 20-30, 64, 80-85, and 200. In some cases, the class 2, type V Cas effector comprises a polypeptide comprising a sequence having at least about 92% identity to any one of SEQ ID NOs: 1, 12, 16, 20-30, 64, 80-85, and 200. In some cases, the class 2, type V Cas effector comprises a polypeptide comprising a sequence having at least about 93% identity to any one of SEQ ID NOs: 1, 12, 16, 20-30, 64, 80-85, and 200. In some cases, the class 2, type V Cas effector comprises a polypeptide comprising a sequence having at least about 94% identity to any one of SEQ ID NOs: 1, 12, 16, 20-30, 64, 80-85, and 200. In some cases, the class 2, type V Cas effector comprises a polypeptide comprising a sequence having at least about 95% identity to any one of SEQ ID NOs: 1, 12, 16, 20-30, 64, 80-85, and 200. In some cases, the class 2, type V Cas effector comprises a polypeptide comprising a sequence having at least about 96% identity to any one of SEQ ID NOs: 1, 12, 16, 20-30, 64, 80-85, and 200. In some cases, the class 2, type V Cas effector comprises a polypeptide comprising a sequence having at least about 97% identity to any one of SEQ ID NOs: 1, 12, 16, 20-30, 64, 80-85, and 200. In some cases, the class 2, type V Cas effector comprises a polypeptide comprising a sequence having at least about 98% identity to any one of SEQ ID NOs: 1, 12, 16, 20-30, 64, 80-85, and 200. In some cases, the class 2, type V Cas effector comprises a polypeptide comprising a sequence having at least about 99% identity to any one of SEQ ID NOs: 1, 12, 16, 20-30, 64, 80-85, and 200. In some cases, the class 2, type V Cas effector comprises a polypeptide comprising a sequence having 100% identity to any one of SEQ ID NOs: 1, 12, 16, 20-30, 64, 80-85, and 200.

In some cases, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, and 65. In some cases, the TnsB component comprises a polypeptide comprising a sequence having at least about 70% identity to any one of SEQ ID NOs: 2, 13, 17, and 65. In some cases, the TnsB component comprises a polypeptide comprising a sequence having at least about 75% identity to any one of SEQ ID NOs: 2, 13, 17, and 65. In some cases, the TnsB component comprises a polypeptide comprising a sequence having at least about 80% identity to any one of SEQ ID NOs: 2, 13, 17, and 65. In some cases, the TnsB component comprises a polypeptide comprising a sequence having at least about 85% identity to any one of SEQ ID NOs: 2, 13, 17, and 65. In some cases, the TnsB component comprises a polypeptide comprising a sequence having at least about 90% identity to any one of SEQ ID NOs: 2, 13, 17, and 65. In some cases, the TnsB component comprises a polypeptide comprising a sequence having at least about 91% identity to any one of SEQ ID NOs: 2, 13, 17, and 65. In some cases, the TnsB component comprises a polypeptide comprising a sequence having at least about 92% identity to any one of SEQ ID NOs: 2, 13, 17, and 65. In some cases, the TnsB component comprises a polypeptide comprising a sequence having at least about 93% identity to any one of SEQ ID NOs: 2, 13, 17, and 65. In some cases, the TnsB component comprises a polypeptide comprising a sequence having at least about 94% identity to any one of SEQ ID NOs: 2, 13, 17, and 65. In some cases, the TnsB component comprises a polypeptide comprising a sequence having at least about 95% identity to any one of SEQ ID NOs: 2, 13, 17, and 65. In some cases, the TnsB component comprises a polypeptide comprising a sequence having at least about 96% identity to any one of SEQ ID NOs: 2, 13, 17, and 65. In some cases, the TnsB component comprises a polypeptide comprising a sequence having at least about 97% identity to any one of SEQ ID NOs: 2, 13, 17, and 65. In some cases, the TnsB component comprises a polypeptide comprising a sequence having at least about 98% identity to any one of SEQ ID NOs: 2, 13, 17, and 65. In some cases, the TnsB component comprises a polypeptide comprising a sequence having at least about 99% identity to any one of SEQ ID NOs: 2, 13, 17, and 65. In some cases, the TnsB component comprises a polypeptide comprising a sequence having 100% identity to any one of SEQ ID NOs: 2, 13, 17, and 65.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, and 66-67. In some cases, the Tn7 type transposase complex comprises at least one polypeptide comprising a sequence having at least about 70% identity to any one of SEQ ID NOs: 3-4, 14-15, 18-19, and 66-67. In some cases, the Tn7 type transposase complex comprises at least one polypeptide comprising a sequence having at least about 75% identity to any one of SEQ ID NOs: 3-4, 14-15, 18-19, and 66-67. In some cases, the Tn7 type transposase complex comprises at least one polypeptide comprising a sequence having at least about 80% identity to any one of SEQ ID NOs: 3-4, 14-15, 18-19, and 66-67. In some cases, the Tn7 type transposase complex comprises at least one polypeptide comprising a sequence having at least about 85% identity to any one of SEQ ID NOs: 3-4, 14-15, 18-19, and 66-67. In some cases, the Tn7 type transposase complex comprises at least one polypeptide comprising a sequence having at least about 90% identity to any one of SEQ ID NOs: 3-4, 14-15, 18-19, and 66-67. In some cases, the Tn7 type transposase complex comprises at least one polypeptide comprising a sequence having at least about 91% identity to any one of SEQ ID NOs: 3-4, 14-15, 18-19, and 66-67. In some cases, the Tn7 type transposase complex comprises at least one polypeptide comprising a sequence having at least about 92% identity to any one of SEQ ID NOs: 3-4, 14-15, 18-19, and 66-67. In some cases, the Tn7 type transposase complex comprises at least one polypeptide comprising a sequence having at least about 93% identity to any one of SEQ ID NOs: 3-4, 14-15, 18-19, and 66-67. In some cases, the Tn7 type transposase complex comprises at least one polypeptide comprising a sequence having at least about 94% identity to any one of SEQ ID NOs: 3-4, 14-15, 18-19, and 66-67. In some cases, the Tn7 type transposase complex comprises at least one polypeptide comprising a sequence having at least about 95% identity to any one of SEQ ID NOs: 3-4, 14-15, 18-19, and 66-67. In some cases, the Tn7 type transposase complex comprises at least one polypeptide comprising a sequence having at least about 96% identity to any one of SEQ ID NOs: 3-4, 14-15, 18-19, and 66-67. In some cases, the Tn7 type transposase complex comprises at least one polypeptide comprising a sequence having at least about 97% identity to any one of SEQ ID NOs: 3-4, 14-15, 18-19, and 66-67. In some cases, the Tn7 type transposase complex comprises at least one polypeptide comprising a sequence having at least about 98% identity to any one of SEQ ID NOs: 3-4, 14-15, 18-19, and 66-67. In some cases, the Tn7 type transposase complex comprises at least one polypeptide comprising a sequence having at least about 99% identity to any one of SEQ ID NOs: 3-4, 14-15, 18-19, and 66-67. In some cases, the Tn7 type transposase complex comprises at least one polypeptide comprising a sequence having 100% identity to any one of SEQ ID NOs: 3-4, 14-15, 18-19, and 66-67.

In some cases, the Tn7 type transposase complex comprises at least a first polypeptide and a second polypeptide each independently comprising a sequence having at least about 70% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 75% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 80% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 85% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 90% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 91% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 92% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 93% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 94% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 95% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 96% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 97% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 98% identity to any one of SEQ ID NOs: 3-4, 14-15, 18-19, and 66-67. In some cases, the Tn7 type transposase complex comprises at least a first polypeptide and a second polypeptide each independently comprising a sequence having at least about 99% identity to any one of SEQ ID NOs: 3-4, 14-15, 18-19, and 66-67. In some cases, the Tn7 type transposase complex comprises at least a first polypeptide and a second polypeptide each independently comprising a sequence having 100% identity to any one of SEQ ID NOs: 3-4, 14-15, 18-19, and 66-67.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 70% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 75% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 80% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 85% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 90% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 91% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 92% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 93% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 94% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 95% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 96% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 97% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 98% identity to any one of SEQ ID NOs: 5-6, 32-33, 94-95, 104-105, and 202. In some cases, the engineered guide polynucleotide comprises a sequence comprising at least about 46-80 consecutive nucleotides having at least about 99% identity to any one of SEQ ID NOs: 5-6, 32-33, 94-95, 104-105, and 202. In some cases, the engineered guide polynucleotide comprises a sequence comprising at least about 46-80 consecutive nucleotides having 100% identity to any one of SEQ ID NOs: 5-6, 32-33, 94-95, 104-105, and 202.

In some cases, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides identical to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 70% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 75% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 80% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 85% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 90% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 91% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 92% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 93% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 94% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 95% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 96% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 97% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 98% identity to any one of SEQ ID NOs: 45-63, 68-75, 96-103, and 165. In some cases, the engineered guide polynucleotide is a guide RNA and comprises a sequence comprising at least about 46-80 consecutive nucleotides having at least about 99% identity to any one of SEQ ID NOs: 45-63, 68-75, 96-103, and 165. In some cases, the engineered guide polynucleotide is a guide RNA and comprises a sequence comprising at least about 46-80 consecutive nucleotides having 100% identity to any one of SEQ ID NOs: 45-63, 68-75, 96-103, and 165.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, and 78. In some cases, the left-hand recombinase sequence comprises a sequence having at least about 70% identity to any one of SEQ ID NOs: 9, 11, 36-38, 76, and 78. In some cases, the left-hand recombinase sequence comprises a sequence having at least about 75% identity to any one of SEQ ID NOs: 9, 11, 36-38, 76, and 78. In some cases, the left-hand recombinase sequence comprises a sequence having at least about 80% identity to any one of SEQ ID NOs: 9, 11, 36-38, 76, and 78. In some cases, the left-hand recombinase sequence comprises a sequence having at least about 85% identity to any one of SEQ ID NOs: 9, 11, 36-38, 76, and 78. In some cases, the left-hand recombinase sequence comprises a sequence having at least about 90% identity to any one of SEQ ID NOs: 9, 11, 36-38, 76, and 78. In some cases, the left-hand recombinase sequence comprises a sequence having at least about 91% identity to any one of SEQ ID NOs: 9, 11, 36-38, 76, and 78. In some cases, the left-hand recombinase sequence comprises a sequence having at least about 92% identity to any one of SEQ ID NOs: 9, 11, 36-38, 76, and 78. In some cases, the left-hand recombinase sequence comprises a sequence having at least about 93% identity to any one of SEQ ID NOs: 9, 11, 36-38, 76, and 78. In some cases, the left-hand recombinase sequence comprises a sequence having at least about 94% identity to any one of SEQ ID NOs: 9, 11, 36-38, 76, and 78. In some cases, the left-hand recombinase sequence comprises a sequence having at least about 95% identity to any one of SEQ ID NOs: 9, 11, 36-38, 76, and 78. In some cases, the left-hand recombinase sequence comprises a sequence having at least about 96% identity to any one of SEQ ID NOs: 9, 11, 36-38, 76, and 78. In some cases, the left-hand recombinase sequence comprises a sequence having at least about 97% identity to any one of SEQ ID NOs: 9, 11, 36-38, 76, and 78. In some cases, the left-hand recombinase sequence comprises a sequence having at least about 98% identity to any one of SEQ ID NOs: 9, 11, 36-38, 76, and 78. In some cases, the left-hand recombinase sequence comprises a sequence having at least about 99% identity to any one of SEQ ID NOs: 9, 11, 36-38, 76, and 78. In some cases, the left-hand recombinase sequence comprises a sequence having 100% identity to any one of SEQ ID NOs: 9, 11, 36-38, 76, and 78.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, and 93. In some cases, the right-hand recombinase sequence comprises a sequence having at least about 70% identity to any one of SEQ ID NOs: 8, 10, 39-44, 77, 79, and 93. In some cases, the right-hand recombinase sequence comprises a sequence having at least about 75% identity to any one of SEQ ID NOs: 8, 10, 39-44, 77, 79, and 93. In some cases, the right-hand recombinase sequence comprises a sequence having at least about 80% identity to any one of SEQ ID NOs: 8, 10, 39-44, 77, 79, and 93. In some cases, the right-hand recombinase sequence comprises a sequence having at least about 85% identity to any one of SEQ ID NOs: 8, 10, 39-44, 77, 79, and 93. In some cases, the right-hand recombinase sequence comprises a sequence having at least about 90% identity to any one of SEQ ID NOs: 8, 10, 39-44, 77, 79, and 93. In some cases, the right-hand recombinase sequence comprises a sequence having at least about 91% identity to any one of SEQ ID NOs: 8, 10, 39-44, 77, 79, and 93. In some cases, the right-hand recombinase sequence comprises a sequence having at least about 92% identity to any one of SEQ ID NOs: 8, 10, 39-44, 77, 79, and 93. In some cases, the right-hand recombinase sequence comprises a sequence having at least about 93% identity to any one of SEQ ID NOs: 8, 10, 39-44, 77, 79, and 93. In some cases, the right-hand recombinase sequence comprises a sequence having at least about 94% identity to any one of SEQ ID NOs: 8, 10, 39-44, 77, 79, and 93. In some cases, the right-hand recombinase sequence comprises a sequence having at least about 95% identity to any one of SEQ ID NOs: 8, 10, 39-44, 77, 79, and 93. In some cases, the right-hand recombinase sequence comprises a sequence having at least about 96% identity to any one of SEQ ID NOs: 8, 10, 39-44, 77, 79, and 93. In some cases, the right-hand recombinase sequence comprises a sequence having at least about 97% identity to any one of SEQ ID NOs: 8, 10, 39-44, 77, 79, and 93. In some cases, the right-hand recombinase sequence comprises a sequence having at least about 98% identity to any one of SEQ ID NOs: 8, 10, 39-44, 77, 79, and 93. In some cases, the right-hand recombinase sequence comprises a sequence having at least about 99% identity to any one of SEQ ID NOs: 8, 10, 39-44, 77, 79, and 93. In some cases, the right-hand recombinase sequence comprises a sequence having 100% identity to any one of SEQ ID NOs: 8, 10, 39-44, 77, 79, and 93.

In some cases, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### Uses

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Kits

In some embodiments, this disclosure provides kits comprising one or more nucleic acid constructs encoding the various components of the fusion protein or genome editing system described herein, e.g., comprising a nucleotide sequence encoding the components of the fusion protein or genome editing system capable of modifying a target DNA sequence. In some embodiments, the nucleotide sequence comprises a heterologous promoter that drives expression of the RNA genome editing system components.

In some embodiments, the class 2, type V effector, the small prokaryotic ribosomal protein subunit S15, the transposase, the single gRNA, or a fusion protein or gene editing system comprising any combination thereof disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1—(General Protocol) PAM Sequence Identification/Confirmation for Systems Described Herein

Putative endonucleases were expressed in an E. coli lysate-based expression system. PAM sequences were determined by sequencing plasmids containing randomly-generated potential PAM sequences that are able to be cleaved by the putative nucleases. In this system, an E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence in the in vitro expression system followed by CRISPR array processing provided active in vitro CRISPR nuclease complexes.

A library of target plasmids containing a spacer sequence matching that in the minimal array preceded by 8N mixed bases (potential PAM sequences) was incubated with the output of the in vitro expression reaction. After 1-3 hr, the reaction was stopped and the DNA was recovered via a DNA clean-up kit. Adapter sequences were blunt-end ligated to DNA with active PAM sequences that were cleaved by the endonuclease, whereas DNA that was not cleaved was inaccessible for ligation. DNA segments comprising active PAM sequences were then amplified by PCR with primers specific to the library and the adapter sequence. The PCR amplification products were resolved on a gel to identify amplicons that correspond to cleavage events. The amplified segments of the cleavage reaction were also used as templates for preparation of an NGS library or as a substrate for Sanger sequencing. Sequencing this resulting library, which is a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template was omitted.

Analysis of the intergenic regions surrounding the Cas effector and CRISPR array identified a potential anti-repeat sequence corresponding to the duplexing sequence of the tracrRNA. TracrRNA and crRNA repeat were folded and trimmed, adding a tetraloop sequence of GAAA to maintain the stem loop region of the crRNA-tracrRNA complex.

### Example 2A—In Vitro Targeted Integrase Activity

Integrase activity was assayed with a previously identified PAM but may be conducted with a PAM library substrate instead, with reduced efficiency. One arrangement of components for in vitro testing involved three plasmids other than that containing the donor sequence: (1) an expression plasmid with effector (or effectors) under a T7 promoter; (2) an expression plasmid with transposase genes under a T7 promoter; a sgRNA or crRNA and tracrRNA; (3) a target plasmid which contained the spacer site and appropriate PAM; and (4) a donor plasmid which contained the required left end (LE) and right end (RE) DNA sequences for transposition around a cargo gene (e.g., a selection marker such as a Tet resistance gene). Using an in vitro transcription/translation system (e.g., E. coli lysate- or reticulocyte lysate-based system), the effector and transposase genes were expressed. After expression, the RNA, target DNA, and donor DNA were added and incubated to allow for transposition to occur. Transposition was detected via PCR across the junction of the transposase site, with one primer on the target DNA and one primer on the donor DNA. The resulting PCR product was sequenced via NGS to determine the exact insertion topology relative to the sgRNA/crRNA targeted site. The primers were located downstream such that a variety of insertion sites were accommodated and detected. Primers were designed such that integration was detected in either orientation of cargo and on either side of the spacer, as the integration direction was also not previously documented.

Integration efficiency was measured via quantitative PCR (qPCR) measurements of the experimental output of target DNA with integrated cargo, normalized to the amount of unmodified target DNA also measured via qPCR.

This assay may be conducted with purified protein components rather than from lysate-based expression. In this case the proteins were expressed in an E. coli protease deficient B strain under a T7 inducible promoter, the cells were lysed using sonication, and the His-tagged protein of interest was purified using Ni-NTA affinity chromatography on an FPLC. Purity was determined using densitometry in of the protein bands resolved on SDS-PAGE and Coomassie stained acrylamide gels. The protein was desalted in storage buffer composed of 50 mM Tris-HCl, 300 mM NaCl, 1 mM TCEP, 5% glycerol; pH 7.5 (or other buffers as determined for maximum stability) and stored at −80° C. After purification the effector(s) and transposase(s) were added to the sgRNA, target DNA, and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol, (final pH 7.5) supplemented with 15 mM Mg(OAc)₂.

### Example 2B—In Vitro Activity

### Targeted Nuclease

In situ expression and protein sequence analyses indicated that some RNA guided effectors are active nucleases. They contained predicted endonuclease-associated domains (matching RuvC and HNH_endonuclease domains), and/or predicted HNH and RuvC catalytic residues.

Candidate activity was tested with engineered single guide RNA sequences using the in vitro expression system and in vitro transcribed RNA. Active proteins that successfully cleaved the library yielded a band around 170 bp in the gel.

### DNA Integration and Transposition

Transposons are predicted to be active when the genomic sequences encoding them contain one or more protein sequences with transposase and/or integrase function within the left and right ends of the transposon. A Tn7 transposon, as defined here, may comprise a catalytic transposase TnsB, but may also contain TnsA, TnsC, TnsD, TnsE, TniQ, and/or other transposase or integrases. The transposon ends comprise predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g., FIG. 4A).

### Targeted DNA Integration

Putative CRISPR-associated transposons (CAST) contain a DNA and/or RNA targeting CRISPR nuclease or effector and proteins with predicted transposase function in the vicinity of a CRISPR array. In some systems, the nuclease is predicted to be active based on the presence of endonuclease-associated catalytic domains and/or catalytic residues.

In some systems, the effector is predicted to have homology with documented CRISPR effector proteins, but to be inactive based on the absence of endonuclease domains and/or catalytic residues. The transposases are predicted to be associated with the effector when the CRISPR loci (inactive CRISPR nuclease and array) and the transposase proteins are located within the predicted transposon left and right ends (FIG. 4A). In this case, the effector is predicted to direct DNA integration to specific genomic locations based on a guide RNA.

CAST activity was tested with five types of components (1) a Cas effector protein expressed by the in vitro expression system, (2) a target DNA fragment or plasmid containing the target sequence and PAM corresponding to the Cas enzyme, (3) a donor DNA fragments containing a marker or fragment of DNA flanked by the LE and RE of the transposase system in a DNA fragment or plasmid (4) any combination of transposase proteins expressed using the in vitro expression system, and (5) an engineered in vitro transcribed single guide RNA sequence. Active systems that successfully transposed the donor fragment were assayed by PCR amplification of the donor-target junction.

After performing the transposition reaction, PCR amplification of the junction showed that proper donor-target formation was made, and the transposition reaction was sg dependent. (FIG. 6). PCR amplification of reactions #3 and #4 indicated that both orientations of the donor relative to the target were made: one where the LE is closer to the PAM, and one where the RE is closer to the PAM. While both transposition orientations were made, there was a preference for donor integration in the target where the LE is closer to the PAM, represented by strong band present for reactions #4 and #5.

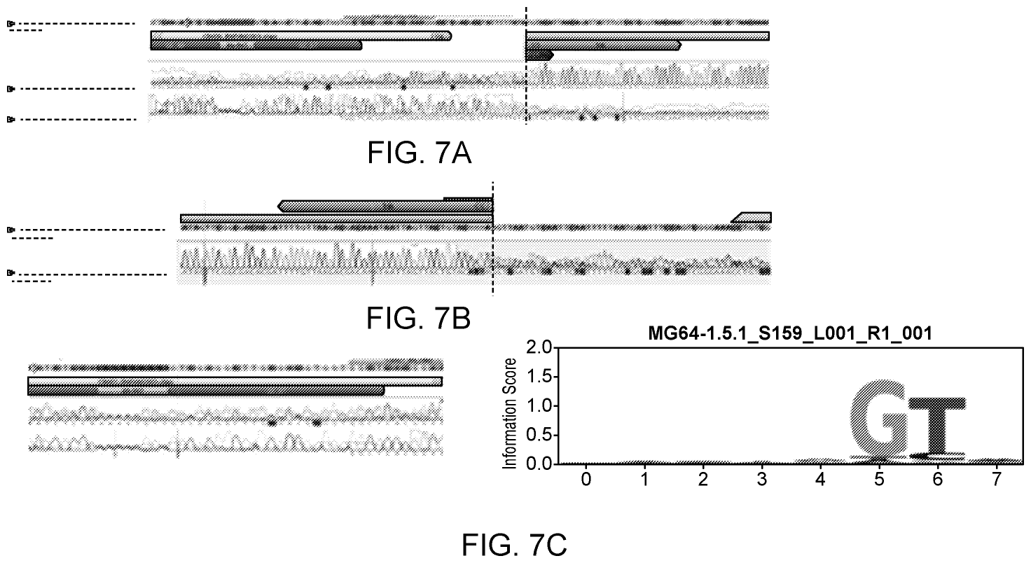

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 7a). Sequencing that originated from the donor over the donor-target junction defined the composition of the essential outer bounds of the LE and RE sequences (FIGS. 7A and 7B). Further investigation of the LE and RE domains will determine the inner limits of the LE and RE sequences that are essential for transposition. Sequencing of the RE on LE-closer-to-PAM products showed a 3 bp duplication downstream of the donor RE (FIG. 7B). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also elucidated that the PAM preference of the MG64-1 effector as a nGTn/nGTt on the 5′ end of the spacer (FIG. 7C). NGS analysis of the PAM library target corroborated the nGTn motif preference at the 5′ end.

### Example 3—Predicted RNA Folding

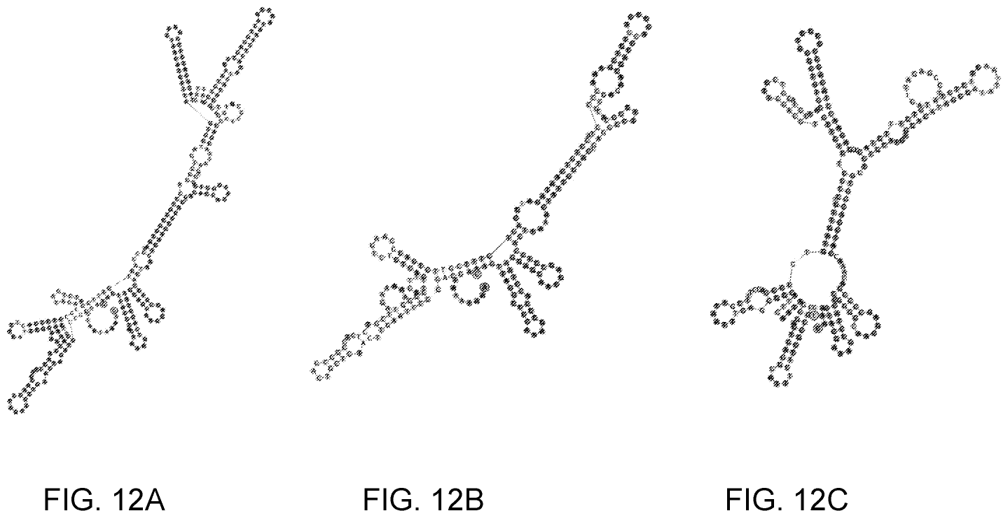

Predicted RNA folding of the active single RNA sequence was computed at 370 using the method of Andronescu 2007. All hairpin-loop secondary structures were singly deleted from the structure and iteratively compiled into a smaller single guide. In a second approach, the tracrRNA of MG64-1 was aligned to documented type Vk tracrRNA, and areas of unique insertions were mutated out of the single guide, and minimized by 57 bases. FIG. 12A depicts the predicted structure of MG64-1 sgRNA. FIG. 12B depicts the predicted structure of MG64-3 sgRNA. FIG. 12C depicts the predicted structure of MG64-5 sgRNA.

### Example 4—Transposon End Verification Via Gel Shift

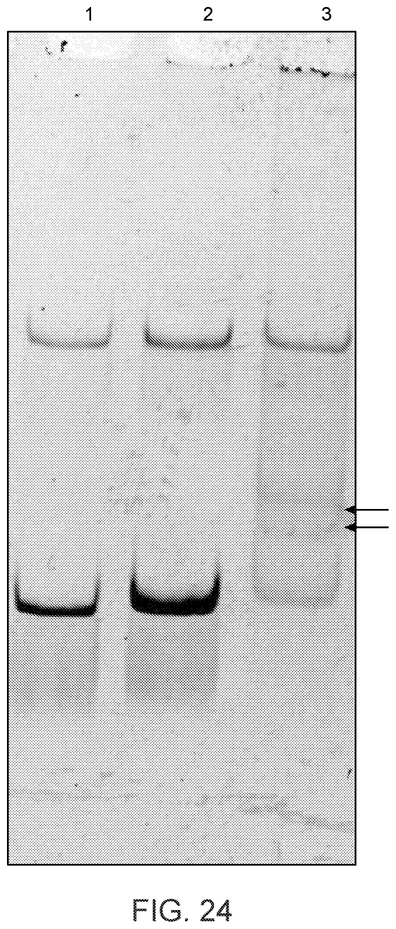

The transposon ends were tested for TnsB binding via an electrophoretic mobility shift assay (EMSA). In this case the potential LE or RE was synthesized as a DNA fragment (100-500 bp) and end-labeled with FAM via PCR with FAM-labeled primers. The TnsB protein was synthesized in an in vitro transcription/translation system. After synthesis, 1 μL of TnsB protein was added to 50 nM of the labeled RE or LE in a 10 μL reaction in binding buffer (20 mM HEPES pH 7.5, 2.5 mM Tris pH 7.5, 10 mM NaCl, 0.0625 mM EDTA, 5 mM TCEP, 0.005% BSA, 1 ug/mL poly(dI-dC), and 5% glycerol). The binding was incubated at 300 for 40 minutes, then 2 uL of 6× loading buffer (60 mM KCl, 10 mM Tris pH 7.6, 50% glycerol) was added. The binding reaction was separated on a 5% TBE gel and visualized. Shifts of the LE or RE in the presence of TnsB were attributed to successful binding and were indicative of transposase activity (FIG. 24).

### Example 5—Integrase Activity in E. coli

As E. coli lacks the capacity to efficiently repair genomic double-stranded DNA breaks, transformation of E. coli by agents able to cause double-stranded breaks in the E. coli genome causes cell death. Exploiting this phenomenon, endonuclease or effector-assisted integrase activity was tested in E. coli by recombinantly expressing either the endonuclease or effector-assisted integrase and a guide RNA (determined e.g., as in Example 3) in a target strain with spacer/target and PAM sequences integrated into its genomic DNA.

Engineered strains were then transformed with a plasmid containing the nuclease or effector with single guide RNA, a plasmid expressing the integrase and accessory genes, and a plasmid containing a temperature sensitive origin of replication with a selectable marker flanked by left end (LE) and right end (RE) transposon motifs for integration. Transformants induced for expression of these genes were then screened for transfer of the marker to the genomic target by selection at restrictive temperature for plasmid replication and the marker integration in the genome was confirmed by PCR.

Off target integrations were screened using an unbiased approach. In brief, purified gDNA was fragmented with Tn5 transposase or shearing, and DNA of interest was then PCR amplified using primers specific to a ligated adaptor and the selectable marker. The amplicons were then prepared for NGS sequencing. Analysis of the resulting sequences were trimmed of the transposon sequences and flanking sequences were mapped to the genome to determine insertion position, and off target insertion rates were determined.

### Example 6—Colony PCR Screen of Transposase Activity

For testing of nuclease or effector assisted integrase activity in bacterial cells, strain MGB0032 was constructed from BL21(DE3) E. coli cells which were engineered to contain the target and corresponding PAM sequence specific to MG64_1. MGB0032 E. coli cells were then transformed with pJL56 (plasmid that expresses the MG64_1 effector and helper suite, ampicillin resistant) and pTCM 641 sg, a chloramphenicol-resistant plasmid that expresses the single guide RNA sequence for the engineered target of interest driven by a T7 promoter.

An MGB0032 culture containing both plasmids was then grown to a saturation, diluted at least 1:10 into growth culture with appropriate antibiotics, and incubated at 37° C. until OD of approximately 1. Cells from this growth stage were made electrocompetent and transformed with streamlined 64_1 pDonor, a plasmid bearing a tetracycline resistance marker flanked by left end (LE) and right end (RE) transposon motifs for integration. Electroporated cells were then recovered for 2 hours on LB medium in the presence or absence of IPTG at a final concentration of 100 μM before being plated on LB-agar-ampicillin-chloramphenicol-tetracycline and incubated 4 days at 37° C. Sterile toothpicks were used to sample each resultant CFU, which was mixed into water. To this solution was added Q5 High Fidelity PCR mastermix and primers LA155 (5′-GCTCTTCCGATCTNNNNNGATGAGCGCATTGTTAGATTTCAT-3′ (SEQ ID NO: 203)) and oJL50 (5′-AAACCGACATCGCAGGCTTC-3′(SEQ ID NO: 204)). These primers flank the predicted insertion junction. The predicted product size was 609 bp. DNA amplified PCR product was visualized on a 2% agarose gel. Sanger sequencing of PCR products confirmed the transposition event.

### Example 7—in Cell Expression/In Vitro Assay

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

10 million cells were centrifuged and washed once with 1×PBS pH7.4. Supernatant wash was aspirated completely to the cell pellet, and flash frozen at −80 C for 16 hrs. After thawing on ice, cell pellet size was measured by mass, and appropriate extraction volumes of cell fractionation and nuclear extraction reagent was used to natively extract proteins in cell fractions. Briefly, cytoplasmic extraction reagent was used at 1:10 mass of cells to volume of extraction reagent. Cell suspension was mixed by vortexing and lysed with non-ionic detergent. Cells were then centrifuged at 16,000×g at 4° C. for 5 minutes. Cytoplasmic extraction supernatant was then decanted and saved for in vitro testing. Nuclear extraction reagent was then added 1:2 original cell mass to nuclear extraction reagent and incubated on ice for 1 hr on ice with intermittent vortexing. Nuclear suspension was then centrifuged at 16,000×g for 10 minutes at 4° C. and supernatant nuclear extract was decanted and tested for in vitro transposition activity. Using 4 μL of each cell and nuclear extract for each condition, the in vitro transposition reaction was performed with a complementary set of in vitro expressed proteins, donor DNA, pTarget, and buffer. Evidence of transposition activity was assayed by PCR amplification of donor-target junctions.

### Example 8—Activity in Mammalian Cells (Prophetic)

To show targeting and cleavage activity in mammalian cells, nuclear localization sequences are fused to the C terminus of each of the nuclease or effector proteins and integrase proteins and the fusion proteins are purified. A single guide RNA targeting a genomic locus of interest is synthesized and incubated with the nuclease/effector protein to form a ribonucleoprotein complex. Cells are transfected with a plasmid containing a selectable neomycin resistance marker (NeoR) or a fluorescent marker flanked by the left end (LE) and right end (RE) motifs, recovered for 4-6 hours, and subsequently electroporated with nuclease RNP and integrase proteins. Integration of a plasmid into the genome is quantified by counting G418-resistant colonies or fluorescence activated cell cytometry. Genomic DNA is extracted 72 hours after electroporation and used for the preparation of an NGS-library. Off target frequency is assayed by fragmenting the genome and preparing amplicons of the transposon marker and flanking DNA for NGS library preparation. At least 40 different target sites are chosen for testing each targeting system's activity.

### Example 9—Activity of Targeted Nuclease

In situ expression and protein sequence analyses suggested that some RNA guided effectors are active nucleases. They contain predicted endonuclease-associated domains (matching RuvC and HNH_endonuclease domains) and predicted HNH and RuvC catalytic residues (FIG. 4A).

Candidate activity was tested with engineered single guide RNA sequences using the in vitro expression system and in vitro transcribed RNA. Active proteins that successfully cleaved the library yielded a band around 170 bp in the gel.

### Example 10—Identification of Transposons

Transposons are predicted to be active when they contain one or more protein sequences with transposase and/or integrase function between the left and right ends of the transposon. A Tn7 transposon, as defined here, comprises a catalytic transposase TnsB, but may also contain TnsA, TnsC, TnsD, TnsE, TniQ, and/or other transposases or integrases. The transposon ends comprise predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g., FIG. 4A and FIG. 5A).

### Example 11—Identification of CRISPR-Associated Transposons

Putative CRISPR-associated transposons (CAST) contain a DNA and/or RNA targeting CRISPR effector and proteins with predicted transposase function in the vicinity of a CRISPR array. In some systems, the effector is predicted to have nuclease activity based on the presence of endonuclease-associated catalytic domains and/or catalytic residues (e.g., FIG. 4A). The transposases were predicted to be associated with the active nucleases when the CRISPR loci (CRISPR nuclease and array) and the transposase proteins are located between the predicted transposon left and right ends (e.g., FIGS. 4B and 4C). In this case, the effector was predicted to direct DNA integration to specific genomic locations based on a guide RNA.

In some systems, the effector was predicted to have homology with documented CRISPR effector proteins, but to be inactive based on the absence of endonuclease domains and/or catalytic residues (FIG. 5A). The transposases were predicted to be associated with the effector when the CRISPR loci (inactive CRISPR nuclease and array) and the transposase proteins were located within the predicted transposon left and right ends (FIGS. 5A and 5B).

### Example 12—CAST Identification

CRISPR-associated transposons (CAST) are systems that comprise a transposon that has evolved to interact with a CRISPR system to promote targeted integration of DNA cargo.

CASTs are genomic sequences encoding one or more protein sequences involved in DNA transposition within the signature left and right ends of the transposon. A Tn7 transposon, as defined here, comprises a catalytic transposase TnsB, but may also contain a catalytic transposase TnsA, a loader protein TnsC or TniB, and target recognition proteins TnsD, TnsE, TniQ, and/or other transposon-associated components. The transposon ends comprise predicted transposase binding sites, which contain direct and/or inverted repeats of 15 bp to 150 bp in length flanking the transposon machinery and other ‘cargo’ genes.

In addition, CASTs also encode a DNA and/or RNA targeting CRISPR nuclease or effector in the vicinity of a CRISPR array. In some systems, the effector was predicted to be an active nuclease based on the presence of endonuclease-associated catalytic domains and/or catalytic residues. In some systems, the effector was predicted to have sequence similarity with documented CRISPR effector proteins, but to be inactive based on the absence of endonuclease domains and/or catalytic residues. The transposons were predicted to be associated with the effector when the CRISPR locus and the transposon-associated proteins were located within the predicted transposon left and right ends. In this case, the effector was predicted to direct DNA integration to specific genomic locations based on a guide RNA.

### Example 13—Class 2 Cas12K CAST

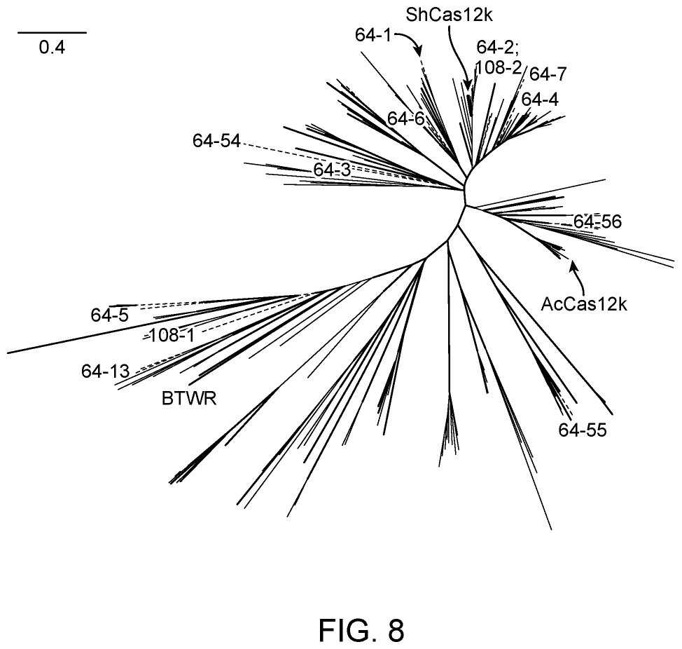

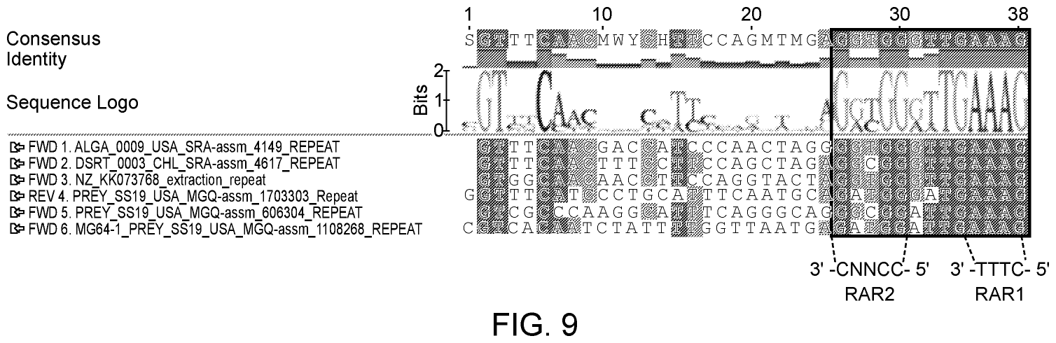

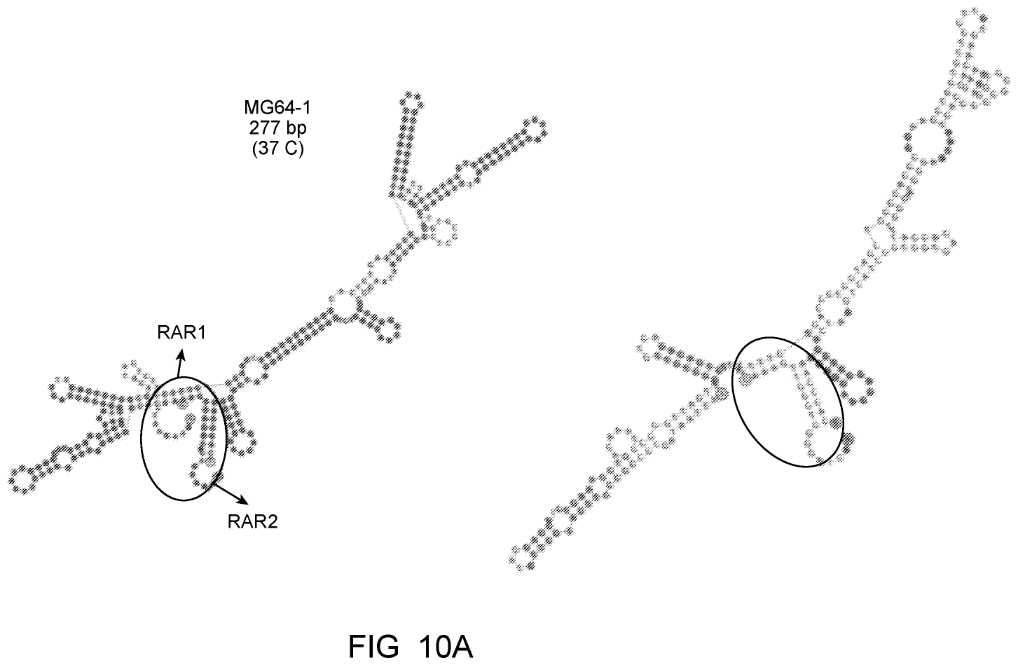

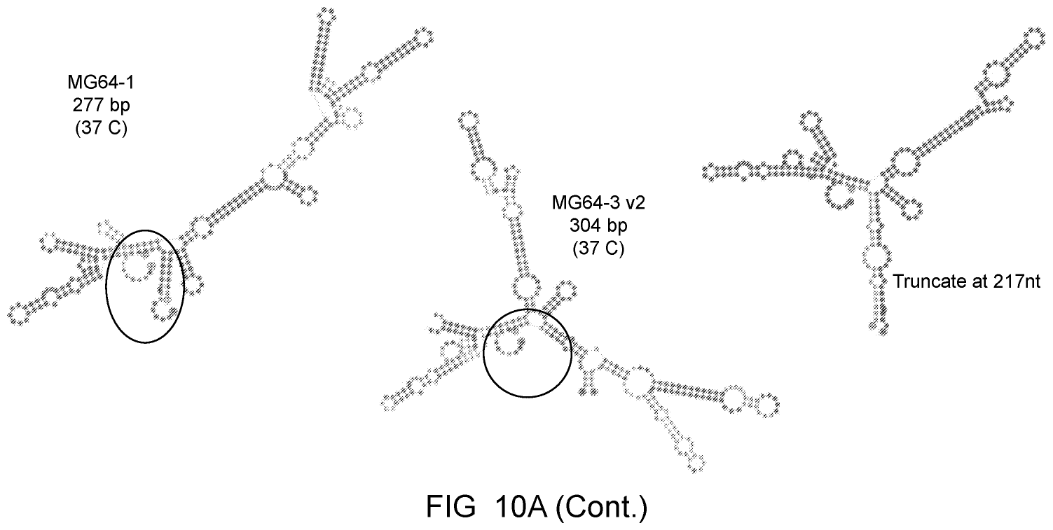

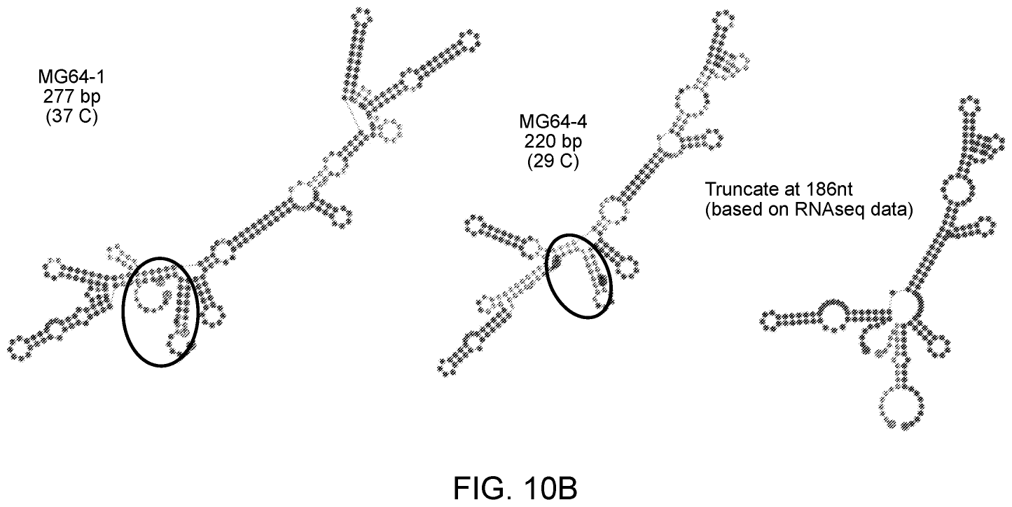

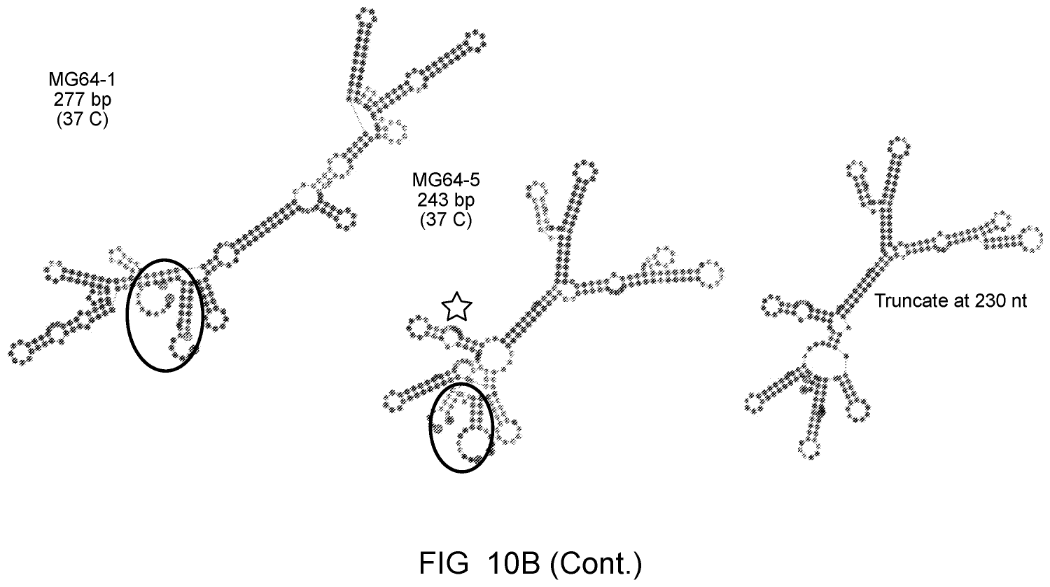

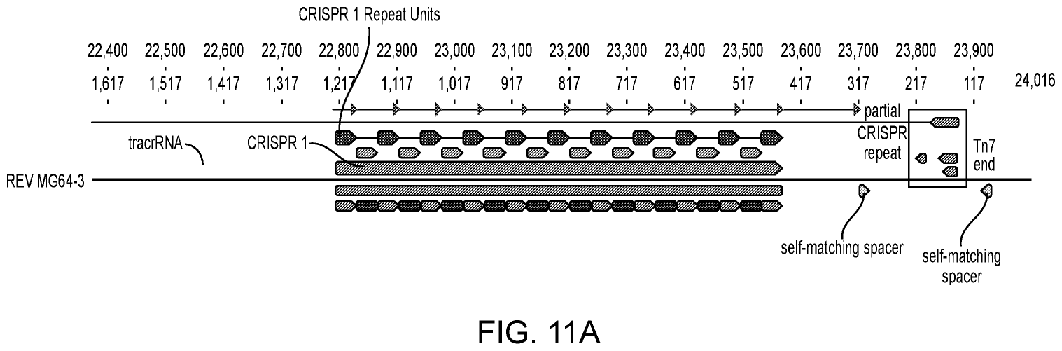

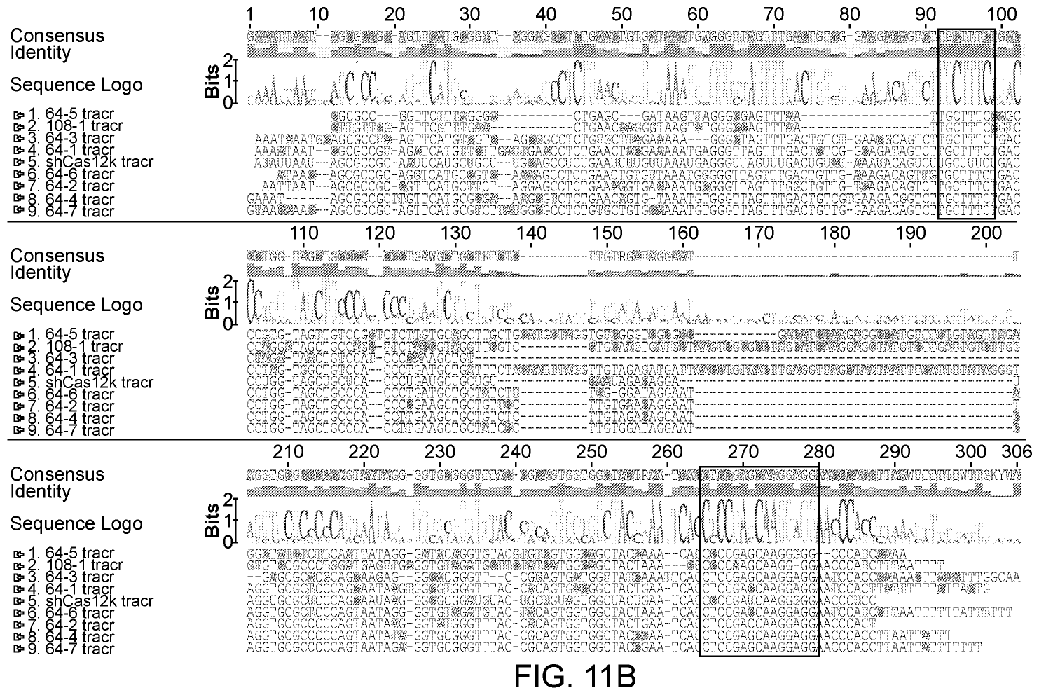

Cas12k CAST systems encode a nuclease-defective CRISPR Cas12k effector, a CRISPR array, a tracrRNA, and Tn7-like transposition proteins. Cas12k effectors are phylogenetically diverse and features that confirm their association with CASTs have been confirmed for several (FIG. 8). For example, the transposon left end was identified downstream from the MG64-3 CRISPR locus, as shown by terminal inverted repeats and self-matching spacer sequences (FIG. 11A). Cas12k CAST CRISPR repeats (crRNA) contain a conserved motif 5′-GNNGGNNTGAAAG-3′ (FIG. 9). Short repeat-antirepeats (RAR) within the crRNA motif aligned with different regions of the tracrRNA (FIG. 9 and FIG. 10), and RAR motifs appeared to define the start and end of the tracrRNA (For example, for MG64-1, the 5′ end of the tracrRNA contained RAR1 (TTTC) and the 3′ end contained RAR2 (CCNNC), (FIG. 10A).

### Example 14—Transposon End Prediction

Transposon ends were estimated from intergenic regions flanking the effector and the transposon machinery. For example, for Cas12k CAST, the intergenic region located directly upstream from TnsB and directly downstream from the CRISPR locus, were predicted as containing the Tn7 transposon left and right ends (LE and RE).

Direct and inverted repeats (DR/IR) of ˜12 bp were predicted on the contig, with up to 2 mismatches. In addition, the Dotplot algorithm was used to find short (˜10-20 bp) DR/IR flanking CAST transposons. Matching DR/IR located in intergenic regions flanking CAST effector and transposon genes are predicted to encode transposon binding sites. LE and RE extracted from intergenic regions, which encode putative transposon binding sites, were aligned to define the transposon ends boundaries. Putative transposon LE and RE ends are regions: a) located within 400 bp upstream and downstream from the first and last predicted transposon encoded genes; b) sharing multiple short inverted repeats; and c) sharing >65% nucleotide id.

### Example 15—Single Guide Design

Analysis of the intergenic regions surrounding the Cas effector and CRISPR array identified a potential anti-repeat sequence and a conserved “CYCC(n6)GGRG” stem loop structure neighboring the antirepeat corresponding to the duplexing sequence of the tracrRNA (FIG. 11B). TracrRNA and crRNA repeat were folded and trimmed, adding a tetraloop sequence of GAAA to maintain the stem loop region of the crRNA-tracrRNA complementary sequence.

### Example 16—In Vitro Integration Activity Using Targeted Nuclease

In situ expression and protein sequence analyses indicated that some RNA guided effectors are active nucleases. They contain predicted endonuclease-associated domains (matching RuvC and HNH_endonuclease domains), and/or predicted HNH and RuvC catalytic residues. Candidate activity was tested with engineered single guide RNA sequences using the in vitro expression system and in vitro transcribed RNA. Active proteins that successfully cleaved the library yielded a band around 170 bp in the gel.

### Example 17—Programmable DNA Integration

CAST activity was tested with five types of components (1) a Cas effector protein (SEQ ID NO: 1) expressed by an in vitro expression system, (2) a target DNA fragment or plasmid containing the target sequence and PAM corresponding to the Cas enzyme (GTN), (3) a donor DNA fragment containing a marker or fragment of DNA flanked by the LE and RE of the transposase system in a DNA fragment or plasmid (SEQ ID NOs: 8-11) (4) any combination of transposase proteins expressed using an in vitro expression system (SEQ ID NO: 2-4), and (5) an engineered in vitro transcribed single guide RNA sequence (SEQ ID NO: 5). Active systems that successfully transposed the donor fragment were assayed by PCR amplification of the donor-target junction.

After performing the transposition reaction, PCR amplification of the junction showed that proper donor-target formation occurred and that the transposition reaction was sg dependent. (FIG. 9). PCR amplification of reactions #3 and #4 indicated that both orientations of the donor relative to the target were made: one where the LE is closer to the PAM, and one where the RE is closer to the PAM. While both transposition orientations occurred, there appeared to be a preference for donor integration in the target where the LE is closer to the PAM, represented by strong band present for reactions #4 and #5.

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 10a). Sequencing that originated from the donor over the donor-target junction defined the composition of the essential outer bounds of the LE and RE sequences (FIG. 10a,b). Sequencing of the RE on LE-closer-to-PAM products showed a 3 bp duplication downstream of the donor RE (FIG. 10b). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also indicated that the PAM preference of the MG64-1 effector as a nGTn/nGTt on the 5′ end of the spacer (FIG. 10c). NGS analysis of the PAM library target corroborated that the nGTn motif preference at the 5′ end.

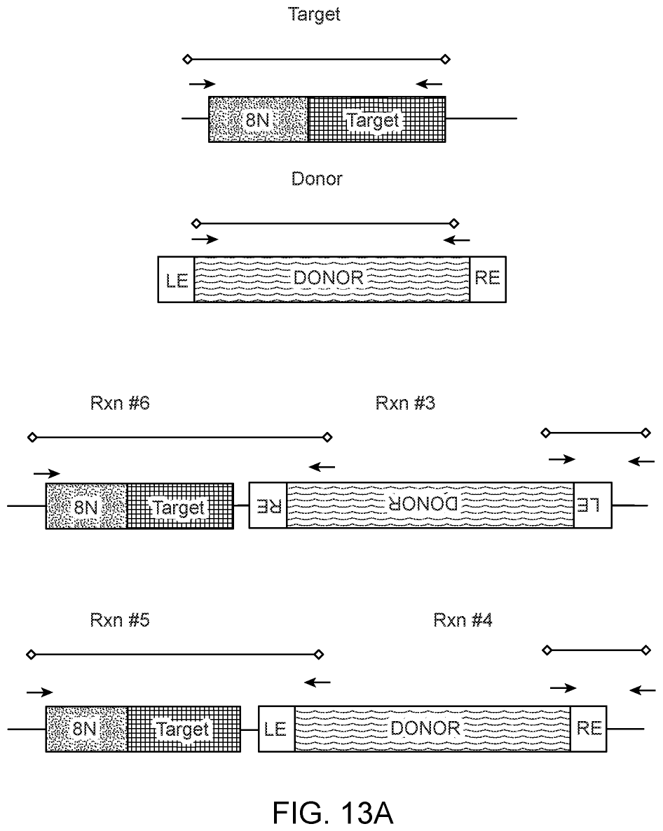

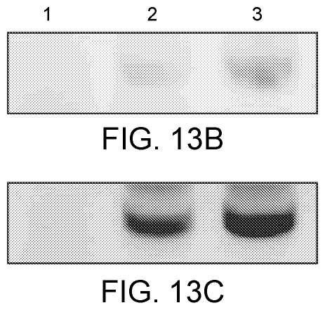

Further development of single guide testing confirmed activity of MG64-1 with a new sgRNA scaffold (FIG. 13).

### Example 18—Integration Window Determination

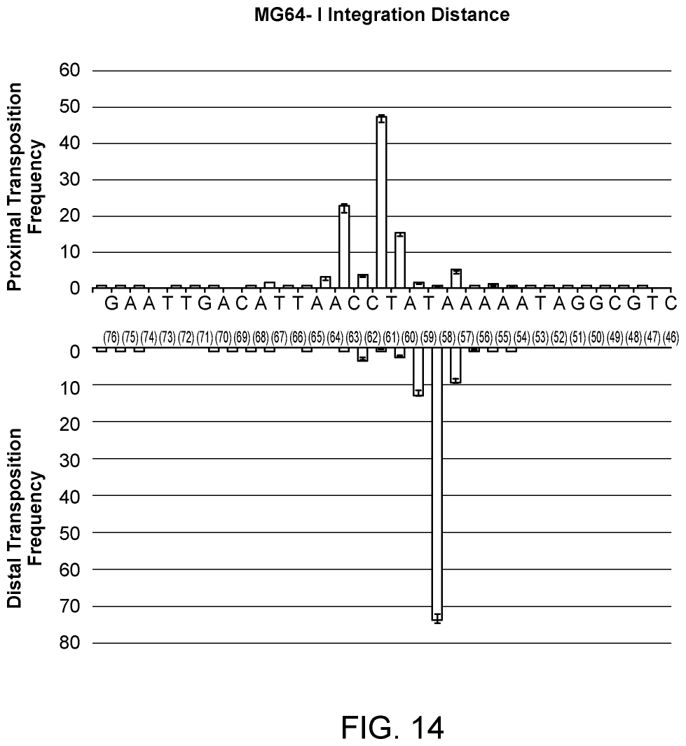

PCR junctions of the PAM that were amplified were indexed for NGS libraries and sequenced. Reads were mapped and quantified using CRISPResso using an amplicon sequence of a putative transposition sequence with a 60 bp distance of integration from the PAM (guideseq=20 bp 3′ end of LE or RE, center of window=0, window size=20) Indel histogram was normalized to total indel reads detected, and frequencies were plotted relative to the 60 bp reference sequence (FIG. 14)

Both PCR reactions 5 (LE proximal to PAM, FIG. 14 top panel) and PCR 4 (RE distal to PAM, FIG. 14 bottom panel) were plotted on the sequence and distance from the PAM for MG64-1. Analysis of the integration window indicates that 95% of the integrations that occurred at the spacer PAM site were within a 10 bp window between 58 and 68 nucleotides away from the PAM. Differences in the integration distance between the distal and the proximal frequencies reflected the integration site duplication—a 3-5 base pair duplication as a result of staggered nuclease activity of the transposase upon integration.

### Example 19—Colony PCR Screen of Transposase Activity

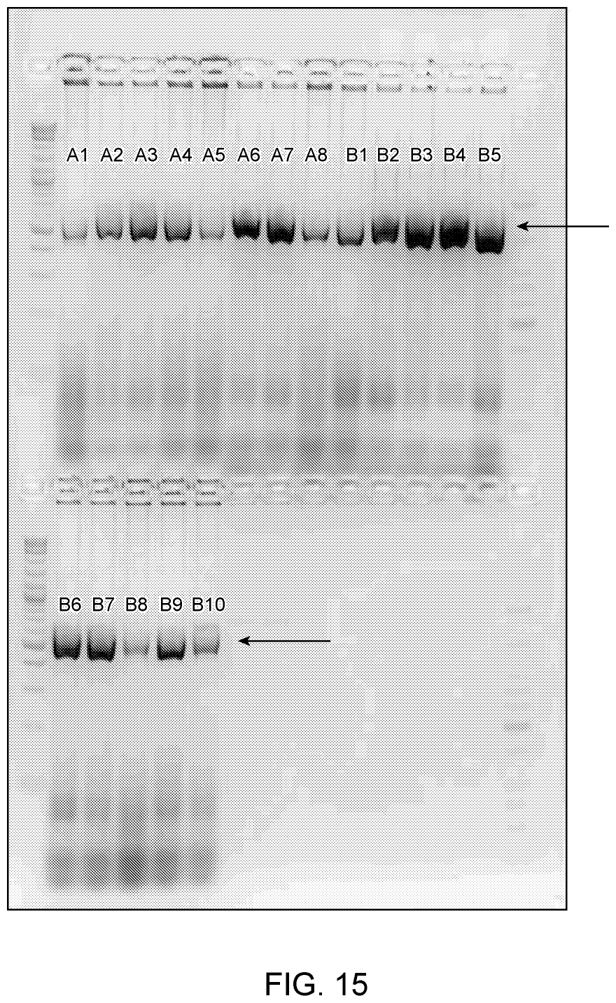

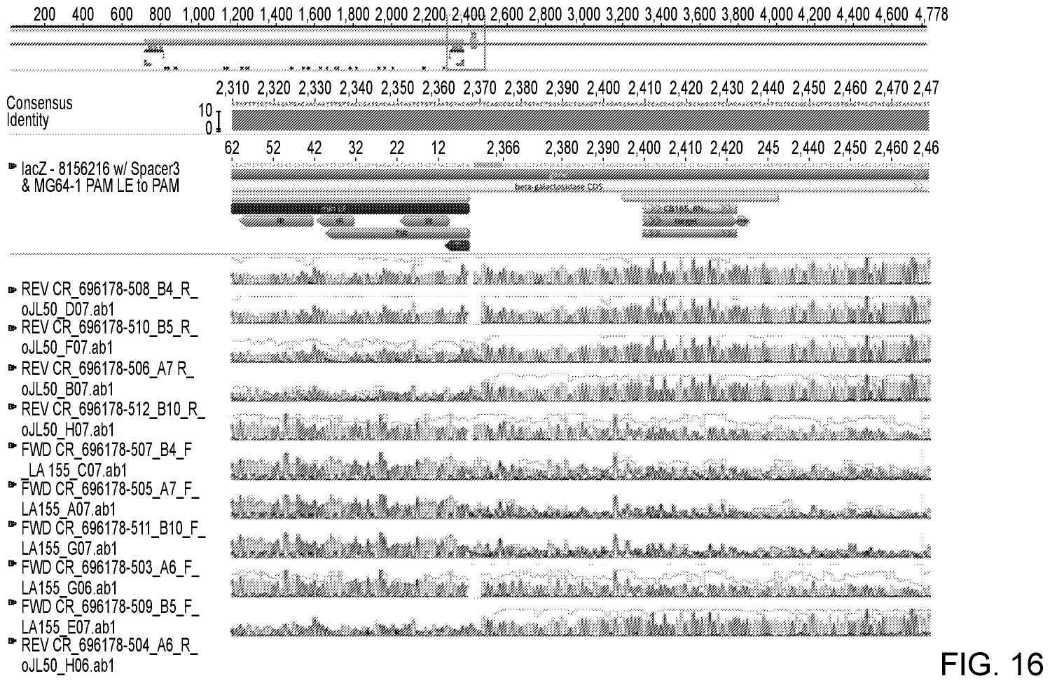

Transposition activity was assayed via a colony PCR screen. After transformation with the pDonor plasmids, E. coli were plated onto LB-agar containing ampicillin, chloramphenicol, and tetracycline. Select CFUs were added to a solution containing PCR reagents and primers that flank the selected insertion junction. PCR reactions of the integration products were visible on a gel (FIG. 15). Sequencing results of select colony PCR products confirmed that they represent transposition events, as they spanned the junction between the LE and the PAM at the engineered target site, which is in the lacZ gene (FIG. 16).

### Example 20—Single Guide Engineering

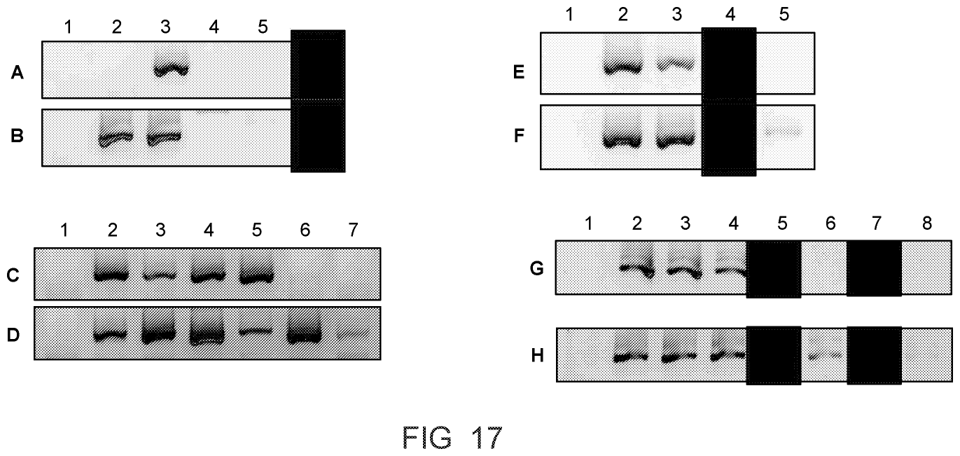

Predicted RNA folding of the active single RNA sequence was computed at 370 using the method of Andronescu 2007. All hairpin-loop secondary structures were single deleted from the construct and iteratively compiled into a smaller single guide. Engineered single guides (esg) 4, 6, 7, 8, 9 were active for donor transposition (FIGS. 17C and 17D), with engineered sgRNAs 8 and 9 being weaker single guides and transposing with PCR5 (FIG. 17D). Engineered guide 5 was able to transpose, however engineered sgRNA 10 weakly transposed with PCR 5 (FIGS. 17E and 17F) Esg 17 is a combination of deletions in esg6 and esg7, and esg 18 is a combination of esg 4 and esg5. Both were able to strongly transpose across both PCR4 and 5 (FIGS. 17G and 17H), However, combinatorial addition of esg 6 and esg 18 making esg 19, resulted in a weaker transposition in PCR5, and addition of esg 7 to esg 19, making esg 20 results in a very weak junction of transposition for PCR 5 (FIGS. 8G and 8H). In a second approach, the tracrRNA of MG64-1 was aligned to documented type Vk tracrRNA, and areas of unique insertions were mutated out of the single guide. sgRNA was minimized by truncation of insertion sequences of the MG64-1 sgRNA (FIG. 14). 2 subsequent deletions, esg 2 and esg 3 were also tested (FIGS. 17A and 17B) but neither esg2 nor esg3 resulted in appreciable transposition, thus the, and single guide was minimized by 57 bases.

### Example 21—LE-RE Minimization

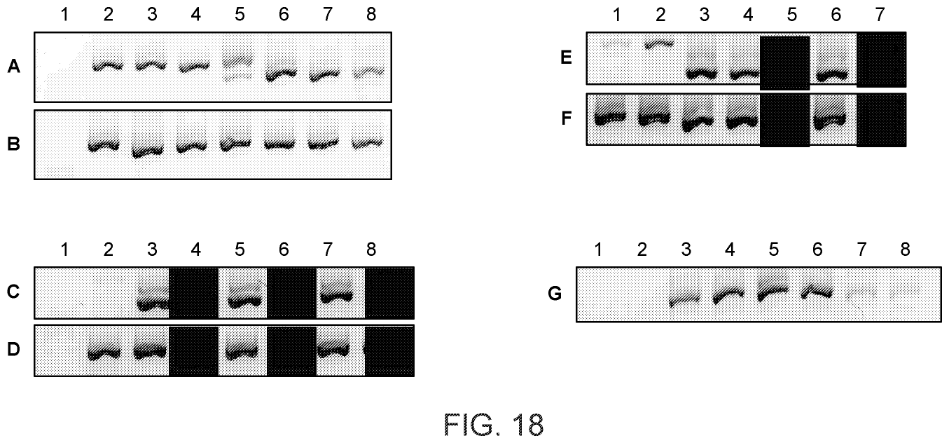

Sequencing of the target-transposition junction aided in identification of the terminal inverted repeats by identifying the outmost sequence from the donor plasmid that was incorporated into the target reaction. By performing repeat analysis of 14 bp with variability of 10%, short repeats contained within the terminal ends were identified and truncations of these minimal ends to preserve the repeats while deleting superfluous sequence were designed. Prediction and cloning was done in multiple iterations, with each interaction tested with in vitro transposition. Initial LE and RE deletions were singly designed and cloned to the 68 bp, 86 bp, and 105 bp for the LE, 178 bp, 196 bp and 242 bp for the RE. The RE of 64-1 also had a noticeable span of sequence without a repeat, so internal deletions of both 50 bp and 81 bp were designed and cloned. Transposition among all single deletions was robust for both PCR 4 and PCR 5 (FIGS. 18A-18B) and internal deletion of 81 bp was subsequently pursued with combinatorial deletions for the RE. Trimmed ends of the former 178, 196 and 212 bp were cloned on the 81 bp internal deletion and transposition was tested. Transposition was active for all constructs designed. In combination with LE of 68 bp, transposition proved active down to a LE region of 68 bp combined with a RE region of 96 bp (FIG. 18E-18F).

### Example 22—Overhang Influence of Transposition

In order to test whether superfluous sequence outside of the TnsB binding motifs were necessary for transposition, oligos designed for the TGTACA motifs of both LE and RE were designed and synthesized with 0, 1, 2, 3, 5 and 10 bp extra base pairs. These synthesized oligos were used to generate donor PCR fragments with overhangs and tested for their ability to transpose into the target site. Most noticeably, PCR6 was rarely detected from the in vitro reactions, (FIG. 18G lanes 1,2) however with a small 0-3 bp overhang, efficient integration at PCR 6 was detected, reflecting a RE proximal to PAM orientation that is not detected with a larger flanking sequence.

### Example 23—CAST NLS Design

Eukaryotic genome editing for therapeutic purposes is largely dependent on the import of editing enzymes into the nucleus. Small polypeptide stretches of larger proteins signal to cellular components for protein import across the nuclear membrane. Placement of these tags is not trivial, as these NLS tags need to provide import function while also maintaining function of the protein to which it is fused. In order to test functional orientations of the NLS to each of the components of the CAST complex, constructs were designed and synthesized fusing Nucleoplasmin NLS to the N-terminus and SV40 NLS to the C-terminus of each of the components of the MG CAST. Protein of these constructs were expressed in cell free in vitro transcription/translation reactions and tested for in vitro transposition activity with a complement set of untagged components. NLS-tagged constructs were assessed for maintenance of activity by PCR of the donor-target junction using PCR 4 (Assessing RE distal transpositions) and the cognate transposition event, PCR 5 (LE to proximal transposition).

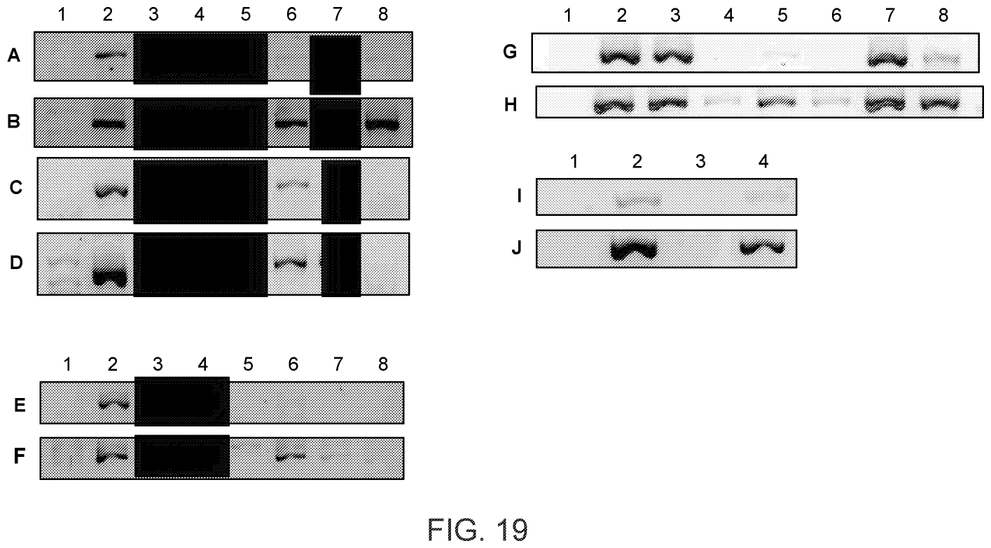

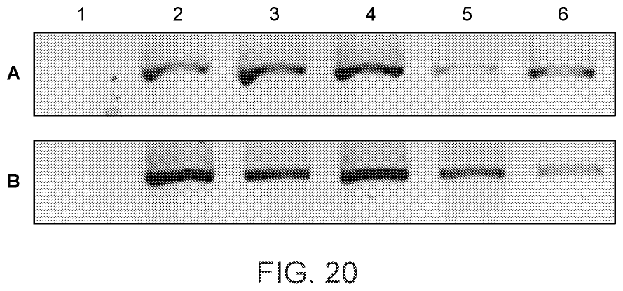

Most components resulted in a single NLS orientation that maintained activity. TnsB was the CAST component that was active with both N-terminal NLS and C terminal NLS by both PCR4 and PCR 5 (FIGS. 19A-19B). TniQ was active with N-terminal NLS tags (FIGS. 19C-19D). And Cas12k component was active with a C-terminal tagged NLS (FIGS. 19E-19F, lanes 5,6). Further development of a Cas12k with both Nucleoplasmin and SV40 NLS tags were tested and found to be active (FIGS. 19 I-19J, Lane 4). TnsC was weakly active with an N-terminal NLS (FIGS. 19E-19F, lane 7), but further exploration of the TnsC tagging identified new working NLS-HA-TnsC and NLS-FLAG-TnsC constructs (FIGS. 19G-19H, lanes 3 and 7, respectively). The end result was a completely NLS-tagged suite of components that were active in vitro with both orientations of NLS-TnsB and TnsB-NLS (FIGS. 20A-20B lanes 5.6).

### Example 24—Cas12k and TniQ Protein Fusion Construct Design and Testing

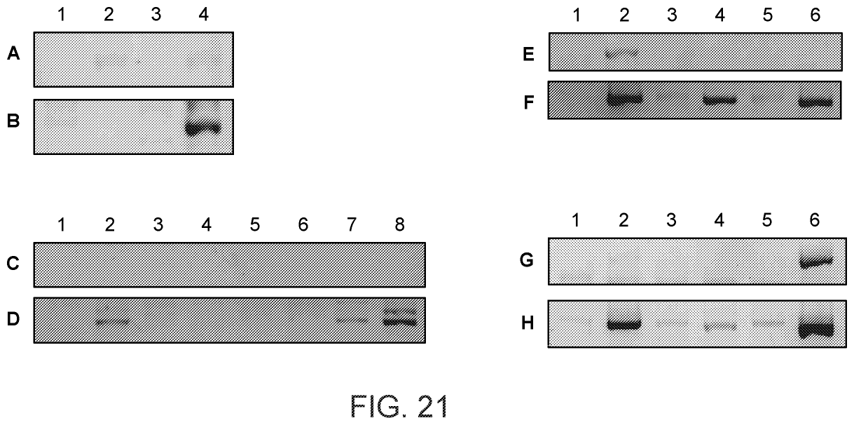

In an effort to simplify the expression of the protein components and minimize delivery of these components into cells, fusion constructs between the Cas12k effector and the TniQ protein were designed, synthesized, and tested. Both orientations of the TniQ fused to the Cas12k were designed and synthesized, a C-terminal fusion, Cas-TniQ, and an N terminal fusion, TniQ-Cas. While both constructs were weakly active for PCR4 (FIG. 21A), when expressed in vitro and assayed for transposition abilities, PCR5 junction was robustly formed by the TniQ-Cas fusion protein (FIG. 21B). Transpositions lengths were assayed with variable linker domains including the original (20 amino acid linker), 48, 68 72 and 77 (FIGS. 21C-21F). NLS tags were then linked to the N terminus of TniQ and the C terminus of the Cas12k and found to still be active by PCR5 (FIGS. 20E-20F).

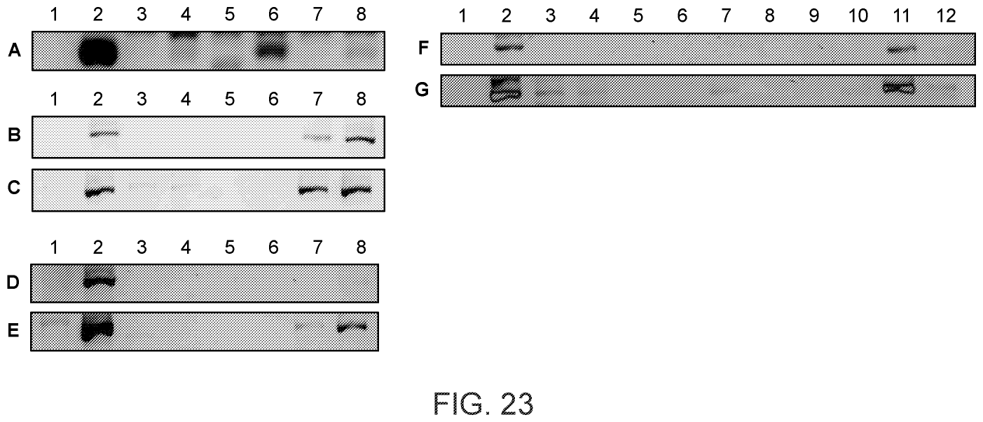

Two other linkers were employed to fuse the effector and TniQ genes. P2A, a self-stopping translation sequence was active in a Cas-NLS-P2A-NLS-TniQ construct (FIGS. 21G-21H, lane 6), and an MCV Internal Ribosome Entry Sequence (IRES) mRNA-based linker allowed for independent translation of the two components in cells (FIGS. 23F-23G).

### Example 25—Intracellular Expression Coupled In Vitro Transposition Testing

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

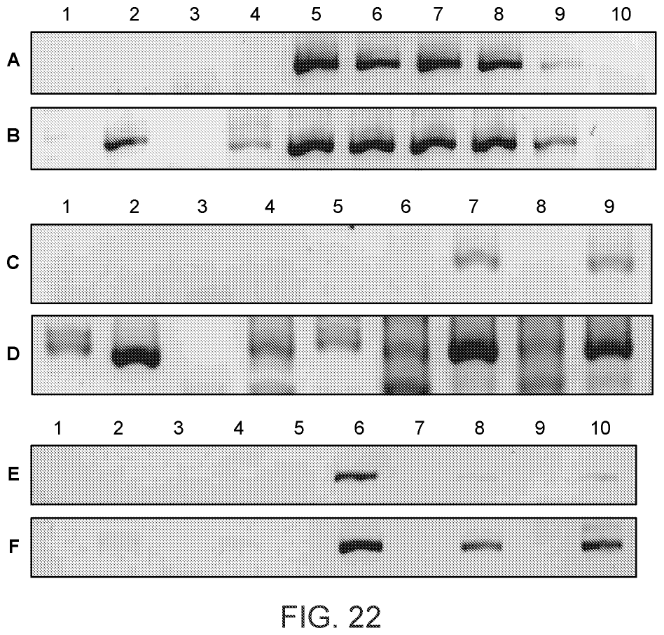

Both NLS-TnsB and TnsB-NLS were tested by cell fractionation and in vitro transposition, and transposition was detected across both cytoplasmic and nuclear fractions, and NLS-TniQ had detectable activity in the cytoplasm (FIGS. 22A-22B). NLS-HA-TnsC and NLS-FLAG-TnsC were both active in both cytoplasmic and nuclear fractions when expressed (FIG. 22D), however PCR4 is formed in the nuclear fraction of both TnsC constructs. (FIG. 22C).

When both NLS-TnsB or TnsB-NLS were linked with NLS-FLAG-TnsC by using an IRES, NLS-TnsB-IRES-NLS-FLAG-TnsC was largely active in the nuclear fraction while TnsB-NLS-IRES-NLS-FLAG-TnsC was active in both cytoplasmic and nuclear fractions. This is indicative that NLS-TnsB has a higher capacity of trafficking to the nucleus (FIG. 21E,F).

Cas12k fusions in the cell were similarly fractionated and tested for transposition. Cas-NLS Cas-NLS-P2A-NLS-TniQ were transduced into cells, fractionated, and tested in vitro for subcellular activity. Cas-NLS-P2A-NLS-TniQ was able to transpose in the cytoplasm with the addition of single guide to the reaction (FIG. 23A). By supplementing holo Cas protein (+sgRNA) or additional TniQ with sgRNA, the Cas-NLS-P2A-NLS-TniQ construct in the nuclear fraction was complemented. This indicates that both Cas-NLS and NLS-TniQ are making it into the nucleus (FIGS. 23B-23C). NLS-TniQ-Cas-NLS fusion protein had similar results, but needed more supplementation with TniQ (FIGS. 23D-23E), and Cas-NLS-IRES-NLS-TniQ needed supplementation from just the holo Cas-NLS (FIGS. 23F-23G) As a whole this indicates that all the components of the CAST have been able to be delivered to the nuclear fraction of the cell.

### Example 26—Transposon End Verification Via Gel Shift

In order to verify the activity of TnsB on the predicted transposon end sequence, the LE of MG64-1 was amplified using FAM labeled oligos. MG64-1 TnsB protein was expressed using a cell free transcription/translation system and incubated with the LE FAM labeled product. After incubation for 30 minutes, binding was observed on a native 5% TBE gel (FIG. 24). Multiple bands of fluorescent product within the co-incubated lane (FIG. 24, lane 3) indicated a minimum of 2 TnsB binding sites.

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Example 27—Defined Domains

Functional domains (FD) are small proteins that are capable of facilitating the interactions of proteins with DNA as in DNA Binding domains (DBD) and chromatin modulating domains (CMD). By using binding domains that are non-specific for DNA sequence, affinity of the functional proteins was increased without adverse effects on function. 4 Functional domains were chosen for their ability to bind to DNA and DNA associated proteins non-specifically: Human histone 1 central globular domain (aa. 22-101) (SEQ ID NO: 112), HMGN1 (1-100) (SEQ ID NO:111), human Cbx5 (1006-1274) (SEQ ID NO:110), and Saccharolobus solfataricus sso7d (1-64) (SEQ ID NO:109).

### Example 28—Cloning

The functional domains in Example 27 were utilized to construct: (a) CAST-derived Cas-FD fusions; and (b) CAST-derived TniQ-FD fusions to investigate whether the functional domain increases the activity of these CAST system components in cells. DNA binding domains were codon optimized for human expression and synthesized or assembled using PCR stitching of oligos. To construct the Cas12k and the TniQ DBD fusion proteins, DBD proteins were amplified using primers and assembled with Cas12k-NLS and NLS-TniQ. DNA sequences of cloned fusion genes were confirmed by Sanger sequencing.

### Example 29—In Vitro Testing for Fusion to Functional Domain

CAST activity will be tested with five types of components: (1) a Cas-NLS effector or Cas12k-FD-NLS protein expressed by an in vitro expression system; (2) a target DNA fragment or plasmid containing the target sequence and PAM corresponding to the Cas enzyme; (3) a donor DNA fragment containing a marker or fragment of DNA flanked by the LE and RE of the transposase system in a DNA fragment or plasmid; (4) any combination of transposase proteins, transposase-NLS proteins, or transposase-FD-NLS proteins expressed using an in vitro expression system; and (5) an engineered in vitro transcribed single guide RNA sequence. Active systems that will successfully transpose the donor fragment will be assayed by PCR amplification of the donor-target junction.

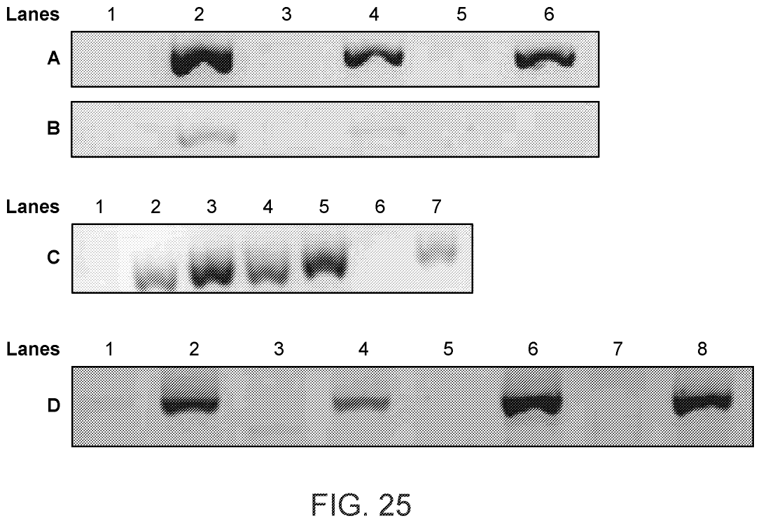

CAST NLS fusion proteins or CAST-FD-NLS fusion proteins were expressed in vitro, and tested for functionality in transposition reactions by swapping out non-FD-fused components for the fusion proteins (e.g., Cas12k-NLS for Cas12k-FD-NLS). When tested individually, sso7d, HMGN1, and Cbx5 fusions with Cas12k were active for transposition (FIGS. 25A and 25B, Lane 6; FIG. 25C Lane 7; and FIG. 25D, Lane 6). TniQ fusions were active for sso7d, HMGN1, H1 core and Cbx5 fusions (FIG. 25C, Lane 3-5; and FIG. 25D, Lane 4)

### Example 30—Nuclear Functioning of Cas-sso7d

Lentiviral cargo vectors containing active Cas12k-DBD-NLS fusion proteins that were active were transfected into 293w cells containing envelope and packaging plasmids. After 72 hrs at 37° C., the supernatant containing the active Lentiviral particles was incubated with K562 cells for viral transduction. Cells were selected for Lentiviral integration by selection on 2 μg/mL Puromycin for 4 days at 37° C. After selection, cell nuclei were extracted and tested for nuclear activity of Cas12k-DBD-NLS fusion proteins.

Cas12k-Cbx5 was poorly active—being able to translocate into the nucleus, but not at a high enough efficiency to have active transposition in extracted nuclei reactions. However, when additional in vitro expressed TniQ was added to the nuclear fractions, transposition was detectable by PCR. This implicates the presence of active Cas12k protein in the nucleus, but stoichiometrically requires additional TniQ to have active transposition.

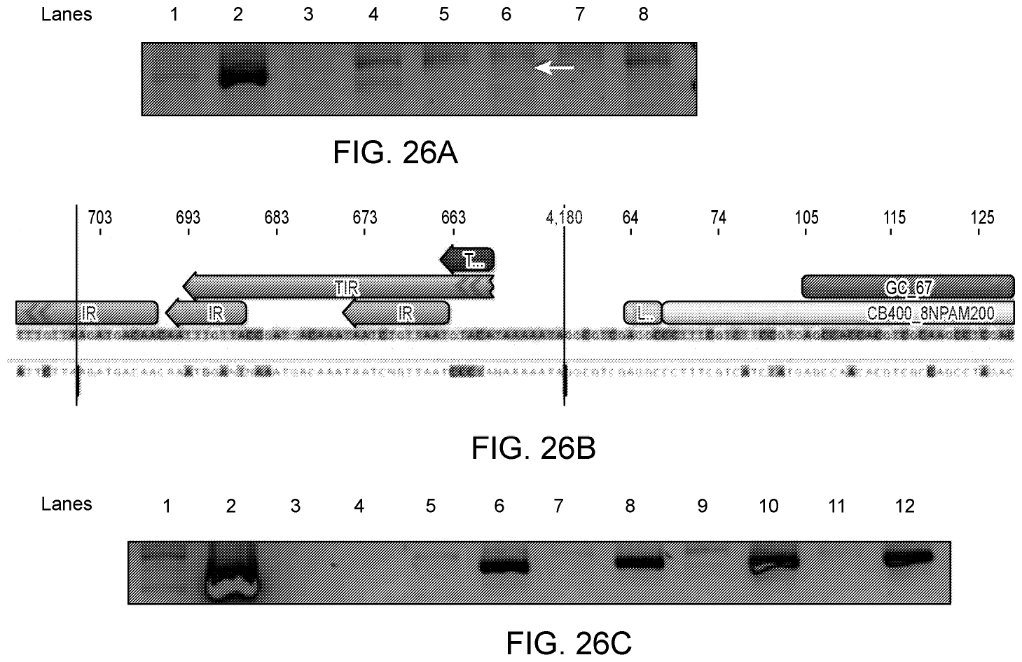

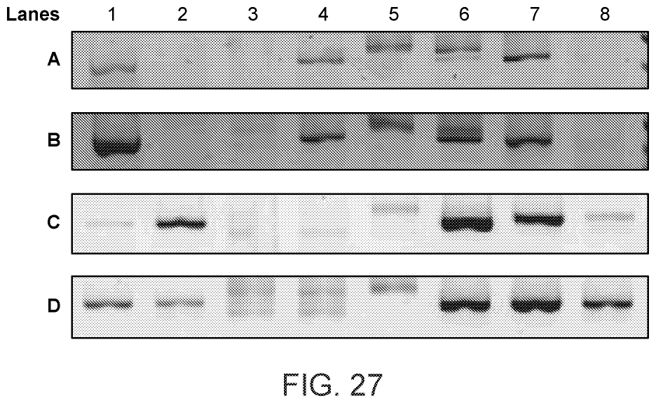

In order to test Cas12k-sso7d, Lentiviral transduced and puromycin selected cells were extracted for nuclear fractions. When transposition junctions were tested using the nuclear extracts, PCR of LE to target junction had a faint band of transposition in the nuclear fraction without the addition of in vitro Cas12k (FIG. 26A Lane 6). Faint signal was sanger sequenced and aligned to predicted junction PCR, and verified for transposition (FIG. 26B). It was concluded from the sequence signal that Cas12k-sso7d was capable of transposition, albeit at a low efficiency.

### Example 31—TniQ with DBD in Nuclear Extracts

In order to establish a functional version of TniQ fused with DBD, sso7d, Cbx5, HMGN1, and H1 core fusion constructs were tested with TniQ. These constructs, along with the WT TniQ, were expressed in vitro and supplemented to a nuclear extraction containing all 4 components of the CAST system. While WT TniQ had a moderate ability transpose donor to fragment, the use of sso7d, cbx5, HMGN1, and H1 core improved the transposition capabilities of the nuclear extract, indicating the ability of all four constructs to improve the function of transposition in vitro (FIG. 26C).

### Example 32—Nuclear Extraction of Cas-sso7d and TniQ Coexpression and Full Suite Expression

Of the four TniQ-DBD fusions that were tested in nuclear extracts and were more active than the WT TniQ, a co-expression construct that uses an IRES to co-express the TniQ DBD fusions with Cas12k and Cas12k-sso7d in a combinatorial fashion was prepared. Once transduced and selected on Puromycin, cells expressing both versions of Cas12k and TniQ were extracted for the nuclear fractions. Exogenous TnsB and TnsC were added to the in vitro reactions. Of the 8 constructs tested, only two versions of the Cas12k and TniQ fusion proteins were active—those cells co-expressing Cas12k-sso7d and HMGN1-TniQ and Cas12k-sso7d and H1 core-TniQ. These nuclear fractions transpositions were detectable and sequence verified using sanger sequencing of the potential PCR junction (FIGS. 27A and 27B).

In order to test the activity of all the components in the cell, a co-expressing construct of TnsB and TnsC was transduced into cells that are known to have high nuclear fraction activity. Both Cas12k-sso7d and HMGN1-TniQ and Cas12k-sso7d and H1 core-TniQ populations were transduced with TnsB and TnsC Lentiviral constructs. Nuclear extract of these cell populations containing all 4 components corroborate the functionality of all protein CAST components in the nuclear extract (FIGS. 27C and 27D).

### Example 33—Exploration of Target DNA Dilutions in In Vitro Transposition Assays In Vitro Targeted Integrase Activity Experiments

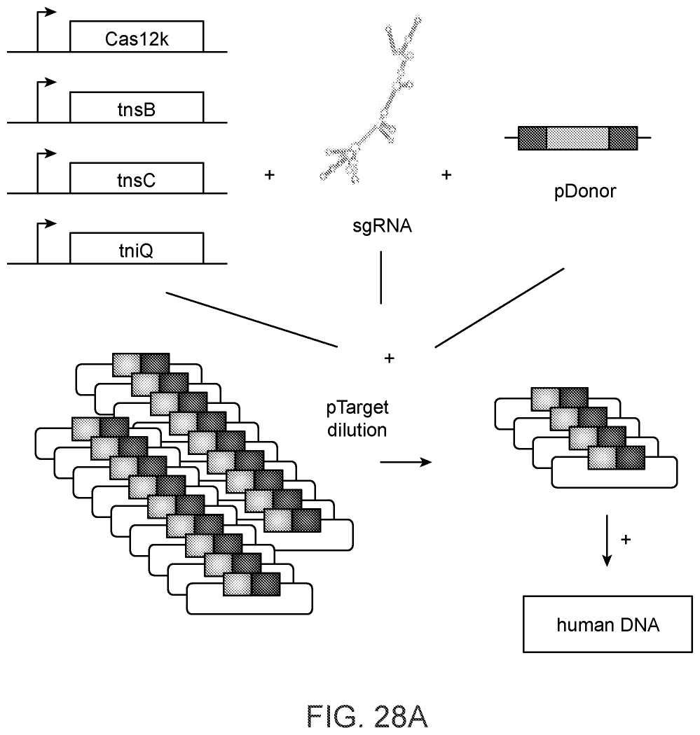

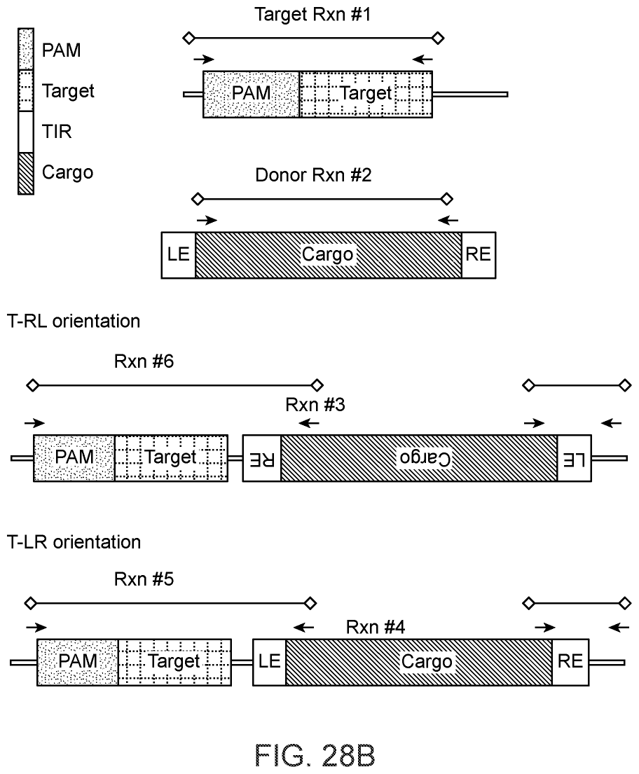

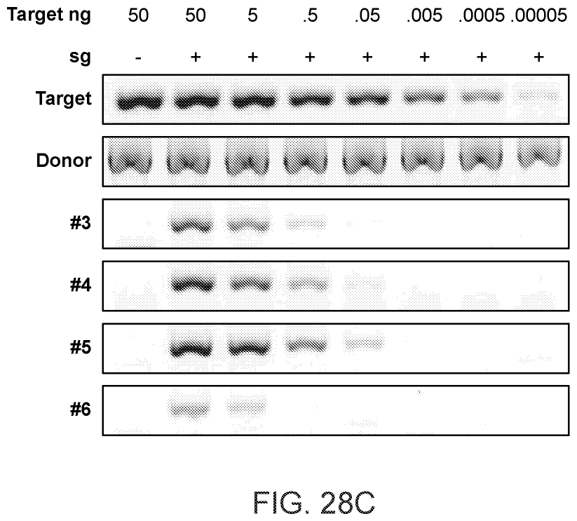

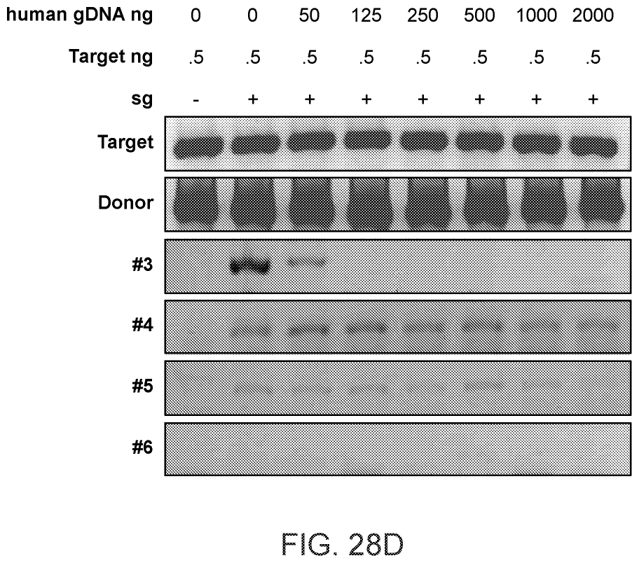

Integrase activity was assayed with a target plasmid containing the PAM adjacent to the protospacer sequence (pTarget) (FIG. 28A). T7 promoter leading gene sequences were introduced by PCR amplification of all transposase, single guide RNA (sgRNA) and effector components, and expressed independently in an in vitro transcription/translation system (FIG. 28A). Purified in vitro transcribed single guide RNA were refolded in duplex buffer (10 mM Tris pH 7.0, 150 mM NaCl, 1 mM MgCl₂) and normalized to 1 μM. Donor fragments were PCR amplified from plasmid pDonor, which contained a kanamycin or tetracycline resistance marker flanked by MG64-1 left end (LE) and right end (RE) transposon motifs, and normalized to 50 ng/μL.

After expression, 1 μL of Cas12k of the in vitro expression reaction was added to 0.5 picomoles of sgRNA and incubated for 20 minutes at 25° C. Individually expressed transposase proteins were then added volumetrically at 1 μL per expression. Target DNA and 50 ng donor DNA were then added to the transposition reaction in a reaction buffer, with final concentrations of 26 mM HEPES pH 7.5, 4.2 mM Tris pH 8, 50 μg/mL BSA, 2 mM ATP, 2.1 mM TCEP, 0.05 mM EDTA, 0.2 mM MgCl₂, 28 mM NaCl, 21 mM KCl, 1.35% glycerol, (final pH 7.5) and 15 mM Mg(OAc)₂. In vitro transposition reactions were performed at 37° C. for 2 hours, transposition reactions were diluted tenfold in water, and used subsequently as a template for junction PCR analysis.

### Junction PCR Analysis

Junction PCR reactions were performed with Q5 polymerase and amplified with primers flanking: Rxn #1 (Target), Rxn #2 (Donor), Rxn #3 (Reverse LE), Rxn #4 (Forward RE), Rxn #5 (Forward LE), and Rxn #6 (Reverse RE) (FIG. 28B). PCR fragments were run on a 2% agarose gel in 1×TAE and analyzed for size discrimination. Appropriately sized bands of each PCR junction were gel-excised, and the PCR fragments were recovered through purification and sanger sequenced using both amplification primers. Resulting Sanger sequencing was mapped to the donor and target sequences to confirm integration approximately 60 bp away from the PAM.

Transposition Reactions with Target Plasmid DNA Dilutions

Because of the known, low availability of single copy target sites in the human genome (for example, Moreb et al., 2020), it was predicted that low availability targets in in vitro experiments could mimic the search space in human genomic DNA by the MG64-1 CAST. To determine the minimum amount of target plasmid (pTarget) necessary to detect targeting by MG64-1 in vitro, the CAST was used in serial dilutions of total target DNA in the transposition reaction. Target plasmid amounts were serially diluted by 10 fold from 50 ng of DNA per reaction (moles) to 0.00005 ng (moles) and then added to transposition reactions containing the MG64-1 suite, sgRNA, and donor plasmid (FIG. 28A). PCR amplification of target-donor junctions across transposition products were then analyzed by gel electrophoresis (FIG. 28B).

### Results: Serial Dilution of Target Plasmid DNA

In vitro transposition experiments with dilutions of target plasmid indicated that single guide RNA (sgRNA) was necessary for targeted integration of donor onto the plasmid at 50 ng of target DNA (FIG. 28C, lanes 1 and 2). As the target was serially diluted, target DNA was still detectable at 0.00005 ng/reaction but with lower intensity (FIG. 28C, “Target” band on lane 8). Reverse LE integration product (Rxn #3) and reverse RE product (Rxn #6) both decreased with the tenfold dilutions, resulting in the detectable presence of Rxn #3 down to 0.5 ng of target DNA (FIG. 28C). Transposition reactions Rxn #4 and #5 were both equally robust and were detectable down to 0.05 ng of target DNA.

Based on the presence of three of four expected transposition reaction products, the 0.5 ng of target plasmid condition was selected to test whether transposition was detected when increasing the complexity of DNA search space. Increasing amounts of exogenous human genomic DNA (gDNA) were added to the reaction with fixed 0.5 ng of target plasmid, MG64-1 CAST, and sgRNA (FIG. 28A and FIG. 28D). When no gDNA was added, transposition experiments confirmed that sgRNA was necessary for targeted integration of donor onto the target plasmid at 0.5 ng of DNA (FIG. 28D, lanes 1 and 2). When gDNA was added to the reaction at increasing amounts (0-2000 ng of DNA per reaction), transposition products were also diluted, as given by the faint bands compared with the no gDNA control (FIG. 28D). For example, the reverse LE integration product (Rxn #3) was visible when adding at most 50 ng of gDNA. In addition, robust transposition products for the forward RE (Rxn #4) and forward LE (Rxn #5) products at 125 to 1000 ng of gDNA with a fixed amount of 0.5 ng of target plasmid (FIG. 28D, lanes 4-8) was detected.

### Example 34—In Vitro Transposition to Human gDNA with MG64-1 CAST

### In Vitro Targeting to High Copy Elements Across the Human Genome

This Example assesses a dilution series of natural targets in the genome for transposition as a function of target frequency.

### Results: Target Site Identification in High Copy Regions of the Human Genome

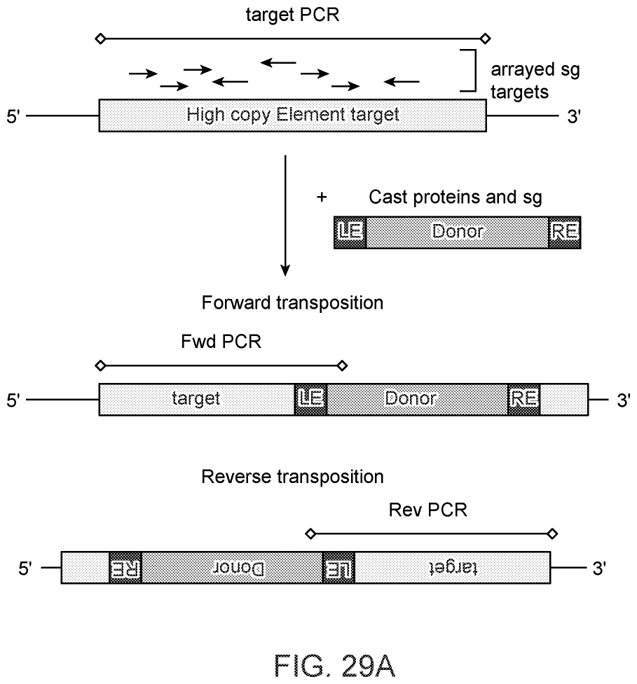

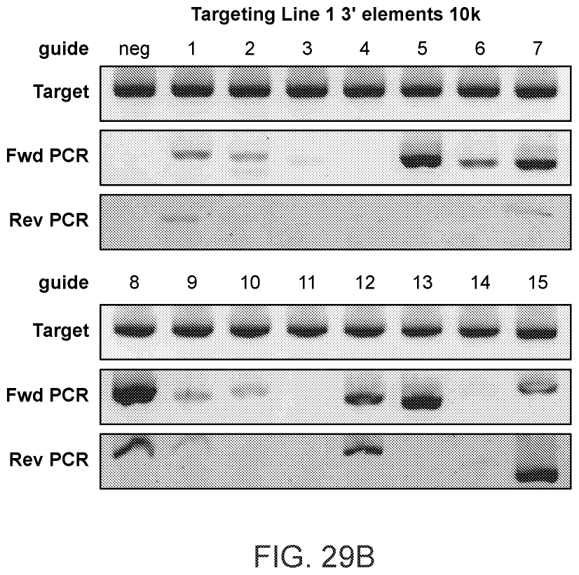

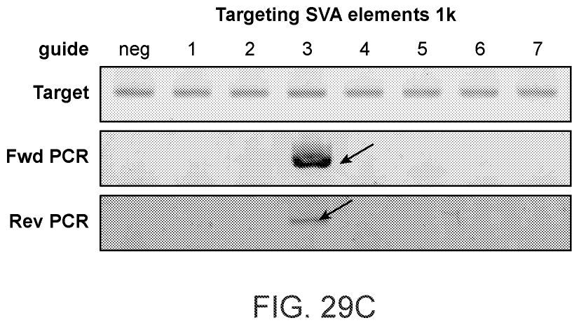

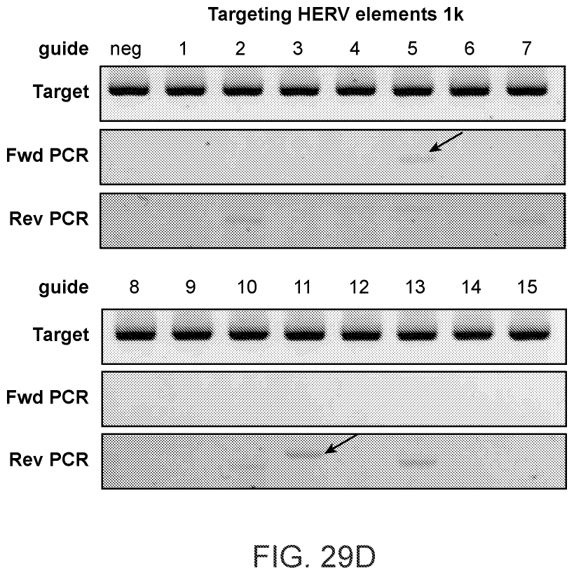

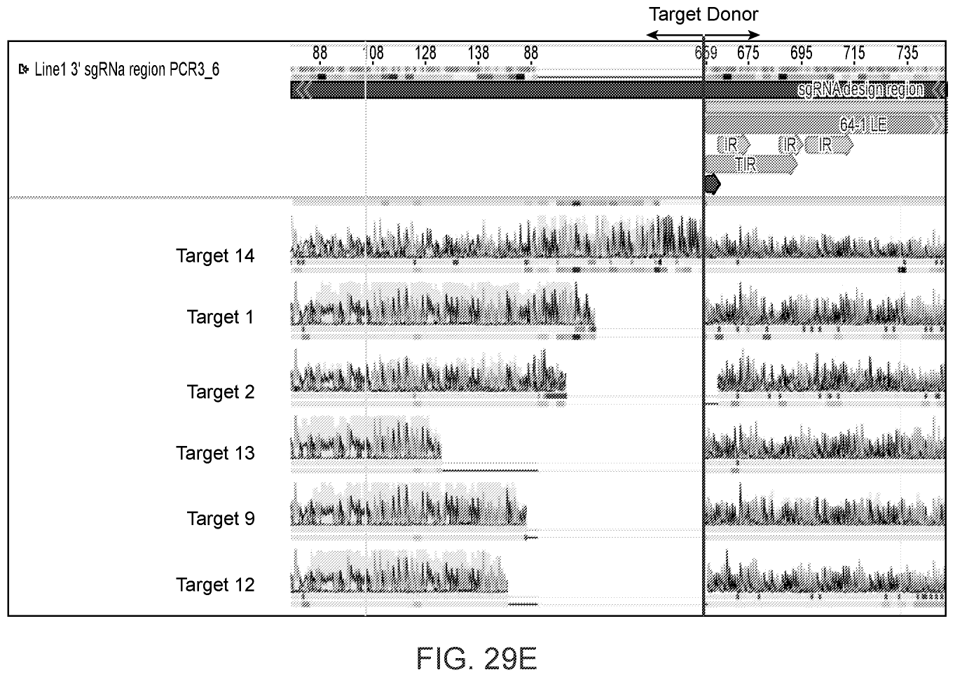

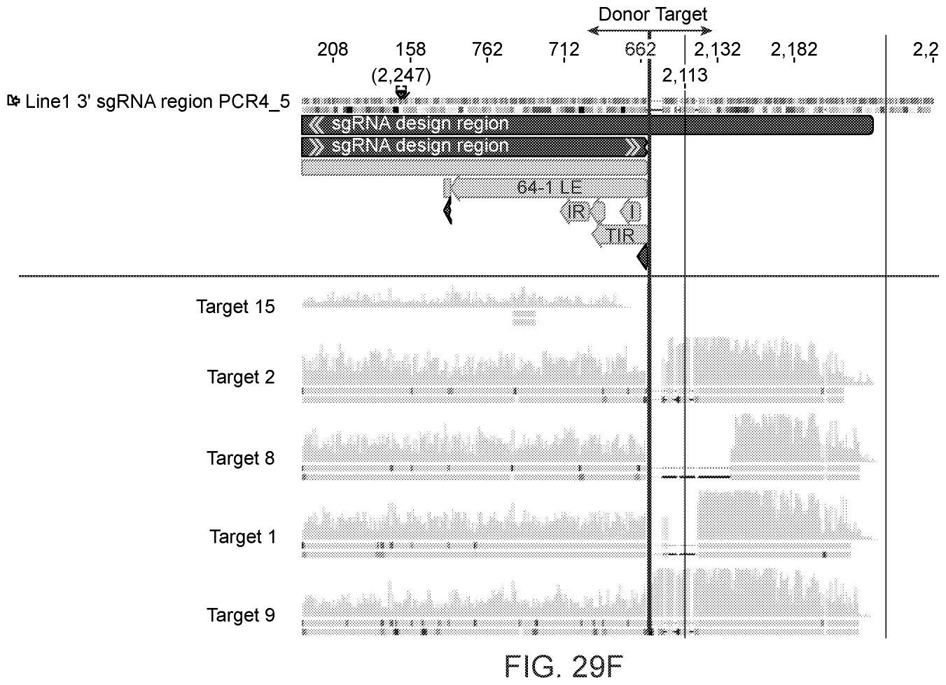

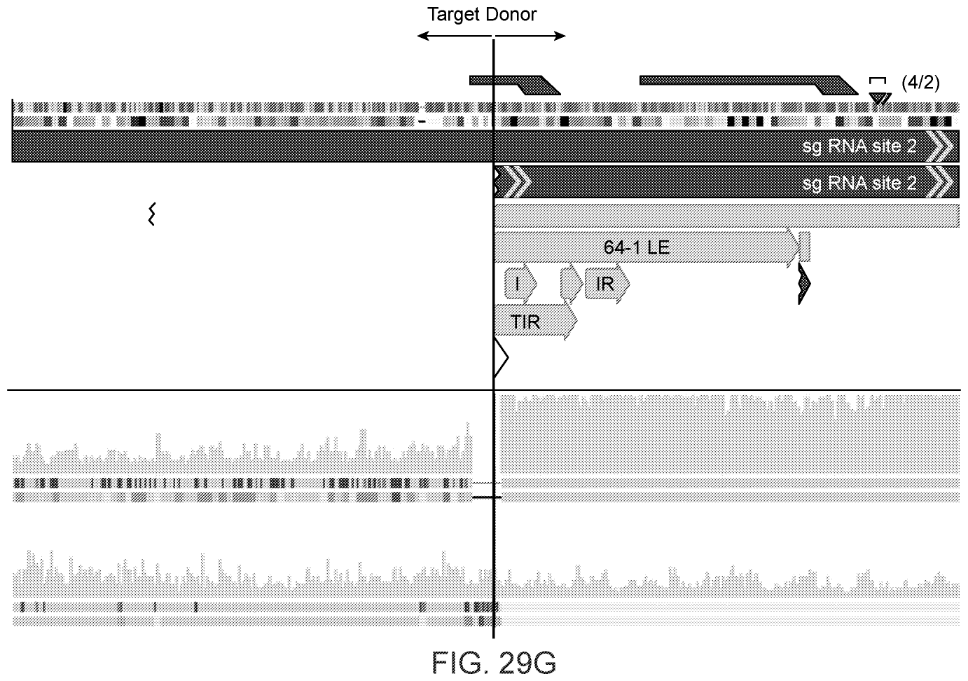

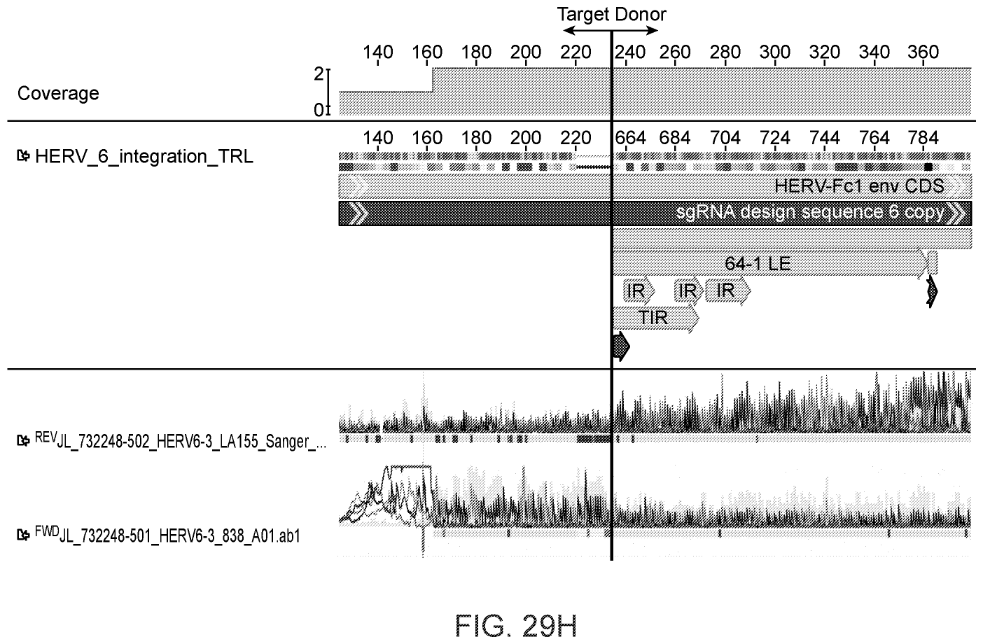

High copy targets were identified in the human genome using a Cas off-target finder. Using a 200-300 bp target sequence in the most conserved spaces of each replicated element, 15 targets sites for each LINE1 3′ and HERV were identified, and 7 target sites were designed for SVA elements with varying GC content, orientations, and permutations of the MG64-1 rGTN PAM (FIG. 29A). Target (spacer) sequences were synthesized as oligos and PCR amplified onto the MG64-1 sgRNA template with a T7 promoter upstream of the single guide backbone. PCR reactions of the MG64-1 sgRNA were then purified and in vitro transcribed. Using NLS-tagged MG64-1 protein components, an in vitro transposition reaction as described above was assembled, with purified HEK293T gDNA at 1 μg/reaction as target DNA.

### Results: In Vitro Targeted Transposition to High Copy Regions of the Human Genome

Successful transposition by MG64-1 was evaluated by the resulting Fwd PCR and Rev PCR junction products at each of the 15 target sites in high copy elements (FIG. 29B). MG64-1 promoted transposition in the forward orientation to LINE1 targets 3, 5, 6, 7, 10, and 13, while targets 1, 2, 8, 9, 12, 14, and 15 were reactive for transposition in both forward and reverse orientations (FIG. 29B). In addition, active transposition into SVA target 3 and HERV target 5 was specific for both forward and reverse orientations (FIG. 29C-29D). Sanger sequencing of transposition reaction products for LINE1, SVA, and HERV confirmed that in vitro transposition was specific and RNA-guided (FIG. 29E-29H).

### Example 35—NLS-Functional Domain Fusions with MG64-1 CAST are Targetable to High Copy Elements

Functional Domains Fused with CAST Components for Targeted Transposition In Vitro to High Copy Elements in the Human Genome

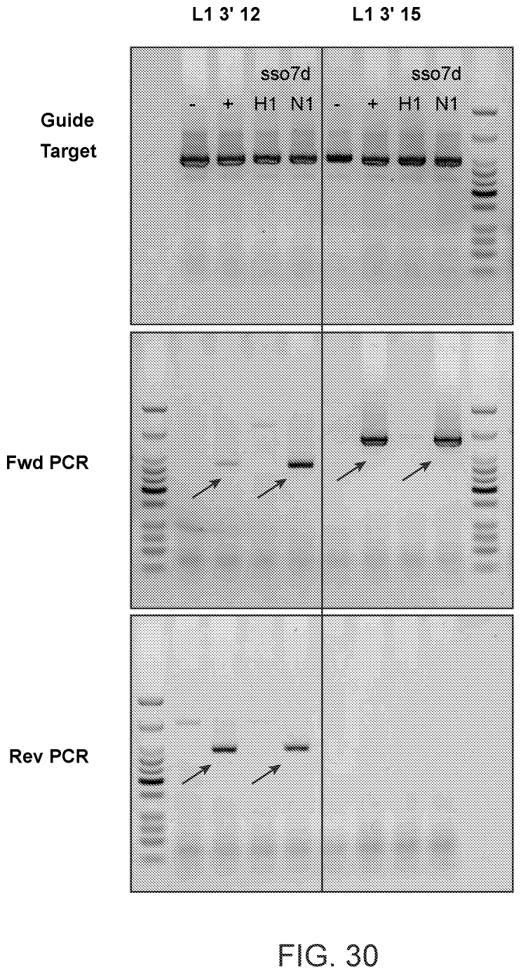

With experiments indicating that high copy elements are efficient targets for integration in vitro, functional domains were tested for their effects on CAST targeting at these sites. Functional domain fusions of Cas12k-sso7d were challenged with H1core-TniQ, or Cas12k-sso7d with HMGN1-TniQ, to transpose donor DNA when in reaction with NLS-TnsB and NLS-TnsC fusions at high copy elements LINE1 target 12 and target 15. Transposition reactions were assembled with either Cas12k alone or with fusion Cas12k-sso7d where indicated, with a no sgRNA (−sg) condition as negative control for transposition, and with sgRNA for target 12 and target 15 of LINE1 3′ elements where indicated (FIG. 30). In addition, transposition reactions were supplemented with translated NLS-TniQ, NLS-H1core-TniQ or NLS-HMGN1-TniQ, NLS-TnsB, NLS-TnsC, pDonor, buffer, and human gDNA for targeting.

### Results

In vitro transposition assays with fusion domains targeting LINE1 target 12 and target 15 indicated successful integration by the MG64-1 no-fusion, positive control (FIG. 30, lanes 3 and 7), as well as with fusions Cas12k-sso7d with HMGN1-TniQ (FIG. 30, lanes 5 and 9). No targeting occurred when no sgRNA was added to the reaction (FIG. 30, lanes 2 and 6).

### Example 36—NLS Fusion to S15 for Targeted Transposition

NLS Fusion with Small Ribosomal Protein Subunit S15 is Necessary for Correct Orientation of Tag

Recently, the small prokaryotic ribosomal protein subunit S15 was deemed necessary for targeted transposition by Cas12k CAST in vitro (Schmidt et al., 2022; Park et al., 2022). Therefore, the need for S15 with and without NLS tags in transposition experiments with MG64-1 was evaluated. Because the in vitro expression reagent already contains S15 (S15 is part of the prokaryotic ribosomal complex needed for protein expression), it is likely that in vitro transposition experiments with CAST proteins that had been expressed with in vitro expression system had S15 carried over from the expression step and was subsequently recruited for CAST targeting.

### Results: S15 Increases Transposition Efficiency in In Vitro Experiments

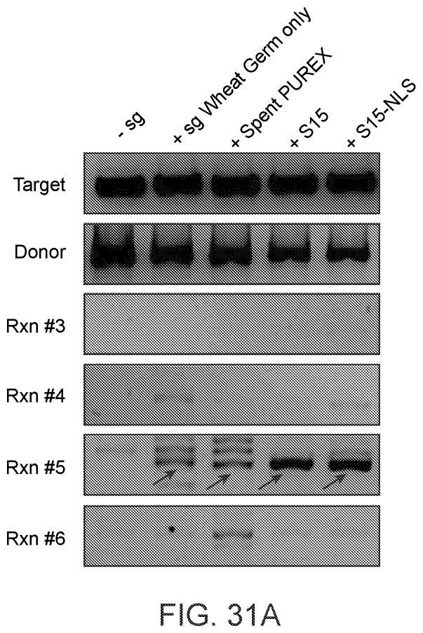

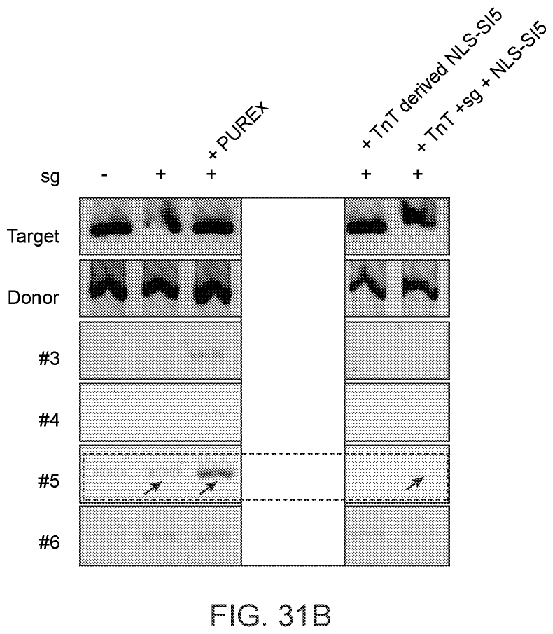

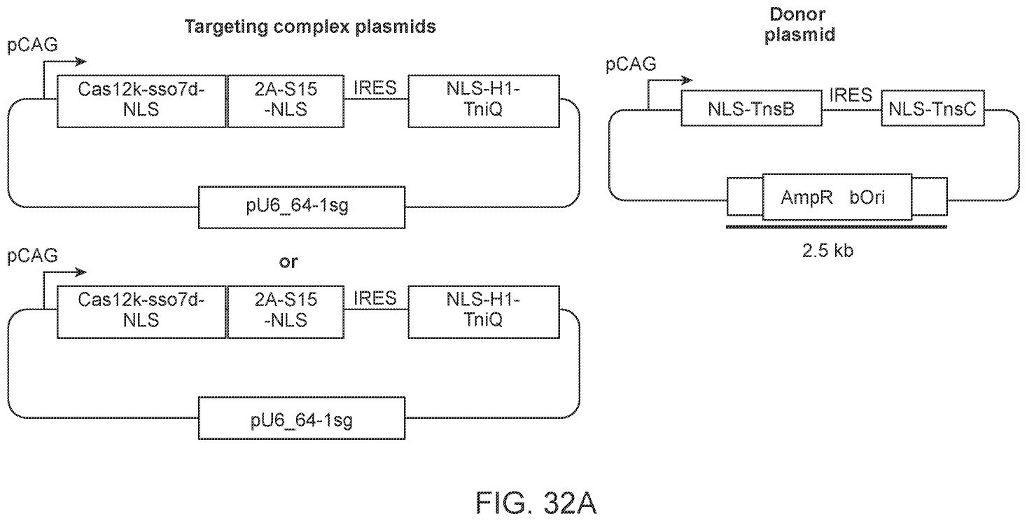

Wheat Germ Extract was used in a eukaryotic transcription/translation system, which does not contain S15, to express MG64-1 CAST components. CAST templates were amplified to contain a T7 promoter and a 40 bp Poly A tail for transcriptional stability of mRNA templates. Proteins were expressed from the dsDNA template via transcription/translation reactions, which were then used in an in vitro transposition reaction, as described above. Results indicate that S15 addition increased targeted transposition efficiency, as shown by the intensity of the bands from junction PCR products (Rxn #5) (FIG. 31A, lanes 4-5).

### Results: The S15-NLS Fusion is the Preferred Orientation for In Vitro Transposition

In eukaryotic conditions, translation of proteins is exclusively performed in the cytoplasm, while transposition reactions mediated by CAST would most likely occur in the nucleus. The necessity of an NLS tag for S15 nuclear localization was evaluated. NLS tags were fused to both N- and C-termini of S15 and tested in the Eukaryotic in vitro transcription/translation reactions and in vitro transposition experiments (FIG. 31A, lane 5, and FIG. 31B, lanes 4 and 5). The results indicate that the S15-NLS was more efficient for transposition than other tested conditions (FIG. 31A, lane 5).

### Example 37—S15 is Necessary for in Cell Translation of CAST

### Design of CAST Vectors

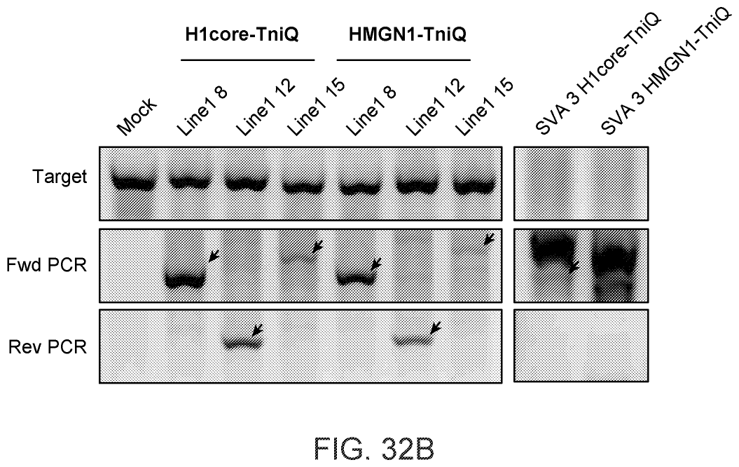

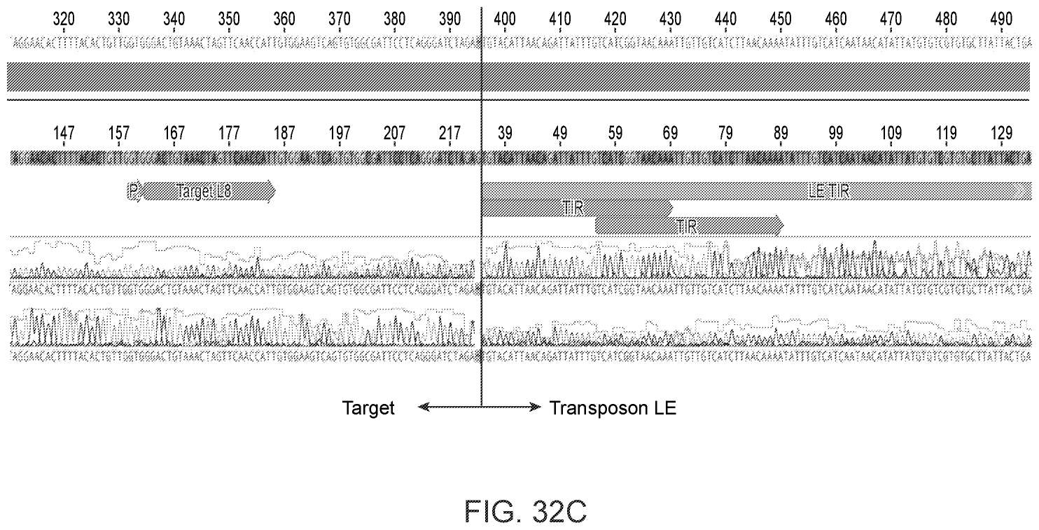

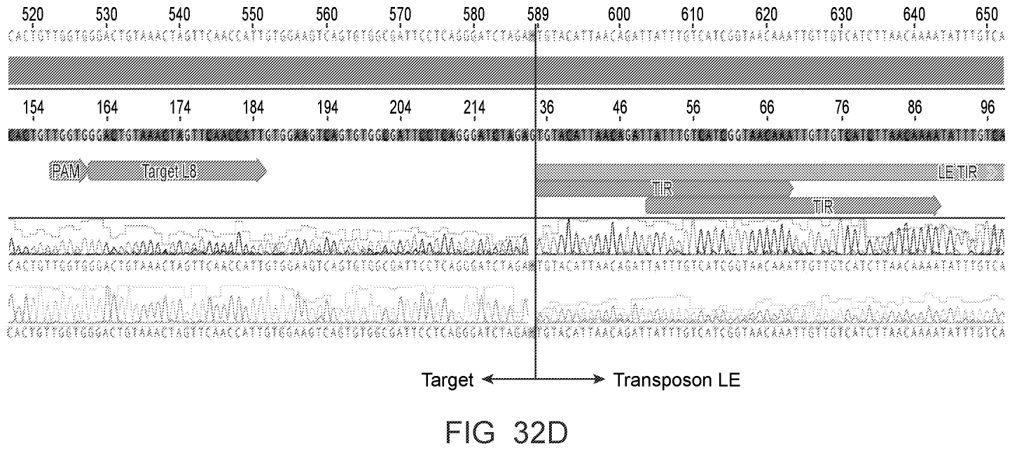

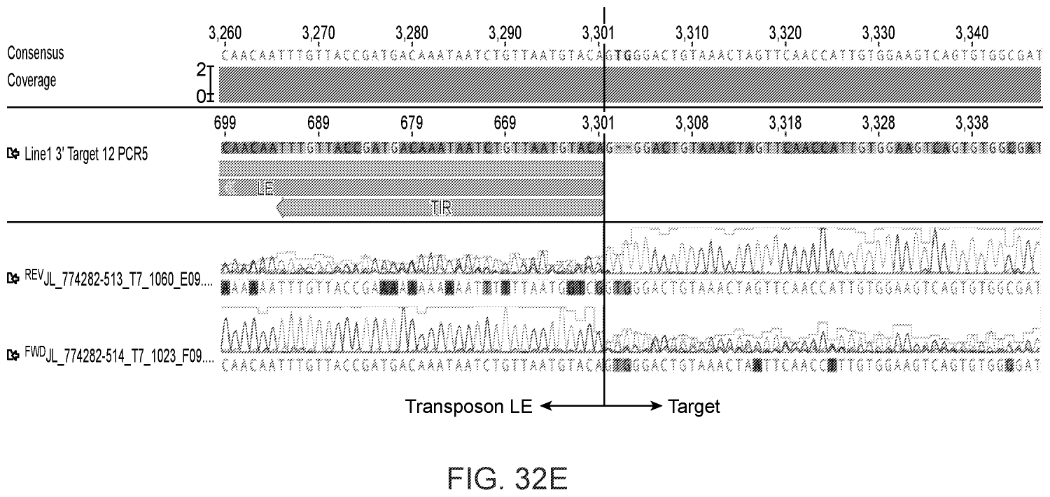

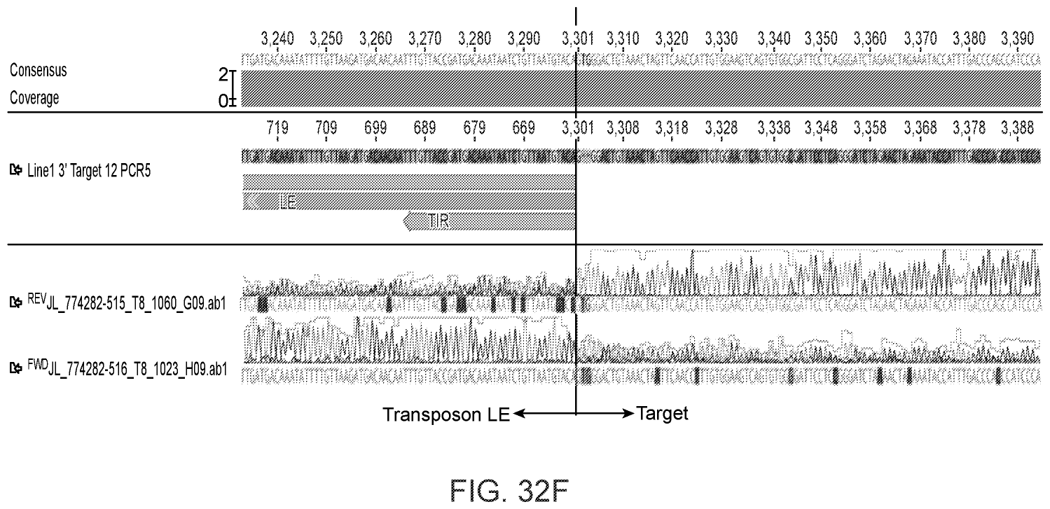

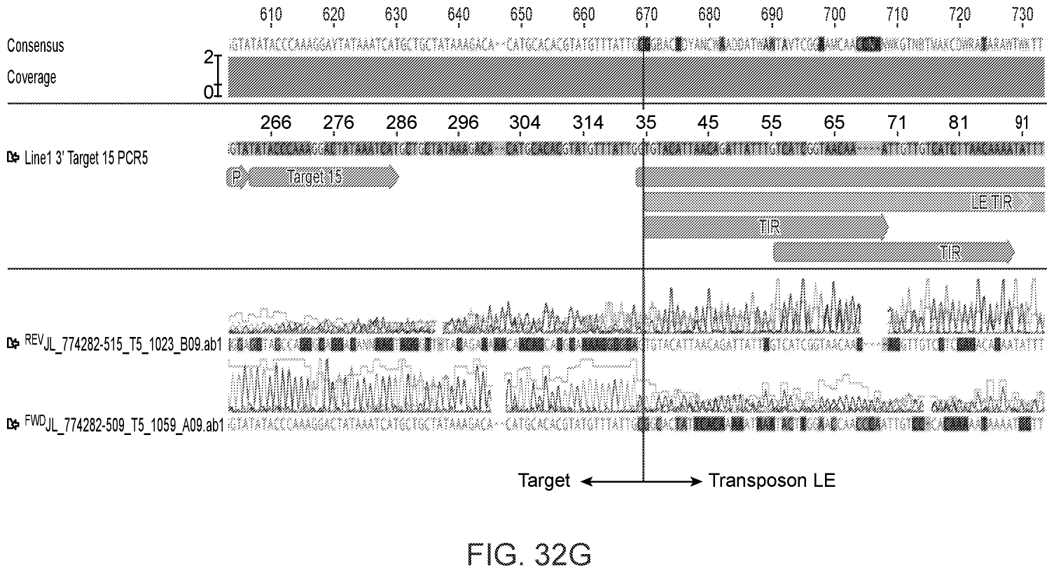

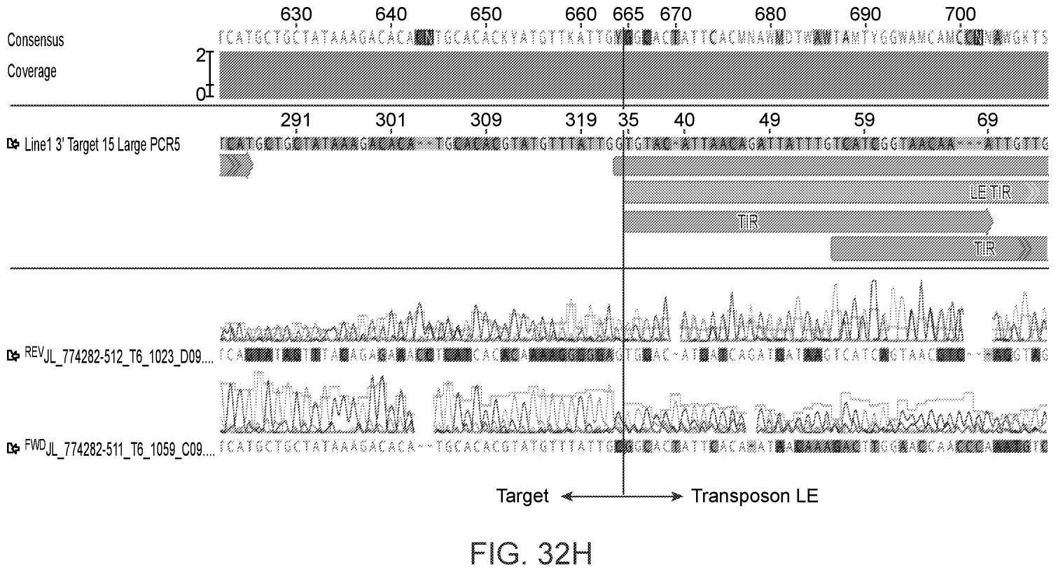

MG64-1 CAST proteins were expressed on two high expression plasmids for transposition experiments in human cells. One plasmid expresses the protein targeting complex under control of a pCAG promoter. Two versions of the protein targeting complex were designed. One version contains a Cas12k-sso7d functional domain fusion, with a 2A peptide fused to S15-NLS, IRES, and NLS-H1core-TniQ (FIG. 32A, top left). A second version contains Cas12k-sso7d-2A-S15-NLS with an NLS-HMGN1-TniQ fusion (FIG. 32A, bottom left). The targeting plasmid also contained a pU6 PolIII promoter driving transcription of a humanized MG64-1 sgRNA for targeting one of LINE1 targets 8, 12, and 15, and SVA target 3. The second plasmid transfected into cells was the donor plasmid containing NLS-TnsB and NLS-TnsC, separated with an IRES under expression of pCAG promoter. On this plasmid, 2.5 kb of DNA cargo was contained between the LE and RE terminal inverted repeats (FIG. 32A, right).

### HEK293T Lipofection

2.5 million HEK293T cells were seeded 24 hours before lipid-based transfection of the two plasmid systems in 9 μg: 9 μg of targeting: donor plasmid. Cells were incubated for 72 hours at 37° C., then harvested by resuspension in 4 mL 1×PBS pH 7.2. 2 mL of resuspended cells were harvested for gDNA and eluted in 200 μL of elution buffer. 5 μL extracted gDNA were assayed for transposition in 100 μL Q5 PCR reactions with primers specific for the high copy element targets: transposition in the forward direction was determined by amplification with primers specific for Fwd PCR, and reverse transposition was determined by amplification with primers specific for Rev PCR. Amplified PCR reactions were visualized on a 2% agarose gel. Transpositions were predicted to transpose at 60 nt away from the PAM as observed in in vitro transposition experiments, and were determined to be active by the presence of a single band for junction PCR amplification at the predicted size. PCR amplicons were Sanger sequenced and NGS sequenced for transposition profile analysis.

### Results

Cells transfected with both versions of targeting complex plasmids, with H1core-TniQ or HMGN1-TniQ, were analyzed for transposition (FIG. 32B). Both versions of the targeting complex plasmid promoted transposition at all four target sites (FIG. 32B, arrows). LINE1 targets 8 and 15 were only detectable in the LE to 5′ target orientation, while LINE1 target 12 was only detectable in the LE to 3′ orientation (FIG. 32B). Results indicate that targeted integration in human cells has a strong preference for directionality from the PAM.

Sanger sequencing of PCR junctions confirmed integration at LINE1 targets 8, 12, and 15, at 59, 62, and 60 nt away from the PAM, respectively (FIGS. 32C-32H). Sequencing signal degradation was observed at the transposition junction, which was due to a mixture of population events. In order to determine single molecule profiles of each integration event, PCR amplicons were sequenced via NGS. Reads resulting from NGS sequencing confirmed targeted integration at LINE1 targets 8, 12, and 15, and SVA target 3. Variation in the target regions indicates the natural diversity of LINE1 and SVA repeated elements in the human genome.

Overall, MG64-1 was a successful system for targeted integration in human cells, with strong directionality preference relative to the PAM.

### Example 38—In Vitro Transposition with Purified MG64-1 Targeting Complex

Construct Design and E. coli Strain Production

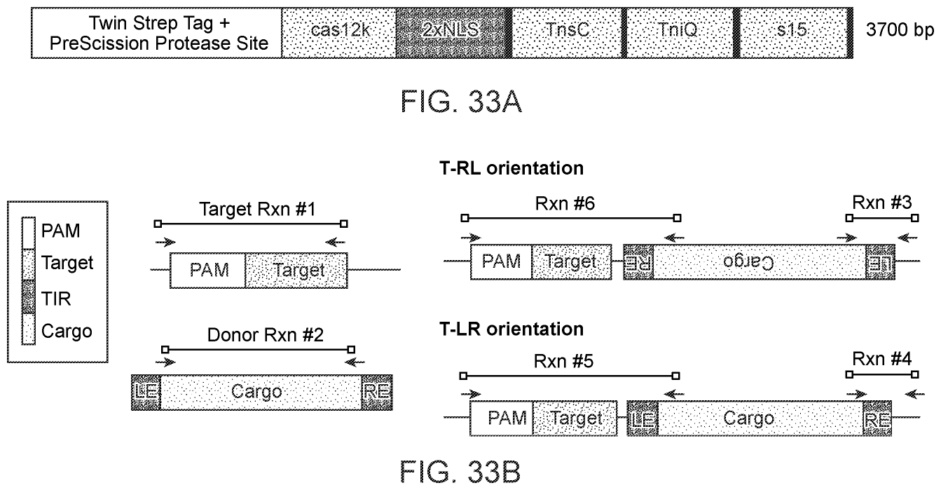

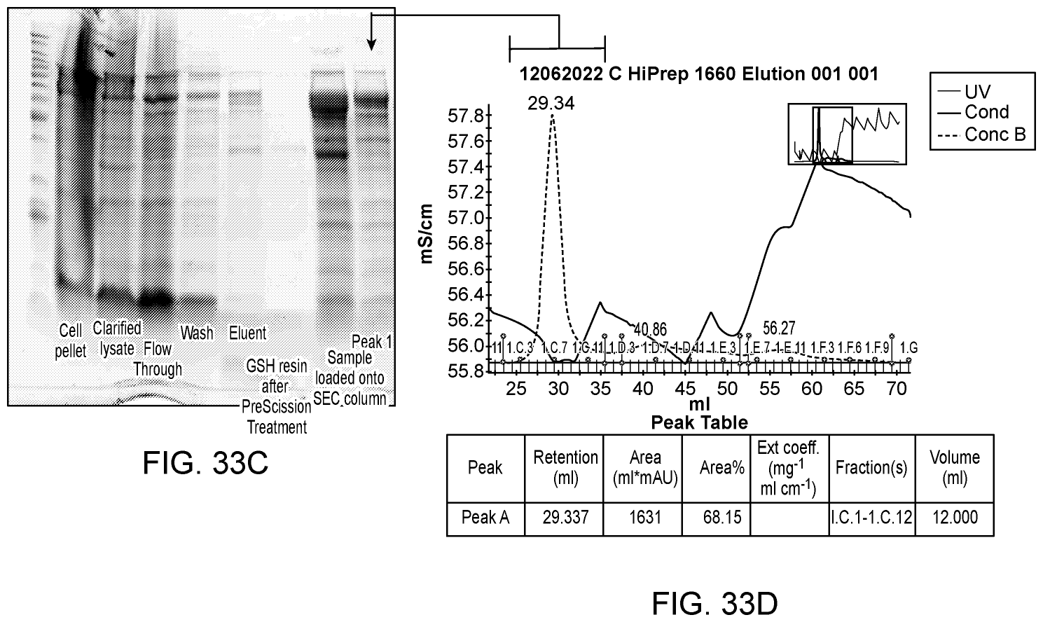

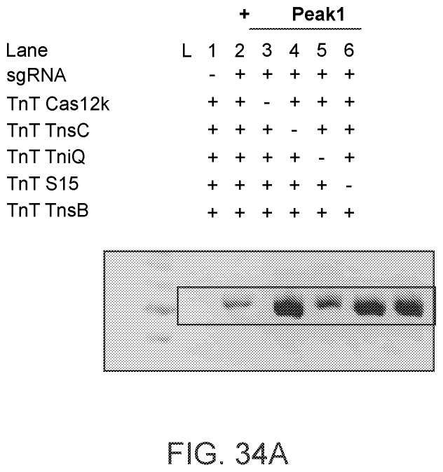

MG64-1 targeting complex was cloned into the BamHI-XhoI sites of the pET-21(+) E. coli expression vector under control of a T7 promoter. Cas12k was expressed with an N-terminal Twin Strep tag and an HRV3C protease site (FIG. 33A). The construct also contained a C-terminal 2×NLS tag on Cas12k, which was expressed in a polycistronic ORF with TniQ, TnsC, and S15 downstream of the Cas12k coding sequence. BL21(DE3) E. coli were transformed with the polycistronic plasmid and co-expressed with an sgRNA containing plasmid under the control of the J23119 constitutively active promoter.

### Purification of Cas12k Targeting Complex

2×1 L cultures of TB were inoculated with 10 mL overnight cultures of the expression construct and guide plasmid grown in LB. The TB medium was supplemented with 50 mM MgCl₂, 1 mL trace elements solution, 100 μg/mL ampicillin, and 15 μg/mL of chloramphenicol per liter. Cultures were grown to OD=0.6, and then induced with addition of IPTG to a final concentration of 0.5 mM. The culture was grown for 3H at 37° C. and then harvested by centrifugation.

50 nt oligos were synthesized and annealed in a final concentration of 100 μM. 5 mL of Streptactin resin was loaded onto a gravity flow column and allowed to drain of storage buffer. The resin was then washed with 20 mL wash buffer (50 mM Tris pH 7.4, 750 mM NaCl, 5% glycerol, 0.5 mM TCEP, 1 mM EDTA, 10 mM MgCl₂). Harvested E. coli pellets were lysed using a Branson 550 sonicator in 30 mL Lysis buffer using 12 cycles of (15 sec on 45 sec off) at 75% amplitude. Lysate was cleared by centrifugation at 30,000×g for 25 minutes at 4° C. Clarified lysate was applied to the column and allowed to flow through. The column was then washed with 25 mL of wash buffer. The holo complex was then eluted with 15 mL Elution Buffer (wash buffer with 2.5 mM desthiobiotin). The eluted protein was quantified using Bradford reagent (Serva). 50 μL of 100 μM Annealed target oligo and 200 μL of PreScission were added to eluate. Protease reaction was incubated in a rotary shaker at 4° C. overnight. To remove the PreScission protease, 5 mL GSH (Pierce ID: 25237) resin was washed with three cycles of centrifugation at 500×g for 5 minutes and resuspension in 30 mL wash buffer. The protease-treated sample was applied to this resin and incubated for 30 minutes at RT with gentle agitation. The resin was sedimented by centrifugation and the supernatant saved. The GSH resin was washed twice more with 30 mL wash buffer, and the supernatant collected. The protease-treated complex was then added to a prepared SEC column that had been pre-washed with distilled water followed by wash buffer. Samples were loaded onto the column and run at 0.5 mL/min in two column volumes of wash buffer. Protein-containing eluent fractions were pooled, concentrated, and assayed for concentration using Bradford reagent. They were diluted 1:1 into storage buffer (50 mM Tris pH 7.4, 750 mM NaCl, 40% glycerol, 1 mM EDTA, 10 mM MgCl₂, 0.5 mM TCEP) such that the final concentration of glycerol was 20% in the stored, concentrated proteins. Select samples from different stages of purification were run on a denaturing SDS PAGE gel (FIGS. 33C-33D).

### Eukaryotic Transcription and Translation (TnT) Reactions

Wheat Germ Extract-based in vitro protein expression reactions were used for expression of CAST proteins from templates amplified to contain a T7 promoter and a 40 bp Poly A tail for transcriptional stability of mRNA templates. PCR-amplified templates were normalized to 200 ng/μL and loaded into in vitro transcription/translation reactions at a final concentration of 20 ng/μL and run for 90 min at 30° C. Crude expressions were then assayed for function by in vitro transposition and used to supplement purified protein fractions.

### In Vitro Transposition

After expression, 1 μL of purified complex (fractions) was added to 0.5 picomoles of sgRNA and incubated for 20 minutes at 25° C. Individually expressed transposase proteins were then added volumetrically at 1 μL per expression with dropouts as noted to test active proteins purified with the complex pulldown. 50 ng of target DNA and 50 ng donor DNA were then added to the transposition reaction in a reaction buffer, with final concentrations of 26 mM HEPES pH 7.5, 4.2 mM TRIS pH 8, 50 μg/mL BSA, 2 mM ATP, 2.1 mM TCEP, 0.05 mM EDTA, 0.2 mM MgCl₂, 28 mM NaCl, 21 mM KCl, 1.35% glycerol (final pH 7.5), and 15 mM Mg(OAc)₂. In vitro transposition reactions were performed at 37° C. for 2 hours, transposition reactions were diluted tenfold in water, and used subsequently as a template for junction PCR analysis (FIG. 33B).

Results: Mass Spectrometry Detection of Cas12k, TnsC, s15, and TniQ in Complex Pulldowns

Proteins were purified from crude lysates according to the protocol described above as holo targeting components (complexed with sgRNA) and fractionated through size exclusion chromatography as described above. Eluted samples of the purified protein complex were submitted for analysis by mass spectrometry. Resulting detection of proteolytic treated protein fragments confirmed that Cas12k, TnsC, TniQ, and s15 were detected (Table 2).

TABLE 2
Identification of components of the MG64-1
holocomplex by mass spectrometry

| Rank | Protein Query | emPAI |
|---|---|---|
| 1 | 64-1_Effector_AA | 23.16 |
| 2 | 64-1_TnsB_AA | 1.33 |
| 3 | 64-1_TnsC_AA | 2.43 |
| 4 | 64-1_TniQ_AA | 1.61 |
| 5 | PreScission_Protease | 0.18 |
| 6 | Nucleoplasmin_NLS | 4.29 |
| 7 | s15_AA | 0.46 |

Tryptic peptides from SEC-eluted protein were searched against a proprietary database of MG64-1 protein sequences. The database also contained reference sequences for PreScission_Protease, NLS and ribosomal protein S15. Multiple components of the MG64-1 transpososome were detected. PreScission_Protease is an expected residual impurity used to cleave the N-terminal strep II tag. emPAI is a relative measure of abundance (Ishihama 2005).

Examples of unique peptide sequences identified by mass spectrometry mapping to the expected sequences of MG64-1 components are presented in Table 3.

TABLE 3
Representative unique peptide sequences
identified by mass spectrometry of tryptic
digests from the purified MG64-1 holocomplex

| | | | SEQ | |
|---|---|---|---|---|
| | Observed | | ID | Peptide |
| | m/z | e-value | NO: | sequence |
| Cas12k | 3624.1189 | 1.30E−18 | 205 | K.ATEILQSYEGT |
| | | | | EQLFNTLFQAYNS |
| | | | | EEDILTR.T |
| TnsB | 2402.2166 | 1.90E−11 | 206 | R.AGQDLSVEYSN |
| | | | | HVWQCDHTR.A |
| TnsC | 2033.2468 | 5.60E−05 | 207 | K.TVVTLSHVEAL |
| | | | | HNWLEGK.R |
| TniQ | 2627.4727 | 4.90E−20 | 208 | R.FIPIPTEEELT |
| | | | | ALSEVVQVEVER. |
| | | | | L |

Significant e-values are included.
Lower scores indicate more significant results and a more precise match.

### Results: In Vitro Testing of Purified CAST Targeting Complex Confirms Functional Cas12k, TniQ, TnsC, and s15

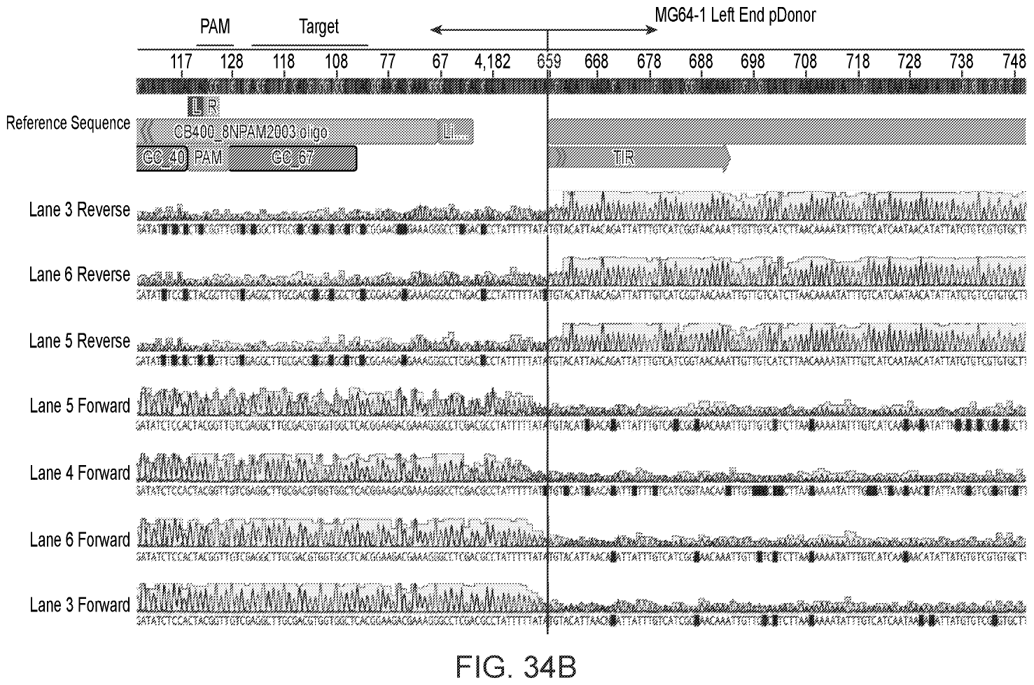

Protein components were purified as a holo targeting complex (with sgRNA) and fractionated through size exclusion chromatography as previously described. Purified complex from Peak 1 was active for transposition without the need for additional Cas12k, TnsC, TniQ nor S15 expressed with eukaryotic TnT in the reaction (FIG. 34A, lane 3-6). Positive transposition bands for the LE to Target junction were sequenced using Sanger sequencing from both donor and target specific primers, and sequencing results confirmed integration of the LE into the target DNA, as evidenced by the signal degradation at the integration site (FIG. 34B).

### Example 39—Novel Ribosomal Protein S15 Homologs for Targeted Integration

Results: Bioinformatic Discovery of S15 from Cyanobacteria

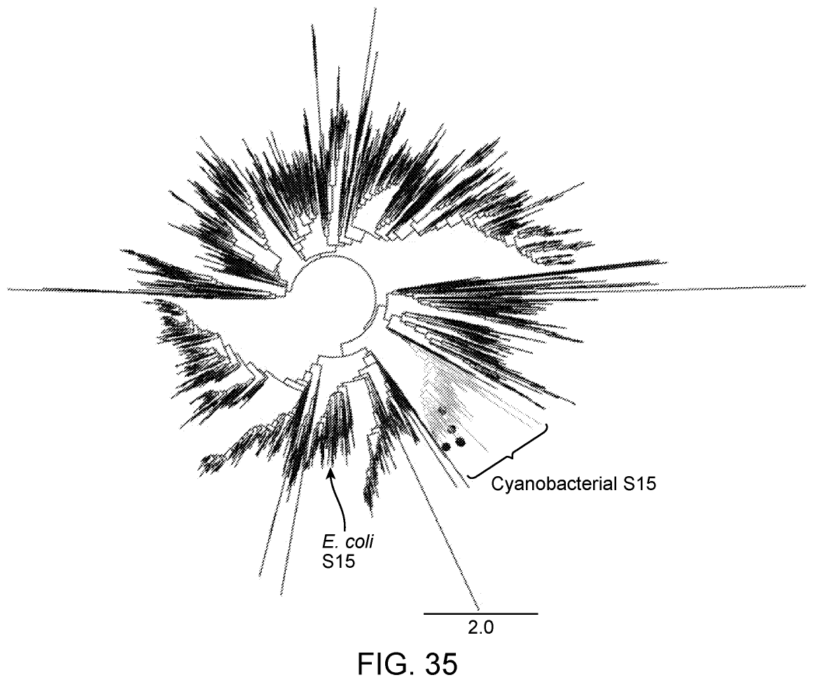

Recently, the small prokaryotic ribosomal protein subunit S15 was deemed necessary for targeted transposition by Cas12k CAST in vitro (Schmidt et al., 2022; Park et al., 2022). Ribosomal protein S15 distant homologs were identified from Pfam PF00312 domain searches with significant e-value of 1e⁻⁵. Of >1 million S15 protein hits, nearly 3,500 full-length, unique S15 sequences were identified in metagenomic assemblies in which Cas12k CAST effectors were also identified. Clustering at 99% average amino acid identity enabled classification of nearly 2,700 S15 cluster members by taxonomic affiliation, of which 11 (SEQ ID NOs: 173-183) were derived from Cyanobacteria (FIG. 35). Five ribosomal protein S15 candidate sequences (MG190-178 through MG190-182, SEQ ID NOs: 173-177) were identified in the same sample in which the MG64-1 CAST was identified (FIG. 35) and are likely associated with this CAST system.

### Example 40—NLS Fusion with S15 of the MG190 Family is Necessary for Transposition (Prophetic)

The need for S15 with and without NLS tags in transposition experiments with MG64-1 or a Cas12k CAST of the MG64 family is evaluated. NLS tags are fused to the N- and/or C-termini of S15 and tested in in vitro transposition experiments. Wheat Germ Extract is used in a Eukaryotic transcription/translation system, which does not contain S15, to express MG64-1 CAST components and NLS-S15 constructs. CAST templates are amplified to contain a T7 promoter and a 40 bp Poly A tail for transcriptional stability of mRNA templates. Proteins are expressed from the dsDNA template via transcription/translation reactions, which are then used in an in vitro transposition reaction as described previously.

### Example 41—in Cell Transposition with CAST and S15 of the MG190 Family (Prophetic)

NLS-tagged CAST proteins are expressed on high expression plasmids for transposition experiments in human cells. A targeting plasmid expresses the protein targeting complex, including S15, under control of a pCAG promoter. The targeting plasmid also contains a pU6 PolIII promoter driving transcription of a humanized sgRNA for in-cell targeted integration. A second donor plasmid containing DNA cargo flanked by the LE and RE terminal inverted repeats is transfected into cells. Cells are seeded 24 hours before lipid-based transfection of the two plasmid system in 9 μg: 9 μg of targeting: donor plasmid. Cells are incubated for 72 hours at 37° C., then harvested by resuspension in 4 mL 1×PBS pH 7.2. 2 mL of resuspended cells are harvested for gDNA extraction and eluted in 200 μL of elution buffer. 5 μL extracted gDNA is assayed for transposition in 100 μL Q5 PCR reactions with primers specific for the target site. Amplified PCR reactions are visualized on a 2% agarose gel. Transpositions are predicted to transpose at 60-65 bp away from the PAM and are determined to be active by the presence of a single band for junction PCR amplification at the predicted size. PCR amplicons are Sanger sequenced and NGS sequenced for transposition profile analysis.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

TABLE 4
Sequence Listing of Protein and Nucleic Acid Sequences Referred to Herein

| | SEQ | | | | | |
|---|---|---|---|---|---|---|
| | ID | | | | Other | |
| Category | NO: | Description | Type | Organism | Information | Sequence |
| DNA | 109 | sso7d | protein | Saccharolobus | | ATVKFKYKGEEKEVDISKIKKVWRVGKMISFTYDEGGGKTGRGAVSE |
| Binding | | | | solfataricus | | KDAPKELLQMLEKQKK |
| Domain | | | | | | |
| DNA | 110 | cbx5 | protein | Saccharomyces | | EEYVVEKVLDRRVVKGQVEYLLKWKGFSEEHNTWEPEKNLDCPELIS |
| Binding | | | | cerevisiae | | EFMK |
| Domain | | | | | | |
| DNA | 111 | HMGN1 | protein | Human | | PKRKVSSAEGAAKEEPKRRSARLSAKPPAKVEAKPKKAAAKDKSSDK |
| Binding | | | | | | KVQTKGKRGAKGKQAEVANQETKEDLPAENGETKTEESPASDEAGE |
| Domain | | | | | | KEAKSD |
| DNA | 112 | H1 core | protein | Human | | STDHPKYSDMIVAAIQAEKNRAGSSRQSIQKYIKSHYKVGENADSQIK |
| Binding | | | | | | LSIKRLVTTGVLKQTKGVGASGSFRLAKSDEP |
| Domain | | | | | | |
| MG64 | 113 | MG64-1 | protein | | | MSQITIQCRLVAKEPIRHTLWQLMADLNTPFINELLQKVAQHPDFEK |
| family | | Cas12k- | | | | WKQRGRLKVKVIEQLGNELKKDPRFLGQPARFYTSGINLVKYIFKSW |
| DBD | | sso7d- | | | | LKLQQRLQQKLDRKRRWLEVLKSDDQLIKDGQTDLETIRQKATEILQ |
| fusion | | NLS | | | | SYEGTEQLFNTLFQAYNSEEDILTRTALNYLLKNRCKLPQKPEDAKKF |
| protein | | active | | | | AKRRRQVEIAIKRLQEQIKARLPQGRDVTNENWLETLNLACYTDPENI |
| | | protein | | | | EEARSWQDKLLTKSSSIPFPINYETNEDLIWSKNEKGHLCVQFNGISDL |
| | | | | | | KFKIYCDKRQLKWFQRFYEDQQIKKSNNNQHSSALFTLRSGRILWQE |
| | | | | | | DKGKGQLWDIHRLTLQCTLDTRTWTQEGTEQVKEEKADEIAGILTRM |
| | | | | | | NEKGDLTKNQQAFIQRKQSTLDKLENPFPRPSRPVYRGQSNILLGVSM |
| | | | | | | ELKKPATIAVIDGMTRKVLTYRNIKQLLGKNYPLLNRQRRQKQLQSH |
| | | | | | | QRNVAQRKEAFNQFGDSELGEYIDRLLAKAIIAIAKQYQARSIVVPHL |
| | | | | | | KDIREAIQSEIQALAEAKIPNCIEAQAEYAKKYRIQVHQWSYGRLIDNI |
| | | | | | | QAQASKLGIVIEESQQPLQGTPLQKAAELAFKAYQSRLSGGGGSGGG |
| | | | | | | GSATVKFKYKGEEKEVDISKIKKVWRVGKMISFTYDEGGGKTGRGA |
| | | | | | | VSEKDAPKELLQMLEKQKKSGGKRPAATKKAGQAKKKKYPYDVPD |
| | | | | | | YAGSGSPKKKRKVDGSPKKKRKVDSG |
| MG64 | 114 | MG64-1 | protein | | | MSQITIQCRLVAKEPIRHTLWQLMADLNTPFINELLQKVAQHPDFEK |
| family | | Cas12k- | | | | WKQRGRLKVKVIEQLGNELKKDPRFLGQPARFYTSGINLVKYIFKSW |
| DBD | | cbx5- | | | | LKLQQRLQQKLDRKRRWLEVLKSDDQLIKDGQTDLETIRQKATEILQ |
| fusion | | NLS | | | | SYEGTEQLFNTLFQAYNSEEDILTRTALNYLLKNRCKLPQKPEDAKKF |
| protein | | active | | | | AKRRRQVEIAIKRLQEQIKARLPQGRDVTNENWLETLNLACYTDPENI |
| | | protein | | | | EEARSWQDKLLTKSSSIPFPINYETNEDLIWSKNEKGHLCVQFNGISDL |
| | | | | | | KFKIYCDKRQLKWFQRFYEDQQIKKSNNNQHSSALFTLRSGRILWQE |
| | | | | | | DKGKGQLWDIHRLTLQCTLDTRTWTQEGTEQVKEEKADEIAGILTRM |
| | | | | | | NEKGDLTKNQQAFIQRKQSTLDKLENPFPRPSRPVYRGQSNILLGVSM |
| | | | | | | ELKKPATIAVIDGMTRKVLTYRNIKQLLGKNYPLLNRQRRQKQLQSH |
| | | | | | | QRNVAQRKEAFNQFGDSELGEYIDRLLAKAIIAIAKQYQARSIVVPHL |
| | | | | | | KDIREAIQSEIQALAEAKIPNCIEAQAEYAKKYRIQVHQWSYGRLIDNI |
| | | | | | | QAQASKLGIVIEESQQPLQGTPLQKAAELAFKAYQSRLSGGGGSEEYV |
| | | | | | | VEKVLDRRVVKGQVEYLLKWKGFSEEHNTWEPEKNLDCPELISEFM |
| | | | | | | KSGGKRPAATKKAGQAKKKKYPYDVPDYAGSGSPKKKRKVDGSPK |
| | | | | | | KKRKVDSG |
| MG64 | 115 | MG64-1 | protein | | | MSQITIQCRLVAKEPIRHTLWQLMADLNTPFINELLQKVAQHPDFEK |
| family | | Cas12k- | | | | WKQRGRLKVKVIEQLGNELKKDPRFLGQPARFYTSGINLVKYIFKSW |
| DBD | | H1core- | | | | LKLQQRLQQKLDRKRRWLEVLKSDDQLIKDGQTDLETIRQKATEILQ |
| fusion | | NLS | | | | SYEGTEQLFNTLFQAYNSEEDILTRTALNYLLKNRCKLPQKPEDAKKF |
| protein | | active | | | | AKRRRQVEIAIKRLQEQIKARLPQGRDVTNENWLETLNLACYTDPENI |
| | | protein | | | | EEARSWQDKLLTKSSSIPFPINYETNEDLIWSKNEKGHLCVQFNGISDL |
| | | | | | | KFKIYCDKRQLKWFQRFYEDQQIKKSNNNQHSSALFTLRSGRILWQE |
| | | | | | | DKGKGQLWDIHRLTLQCTLDTRTWTQEGTEQVKEEKADEIAGILTRM |
| | | | | | | NEKGDLTKNQQAFIQRKQSTLDKLENPFPRPSRPVYRGQSNILLGVSM |
| | | | | | | ELKKPATIAVIDGMTRKVLTYRNIKQLLGKNYPLLNRQRRQKQLQSH |
| | | | | | | QRNVAQRKEAFNQFGDSELGEYIDRLLAKAIIAIAKQYQARSIVVPHL |
| | | | | | | KDIREAIQSEIQALAEAKIPNCIEAQAEYAKKYRIQVHQWSYGRLIDNI |
| | | | | | | QAQASKLGIVIEESQQPLQGTPLQKAAELAFKAYQSRLSGGGGSSTDH |
| | | | | | | PKYSDMIVAAIQAEKNRAGSSRQSIQKYIKSHYKVGENADSQIKLSIK |
| | | | | | | RLVTTGVLKQTKGVGASGSFRLAKSDEPSGGKRPAATKKAGQAKKK |
| | | | | | | KYPYDVPDYAGSGSPKKKRKVDGSPKKKRKVDSG |
| MG64 | 116 | MG64-1 | protein | | | MSQITIQCRLVAKEPIRHTLWQLMADLNTPFINELLQKVAQHPDFEK |
| family | | Cas12k- | | | | WKQRGRLKVKVIEQLGNELKKDPRFLGQPARFYTSGINLVKYIFKSW |
| DBD | | HMGN1 | | | | LKLQQRLQQKLDRKRRWLEVLKSDDQLIKDGQTDLETIRQKATEILQ |
| fusion | | -NLS | | | | SYEGTEQLFNTLFQAYNSEEDILTRTALNYLLKNRCKLPQKPEDAKKF |
| protein | | active | | | | AKRRRQVEIAIKRLQEQIKARLPQGRDVTNENWLETLNLACYTDPENI |
| | | protein | | | | EEARSWQDKLLTKSSSIPFPINYETNEDLIWSKNEKGHLCVQFNGISDL |
| | | | | | | KFKIYCDKRQLKWFQRFYEDQQIKKSNNNQHSSALFTLRSGRILWQE |
| | | | | | | DKGKGQLWDIHRLTLQCTLDTRTWTQEGTEQVKEEKADEIAGILTRM |
| | | | | | | NEKGDLTKNQQAFIQRKQSTLDKLENPFPRPSRPVYRGQSNILLGVSM |
| | | | | | | ELKKPATIAVIDGMTRKVLTYRNIKQLLGKNYPLLNRQRRQKQLQSH |
| | | | | | | QRNVAQRKEAFNQFGDSELGEYIDRLLAKAIIAIAKQYQARSIVVPHL |
| | | | | | | KDIREAIQSEIQALAEAKIPNCIEAQAEYAKKYRIQVHQWSYGRLIDNI |
| | | | | | | QAQASKLGIVIEESQQPLQGTPLQKAAELAFKAYQSRLSGGGGSPKRK |
| | | | | | | VSSAEGAAKEEPKRRSARLSAKPPAKVEAKPKKAAAKDKSSDKKVQ |
| | | | | | | TKGKRGAKGKQAEVANQETKEDLPAENGETKTEESPASDEAGEKEA |
| | | | | | | KSDSGGKRPAATKKAGQAKKKKYPYDVPDYAGSGSPKKKRKVDGSP |
| | | | | | | KKKRKVDSG |
| MG64 | 117 | NLS- | protein | | | MKRPAATKKAGQAKKKKGGGGSGGGGSGGGGSATVKFKYKGEEKE |
| family | | sso7d- | | | | VDISKIKKVWRVGKMISFTYDEGGGKTGRGAVSEKDAPKELLQMLE |
| DBD | | TniQ | | | | KQKKSGGYPYDVPDYANATMESREIQPWWFLVEPLAGESISHFLGRF |
| fusion | | active | | | | RRENELTVTMMGKITGLGGTITRWEKFRFIPIPTEEELTALSEVVQVEV |
| protein | | protein | | | | ERLWQMFPPKGVGMKHQPIRLCGACYEEERCHKIEWQLKTTQFCSQ |
| | | | | | | HGLTLLSECPNCGARFQFPALWVNGWCHRCFLTFGEMVEGQSNKKK |
| | | | | | | YL |
| MG64 | 118 | NLS- | protein | | | MKRPAATKKAGQAKKKKGGGGSEEYVVEKVLDRRVVKGQVEYLLK |
| family | | Cbx5- | | | | WKGFSEEHNTWEPEKNLDCPELISEFMKGGGGSGGGGSGGGGSYPY |
| DBD | | TniQ | | | | DVPDYANATMESREIQPWWFLVEPLAGESISHFLGRFRRENELTVTM |
| fusion | | active | | | | MGKITGLGGTITRWEKFRFIPIPTEEELTALSEVVQVEVERLWQMFPPK |
| protein | | protein | | | | GVGMKHQPIRLCGACYEEERCHKIEWQLKTTQFCSQHGLTLLSECPN |
| | | | | | | CGARFQFPALWVNGWCHRCFLTFGEMVEGQSNKKKYL |
| MG64 | 119 | NLS- | protein | | | MKRPAATKKAGQAKKKKGGGGSSTDHPKYSDMIVAAIQAEKNRAGS |
| family | | H1core- | | | | SRQSIQKYIKSHYKVGENADSQIKLSIKRLVTTGVLKQTKGVGASGSF |
| DBD | | TniQ | | | | RLAKSDEPGGGGSGGGGSGGGGSYPYDVPDYANATMESREIQPWWF |
| fusion | | active | | | | LVEPLAGESISHFLGRFRRENELTVTMMGKITGLGGTITRWEKFRFIPIP |
| protein | | protein | | | | TEEELTALSEVVQVEVERLWQMFPPKGVGMKHQPIRLCGACYEEERC |
| | | | | | | HKIEWQLKTTQFCSQHGLTLLSECPNCGARFQFPALWVNGWCHRCFL |
| | | | | | | TFGEMVEGQSNKKKYL |
| MG64 | 120 | NLS- | protein | | | MKRPAATKKAGQAKKKKGGGGSPKRKVSSAEGAAKEEPKRRSARLS |
| family | | HMGN1 | | | | AKPPAKVEAKPKKAAAKDKSSDKKVQTKGKRGAKGKQAEVANQET |
| DBD | | -TniQ | | | | KEDLPAENGETKTEESPASDEAGEKEAKSDGGGGSGGGGSGGGGSYP |
| fusion | | active | | | | YDVPDYANATMESREIQPWWFLVEPLAGESISHFLGRFRRENELTVT |
| protein | | protein | | | | MMGKITGLGGTITRWEKFRFIPIPTEEELTALSEVVQVEVERLWQMFP |
| | | | | | | PKGVGMKHQPIRLCGACYEEERCHKIEWQLKTTQFCSQHGLTLLSEC |
| | | | | | | PNCGARFQFPALWVNGWCHRCFLTFGEMVEGQSNKKKYL |
| Genomic | 121 | Line1 | nucleotide | artificial | | AATGCTCATCATCACTGGCCATCAGAGAAATGCAAATCAAAACCA |
| DNA | | Target | | sequence | | CTATGAGATATCATCTCACACCAGTTAGAATGGCAATCATTAAAA |
| targets | | | | | | AGTCAGGAAACAACAGGTGCTGGAGAGGATGCGGAGAAATAGGA |
| | | | | | | ACACTTTTACACTGTTGGTGGGACTGTAAACTAGTTCAACCATTGT |
| | | | | | | GGAAGTCAGTGTGGCGATTCCTCAGGGATCTAGAACTAGAAATAC |
| | | | | | | CATTTGACCCAGCCATCCCATTACTGGGTATATACCCAAAGGACTA |
| | | | | | | TAAATCATGCTGCTATAAAGACACATGCACACGTATGTTTATTGCG |
| | | | | | | GCACTATTCACAATAGCA |
| Genomic | 122 | SVA | nucleotide | artificial | | CTCTCCCTCTCCCTCTCCCTCTCCCTCTCCCTCTCCCTCTCCCTCTCC |
| DNA | | Target | | sequence | | CCTCCCTCCACGGTCTCCCTCTGATGCCGAGCCAAAGCTGGACGGT |
| targets | | | | | | ACTGCTGCCATCTCGGCTCACTGCAACCTCCCTGCCTGATTCTCCT |
| | | | | | | GCCTCAGCCTGCCGAGTGCCTGCGATTGCAGGCGCGCGCCGCCAC |
| | | | | | | GCCTGACTGGTTTTCGTTTTTTTTTGGTGGAGACGGGGTCTCGCTGT |
| | | | | | | GTTGNCCGGGCTGGTCTCCAGCTCCTGGCCGCGAGTGATCCGCCNG |
| | | | | | | CCTCGGCCTCCCGAGGTGCCGGGATTGCAGACGGAGTCTCGTTCAC |
| | | | | | | TCAGTGCTCAATGGTGCCCAGGCTGGAGTGCAGTGGCGTGATCTC |
| | | | | | | GGCTCGCTACAACCACCTCCCAGCCGCCTGCCTTGGCCTCCCAAAG |
| | | | | | | AGCCGGATTGCAGCCTCTGCCCGGCCGCCGCCCCGTCCGGGAGGT |
| | | | | | | GAGGAGCGCCTCTGCCCGGCCGCCC |
| Genomic | 123 | HERV | nucleotide | artificial | | TTCAACCGGCAGCTGACCAGGCCAAGTCCTCCCCCTTCTCGTGGTT |
| DNA | | Target | | sequence | | AACTTTAATCTCAGAAGGTGCACAATTGCTCCAATCCACAGGGGT |
| targets | | | | | | ACAAAACCTCTCCCACTGCTTCCTCTGTGCAGCCCTCGGAAGACCT |
| | | | | | | CCCTTAGTAGCAGTTCCTCTCCCTACCCCCTTTAATTATACAAGAA |
| | | | | | | ATTCATCCACCCCTATACCACCGGTCCCGAAAGGACAGGTCCCACT |
| | | | | | | ATTCTCAGACCCTACAAGACACAAGTTCCCGTTCTGTTACTCTACC |
| | | | | | | CCAAATGCCTCTTGGTGTAACCAGACCAGGATGCTTACCAGCGCCC |
| | | | | | | CG |
| Target | 124 | Line1 | nucleotide | artificial | | ATGGGATGGCTGGGTCAAATGGTA |
| guides | | Target 1 | | sequence | | |
| Target | 125 | Line1 | nucleotide | artificial | | AAATGGTATTTCTAGTTCTAGATC |
| guides | | Target 2 | | sequence | | |
| Target | 126 | Line1 | nucleotide | artificial | | GAACTAGTTTACAGTCCCACCAAC |
| guides | | Target 3 | | sequence | | |
| Target | 127 | Line1 | nucleotide | artificial | | TATACCCAGTAATGGGATGGCTGG |
| guides | | Target 4 | | sequence | | |
| Target | 128 | Line1 | nucleotide | artificial | | AGGAAACAACAGGTGCTGGAGAGG |
| guides | | Target 5 | | sequence | | |
| Target | 129 | Line1 | nucleotide | artificial | | TACAGTCCCACCAACAGTGTAAAA |
| guides | | Target 6 | | sequence | | |
| Target | 130 | Line1 | nucleotide | artificial | | CACCAACAGTGTAAAAGTGTTCC |
| guides | | Target 7 | | sequence | | |
| Target | 131 | Line1 | nucleotide | artificial | | GGACTGTAAACTAGTTCAACCATT |
| guides | | Target 8 | | sequence | | |
| Target | 132 | Line1 | nucleotide | artificial | | TTTCTAGTTCTAGATCCCTGAGGA |
| guides | | Target 9 | | sequence | | |
| Target | 133 | Line1 | nucleotide | artificial | | CAACCATTGTGGAAGTCAGTGTGG |
| guides | | Target | | sequence | | |
| | | 10 | | | | |
| Target | 134 | Line1 | nucleotide | artificial | | TGGCGATTCCTCAGGGATCTAGAA |
| guides | | Target | | sequence | | |
| | | 11 | | | | |
| Target | 135 | Line1 | nucleotide | artificial | | CTAGATCCCTGAGGAATCGCCACA |
| guides | | Target | | sequence | | |
| | | 12 | | | | |
| Target | 136 | Line1 | nucleotide | artificial | | AGAATGGCAATCATTAAAAAGTCA |
| guides | | Target | | sequence | | |
| | | 13 | | | | |
| Target | 137 | Line1 | nucleotide | artificial | | AGTGTGGCGATTCCTCAGGGATCT |
| guides | | Target | | sequence | | |
| | | 14 | | | | |
| Target | 138 | Line1 | nucleotide | artificial | | TATACCCAAAGGACTATAAATCAT |
| guides | | Target | | sequence | | |
| | | 15 | | | | |
| Target | 139 | SVA | nucleotide | artificial | | GTAGCGAGCCGAGATCACGCCACT |
| guides | | Target 1 | | sequence | | |
| Target | 140 | SVA | nucleotide | artificial | | CTCAATGGTGCCCAGGCTGGAGTG |
| guides | | Target 2 | | sequence | | |
| Target | 141 | SVA | nucleotide | artificial | | CCCAGGCTGGAGTGCAGTGGCGTG |
| guides | | Target 3 | | sequence | | |
| Target | 142 | SVA | nucleotide | artificial | | GCGTGATCTCGGCTCGCTACAACC |
| guides | | Target 4 | | sequence | | |
| Target | 143 | SVA | nucleotide | artificial | | CAGTGGCGTGATCTCGGCTCGCTA |
| guides | | Target 5 | | sequence | | |
| Target | 144 | SVA | nucleotide | artificial | | TCGTTCACTCAGTGCTCAATGGTG |
| guides | | Target 6 | | sequence | | |
| Target | 145 | SVA | nucleotide | artificial | | AACGAGACTCCGTCTGCAATCCCG |
| guides | | Target 7 | | sequence | | |
| Target | 146 | HERV | nucleotide | artificial | | CCTCTCCCTACCCCCTTTAATTAT |
| guides | | Target 1 | | sequence | | |
| Target | 147 | HERV | nucleotide | artificial | | AGCATCCTGGTCTGGTTACACCAA |
| guides | | Target 2 | | sequence | | |
| Target | 148 | HERV | nucleotide | artificial | | ACAGAACGGGAACTTGTGTCTTGT |
| guides | | Target 3 | | sequence | | |
| Target | 149 | HERV | nucleotide | artificial | | GTATAGGGGTGGATGAATTTCTTG |
| guides | | Target 4 | | sequence | | |
| Target | 150 | HERV | nucleotide | artificial | | TAGGGGTGGATGAATTTCTTGTAT |
| guides | | Target 5 | | sequence | | |
| Target | 151 | HERV | nucleotide | artificial | | GATGAATTTCTTGTATAATTAAAG |
| guides | | Target 6 | | sequence | | |
| Target | 152 | HERV | nucleotide | artificial | | ACACCAAGAGGCATTTGGGGTAGA |
| guides | | Target 7 | | sequence | | |
| Target | 153 | HERV | nucleotide | artificial | | TAACCAGACCAGGATGCTTACCAG |
| guides | | Target 8 | | sequence | | |
| Target | 154 | HERV | nucleotide | artificial | | TGGTTACACCAAGAGGCATTTGGG |
| guides | | Target 9 | | sequence | | |
| Target | 155 | HERV | nucleotide | artificial | | GAGTAACAGAACGGGAACTTGTGT |
| guides | | Target | | sequence | | |
| | | 10 | | | | |
| Target | 156 | HERV | nucleotide | artificial | | GGGAGAGGAACTGCTACTAAGGGA |
| guides | | Target | | sequence | | |
| | | 11 | | | | |
| Target | 157 | HERV | nucleotide | artificial | | AACTTTAATCTCAGAAGGTGCACA |
| guides | | Target | | sequence | | |
| | | 12 | | | | |
| Target | 158 | HERV | nucleotide | artificial | | TGAGAATAGTGGGACCTGTCCTTT |
| guides | | Target | | sequence | | |
| | | 13 | | | | |
| Target | 159 | HERV | nucleotide | artificial | | CCGAAAGGACAGGTCCCACTATTC |
| guides | | Target | | sequence | | |
| | | 14 | | | | |
| Target | 160 | HERV | nucleotide | artificial | | CACAATTGCTCCAATCCACAGGGG |
| guides | | Target | | sequence | | |
| | | 15 | | | | |
| S15 fusion | 161 | NLS- | nucleotide | artificial | | ATGAAGAGGCCCGCAGCGACTAAGAAAGCAGGACAGGCGAAGAA |
| constructs | | S15 | | sequence | | GAAGAAGTACCCATATGACGTGCCAGATTATGCTGGTGGCGGCGG |
| | | | | | | CTCTGGTGGTGGCGGCAGCGGTGGCGGCGGCAGTTCTCTGTCAAC |
| | | | | | | GGAAGCAACCGCAAAGATTGTATCAGAGTTTGGCAGAGACGCGAA |
| | | | | | | CGATACGGGGAGTACGGAAGTGCAGGTCGCCCTGCTCACCGCACA |
| | | | | | | AATTAACCACCTTCAGGGACATTTCGCTGAACATAAAAAAGATCA |
| | | | | | | TCATTCGCGCAGAGGTTTGCTTCGTATGGTGTCGCAGCGTAGAAAA |
| | | | | | | CTTTTAGATTATCTGAAACGGAAAGATGTGGCACGCTATACGCAGT |
| | | | | | | TAATAGAACGACTGGGTCTGCGTCGC |
| S15 fusion | 162 | S15- | nucleotide | artificial | | ATGTCTCTGTCAACGGAAGCAACCGCAAAGATTGTATCAGAGTTT |
| constructs | | NLS | | sequence | | GGCAGAGACGCGAACGATACGGGGAGTACGGAAGTGCAGGTCGC |
| | | | | | | CCTGCTCACCGCACAAATTAACCACCTTCAGGGACATTTCGCTGAA |
| | | | | | | CATAAAAAAGATCATCATTCGCGCAGAGGTTTGCTTCGTATGGTGT |
| | | | | | | CGCAGCGTAGAAAACTTTTAGATTATCTGAAACGGAAAGATGTGG |
| | | | | | | CACGCTATACGCAGTTAATAGAACGACTGGGTCTGCGTCGCGGTG |
| | | | | | | GCGGCGGCTCTGGTGGTGGCGGCAGCGGTGGCGGCGGCAGTTATC |
| | | | | | | CATATGATGTTCCAGACTATGCAGGCAGCGGCAGCCCTAAGAAGA |
| | | | | | | AGCGAAAAGTCGACGGAAGCCCTAAGAAGAAGCGCAAGGTCGAC |
| | | | | | | AGTGGATGA |
| S15 fusion | 163 | 2A-S15- | nucleotide | artificial | | GGCGCCACCAACTTCTCCCTGCTGAAGCAGGCCGGCGACGTGGAG |
| constructs | | NLS | | sequence | | GAGAACCCCGGCCCCTCTCTGTCAACGGAAGCAACCGCAAAGATT |
| | | | | | | GTATCAGAGTTTGGCAGAGACGCGAACGATACGGGGAGTACGGAA |
| | | | | | | GTGCAGGTCGCCCTGCTCACCGCACAAATTAACCACCTTCAGGGA |
| | | | | | | CATTTCGCTGAACATAAAAAAGATCATCATTCGCGCAGAGGTTTGC |
| | | | | | | TTCGTATGGTGTCGCAGCGTAGAAAACTTTTAGATTATCTGAAACG |
| | | | | | | GAAAGATGTGGCACGCTATACGCAGTTAATAGAACGACTGGGTCT |
| | | | | | | GCGTCGCGGTGGCGGCGGCTCTGGTGGTGGCGGCAGCGGTGGCGG |
| | | | | | | CGGCAGTTATCCATATGATGTTCCAGACTATGCAGGCAGCGGCAG |
| | | | | | | CCCTAAGAAGAAGCGAAAAGTCGACGGAAGCCCTAAGAAGAAGC |
| | | | | | | GCAAGGTCGACAGTGGATGA |
| Donor | 164 | Amp- | nucleotide | artificial | | TTTACAGAGAAACCTCCTCACACAAAAGGCGTAGTGTACATTAAC |
| constructs | | bOri | | sequence | | AGATTATTTGTCATCGGTAACAAATTGTTGTCATCTTAACAAAATA |
| | | donor | | | | TTTGTCATCAATAACATATTATGTGTCGTGTGCTTATTACTGAAACT |
| | | flanked | | | | AATCCTAGACGATGGTAAAAAATCCTGCAGGTTAATTAAATTTAA |
| | | by LE | | | | ATCATGTGAGCAAAAGGCCAGCAAAAGGCCAGGAACCGTAAAAA |
| | | and RE | | | | GGCCGCGTTGCTGGCGTTTTTCCATAGGCTCCGCCCCCCTGACGAG |
| | | | | | | CATCACAAAAATCGACGCTCAAGTCAGAGGTGGCGAAACCCGACA |
| | | | | | | GGACTATAAAGATACCAGGCGTTTCCCCCTGGAAGCTCCCTCGTGC |
| | | | | | | GCTCTCCTGTTCCGACCCTGCCGCTTACCGGATACCTGTCCGCCTTT |
| | | | | | | CTCCCTTCGGGAAGCGTGGCGCTTTCTCATAGCTCACGCTGTAGGT |
| | | | | | | ATCTCAGTTCGGTGTAGGTCGTTCGCTCCAAGCTGGGCTGTGTGCA |
| | | | | | | CGAACCCCCCGTTCAGCCCGACCGCTGCGCCTTATCCGGTAACTAT |
| | | | | | | CGTCTTGAGTCCAACCCGGTAAGACACGACTTATCGCCACTGGCA |
| | | | | | | GCAGCCACTGGTAACAGGATTAGCAGAGCGAGGTATGTAGGCGGT |
| | | | | | | GCTACAGAGTTCTTGAAGTGGTGGCCTAACTACGGCTACACTAGA |
| | | | | | | AGAACAGTATTTGGTATCTGCGCTCTGCTGAAGCCAGTTACCTTCG |
| | | | | | | GAAAAAGAGTTGGTAGCTCTTGATCCGGCAAACAAACCACCGCTG |
| | | | | | | GTAGCGGTGGTTTTTTTGTTTGCAAGCAGCAGATTACGCGCAGAAA |
| | | | | | | AAAAGGATCTCAAGAAGATCCTTTGATCTTTTCTACGGGGTCTGAC |
| | | | | | | GCTCAGTGGAACGAAAACTCACGTTAAGGGATTTTGGTCATGAGA |
| | | | | | | TTATCAAAAAGGATCTTCACCTAGATCCTTTTAAATTAAAAATGAA |
| | | | | | | GTTTTAAATCAATCTAAAGTATATATGAGTAAACTTGGTCTGACAG |
| | | | | | | TTACCAATGCTTAATCAGTGAGGCACCTATCTCAGCGATCTGTCTA |
| | | | | | | TTTCGTTCATCCATAGTTGCATTTAAATGGCCGGCCTGGCGCGCCG |
| | | | | | | TTTAAACCTAGATATTGATAGTCTGATCGGTCAACGTATAATCGAG |
| | | | | | | TCCTAGCTTTTGCAAACATCTATCAAGAGACAGGATCAGCAGGAG |
| | | | | | | GCTTTCGCATGAGTATTCAACATTTCCGTGTCGCCCTTATTCCCTTT |
| | | | | | | TTTGCGGCATTTTGCCTTCCTGTTTTTGCTCACCCAGAAACGCTGGT |
| | | | | | | GAAAGTAAAAGATGCTGAAGATCAGTTGGGTGCGCGAGTGGGTTA |
| | | | | | | CATCGAACTGGATCTCAACAGCGGTAAGATCCTTGAGAGTTTTCGC |
| | | | | | | CCCGAAGAACGCTTTCCAATGATGAGCACTTTTAAAGTTCTGCTAT |
| | | | | | | GTGGCGCGGTATTATCCCGTATTGACGCCGGGCAAGAGCAACTCG |
| | | | | | | GTCGCCGCATACACTATTCTCAGAATGACTTGGTTGAGTATTCACC |
| | | | | | | AGTCACAGAAAAGCATCTTACGGATGGCATGACAGTAAGAGAATT |
| | | | | | | ATGCAGTGCTGCCATAACCATGAGTGATAACACTGCGGCCAACTT |
| | | | | | | ACTTCTGACAACGATTGGAGGACCGAAGGAGCTAACCGCTTTTTTG |
| | | | | | | CACAACATGGGGGATCATGTAACTCGCCTTGATCGTTGGGAACCG |
| | | | | | | GAGCTGAATGAAGCCATACCAAACGACGAGCGTGACACCACGATG |
| | | | | | | CCTGTAGCAATGGCAACAACCTTGCGTAAACTATTAACTGGCGAA |
| | | | | | | CTACTTACTCTAGCTTCCCGGCAACAGTTGATAGACTGGATGGAGG |
| | | | | | | CGGATAAAGTTGCAGGACCACTTCTGCGCTCGGCCCTTCCGGCTGG |
| | | | | | | CTGGTTTATTGCTGATAAATCTGGAGCCGGTGAGCGTGGGTCTCGC |
| | | | | | | GGTATCATTGCAGCACTGGGGCCAGATGGTAAGCCCTCCCGTATC |
| | | | | | | GTAGTTATCTACACGACGGGGAGTCAGGCAACTATGGATGAACGA |
| | | | | | | AATAGACAGATCGCTGAGATAGGTGCCTCACTGATTAAGCATTGG |
| | | | | | | TAACCGATTCTAGGTGCATTGGCGCAGAAAAAAATGCCTGATGCG |
| | | | | | | ACGCTGCGCGTCTTATACTCCCACATATGCCAGATTCAGCAACGGA |
| | | | | | | TACGGCTTCCCCAACTTGCCCACTTCCATACGTGTCCTCCTTACCA |
| | | | | | | GAAATTTATCCTTAAGAATGAACAAAAATGTCTGATTATTACATAA |
| | | | | | | TTGTTTATTTAATATAATTGTATCGTAATACTTGAAGTTTGGAGAC |
| | | | | | | AAGTAATTTGTTAATACTGCTCCAGTCCCTAAAAAAGTGCCATTCG |
| | | | | | | GGTAAATGACACTTAATCTGTTAATTTACTGGAAAATGACAGTTAA |
| | | | | | | TTTGTTAATATAGTAAGCAATAACTTTTGTCAAAGATTAATGCTAT |
| | | | | | | AATTCAGCTAAAGCAGTGATTATATAAAGCTTTCACTCTCAAATAG |
| | | | | | | TTCGGCGACACGATTTTGTTAAGACGACAAATAATTAGTTACTGTA |
| | | | | | | CA |
| MG64-1 | 165 | Humani | nucleotide | artificial | | GAAATAATCGCGCCGTAGATCATGTTCTTGATTGAACCTCTGAACT |
| sgRNA | | zed | | sequence | | ACGAAAAATGAGGGTTAGTTTGACTCTCGGCAGATAGTCTTGCTTT |
| | | MG64-1 | | | | CTGACCCTAGTGGCTGTCCACCCTGATGCTGATTTCTACAATTTAG |
| | | sgRNA | | | | GTTGTAGAGATGATTAACCTGTAACTTGAGGTTAGCTAATAATTTC |
| | | | | | | ATCTTATAGGGTAGGTGCGCTCCCAGCAATAAGTGGCGTGGGTTTA |
| | | | | | | CCACAGTGACGGCTACTGAATCACCTCCGACCAAGGAGGAATCCA |
| | | | | | | CTGAAAAGATGGATTGAAAG |
| linkers | 166 | IRES | nucleotide | artificial | | TGACTCGAGCTTATTCCAGATGCGTGCGGATGGAATTCGAGCTCGG |
| | | | | sequence | | TACCATGCCAAAAGCAAAGCGCTATCGCGCCTTACGTTACTGGCC |
| | | | | | | GAAGCCGCTTGGAATAAGGCCGGTGTGCGTTTGTCTATATGTTATT |
| | | | | | | TTCCACCATATTGCCGTCTTTTGGCAATGTGAGGGCCCGGAAACCT |
| | | | | | | GGCCCTGTCTTCTTGACGAGCATTCCTAGGGGTCTTTCCCCTCTCGC |
| | | | | | | CAAAGGAATGCAAGGTCTGTTGAATGTCGTGAAGGAAGCAGTTCC |
| | | | | | | TCTGGAAGCTTCTTGAAGACAAACAACGTCTGTAGCGACCCTTTGC |
| | | | | | | AGGCAGCGGAACCCCCCACCTGGCGACAGGTGCCTCTGCGGCCAA |
| | | | | | | AAGCCACGTGTATAAGATACACCTGCAAAGGCGGCACAACCCCAG |
| | | | | | | TGCCACGTTGTGAGTTGGATAGTTGTGGAAAGAGTCAAATGGCTC |
| | | | | | | CCCTCAAGCGTATTCAACAAGGGGCTGAAGGATGCCCAGAAGGTA |
| | | | | | | CCCCATTGTATGGGATCTGATCTGGGGCCTCGGTGCACATGCTTTT |
| | | | | | | CATGTGTTTAGTCGAGGTTAAAAAACGTCTAGGCCCCCCGAACCA |
| | | | | | | CGGGGACGTGGTTTTCCTTTGAAAAACACGATGATAATAAC |
| S15 fusion | 167 | NLS- | protein | artificial | | MKRPAATKKAGQAKKKKYPYDVPDYAGGGGSGGGGSGGGGSSLST |
| proteins | | S15 | | sequence | | EATAKIVSEFGRDANDTGSTEVQVALLTAQINHLQGHFAEHKKDHHS |
| | | | | | | RRGLLRMVSQRRKLLDYLKRKDVARYTQLIERLGLRR |
| S15 fusion | 168 | S15- | protein | artificial | | MSLSTEATAKIVSEFGRDANDTGSTEVQVALLTAQINHLQGHFAEHK |
| proteins | | NLS | | sequence | | KDHHSRRGLLRMVSQRRKLLDYLKRKDVARYTQLIERLGLRRGGGG |
| | | | | | | SGGGGSGGGGSYPYDVPDYAGSGSPKKKRKVDGSPKKKRKVDSG |
| S15 fusion | 169 | 2A-S15- | protein | artificial | | GATNFSLLKQAGDVEENPGPSLSTEATAKIVSEFGRDANDTGSTEVQV |
| proteins | | NLS | | sequence | | ALLTAQINHLQGHFAEHKKDHHSRRGLLRMVSQRRKLLDYLKRKDV |
| | | | | | | ARYTQLIERLGLRRGGGGSGGGGSGGGGSYPYDVPDYAGSGSPKKK |
| | | | | | | RKVDGSPKKKRKVDSG |
| Promoters | 170 | pCAG | nucleotide | artificial | | AACTGCTGATCGAGTGTAGCCAGATCTAGTAATCAATTACGGGGT |
| | | promoter | | sequence | | CATTAGTTCATAGCCCATATATGGAGTTCCGCGTTACATAACTTAC |
| | | | | | | GGTAAATGGCCCGCCTGGCTGACCGCCCAACGACCCCCGCCCATT |
| | | | | | | GACGTCAATAATGACGTATGTTCCCATAGTAACGCCAATAGGGAC |
| | | | | | | TTTCCATTGACGTCAATGGGTGGAGTATTTACGGTAAACTGCCCAC |
| | | | | | | TTGGCAGTACATCAAGTGTATCATATGCCAAGTACGCCCCCTATTG |
| | | | | | | ACGTCAATGACGGTAAATGGCCCGCCTGGCATTATGCCCAGTACA |
| | | | | | | TGACCTTATGGGACTTTCCTACTTGGCAGTACATCTACGTATTAGT |
| | | | | | | CATCGCTATTACCATGCTGATGCGGTTTTGGCAGTACATCAATGGG |
| | | | | | | CGTGGATAGCGGTTTGACTCACGGGGATTTCCAAGTCTCCACCCCA |
| | | | | | | TTGACGTCAATGGGAGTTTGTTTTGGCACCAAAATCAACGGGACTT |
| | | | | | | TCCAAAATGTCGTAACAACTCCGCCCCATTGACGCAAATGGGCGG |
| | | | | | | TAGGCGTGTACGGTGGGAGGTCTATATAAGCAGAGCTGGTTTAGT |
| | | | | | | GAACCGTCAGATCGCCTGGAGACGCCATCCACGCTGTTTTGACCTC |
| | | | | | | CATAGAAGACACCGGGACCGATCCAGCCTCCCCTCGAAGCTTACA |
| | | | | | | TGTGGTACCGAGCTCGGATCCTGAGAACTTCAGGGTGAGTCTATG |
| | | | | | | GGACCCTTGATGTTTTCTTTCCCCTTCTTTTCTATGGTTAAGTTCAT |
| | | | | | | GTCATAGGAAGGGGAGAAGTAACAGGGTACACATATTGACCAAAT |
| | | | | | | CAGGGTAATTTTGCATTTGTAATTTTAAAAAATGCTTTCTTCTTTTA |
| | | | | | | ATATACTTTTTTGTTTATCTTATTTCTAATACTTTCCCTAATCTCTTT |
| | | | | | | CTTTCAGGGCAATAATGATACAATGTATCATGCCTCTTTGCACCAT |
| | | | | | | TCTAAAGAATAACAGTGATAATTTCTGGGTTAAGGCAATAGCAAT |
| | | | | | | ATTTCTGCATATAAATATTTCTGCATATAAATTGTAACTGATGTAA |
| | | | | | | GAGGTTTCATATTGCTAATAGCAGCTACAATCCAGCTACCATTCTG |
| | | | | | | CTTTTATTTTATGGTTGGGATAAGGCTGGATTATTCTGAGTCCAAG |
| | | | | | | CTAGGCCCTTTTGCTAATCATGTTCATACCTCTTATCTTCCTCCCAC |
| | | | | | | AGCTCCTGGGCAACGTGCTGGTCTGTGTGCTGGCCCATCACTTTGG |
| | | | | | | CAAAGGCGGCCGCTGCCAAGCTTCCGAGCTCTCGAATTCAAAGGA |
| | | | | | | GGTACCGCGATATCTACCTCGAG |
| Promoters | 171 | pU6 | nucleotide | artificial | | CAACCTACATCCTCAATCCCAATTAAGGTCGGGCAGGAAGAGGGC |
| | | promoter | | sequence | | CTATTTCCCATGATTCCTTCATATTTGCATATACGATACAAGGCTGT |
| | | | | | | TAGAGAGATAATTAGAATTAATTTGACTGTAAACACAAAGATATT |
| | | | | | | AGTACAAAATACGTGACGTAGAAAGTAATAATTTCTTGGGTAGTTT |
| | | | | | | GCAGTTTTAAAATTATGTTTTAAAATGGACTATCATATGCTTACCG |
| | | | | | | TAACTTGAAAGTATTTCGATTTCTTGGCTTTATATATCTTGTGGAAA |
| | | | | | | GGACGAAACACC |
| MG64 | 188 | MG64-1 | nucleotide | artificial | | GAAATAATTTTGTTTAACTTTAAGAAGGAGATATACATATGGCTAG |
| expression | | Cas12k, | | sequence | | CGCATGGAGTCATCCTCAATTCGAGAAAGGTGGAGGTTCTGGCGG |
| constructs | | TnsC, | | | | TGGATCGGGAGGTTCAGCGTGGAGCCACCCGCAGTTCGAAAAAGG |
| | | TniQ, | | | | TGGAGGTTCTCTTGAAGTCCTCTTTCAGGGACCCATGTCCCAGATC |
| | | S15 | | | | ACTATTCAATGCCGACTTGTGGCCAAGGAACCTATTCGTCATACGC |
| | | expression | | | | TTTGGCAACTGATGGCCGACCTTAACACTCCCTTCATAAATGAGTT |
| | | construct | | | | GTTACAGAAAGTAGCACAACACCCAGATTTCGAGAAGTGGAAACA |
| | | | | | | ACGTGGGCGCCTCAAGGTAAAGGTAATTGAACAGTTGGGAAACGA |
| | | | | | | GCTGAAGAAGGATCCTAGATTCTTAGGTCAACCTGCCCGTTTCTAC |
| | | | | | | ACATCGGGCATCAACTTGGTCAAGTACATCTTCAAGTCCTGGCTGA |
| | | | | | | AATTACAACAACGTCTTCAGCAGAAGCTCGACCGAAAGCGGCGCT |
| | | | | | | GGTTGGAAGTCCTCAAGTCAGACGACCAGCTCATCAAGGACGGAC |
| | | | | | | AGACTGACCTGGAGACTATACGCCAAAAGGCCACGGAGATCCTGC |
| | | | | | | AAAGTTATGAGGGAACTGAACAGCTTTTCAACACACTGTTCCAAG |
| | | | | | | CATACAACTCCGAGGAAGACATCCTGACGCGGACAGCACTTAACT |
| | | | | | | ATCTGTTAAAGAACAGATGCAAATTGCCTCAAAAGCCCGAAGATG |
| | | | | | | CTAAGAAGTTCGCCAAGCGCCGCAGACAAGTCGAGATCGCCATAA |
| | | | | | | AGAGATTGCAGGAGCAAATAAAGGCTAGATTACCGCAGGGGCGTG |
| | | | | | | ACGTGACAAACGAGAACTGGCTGGAGACGCTGAACCTCGCATGCT |
| | | | | | | ACACTGATCCTGAAAACATCGAAGAAGCTCGTTCGTGGCAGGACA |
| | | | | | | AGTTATTAACCAAAAGCAGTTCAATCCCATTCCCTATAAATTACGA |
| | | | | | | GACGAATGAGGATCTGATATGGAGCAAGAATGAGAAGGGTCACTT |
| | | | | | | GTGTGTACAGTTTAATGGGATCTCAGACCTGAAGTTTAAGATATAT |
| | | | | | | TGTGACAAAAGACAGCTCAAGTGGTTTCAACGCTTCTACGAAGAC |
| | | | | | | CAACAAATTAAGAAGAGCAATAATAACCAGCATAGTTCGGCTCTG |
| | | | | | | TTCACGCTGCGCTCCGGACGGATACTGTGGCAGGAAGATAAGGGC |
| | | | | | | AAGGGACAGCTCTGGGATATACATAGACTGACATTACAATGTACG |
| | | | | | | CTTGACACACGGACATGGACACAAGAGGGAACGGAGCAGGTAAA |
| | | | | | | AGAAGAAAAGGCCGACGAAATTGCCGGTATACTTACGCGGATGAA |
| | | | | | | CGAGAAGGGCGACCTGACGAAGAACCAACAAGCCTTCATACAAA |
| | | | | | | GAAAGCAGTCTACTCTGGACAAGTTGGAAAATCCATTCCCTCGCCC |
| | | | | | | TTCGCGACCGGTATACCGTGGTCAGAGCAACATCTTACTTGGTGTT |
| | | | | | | TCGATGGAATTGAAGAAACCCGCAACTATTGCGGTTATCGATGGT |
| | | | | | | ATGACCCGTAAAGTGCTGACATATCGTAACATAAAGCAGTTATTG |
| | | | | | | GGTAAGAATTACCCTCTCTTGAATCGCCAACGTCGGCAGAAACAA |
| | | | | | | CTGCAAAGCCACCAGCGCAATGTGGCCCAACGAAAAGAGGCTTTC |
| | | | | | | AACCAATTCGGAGACAGCGAACTGGGAGAGTATATAGACCGTCTG |
| | | | | | | TTAGCCAAAGCGATCATAGCAATCGCCAAACAATATCAAGCTCGT |
| | | | | | | TCTATTGTGGTGCCCCACCTGAAAGACATTAGAGAGGCCATCCAA |
| | | | | | | AGCGAAATACAGGCACTGGCTGAGGCCAAGATACCTAACTGTATT |
| | | | | | | GAGGCACAAGCCGAATACGCCAAGAAGTACCGTATTCAAGTTCAT |
| | | | | | | CAATGGTCCTACGGTCGCCTGATCGACAACATCCAAGCCCAAGCA |
| | | | | | | AGCAAGCTCGGTATTGTTATCGAAGAGTCACAACAGCCGCTTCAG |
| | | | | | | GGTACCCCGCTCCAAAAGGCTGCAGAGCTGGCGTTCAAGGCTTAT |
| | | | | | | CAAAGTCGGCTTTCTGGGAGCGGTTCCCCCAAGAAGAAACGCAAG |
| | | | | | | GTAGACGGTTCTCCTAAGAAGAAACGCAAGGTTGACAGCGGATGA |
| | | | | | | ATGACCATTCAAGAAGCACAAGCGGTTGCCAAACAGTTAGGGGAT |
| | | | | | | ATTCAATTAACCTCAGAGAAGTTACAGGCAGAAATTCAGCGATTA |
| | | | | | | AACCGAAAAACCGTTGTTACCCTTTCCCATGTAGAAGCATTGCATA |
| | | | | | | ATTGGTTAGAAGGAAAGCGTCAGGCTAAACAATCTTGTCGGGTGG |
| | | | | | | TGGGGGAGTCACGCACCGGAAAAACCATTGCTTGTAATGCTTATC |
| | | | | | | GTTTGAGACATAAACCCATTCAAACCCCTGGCAAACCGCCTATCGT |
| | | | | | | TCCAGTGGTGTATATTCAAGTAACTCAAGAATGTGGGGCTAAAGA |
| | | | | | | TTTATTTGGGGCAATTATTGAGCATTTAAAGTATCAAATGACCAAA |
| | | | | | | GGAACTGTTGCAGAAATCCGTCAACGCACCTTTAAAGTGTTGCAA |
| | | | | | | CGGTGTGGCGTAGAAATGCTGATTATTGATGAAGCGGATAGGTTG |
| | | | | | | AAACCGAAGACGTTTGCAGAAGTTAGAGATATTTTTGATAAGTTA |
| | | | | | | AATATTGCAGTGGTATTAGTGGGAACGGATCGCTTAGATGCGGTG |
| | | | | | | ATTAAGCGAGATGAGCAAGTTTATAACCGTTTTCGGGCTTGTCATC |
| | | | | | | GCTTTGGTAAGTTAGCAGGGGATGAGTTTAGTCAAACGGTGAATA |
| | | | | | | TTTGGGAACGTCAAGTGTTGAAGTTGCCTGTAGCGTCTAATTTAAG |
| | | | | | | CAGTAAGCGAATGTTAAAGATTTTGGGACAAGCTACGGGGGGTTA |
| | | | | | | TCTTGGCTTATTGGATATGATTTTACGAGAATCTGCTATTAGAGCG |
| | | | | | | TTGAAAAAAGGGTTACAGAAGATTGATTTAGACACTTTGAAGGAA |
| | | | | | | GTCACGGAGGAGTACCGATAATGGAAAGTCGAGAGATTCAACCTT |
| | | | | | | GGTGGTTCCTCGTTGAGCCCTTGGCTGGTGAGTCTATTTCTCACTTC |
| | | | | | | TTGGGTCGATTCAGACGCGAGAACGAACTTACCGTTACCATGATG |
| | | | | | | GGTAAGATTACCGGACTGGGTGGAACGATCACACGTTGGGAAAAG |
| | | | | | | TTTCGATTCATACCGATCCCAACGGAAGAAGAACTTACGGCACTGT |
| | | | | | | CAGAAGTAGTTCAAGTTGAGGTTGAGCGCCTGTGGCAGATGTTCC |
| | | | | | | CACCCAAGGGTGTAGGTATGAAACACCAGCCTATCCGACTGTGTG |
| | | | | | | GAGCGTGCTACGAAGAGGAGCGCTGCCACAAAATAGAGTGGCAA |
| | | | | | | CTGAAAACTACCCAATTCTGTTCCCAACACGGCCTCACTTTACTGA |
| | | | | | | GCGAATGCCCGAACTGCGGCGCTCGCTTCCAATTCCCAGCACTGTG |
| | | | | | | GGTAAACGGCTGGTGCCACAGATGCTTCTTGACGTTCGGAGAAAT |
| | | | | | | GGTAGAGGGACAAAGTAACAAGAAAAAATATTTATGAATGTCACT |
| | | | | | | TAGCACAGAGGCGACTGCTAAAATCGTTAGCGAATTCGGACGAGA |
| | | | | | | TGCTAATGACACTGGTTCCACAGAGGTTCAAGTTGCACTTTTGACA |
| | | | | | | GCCCAGATCAATCATCTGCAAGGTCACTTTGCGGAGCACAAGAAG |
| | | | | | | GACCACCACAGTCGGCGCGGGCTGCTGCGCATGGTTTCTCAACGC |
| | | | | | | CGCAAGCTGCTGGACTACCTCAAGCGTAAGGACGTCGCGCGTTAC |
| | | | | | | ACTCAATTGATCGAGCGGCTTGGATTGCGACGTTAA |
| MG190 | 189 | MG190- | protein | unknown | uncultivated | MALTQQRKQEIISGFQVHETDTGSADVQIAMLTDRINRLSKHLQANK |
| ribosomal | | 178 | | | organism | KDYSSRRGLLKMIGQRKRLLAYVQENSREKYQQLISRLGIRG |
| proteins | | ribosomal | | | | |
| | | protein | | | | |
| | | S15 | | | | |
| MG190 | 190 | MG190- | protein | unknown | uncultivated | MSLTASEKQELMSSYQVHETDTGSPDLQVALLTKRISQLTGHLQKNP |
| ribosomal | | 179 | | | organism | KDHASRRGLLKMIGRRRRLLAYINSKDTNRYKALIKSLGIRR |
| proteins | | ribosomal | | | | |
| | | protein | | | | |
| | | S15 | | | | |
| MG190 | 191 | MG190- | protein | unknown | uncultivated | MALIQERKQELISEYQVHETDTGSAEVQVAMLTERINKLSQHLRENK |
| ribosomal | | 180 | | | organism | KDYSSRRGLLKMIGRRKRLLSYISKKDVERYRELIGRLGIRR |
| proteins | | ribosomal | | | | |
| | | protein | | | | |
| | | S15 | | | | |
| MG190 | 192 | MG190- | protein | unknown | uncultivated | MSITQERKQELISEYQVHGTDTGSSDVQVAILSDRINSLTQHLKVNKK |
| ribosomal | | 181 | | | organism | DHASRLGLLKLIGRRRRLLTYIQQHDYEHYQQLIRRLGIRR |
| proteins | | ribosomal | | | | |
| | | protein | | | | |
| | | S15 | | | | |
| MG190 | 193 | MG190- | protein | unknown | uncultivated | MSLTANEKQSLISEYQVHETDTGSADLQVAILTKRITQLTDHLKQNPK |
| ribosomal | | 182 | | | organism | DHASRRGLLKMIGRRRRLLAYINGKDTNRYQALIKRLGIRR |
| proteins | | ribosomal | | | | |
| | | protein | | | | |
| | | S15 | | | | |
| MG190 | 194 | MG190- | protein | unknown | uncultivated | MALLQERKQELISEYQVHETDTGSAEVQVAMLTERINKLSQHLQGNK |
| ribosomal | | 183 | | | organism | KDYSSRRGLLKMIGRRKRLLSYIAKKDVNQYRALIGRLGIRR |
| proteins | | ribosomal | | | | |
| | | protein | | | | |
| | | S15 | | | | |
| MG190 | 195 | MG190- | protein | unknown | uncultivated | MTLLQERKQELISEYQIHETDTGSVDLQIAMLTERINRLSAHLQKNKK |
| ribosomal | | 184 | | | organism | DYSSRRGLLKMIGQRKRLMAYLLKQDTERYRNLIQKLGIRG |
| proteins | | ribosomal | | | | |
| | | protein | | | | |
| | | S15 | | | | |
| MG190 | 196 | MG190- | protein | unknown | uncultivated | MALLQERKQELISEYQVHETYTGSAEVHVAMLTERINKLSQHLQSNK |
| ribosomal | | 185 | | | organism | KDYSSRRGLLKMIGRRKRLLSYIAKKDVNQYRELIGRLGIRR |
| proteins | | ribosomal | | | | |
| | | protein | | | | |
| | | S15 | | | | |
| MG190 | 197 | MG190- | protein | unknown | uncultivated | MTLLQERKQEIMSEYQIHETDTGSVDLQIAMLTERINRLSAHLQKNKK |
| ribosomal | | 186 | | | organism | DYSSRRGLLKMIGQRKRLMAYLLKQDTDRYRDLIKKLGIRG |
| proteins | | ribosomal | | | | |
| | | protein | | | | |
| | | S15 | | | | |
| MG190 | 198 | MG190- | protein | unknown | uncultivated | MALLQERKQELISEYQVHETDTGSAEVQVAMLTERINKLSQHLQSNK |
| ribosomal | | 187 | | | organism | KDYSSRRGLLKMIGRRKRLLSYIAKKDVNQYRELIGRLGIRR |
| proteins | | ribosomal | | | | |
| | | protein | | | | |
| | | S15 | | | | |
| MG190 | 199 | MG190- | protein | unknown | uncultivated | MALTQEDKQQIMAEHQAHETDTGSSDVQVAMLTERINRLSAHLKAN |
| ribosomal | | 188 | | | organism | KKDHSSRRGLLQMIGRRKRLLAYIYKQDQQRYRALITRLGIRG |
| proteins | | ribosomal | | | | |
| | | protein | | | | |
| | | S15 | | | | |
| MG64 | 200 | MG64- | protein | unknown | uncultivated | MSQKTIRCRLIASQKSRKAIWKLMAERNTPLVNEVLRQLPEHPDFAK |
| effectors | | 128 | | | organism | WQQKGNLPDVAVKRIIDALKSDPHFSDQPFWYYTSAQKQVTYTFKS |
| | | effector | | | | WLSIQRRKQWRLQGKRWWLEILLPDAELAKLVKCSVEKLRTEAAKIL |
| | | | | | | AKVGDVDPFKHLLEQYRHEQKLLRKCAIAFLLKRNTEIDREEDLEQL |
| | | | | | | KQRSRRVELQIRRLEIQLQASLPKGRDLTGERQAAALAQSVLASPDDD |
| | | | | | | ESYELWRNTVTREPAQFPFPVICETSEWLKWQRDHNGRISVGFSALSE |
| | | | | | | HVFKIYCDKPHQHWFDRFFEDQETKRTGGKQHSAGLFTLRSAKLTW |
| | | | | | | VPSNKHANASEPWNCYYLNLSCTVDTRLWTQEGTQIVIQEKAAEKA |
| | | | | | | GKLESMRRKENLSKTQQGYIKRLEATLEKLQTPYPRPSRPLYSGKTNI |
| | | | | | | LVGVSMGLDKPATVAVVDALAGEVLTYRSVKQLLGNDYRLLKRAQ |
| | | | | | | TEKTRIHKNHRRGGRRVSEESNIAQQVDRLLAKSIIDIAREYEASSIVV |
| | | | | | | PDLANIREIVETEVKARAQDKVPDFVEGQKQYAKAYRTQVHQWSYR |
| | | | | | | RLQEAVRTKAEQSGITIEVVRQGLSGTQHEKAKALALQGYEKRIREH |
| | | | | | | VEMA |
| MG64 | 201 | MG64- | nucleotide | artificial | | GACGCAATGAGCTTTTAAGGCTCATGAGGATTGAAAC |
| effectors | | 128 | | sequence | | |
| crRNA | | crRNA | | | | |
| sequence | | sequence | | | | |
| MG64 | 202 | MG64- | nucleotide | artificial | | GTATATTTGTACAAAAGCGCCGCAGATCATGCGCAAGCCTCTGTTC |
| effectors | | 128 | | sequence | | TGTGAAAAATGAGGGTTTGTTTGACTGTGTGATAGCGGTCTTACTT |
| putative | | predicted | | | | TCTGACCCTGGTAGCTGACCACTCCGATGCTGCTGTTGAAAGTGAA |
| tracrRNA | | tracrRNA | | | | CTTGTTAGGACTTGGGCACCATAAAACAGGAGAGGGGCGCACCCA |
| | | sequence | | | | GCAAGAGAGAACGGACTTACTGTAGTGTTGGCTGCCGAATCAACT |
| | | | | | | CCGATCAAGGAGTAATCCATGCACCCATACCCATATA |

What is claimed is:

1. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide polynucleotide configured to hybridize to the target nucleic acid site;
b) a Tn7 type transposase complex configured to bind the Cas effector complex and comprising a TnsB, TnsC, and TniQ component;
c) a double-stranded nucleic acid configured to interact with the Tn7 type transposase complex and comprising the cargo nucleotide sequence; and
d) a functional domain comprising a DNA Binding domain (DBD) or a chromatin modulating domain (CMD).

2. The system of claim 1, wherein the Cas effector complex binds non-covalently to the Tn7 type transposase complex.

3. The system of claim 1, wherein the Cas effector complex is covalently linked to the Tn7 type transposase complex.

4. The system of claim 1, wherein the Cas effector complex is fused to the Tn7 type transposase complex.

5. The system of any one of claims 1-4, wherein the cargo nucleotide sequence is flanked by a left-hand transposase recognition sequence and a right-hand transposase recognition sequence recognized by the Tn7 type transposase complex.

6. The system of claim 5, wherein the left-hand recombinase sequence comprises a sequence having at least 80% identity to any one of SEQ ID NOs: 9, 11, 36-38, 76, and 78.

7. The system of any one of claim 5, wherein the right-hand recombinase sequence comprises a sequence having at least 80% identity to any one of SEQ ID NOs: 8, 10, 39-44, 77, 79, and 93.

8. The system of claim 1-7, the target nucleic acid comprises a PAM sequence compatible with the Cas effector complex.

9. The system of claim 8, wherein the PAM sequence comprises SEQ ID NO: 31.

10. The system of any one of claims 8-9, wherein the PAM sequence is located about 50 to about 70 base pairs from the target nucleic acid site.

11. The system of claim 10, wherein the PAM sequence is located 3′ of the target nucleic acid site.

12. The system of claim 10, wherein the PAM sequence is located 5′ of the target nucleic acid site.

13. The system of any one of claims 1-12, wherein the class 2, type V Cas effector is a Cas12k effector.

14. The system of any one of claims 1-12, wherein the class 2, type V Cas effector comprises a polypeptide comprising a sequence having at least 80% identity to any one of SEQ ID NOs: 1, 12, 16, 20-30, 64, 80-85, and 200.

15. The system of any one of claims 1-12, wherein the class 2, type V Cas effector comprises a polypeptide comprising a sequence having at least 90% identity to any one of SEQ ID NOs: 1, 12, 16, 20-30, 64, 80-85, and 200.

16. The system of any one of claims 1-12, wherein the class 2, type V Cas effector comprises a polypeptide comprising a sequence of any one of SEQ ID NOs: 1, 12, 16, 20-30, 64, 80-85, and 200.

17. The system of any one of claims 1-16, wherein the TnsB component comprises a polypeptide having a sequence having at least 80% identity to any one of SEQ ID NOs: 2, 13, 17, and 65.

18. The system of any one of claims 1-16, wherein the TnsB component comprises a polypeptide having a sequence having at least 90% identity to any one of SEQ ID NOs: 2, 13, 17, and 65.

19. The system of any one of claims 1-16, wherein the TnsB component comprises a polypeptide having a sequence of any one of SEQ ID NOs: 2, 13, 17, and 65.

20. The system of any one of claims 1-19, wherein the Tn7 type transposase complex comprises at least a first polypeptide and a second polypeptide each independently comprising a sequence having at least 80% identity to any one of SEQ ID NOs: 3-4, 14-15, 18-19, and 66-67.

21. The system of any one of claims 1-19, wherein the Tn7 type transposase complex comprises at least a first polypeptide and a second polypeptide each independently comprising a sequence having at least 90% identity to any one of SEQ ID NOs: 3-4, 14-15, 18-19, and 66-67.

22. The system of any one of claims 1-19, wherein the Tn7 type transposase complex comprises at least a first polypeptide and a second polypeptide each independently comprising a sequence of any one of SEQ ID NOs: 3-4, 14-15, 18-19, and 66-67.

23. The system of any one of claims 1-22, wherein the engineered guide polynucleotide comprises a sequence comprising at least about 46-80 consecutive nucleotides having at least 80% identity to any one of SEQ ID NOs: 5-6, 32-33, 94-95, 104-105, and 202.

24. The system of any one of claims 1-22, wherein the engineered guide polynucleotide comprises a sequence having at least 80% sequence identity to any one of SEQ ID NOs: 106, 107, 108, 5, 45-63, 68-75, 96-103, and 165.

25. The system of any one of claims 1-24, wherein the functional domain is derived from a Human histone 1 central globular domain, HMGN1, cbx5, or Sacecharolobus so/ft eicus sso7d.

26. The system of any one of claims 1-25, wherein the functional domain comprises a sequence having at least 80% sequence identity to any one of SEQ ID NOs: 109-112.

27. The system of any one of claims 1-26, wherein the class 2, type V Cas effector is fused to the functional domain to form a fusion protein.

28. The system of claim 27, wherein the fusion protein comprises a sequence having at least 80% identity to any one of SEQ ID NOs: 113-116.

29. The system of any one of claims 1-26, wherein the Tn7 transposase complex comprises a TniQ protein.

30. The system of claim 29, wherein the TniQ protein is fused to the functional domain to form a fusion protein.

31. The system of claim 30, wherein the TniQ protein comprises a sequence having at least 80% sequence identity to a TniQ domain of any one of SEQ ID NOs: 117-120.

32. The system of any one of claims 1-31, wherein the small prokaryotic ribosomal protein subunit S15 comprises a sequence having at least 80% sequence identity to any one of SEQ ID NOs: 167-169.

33. The system of any one of claims 1-31, wherein the small prokaryotic ribosomal protein subunit S15 is encoded by a sequence having at least 80% sequence identity to any one of SEQ ID NOs: 161-163.

34. The system of any one of claims 1-33, wherein the class 2, type V Cas effector and the Tn7 type transposase complex are encoded by polynucleotide sequences comprising fewer than about 10 kilobases.

35. A system for transposing a cargo nucleotide sequence into a target nucleic acid site in a target nucleic acid comprising:
a) a Cas effector complex comprising a class 2, type V Cas effector and an engineered guide polynucleotide configured to hybridize to the target nucleic acid site, wherein the Cas effector complex comprises a polypeptide comprising a sequence having at least 80% sequence identity to any one of SEQ ID NOs: 1, 12, 16, 20-30, 64, 80-85, and 200;
b) a Tn7 type transposase complex configured to bind the Cas effector complex and comprising a TnsB, TnsC, and TniQ component, the TnsB, TnsC, or TniQ component comprising a sequence having at least 80% sequence identity to any one of SEQ ID NOs: 2-4, 13-15, 17-19, and 65-67;
c) a double-stranded nucleic acid configured to interact with the Tn7 type transposase complex and comprising the cargo nucleotide sequence; and
d) a functional domain comprising a DNA Binding domain (DBD) or a chromatin modulating domain (CMD).

36. The system of claim 35, wherein the Cas effector complex binds non-covalently to the Tn7 type transposase complex.

37. The system of claim 35, wherein the Cas effector complex is covalently linked to the Tn7 type transposase complex.

38. The system of claim 35, wherein the Cas effector complex is fused to the Tn7 type transposase complex.

39. The system of any one of claims 35-38, wherein the cargo nucleotide sequence is flanked by a left-hand transposase recognition sequence and a right-hand transposase recognition sequence recognized by the Tn7 type transposase complex.

40. The system of claim 39, wherein the left-hand recombinase sequence comprises a sequence having at least 80% identity to any one of SEQ ID NOs: 9, 11, 36-38, 76, and 78.

41. The system of any one of claim 39, wherein the right-hand recombinase sequence comprises a sequence having at least 80% identity to any one of SEQ ID NOs: 8, 10, 39-44, 77, 79, and 93.

42. The system of claim 35-41, the target nucleic acid comprises a PAM sequence compatible with the Cas effector complex.

43. The system of claim 42, wherein the PAM sequence comprises SEQ ID NO: 31.

44. The system of any one of claims 42-43, wherein the PAM sequence is located about 50 to about 70 base pairs from the target nucleic acid site.

45. The system of claim 44, wherein the PAM sequence is located 3′ of the target nucleic acid site.

46. The system of claim 44, wherein the PAM sequence is located 5′ of the target nucleic acid site.

47. The system of any one of claims 35-46, wherein the class 2, type V Cas effector is a Cas12k effector.

48. The system of any one of claims 35-46, wherein the class 2, type V Cas effector comprises a polypeptide comprising a sequence having at least 90% identity to any one of SEQ ID NOs: 1, 12, 16, 20-30, 64, 80-85, and 200.

49. The system of any one of claims 35-46, wherein the class 2, type V Cas effector comprises a polypeptide comprising a sequence of any one of SEQ ID Nos: 1, 12, 16, 20-30, 64, 80-85, and 200.

50. The system of any one of claims 35-46, wherein the TnsB, TnsC, or TniQ component comprises a sequence having at least 90% sequence identity to any one of SEQ ID NOs: 2-4, 13-15, 17-19, and 65-67.

51. The system of any one of claims 35-46, wherein the TnsB, TnsC, or TniQ component comprises a sequence of any one of SEQ ID NOs: 2-4, 13-15, 17-19, and 65-67.

52. The system of any one of claims 35-51, wherein the engineered guide polynucleotide comprises a sequence comprising at least about 46-80 consecutive nucleotides having at least 80% identity to any one of SEQ ID NOs: 5-6, 32-33, 94-95, 104-105, and 202.

53. The system of any one of claims 35-51, wherein the engineered guide polynucleotide comprises a sequence having at least 80% sequence identity to any one of SEQ ID NOs: 106, 107, 108, 5, 45-63, 68-75, 96-103, and 165.

54. The system of any one of claims 35-53, wherein the functional domain is derived from a Human histone 1 central globular domain, HMGN1, cbx5, or Saccharolobus solfataricus sso7d.

55. The system of any one of claims 35-54, wherein the functional domain comprises a sequence having at least 80% sequence identity to any one of SEQ ID NOs: 109-112.

56. The system of any one of claims 35-55, wherein the class 2, type V Cas effector is fused to the functional domain to form a fusion protein.

57. The system of claim 56, wherein the fusion protein comprises a sequence having at least 80% identity to any one of SEQ ID NOs: 113-116.

58. The system of any one of claims 35-57, wherein the Tn7 transposase complex comprises a TniQ protein.

59. The system of claim 58, wherein the TniQ protein is fused to the functional domain to form a fusion protein.

60. The system of claim 59, wherein the TniQ protein comprises a sequence having at least 80% sequence identity to a TniQ domain of any one of SEQ ID NOs: 117-120.

61. The system of any one of claims 35-60, wherein the Cas effector complex further comprises a small prokaryotic ribosomal protein subunit S15.

62. The system of claim 61, wherein the small prokaryotic ribosomal protein subunit S15 comprises a sequence having at least 80% sequence identity to any one of SEQ ID NOs: 167-169.

63. The system of claim 61, wherein the small prokaryotic ribosomal protein subunit S15 is encoded by a sequence having at least 80% sequence identity to any one of SEQ ID NOs: 161-163.

64. The system of any one of claims 35-63, wherein the class 2, type V Cas effector and the Tn7 type transposase complex are encoded by polynucleotide sequences comprising fewer than about 10 kilobases.

65. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a sequence having at least 80% sequence identity to any one of SEQ ID NOs: 1, 81, 82, 83, and 85; and
ii) an engineered guide polynucleotide comprising having at least 80% identity to any one of SEQ ID NOs: 5, 6, 45-63, 68-75, and 96-103;
b) a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 2-4;
c) a double-stranded nucleic acid configured to interact with the Tn7 type transposase complex and comprising in 5′ to 3′ order:
i) a left-hand recombinase sequence comprising a sequence having at least 80% sequence identity to any one of SEQ ID NOs: 9, 11, 36, 37, and 38;
ii) the cargo nucleotide sequence; and
ii) a right-hand recombinase sequence comprising a sequence having at least 80% identity to any one of SEQ ID NOs: 8, 39-44, and 93; and
d) a functional domain comprising a DNA Binding domain (DBD) or a chromatin modulating domain (CMD).

66. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a sequence having at least 80% sequence identity to SEQ ID NO: 12; and
ii) an engineered guide polynucleotide comprising having at least 80% identity to any one of SEQ ID NOs: 32, 102, 104, and 107;
b) a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 13-15;
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 76;
ii) the cargo nucleotide sequence; and
iii) a right-hand recombinase sequence comprising a sequence having at least 80% identity to SEQ ID NO: 77; and
d) a functional domain comprising a DNA Binding domain (DBD) or a chromatin modulating domain (CMD).

67. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a sequence having at least 80% sequence identity to SEQ ID NO: 16; and
ii) an engineered guide polynucleotide comprising having at least 80% identity to any one of SEQ ID NOs: 33, 103, 105, and 108;
b) a Tn7 type transposase complex configured to bind the Cas effector complex and comprising a TnsB, TnsC, and TniQ component, the TnsB, TnsC, or TniQ component comprising a sequence having at least 80% identity to any one of SEQ ID NOs: 17-19;
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 78;
ii) the cargo nucleotide sequence; and
iii) a right-hand recombinase sequence comprising a sequence having at least 80% identity to SEQ ID NO: 79; and
d) a functional domain comprising a DNA Binding domain (DBD) or a chromatin modulating domain (CMD).

68. The system of any one of claims 65-67, wherein the target nucleic acid comprises a PAM sequence compatible with the Cas effector complex.

69. The system of claim 68, wherein the PAM sequence comprises SEQ ID NO: 31.

70. The system of any one of claims 68-69, wherein the PAM sequence is located about 50 to about 70 base pairs from the target nucleic acid site.

71. The system of claim 70, wherein the PAM sequence is located 3′ of the target nucleic acid site.

72. The system of claim 70, wherein the PAM sequence is located 5′ of the target nucleic acid site.

73. The system of any one of claims 65-72, wherein the functional domain is derived from a Human histone 1 central globular domain, HMGN1, cbx5, or Saccharolobus solfataricus sso7d.

74. The system of any one of claims 65-73, wherein the functional domain comprises a sequence having at least 80% sequence identity to any one of SEQ ID NOs: 109-112.

75. The system of any one of claims 65-74, wherein the class 2, type V Cas effector is fused to the functional domain to form a fusion protein.

76. The system of claim 75, wherein the fusion protein comprises a sequence having at least 80% identity to any one of SEQ ID NOs: 113-116.

77. The system of claim 65, wherein the Tn7 transposase complex comprises a TniQ protein.

78. The system of any one of claims 65-77, wherein the TniQ protein is fused to the functional domain to form a fusion protein.

79. The system of claim 78, wherein the TniQ protein comprises a sequence having at least 80% sequence identity to a TniQ domain of any one of SEQ ID NOs: 117-120.

80. The system of any one of claims 65-79, wherein the Cas effector complex further comprises a small prokaryotic ribosomal protein subunit S15.

81. The system of claim 80, wherein the small prokaryotic ribosomal protein subunit S15 comprises a sequence having at least 80% sequence identity to any one of SEQ ID NOs: 167-169.

82. The system of claim 80, wherein the small prokaryotic ribosomal protein subunit S15 is encoded by a sequence having at least 80% sequence identity to any one of SEQ ID NOs: 161-163.

83. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide, the engineered guide polynucleotide capable of hybridizing to the target nucleic acid;
b) a Tn7 type transposase complex operably linked to the Cas effector complex and comprising a TnsB, TnsC, and TniQ component;
c) a double-stranded nucleic acid comprising in 5′ to 3′ order:
i) a left-hand recombinase recognition sequence;
ii) the cargo nucleotide sequence; and
iii) a right-hand recombinase recognition sequence, the left-hand recombinase recognition sequence and the right-hand recombinase recognition sequence capable of being recognized by the Tn7 type transposase complex; and
d) a functional domain comprising a DNA Binding domain (DBD) or a chromatin modulating domain (CMD).

84. An engineered nuclease system comprising:
a) an endonuclease comprising a RuvC domain, the endonuclease being derived from an uncultivated microorganism and is a Class 2, type V-K Cas effector comprising at least 80% identity to any one of SEQ ID NOs: 1, 12, 16, 20-30, 64, 80-85, and 200; and
b) an engineered guide RNA configured to form a complex with the endonuclease and comprising a spacer sequence configured to hybridize to a target nucleic acid sequence.

85. The engineered nuclease system of claim 84, wherein the engineered guide polynucleotide comprises a sequence comprising at least about 46-80 consecutive nucleotides having at least 80% identity to any one of SEQ ID NOs: 5-6, 32-33, 94-95, 104-105, and 202.

86. The engineered nuclease system of claim 84 or 85, wherein the engineered guide polynucleotide comprises a sequence having at least 80% identity to any one of SEQ ID NOs: 106, 107, 108, 5, 45-63, 68-75, 96-103, and 165.

87. A method for transposing a cargo nucleotide sequence into a target nucleic acid site comprising introducing the system of any one of claims 1-86 to a cell.

88. A cell comprising the system of any one of claims 1-86.

89. The cell of claim 88, wherein the cell is a eukaryotic cell.

90. The cell of claim 88, wherein the cell is a mammalian cell.

91. The cell of claim 88, wherein the cell is an immortalized cell.

92. The cell of claim 88, wherein the cell is an insect cell.

93. The cell of claim 88, wherein the cell is a yeast cell.

94. The cell of claim 88, wherein the cell is a plant cell.

95. The cell of claim 88, wherein the cell is a fungal cell.

96. The cell of claim 88, wherein the cell is a prokaryotic cell.

97. The cell of claim 88, wherein the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof.

98. The cell of claim 88, wherein the cell is an engineered cell.

99. The cell of claim 88, wherein the cell is a stable cell.
